(19) United States
(12) Patent Application Publication
(10) Pub. No.: US 2012/0100140 A1
(43) Pub. Date: Apr. 26, 2012

(54) STABILIZED FC POLYPEPTIDES WITH REDUCED EFFECTOR FUNCTION AND METHODS OF USE

(75) Inventors: Christopher L. Reyes, San Diego, CA (US); Eric Chan, San Diego, CA (US); Frederick R. Taylor, Milton, MA (US); Ellen Garber, Cambridge, MA (US); Brian Robert Miller, San Diego, CA (US); Stephen Demarest, San Diego, CA (US); Scott Glaser, San Diego, CA (US)

(73) Assignee: BIOGEN IDEC MA INC., Cambridge, MA (US)

(21) Appl. No.: 13/145,994

(22) PCT Filed: Jan. 22, 2010
(86) PCT No.: PCT/US10/21853

§ 371 Date: Dec. 5, 2011

(30) Foreign Application Priority Data
Jan. 23, 2009 (US) 61146950

(51) Int. Cl. A61K 39/395; C12N 5/10; C07K 16/00; C12N 1/19; C12N 15/63; C12P 21/06; C12N 1/21

(52) U.S. Cl. 424/134.1; 435/69.6; 435/325; 435/352; 435/354; 435/358; 435/364; 435/365; 435/366; 435/369; 435/372; 435/419; 435/252.3; 435/252.31; 435/252.33; 435/252.35; 435/254.2; 435/254.21; 435/254.23; 435/320.1; 530/387.3; 536/23.4

(57) ABSTRACT

A method of producing Fc-containing polypeptides, such as antibodies, having stabilized Fc regions is provided, together with stabilized Fc polypeptides produced according to these methods as well as methods of using such antibodies as therapeutics.

### RELATED APPLICATION

This patent application claims the benefit of U.S. Provisional Patent Application Ser. No. 61/146,950, entitled “STABILIZED Fc POLYPEPTIDES WITH REDUCED EFFECTOR FUNCTION AND METHODS OF USE”, filed Jan. 23, 2009. The entire contents of the above-referenced provisional patent application are incorporated herein by reference.

### BACKGROUND OF THE INVENTION

The acquired immune response is a mechanism by which the body defends itself against foreign organisms that invade it causing infection or disease. One mechanism is based on the ability of antibodies produced or administered to the host to bind the antigen though its variable region. Once the antigen is bound by the antibody, the antigen is targeted for destruction, often mediated, at least in part, by the constant region or Fc region of the antibody.

There are several effector functions or activities mediated by the Fc region of an antibody. One effector function is the ability to bind complement proteins which can assist in lysing the target antigen, for example, a cellular pathogen, in a process termed complement-dependent cytotoxicity (CDC). Another effector activity of the Fc region is to bind to Fc receptors (e.g., FcγRs) on the surface of immune cells, or so-called effector cells, which have the ability to trigger other immune effects. These immune effects (e.g., antibody-dependent cell cytotoxicity (ADCC) and antibody-dependent cell phagocytosis (ADCP)), act in the removal of pathogens/antigens by, for example, releasing immune activators and regulating antibody production, endocytosis, phagocytosis, and cell killing. In some clinical applications these responses are crucial for the efficacy of the antibody while in other cases they provoke unwanted side effects. One example of an effector-mediated side effect is the release of inflammatory cytokines causing an acute fever reaction. Another example is the long term deletion of antigen-bearing cells.

The effector function of an antibody can be avoided by using antibody fragments lacking the Fc region (e.g., such as a Fab, F(ab′)₂, or single chain Fv (scFv)). However, these fragments have reduced half-lives due to rapid clearance through the kidneys; in the case of Fab and scFv fragments have only one antigen binding site instead of two potentially compromising any advantages due to binding avidity; and can present challenges in manufacturing.

Alternative approaches aim to reduce the effector functions of a full-length antibody while retaining other valuable attributes of the Fc region (e.g., prolonged half-life and heterodimerization). One approach to reduce effector function is generate so-called aglycosylated antibodies by removing sugars that are linked to particular residues in the Fc region. Aglycosylated antibodies can be generated by, for example, deleting or altering the residue the sugar is attached to, removing the sugars enzymatically, producing the antibody in cells cultured in the presence of a glycosylation inhibitor, or by expressing the antibody in cells unable to glycosylate proteins (e.g., bacterial host cells). Another approach is to employ Fc regions from an IgG4 antibody, instead of IgG1. It is well known that IgG4 antibodies are characterized by having lower levels of complement activation and antibody-dependent cellular cytotoxicity than IgG1.

Despite the advantages of these alternative approaches, it is now well established that removal of the oligosaccharides from the Fc region of antibody has significant adverse affects on its conformation and stability. Additionally, IgG4 antibodies have lower stability in general since the CH3 domain of IgG4 lacks comparable stability to the CH3 domain of IgG1. In all cases, loss of or decreased antibody stability can present process development challenges adversely effecting antibody drug development.

Accordingly, a need exists for improved antibodies and other Fc-containing polypeptides with altered or reduced effector function and improved stability and methods of making these molecules.

### SUMMARY OF THE INVENTION

The invention solves the problems of prior art “effector-less” antibodies, indeed of any “effector-less” Fc-containing protein, by providing improved methods for enhancing the stability of an Fc region. For example, the invention provides stability-engineered Fc polypeptides, e.g., stabilized IgG antibodies or other Fc-containing binding molecules, which comprise stabilizing amino acids in the Fc region of the polypeptide. In one embodiment, the invention provides a method for introducing mutations at specific amino acid residue positions in the Fc region of a parental Fc polypeptide which result in the enhanced stability of the Fc region. Preferably, the stabilized Fc polypeptides have an altered or reduced effector function (as compared to a polypeptide which does not comprise the stabilizing amino acid(s)) and exhibits enhanced stability as compared to the parental Fc polypeptide.

Accordingly, the invention has several advantages which include, but are not limited to, the following:
providing stabilized aglycosylated Fc polypeptides comprising stabilized aglycosylated Fc regions, for example, stabilized fusion proteins or aglycosylated IgG antibodies, suitable as therapeutics because of their reduced effector function;
providing stabilized Fc polypeptides comprising Fc regions derived from IgG4 antibodies, for example, stabilized glycosylated or aglycosylated fusion proteins or IgG4 antibodies, suitable as therapeutics because of their reduced effector function;
an efficient method of producing stabilized Fc polypeptides with minimal alterations to the polypeptide (e.g., by introducing changes into an unstabilized parent polypeptide or by expressing a nucleic acid molecule encoding a stabilized Fc polypeptide);
a method of enhancing the stability of an Fc polypeptide while avoiding any increase in immunogenicity and/or effector function;
methods for enhancing the scalability, manufacturing, and/or long-term stability of an Fc polypeptide; and
methods for treating a subject in need of therapy with a stabilized Fc polypeptide of the invention.

In one aspect, the invention pertains to a stabilized polypeptide comprising a chimeric Fc region, wherein said stabilized polypeptide comprises at least one constant domain derived from a human IgG4 antibody and at least one constant domain derived from a human IgG1 antibody.

In one embodiment, the Fc region is a glycosylated Fc region.

In one embodiment, the Fc region is an aglycosylated Fc region.

In one embodiment, the Fc region is an aglycosylated Fc region comprises a glutamine (Q) at position 297 or an alanine (A) at position 299 of the Fc region (EU numbering convention).

In another aspect, the invention pertains to a stabilized polypeptide comprising an aglycosylated Fc region, wherein said stabilized polypeptide comprises one or more stabilizing Fc amino acids at one or more amino acid positions in at least one Fc moiety of said Fc region, wherein said amino acid positions are selected from the group consisting of 297, 299, 307, 309, 399, 409 and 427 (EU Numbering Convention).

In one embodiment, the chimeric Fc region comprises a CH2 domain from an IgG antibody of the IgG4 isotype and a CH3 domain from an IgG antibody of the IgG1 isotype.

In one embodiment, the chimeric Fc region comprises a hinge, CH1 and CH2 domains from an IgG antibody of the IgG4 isotype and a CH3 domain from an IgG antibody of the IgG1 isotype, and wherein the antibody comprises a proline at amino acid position 228, EU numbering.

In another aspect, the invention pertains to a stabilized polypeptide comprising a CH2 moiety from an Fc region of an IgG4 antibody, wherein said stabilized polypeptide comprises one or more stabilizing amino acids at one or more amino acid positions selected from the group consisting of 240F, 262L, 264T, 266F, 297Q, 299A, 299K, 307P, 309K, 309M, 309P, 323F, 399S, and 427F (EU Numbering Convention).

In one embodiment, a stabilized polypeptide comprises a Gln at amino acid position 297.

In one aspect, the invention pertains to a stabilized polypeptide comprising a CH2 moiety from an Fc region of an IgG1 antibody, wherein said stabilized polypeptide comprises one or more stabilizing amino acids at one or more amino acid positions selected from the group consisting of 299K and 297D (EU Numbering Convention).

In one embodiment, a stabilized polypeptide of the invention comprises a Lys at amino acid position 299.

In another embodiment, a stabilized polypeptide of the invention comprises a Lys at amino acid position 299 and an Asp at amino acid position 297.

In one embodiment, the Fc region is an aglycosylated Fc region.

In one embodiment, IgG antibody is a human antibody.

In one embodiment, the melting temperature (Tm) of the stabilized polypeptide is enhanced by at least 1° C. relative to a parental polypeptide lacking the stabilizing amino acid.

In one embodiment, the melting temperature (Tm) of the stabilized Fc polypeptide is enhanced by about 1° C. or more, about 2° C. or more, about 3° C. or more, about 4° C. or more, about 5° C. or more, about 6° C. or more, about 7° C. or more, about 8° C. or more, about 9° C. or more, about 10° C. or more, about 15° C. or more, and about 20° C. or more.

In one embodiment, the melting temperature (Tm) is enhanced at a neutral pH (about 6.5 to about 7.5).

In another embodiment, the melting temperature (Tm) is enhanced at an acidic pH of about 6.5 or less, about 6.0 or less, about 5.5 or less, about 5.0 or less, about 4.5 or less, and about 4.0 or less.

In one embodiment, the stabilized polypeptide is expressed at higher yield relative to a parental polypeptide lacking the stabilizing mutation.

In another embodiment, the stabilized Fc polypeptide is expressed in cell culture at a yield of about 5 mg/L or more, about 10 mg/L or more, about 15 mg/L or more, about 20 mg/L or more.

In one embodiment, the turbidity of the stabilized polypeptide is reduced relative to a parental polypeptide lacking the stabilizing amino acid.

In another embodiment, the turbidity is reduced by a factor selected from the group consisting of about 1-fold or more, about 2-fold or more, about 3-fold or more, about 4-fold or more, about 5-fold or more, about 6-fold or more, about 7-fold or more, about 8-fold or more, about 9-fold or more, about 10-fold or more, about 15-fold or more, about 50-fold or more, and about 100-fold or more.

In another embodiment, said stabilized polypeptide has reduced effector function as compared to a parental Fc polypeptide lacking the stabilizing mutation.

In one embodiment, the reduced effector function is reduced ADCC activity.

In another embodiment, the reduced effector function is reduced binding to an Fc receptor (FcR) selected from the group consisting of FcγRI, FcγRII, and FcγRIII.

In one embodiment, the effector function is reduced by a factor selected from the group consisting of about 1-fold or more, about 2-fold or more, about 3-fold or more, about 4-fold or more, about 5-fold or more, about 6-fold or more, about 7-fold or more, about 8-fold or more, about 9-fold or more, about 10-fold or more, about 15-fold or more, about 50-fold or more, and about 100-fold or more.

In one embodiment, said stabilized polypeptide has enhanced half-life as compared to a parental Fc polypeptide.

In another embodiment, the enhanced half-life is due to enhanced binding to the neonatal receptor (FcRn).

In one embodiment, the half-life is enhanced by a factor selected from the group consisting of about 1-fold or more, about 2-fold or more, about 3-fold or more, about 4-fold or more, about 5-fold or more, about 6-fold or more, about 7-fold or more, about 8-fold or more, about 9-fold or more, about 10-fold or more, about 15-fold or more, about 50-fold or more, and about 100-fold or more.

In one embodiment, the Fc region is a dimeric Fc region.

In another embodiment, the Fc region is a single chain Fc region.

In one embodiment, all of the Fc moieties of the Fc region are aglycosylated.

In one embodiment, the aglycosylated Fc region comprises a substitution at position 299 of the Fc region (EU numbering convention).

In another embodiment, the aglycosylated Fc region is aglycosylated as a result of its production in a bacterial host cell. In one embodiment, the aglycosylated Fc region is aglycosylated as a result of deglycosylation by chemical or enzymatic means. In one embodiment, the aglycosylated Fc region comprises a chimeric hinge domain.

In one embodiment, the chimeric hinge domain comprises a substitution with proline residue at amino acid position 228 (EU numbering convention).

In one embodiment, the stabilizing amino acid(s) are independently selected from the group consisting of (i) an uncharged amino acid at position 297, ii) a positively charged amino acid at position 299, (iii) a polar amino acid at position 307, (iv) a positively charged or polar amino acid at position 309, (v) a polar amino acid at position 399, (vi) a positively charged or polar amino acid at position 409, and (vii) a polar amino acid at position 427.

In one embodiment, at least one stabilizing amino acid is a Gln at amino acid position 297 (EU numbering).

In one embodiment, at least one of the stabilizing amino acids is a lysine (K) or tyrosine (Y) at position 299.

In one embodiment, at least one of the stabilizing amino acids is a proline (P) or methionine (M) at position 307.

In one embodiment, at least one of the stabilizing amino acids is a proline (P), methionine (M) or lysine (K) at position 309.

In one embodiment, at least one of the stabilizing mutations is a serine (S) at position 399.

In one embodiment, at least one of the stabilizing mutations is a phenylalanine (F) at position 240.

In one embodiment, at least one of the stabilizing mutations is a leucine (L) at position 262.

In one embodiment, at least one of the stabilizing mutations is a threonine (T) at position 264.

In one embodiment, at least one of the stabilizing mutations is a phenylalanine (F) at position 266.

In one embodiment, at least one of the stabilizing mutations is a phenylalanine (F) at position 323.

In one embodiment, at least one of the stabilizing mutations is a lysine (K) or methionine (M) at position 409.

In one embodiment, at least one of the stabilizing mutations is a phenylalanine (F) at position 427.

In one embodiment, a stabilized polypeptide of the invention comprises two or more stabilizing mutations comprising (i) an alanine (A) or lysine (K) at position 299 and (ii) a phenylalanine (F) at position 266.

In one embodiment, a stabilized polypeptide of the invention comprises two or more stabilizing mutations comprising (i) an alanine (A) or lysine (K) at position 299 and (ii) a proline (P) at position 307.

In one embodiment, a stabilized polypeptide of the invention comprises two or more stabilizing mutations comprising (i) a lysine (K) at position 299 and (ii) a serine (s) at position 399.

In one embodiment, a stabilized polypeptide of the invention comprises two or more stabilizing mutations comprising (i) a lysine (K) at position 299 and (ii) a phenylalanine (F) at position 427.

In one embodiment, a stabilized polypeptide of the invention comprises three or more stabilizing mutations comprising (i) an alanine (A) or lysine (K) at position 299, (ii) a leucine (L) at position 262, and (iii) threonine (T) at position 264.

In one embodiment, a stabilized polypeptide of the invention comprises three or more stabilizing mutations comprising (i) a lysine (K) at position 299, (ii) a proline (P) at position 307, and (iii) a serine (S) at position 399.

In one embodiment, a stabilized polypeptide of the invention comprises three or more stabilizing mutations comprising (i) a lysine (K) at position 299, (ii) a lysine (K) at position 309, and (iii) a serine (S) at position 399.

In one embodiment, a stabilized polypeptide of the invention comprises three or more stabilizing mutations comprising (i) a lysine (K) at position 299, (ii) a phenylalanine (F) at position 348, and (iii) a phenylalanine (F) at position 427.

In one embodiment, a stabilized polypeptide of the invention comprises three or more stabilizing mutations comprising (i) a lysine (K) at position 299, (ii) a serine (S) at position 399, and (iii) a phenylalanine (F) at position 427.

In one embodiment, a stabilized polypeptide of the invention comprises four or more stabilizing mutations comprising (i) an alanine (A) or lysine (K) at position 299, (ii) a leucine (L) at position 262, (iii) threonine (T) at position 264, and (iv) a phenylalanine (F) at position 266.

In one embodiment, a stabilized polypeptide of the invention comprises two or more stabilizing mutations comprising (i) a proline (P) at position 307 and (ii) a serine (S) at position 276.

In one embodiment, a stabilized polypeptide of the invention comprises two or more stabilizing mutations comprising (i) a proline (P) at position 307 and (ii) a threonine (T) at position 286.

In one embodiment, a stabilized polypeptide of the invention comprises two or more stabilizing mutations comprising (i) a proline (P) at position 307 and (ii) a phenylalanine (F) at position 323.

In one embodiment, a stabilized polypeptide of the invention comprises two or more stabilizing mutations comprising (i) a proline (P) at position 307 and (ii) a proline (P), lysine (K) or methionine (M) at position 309.

In one embodiment, a stabilized polypeptide of the invention comprises two or more stabilizing mutations comprising (i) a proline (P) at position 307 and (ii) a serine (S) at position 399.

In one embodiment, a stabilized polypeptide of the invention comprises two or more stabilizing mutations comprising (i) a proline (P) at position 307 and (ii) a phenylalanine (F) at position 427.

In one embodiment, a stabilized polypeptide of the invention comprises three or more stabilizing mutations comprising (i) a proline (P) at position 307, (ii) a serine (S) at position 276, and (iii) a threonine (T) at position 286.

In one embodiment, a stabilized polypeptide of the invention comprises three or more stabilizing mutations comprising (i) a proline (P) at position 307, (ii) a proline (P), lysine (K) or methionine (M) at position 309, and (iii) a serine (S) at position 399.

In one embodiment, a stabilized polypeptide of the invention comprises three or more stabilizing mutations comprising (i) a proline (P) at position 307 and (ii) a serine (S) at position 399, and (iii) a phenylalanine (F) at position 427.

In one embodiment, a stabilized polypeptide of the invention comprises three or more stabilizing mutations comprising (i) a proline (P), lysine (K) or methionine (M) at position 309 and (ii) a isoleucine (I) at position 308.

In one embodiment, a stabilized polypeptide of the invention comprises two or more stabilizing mutations comprising (i) a proline (P), lysine (K) or methionine (M) at position 309 and (ii) a serine (S) at position 399 In one embodiment, the Fc region is operably linked to a binding site.

In one embodiment, the binding site is selected from an antigen binding site, a ligand binding portion of a receptor, or a receptor binding portion of a ligand.

In one embodiment, the binding site is derived from a modified antibody selected from the group consisting of an scFv, a Fab, a minibody, a diabody, a triabody, a nanobody, a camelid antibody, and a Dab

In one embodiment, the stabilized polypeptide is a stabilized full length antibody.

In one embodiment, the antibody is selected from the group consisting of a monoclonal antibody, a chimeric antibody, a human antibody, and a humanized antibody.

In one embodiment, at least one binding site comprises six CDRs, a variable heavy and variable light region, or antigen binding site from an antibody selected from the group consisting of Rituximab, Daclizumab, Galiximab, CB6, Li33, 5c8, CBE11, BDA8, 14A2, B3F6, 2B8, Lym 1, Lym 2, LL2, Her2, 5E8, B1, MB1, BH3, B4, B72.3, CC49, and 5E10.

In one embodiment, the stabilized full length antibody is fused to a conventional or stabilized scFv molecule.

In one embodiment, the stabilized polypeptide is a stabilized immunoadhesin.

In one embodiment, a binding site is veneered onto the surface of the Fc region of the stabilized polypeptide.

In one embodiment, the binding site is derived from a non-immunoglobulin binding molecule.

In one embodiment, non-immunogloublin binding molecule is selected from the group consisting of an adnectin, an affibody, a DARPin and an anticalin.

In one embodiment, said ligand binding portion of a receptor is derived a receptor selected from the group consisting of a receptor of the Immunoglobulin (Ig) superfamily, a receptor of the TNF receptor superfamily, a receptor of the G-protein coupled receptor (GPCR) superfamily, a receptor of the Tyrosine Kinase (TK) receptor superfamily, a receptor of the Ligand-Gated (LG) superfamily, a receptor of the chemokine receptor superfamily, IL-1/Toll-like Receptor (TLR) superfamily, a receptor of the glial glial-derived neurotrophic factor (GDNF) receptor family, and a cytokine receptor superfamily.

In one embodiment, said receptor binding portion of a ligand is derived from an inhibitory ligand.

In one embodiment, said receptor binding portion of a ligand is derived from an activating ligand.

In one embodiment, said ligand binds a receptor selected from the group consisting of a receptor of the Immunoglobulin (Ig) superfamily, a receptor of the TNF receptor superfamily, a receptor of the G-protein coupled receptor (GPCR) superfamily, a receptor of the Tyrosine Kinase (TK) receptor superfamily, a receptor of the Ligand-Gated (LG) superfamily, a receptor of the chemokine receptor superfamily, IL-1/Toll-like Receptor (TLR) superfamily, and a cytokine receptor superfamily.

In one embodiment, the invention pertains to a composition comprising a stabilized polypeptide of the invention and a pharmaceutically acceptable carrier.

In one aspect, the invention pertains to a method for stabilizing a parental Fc polypeptide comprising an aglycosylated, chimeric Fc region or portion thereof, the method comprising substituting an elected amino acid in at least one Fc moiety of the Fc region with a stabilizing amino acid to produce a stabilized Fc polypeptide with enhanced stability relative to said starting polypeptide, wherein the substitution is made an amino acid position of the Fc moiety selected from the group consisting of 297, 299, 307, 309, 399, 409 and 427 (EU Numbering Convention).

In one embodiment, the chimeric Fc region comprises a CH2 domains from an IgG antibody of the IgG4 isotype and a CH3 domain from an IgG antibody of the IgG1 isotype.

In one embodiment, the amino acid position and the amino acid present in the stabilized Fc polypeptide is selected from the group consisting of 297Q, 299A, 299K, 307P, 309K, 309M, 309P, 323F, 399E, 399S, 409K, 409M and 427F.

In one embodiment, the stabilized Fc polypeptide comprises a Gln at position 297 (EU numbering).

In another aspect, the invention pertains to a method for enhancing the yield of a parental Fc polypeptide comprising an Fc region or portion thereof, the method comprising substituting an elected amino acid in at least one Fc moiety of the Fc region with one or more stabilizing amino acids to produce a stabilized Fc polypeptide with enhanced yield relative to said parental polypeptide, wherein the stabilizing amino acids are independently selected from the group consisting of 240F, 262L, 264T, 266F, 299K, 307P, 309K, 309M, 309P, 323F, 399S, and 427F (EU Numbering Convention).

In one embodiment, the starting Fc region is an IgG1 Fc region.

In one embodiment, a stabilized polypeptide of the invention the starting Fc region is an IgG4 Fc region.

In one embodiment, the starting Fc region is an aglycosylated IgG1 Fc region.

In another embodiment, the starting Fc region is an aglycosylated IgG4 Fc region.

In one embodiment, the stabilized Fc polypeptide comprises two or more stabilizing amino acids. In one embodiment, the stabilized Fc polypeptide comprises three or more stabilizing amino acids.

In another aspect, the invention pertains to a nucleic acid molecule comprising a nucleotide sequence encoding a stabilized binding polypeptide of any one of the proceeding claims.

In one embodiment, the nucleic acid molecule comprises a nucleotide sequence encoding a polypeptide chain of a stabilized binding polypeptide.

In one embodiment, the invention pertains to a vector comprising the nucleic acid molecule encoding a stabilized binding polypeptide or polypeptide chain thereof.

In one embodiment, the invention pertains to a host cell expressing a vector.

In one embodiment, the invention pertains to a method of producing a stabilized Fc polypeptide of the invention comprising culturing the host cell in culture medium such that the stabilized Fc polypeptide is produced.

In one aspect, the invention pertains to a method for large scale manufacture of a polypeptide comprising a stabilized Fc region, the method comprising:

(d) genetically fusing at least one stabilized Fc moiety to a polypeptide to form a stabilized fusion protein;

(e) transfecting a mammalian host cell with a nucleic acid molecule encoding the stabilized fusion protein,

(f) culturing the host cell of step (f) in 10 L or more of culture medium under conditions such that the stabilized fusion protein is expressed;

to thereby produce a stabilized fusion protein.

In one embodiment, the stabilized Fc region is chimeric Fc comprising a CH2 domains from an IgG antibody of the IgG4 isotype and a CH3 domain from an IgG antibody of the IgG1 isotype.

In one embodiment, the stabilized Fc region comprises a Gln at amino acid position 297 (EU numbering).

In one embodiment, the invention pertains to a method for treating or preventing a disease or disorder in a subject, comprising a binding molecule of the invention or a composition comprising such a binding molecule to a subject suffering from said disease or disorder to thereby treat or prevent a disease or disorder.

In one embodiment, the disease or disorder is selected from the group consisting of an inflammatory disorder, a neurological disorder, an autoimmune disorder, and a neoplastic disorder.

Other features and advantages of the invention will be apparent from the following detailed description and claims.

### BRIEF DESCRIPTION OF THE DRAWINGS

FIG. 1 depicts the structure of a typical antigen binding polypeptide (IgG antibody) and the functional properties of antigen binding and effector function (e.g., Fc receptor (FcR) binding) of an antibody. Also shown is how the presence of sugars (glycosylation) in the CH2 domain of the antibody alters effector function (FcR binding) but does not affect antigen binding.

FIG. 2 depicts the two interacting CH3 domains (Panel A) from an IgG1 x-ray crystal structure (pdb code 1hzh). Highlighted are IgG1 residues K409 and D399. Panel 2B depicts the alignment of human IgG1/kappa constant domain sequences using a structure-based HMM (Wang, N., Smith, W., Miller, B., Aivazian, D., Lugovskoy, A., Reff, M., Glaser, S., Croner, L., Demarest, S. (2008) Conserved amino acid networks involved in antibody variable domain interactions. Proteins: Struct. Funct. Bioinform. In Press.). Residue positions of C_{L}, C_{H}1, and C_{H}3 that are involved in inter-domain interactions and amino acid positions that covary strongly with those amino acids in direct contact with the carbohydrate are highlighted in grey. Kabat and EU number are provided below the alignment. Panel 2C depicts the ribbon diagram of the structure of the IgG1-C_{H}2 domain (Sondermann et al., 2000). The valine residues buried by the N-linked carbohydrate and the unique 6 amino acid loop within the C_{H}2 domain are labeled. Panel 2D depicts the alignment of the native IgG1-C_{H}2 sequence and the fully modified IgG1-C_{H}2 sequence. Residue positions that were modified are shown in black. The EU number of the modified positions is shown above the alignment.

FIG. 3 depicts the turbidity (Panel A) and % monomer content (Panel B) of exemplary IgG Fc constructs of the invention following agitation.

FIG. 4 depicts relative peak height over time of IgG Fc constructs at a low pH hold (pH 3.1).

FIG. 5 depicts initial binding rates of exemplary IgG1 and IgG4 Fc constructs of the invention to Fcγ receptors as measured by solution affinity surface plasmon resonance.

FIG. 6 depicts the titration curves used to calculate IC50s for binding of exemplary IgG1 and IgG4 Fc constructs of the invention to CD64 (FcγRI) (FIG. 6A) and CD16 (FcγRIIIa V158) (FIG. 6B).

FIG. 7 depicts the titration curves highlighting the reduction in binding of the IgG1 T299K compared to IgG1 T299A and IgG1 wild type for CD64 (FcγRI) (FIG. 7A) and the binding of exemplary IgG4 Fc constructs incorporating the T299K mutation compared to other exemplary IgG4 Fc constructs incorporating the T299A mutation and IgG1 wild type for CD64 (FcγRI) (FIG. 7B).

FIG. 8 depicts the binding of exemplary IgG4 Fc constructs incorporating the T299K mutation compared to other exemplary IgG4 Fc constructs incorporating the T299A mutation and IgG1 wild type for CD16 (FcγRIIIa V158).

FIG. 9 depicts the titration curves used to evaluate binding of exemplary IgG1 and IgG4 Fc constructs of the invention to complement factor C1q.

FIG. 10 panels A and B illustrate the titration curves used to evaluate binding of various Fc constructs to CD64 and CD16, respectively. Panel C illustrates that the N297Q IgG4-CH2/IgG1-CH3 has the same half-life as the T299A antibody (which was slightly shorter than the aglycosylated IgG1).

FIG. 11 panel A illustrates titration curves used to evaluate binding of T299X constructs to CD64 and that positively charged side chains T299R and T299K impart low affinities for CD64. Panel B illustrates titration curves used to evaluate binding of CD64 to various alternative constructs. Panels C and D illustrate titration curves used to evaluate binding of constructs to CD32aR and panels E and F illustrate binding of constructs to CD16. Panels G and H illustrate the results of a C1q ELISA.

FIG. 12 panel A illustrates titration curves used to evaluate binding of constructs to CD64 and panel B illustrates titration curves used to evaluate the binding of constructs to CD16.

### DETAILED DESCRIPTION OF THE INVENTION

A method has been developed to produce stabilized Fc polypeptides with reduced effector function, for example, aglycosylated antibodies or IgG4 antibodies, by including one or more stabilizing amino acids in the Fc region of Fc polypeptide. The method is especially well suited for producing therapeutic Fc-containing polypeptides in eukaryotic cells with only minimal amino acid alterations to the polypeptide. The methods of the present invention thereby avoid introducing amino acid sequence into the polypeptide that can be immunogenic. Preferably, the stabilizing amino acids stabilize the Fc region of the polypeptide without the influencing the glycosylation and/or effector function of the polypeptide, and do not significantly alter other desired functions of the polypeptide (e.g., antigen binding affinity or half-life).

In order to provide a clear understanding of the specification and claims, the following definitions are conveniently provided below.

### DEFINITIONS

As used herein, the term “effector function” refers to the functional ability of the Fc region or portion thereof to bind proteins and/or cells of the immune system and mediate various biological effects. Effector functions may be antigen-dependent or antigen-independent. A decrease in effector function refers to a decrease in one or more effector functions, while maintaining the antigen binding activity of the variable region of the antibody (or fragment thereof). Increase or decreases in effector function, e.g., Fc binding to an Fc receptor or complement protein, can be expressed in terms of fold change (e.g., changed by 1-fold, 2-fold, and the like) and can be calculated based on, e.g., the percent changes in binding activity determined using assays the are well-known in the art.

As used herein, the term “antigen-dependent effector function” refers to an effector function which is normally induced following the binding of an antibody to a corresponding antigen. Typical antigen-dependent effector functions include the ability to bind a complement protein (e.g. C1q). For example, binding of the C1 component of complement to the Fc region can activate the classical complement system leading to the opsonization and lysis of cell pathogens, a process referred to as complement-dependent cytotoxicity (CDCC). The activation of complement also stimulates the inflammatory response and may also be involved in autoimmune hypersensitivity.

Other antigen-dependent effector functions are mediated by the binding of antibodies, via their Fc region, to certain Fc receptors (“FcRs”) on cells. There are a number of Fc receptors which are specific for different classes of antibody, including IgG (gamma receptors, or IgγRs), IgE (epsilon receptors, or IgεRs), IgA (alpha receptors, or IgαRs) and IgM (mu receptors, or IgμRs). Binding of antibody to Fc receptors on cell surfaces triggers a number of important and diverse biological responses including endocytosis of immune complexes, engulfment and destruction of antibody-coated particles or microorganisms (also called antibody-dependent phagocytosis, or ADCP), clearance of immune complexes, lysis of antibody-coated target cells by killer cells (called antibody-dependent cell cytotoxicity, or ADCC), release of inflammatory mediators, regulation of immune system cell activation, placental transfer and control of immunoglobulin production.

Certain Fc receptors, the Fc gamma receptors (FcγRs), play a critical role in either abrogating or enhancing immune recruitment. FcγRs are expressed on leukocytes and are composed of three distinct classes: FcγRI, FcγRII, and FcγRIII (Gessner et al., Ann. Hematol., (1998), 76: 231-48). Structurally, the FcγRs are all members of the immunoglobulin superfamily, having an IgG-binding α-chain with an extracellular portion composed of either two or three Ig-like domains. Human FcγRI (CD64) is expressed on human monocytes, exhibits high affinity binding (Ka=10⁸-10⁹ M⁻¹) to monomeric IgG1, IgG3, and IgG4. Human FcγRII (CD32) and FcγRIII (CD16) have low affinity for IgG1 and IgG3 (Ka <10⁷ M⁻¹), and can bind only complexed or polymeric forms of these IgG isotypes. Furthermore, the FcγRII and FcγRIII classes comprise both “A” and “B” forms. FcγRIIa (CD32a) and FcγRIIIa (CD16a) are bound to the surface of macrophages, NK cells and some T cells by a transmembrane domain while FcγRIIb (CD32b) and FcγRIIIb (CD16b) are selectively bound to cell surface of granulocytes (e.g. neutrophils) via a phosphatidyl inositol glycan (GPI) anchor. The respective murine homologs of human FcγRI, FcγRII, and FcγRIII are FcγRIIa, FcγRIIb/1, and FcγR1o.

As used herein, the term “antigen-independent effector function” refers to an effector function which may be induced by an antibody, regardless of whether it has bound its corresponding antigen. Typical antigen-independent effector functions include cellular transport, circulating half-life and clearance rates of immunoglobulins, and facilitation of purification. A structurally unique Fc receptor, the “neonatal Fc receptor” or “FcRn”, also known as the salvage receptor, plays a critical role in regulating half-life and cellular transport. Other Fc receptors purified from microbial cells (e.g. Staphylococcal Protein A or G) are capable of binding to the Fc region with high affinity and can be used to facilitate the purification of the Fc-containing polypeptide.

Unlike FcγRs which belong to the Immunoglobulin superfamily, human FcRns structurally resemble polypeptides of Major Histocompatibility Complex (MHC) Class I (Ghetie and Ward, Immunology Today, (1997), 18(12): 592-8). FcRn is typically expressed as a heterodimer consisting of a transmembrane a or heavy chain in complex with a soluble β or light chain (β2 microglobulin). FcRn shares 22-29% sequence identity with Class I MHC molecules and has a non-functional version of the MHC peptide binding groove (Simister and Mostov, Nature, (1989), 337: 184-7. Like MHC, the α chain of FcRn consists of three extracellular domains (α1, α2, α3) and a short cytoplasmic tail anchors the protein to the cell surface. The α1 and α2 domains interact with FcR binding sites in the Fc region of antibodies (Raghavan et al., Immunity, (1994), 1: 303-15). FcRn is expressed in the maternal placenta or yolk sac of mammals and it is involved in transfer of IgGs from mother to fetus. FcRn is also expressed in the small intestine of rodent neonates, where it is involved in the transfer across the brush border epithelia of maternal IgG from ingested colostrum or milk. FcRn is also expressed in numerous other tissues across numerous species, as well as in various endothelial cell lines. It is also expressed in human adult vascular endothelium, muscle vasculature, and hepatic sinusoids. FcRn is thought to play an additional role in maintaining the circulatory half-life or serum levels of IgG by binding it and recycling it to the serum. The binding of FcRn to IgG molecules is strictly pH-dependent with an optimum binding at a pH of less than 7.0.

As used herein, the term “half-life” refers to a biological half-life of a particular binding polypeptide in vivo. Half-life may be represented by the time required for half the quantity administered to a subject to be cleared from the circulation and/or other tissues in the animal. When a clearance curve of a given binding polypeptide is constructed as a function of time, the curve is usually biphasic with a rapid α-phase and longer β-phase. The α-phase typically represents an equilibration of the administered Fc polypeptide between the intra- and extra-vascular space and is, in part, determined by the size of the polypeptide. The β-phase typically represents the catabolism of the binding polypeptide in the intravascular space. Therefore, in a preferred embodiment, the term half-life as used herein refers to the half-life of the binding polypeptide in the β-phase. The typical β phase half-life of a human antibody in humans is 21 days.

As used herein, the term “polypeptide” refers to a polymer of two or more of the natural amino acids or non-natural amino acids. The term “Fc polypeptide” refers to a polypeptide comprising an Fc region or a portion thereof (e.g., an Fc moiety). In preferred embodiments, the Fc polypeptide is stabilized according to the methods of the invention. In optional embodiments, the Fc polypeptide further comprises a binding site which is operably linked or fused to the Fc region (or portion thereof) of the Fc polypeptide.

As used herein, the term “protein” refers to a polypeptide (e.g., an Fc polypeptide) or a composition comprising more than one polypeptide. Accordingly, proteins may be either monomers (e.g., a single Fc polypeptide) or multimers. For example, in one embodiment, a protein of the invention is a dimer. In one embodiment, the dimers of the invention are homodimers, comprising two identical monomeric subunits or polypeptides (e.g., two identical Fc polypeptides). In another embodiment, the dimers of the invention are heterodimers, comprising two non-identical monomeric subunits or polypeptides (e.g., two non-identical Fc polypeptides or an Fc polypeptide and a second polypeptide other than an Fc polypeptide). The subunits of the dimer may comprise one or more polypeptide chains, wherein at least one of the polypeptide chains is an Fc polypeptide. For example, in one embodiment, the dimers comprise at least two polypeptide chains (e.g, at least two Fc polypeptide chains). In one embodiment, the dimers comprise two polypeptide chains, wherein one or both of the chains are Fc polypeptide chains. In another embodiment, the dimers comprise three polypeptide chains, wherein one, two or all of the polypeptide chains are Fc polypeptide chains. In another embodiment, the dimers comprise four polypeptide chains, wherein one, two, three, or all of the polypeptide chains are Fc polypeptide chains.

As used herein, the terms “linked”, “fused”, or “fusion”, are used interchangeably. These terms refer to the joining together of two more elements or components, by whatever means including chemical conjugation or recombinant means. Methods of chemical conjugation (e.g., using heterobifunctional crosslinking agents) are known in the art. As used herein, the term “genetically fused” or “genetic fusion” refers to the co-linear, covalent linkage or attachment of two or more proteins, polypeptides, or fragments thereof via their individual peptide backbones, through genetic expression of a single polynucleotide molecule encoding those proteins, polypeptides, or fragments. Such genetic fusion results in the expression of a single contiguous genetic sequence. Preferred genetic fusions are in frame, i.e., two or more open reading frames (ORFs) are fused to form a continuous longer ORF, in a manner that maintains the correct reading frame of the original ORFs. Thus, the resulting recombinant fusion protein is a single polypeptide containing two or more protein segments that correspond to polypeptides encoded by the original ORFs (which segments are not normally so joined in nature). Although the reading frame is thus made continuous throughout the fused genetic segments, the protein segments may be physically or spatially separated by, for example, an in-frame polypeptide linker.

As used herein, the term “Fc region” shall be defined as the portion of a immunoglobulin formed by two or more Fc moieties of antibody heavy chains. In certain embodiments, the Fc region is a dimeric Fc region. A “dimeric Fc region” or “dcFc” refers to the dimer formed by the Fc moieties of two separate immunoglobulin heavy chains. The dimeric Fc region may be a homodimer of two identical Fc moieties (e.g., an Fc region of a naturally occurring immunoglobulin) or a heterodimer of two non-identical Fc moieties. In other embodiments, the Fc region is monomeric or “single-chain” Fc region (i.e., a scFc region). Single chain Fc regions are comprised of Fc moieties genetically linked within a single polypeptide chain (i.e., encoded in a single contiguous genetic sequence). Exemplary scFc regions are disclosed in PCT Application No. PCT/US2008/006260, filed May 14, 2008, which is incorporated by reference herein.

As used herein, the term “Fc moiety” refers to a sequence derived from the portion of an immunoglobulin heavy chain beginning in the hinge region just upstream of the papain cleavage site (i.e., residue 216 in IgG, taking the first residue of heavy chain constant region to be 114) and ending at the C-terminus of the immunoglobulin heavy chain. Accordingly, an Fc moiety may be a complete Fc moiety or a portion (e.g., a domain) thereof. A complete Fc moiety comprises at least a hinge domain, a CH2 domain, and a CH3 domain (e.g., EU amino acid positions 216-446). An additional lysine residue (K) is sometimes present at the extreme C-terminus of the Fc moiety, but is often cleaved from a mature antibody. Each of the amino acid positions within an Fc region have been numbered according to the art-recognized EU numbering system of Kabat, see e.g., by Kabat et al., in “Sequences of Proteins of Immunological Interest”, U.S. Dept. Health and Human Services, 1983 and 1987.

In certain embodiments, an Fc moiety comprises at least one of: a hinge (e.g., upper, middle, and/or lower hinge region) domain, a CH2 domain, a CH3 domain, or a variant, portion, or fragment thereof. In preferred embodiments, an Fc moiety comprises at least a CH2 domain or a CH3 domain. In certain embodiments, the Fc moiety is a complete Fc moiety. In other embodiments, the Fc moiety comprises one or more amino acid insertions, deletions, or substitutions relative to a naturally-occurring Fc moiety. For example, at least one of a hinge domain, CH2 domain or CH3 domain (or portion thereof) may be deleted. For example, an Fc moiety may comprise or consist of: (i) hinge domain (or portion thereof) fused to a CH2 domain (or portion thereof), (ii) a hinge domain (or portion thereof) fused to a CH3 domain (or portion thereof), (iii) a CH2 domain (or portion thereof) fused to a CH3 domain (or portion thereof), (iv) a CH2 domain (or portion thereof), and (v) a CH3 domain or portion thereof.

As set forth herein, it will be understood by one of ordinary skill in the art that the Fc moiety may be modified such that it varies in amino acid sequence from the complete Fc moiety of a naturally occurring immunoglobulin molecule, while retaining at least one desirable function conferred by the naturally-occurring Fc moiety. For example, the Fc moiety may comprise or consist of at least the portion of an Fc moiety that is known in the art to be required for FcRn binding or extended half-life. In another embodiment, an Fc moiety comprises at least the portion known in the art to be required for FcγR binding. In one embodiment, an Fc region of the invention comprises at least the portion of known in the art to be required for Protein A binding. In one embodiment, an Fc moiety of the invention comprises at least the portion of an Fc molecule known in the art to be required for protein G binding.

In certain embodiments, the Fc moieties of Fc region are of the same isotype. For example, the Fc moieties may be derived from an immunoglobulin (e.g., a human immunoglobulin) of an IgG1 or IgG4 isotype. However, the Fc region (or one or more Fc moieties of an Fc region) may also be chimeric. A chimeric Fc region may comprise Fc moieties derived from different immunoglobulin isotypes. In certain embodiments, at least two of the Fc moieties of a dimeric or single-chain Fc region may be from different immunoglobulin isotypes. In additional or alternative embodiments, the chimeric Fc regions may comprise one or more chimeric Fc moieties. For example, the chimeric Fc region or moiety may comprise one or more portions derived from an immunoglobulin of a first isotype (e.g., an IgG1, IgG2, or IgG3 isotype) while the remainder of the Fc region or moiety is of a different isotype. For example, an Fc region or moiety of an Fc polypeptide may comprise a CH2 and/or CH3 domain derived from an immunoglobulin of a first isotype (e.g., an IgG1, IgG2 or IgG4 isotype) and a hinge region from an immunoglobulin of a second isotype (e.g., an IgG3 isotype). In another embodiment, the Fc region or moiety comprises a hinge and/or CH2 domain derived from an immunoglobulin of a first isotype (e.g., an IgG4 isotype) and a CH3 domain from an immunoglobulin of a second isotype (e.g., an IgG1, IgG2, or IgG3 isotype). In another embodiment, the chimeric Fc region comprises an Fc moiety (e.g., a complete Fc moiety) from an immunoglobulin for a first isotype (e.g., an IgG4 isotype) and an Fc moiety from an immunoglobulin of a second isotype (e.g., an IgG1, IgG2 or IgG3 isotype). In one exemplary embodiment, the Fc region or moiety comprises a CH2 domain from an IgG4 immunoglobulin and a CH3 domain from an IgG1 immunoglobulin. In another embodiment, the Fc region or moiety comprises a CH1 domain and a CH2 domain from an IgG4 molecule and a CH3 domain from an IgG1 molecule. In another embodiment, the Fc region or moiety comprises a portion of a CH2 domain from a particular isotype of antibody, e.g., EU positions 292-340 of a CH2 domain. For example, in one embodiment, an Fc region or moiety comprises amino acids a positions 292-34 of CH2 derived from an IgG4 moiety and the remainder of CH2 derived from an IgG1 moiety (alternatively, 292-34 of CH2 may be derived from an IgG1 moiety and the remainder of CH2 derived from an IgG4 moiety).

In other embodiments, an Fc region or moiety can comprise a chimeric hinge region. The chimeric hinge may be derived, in part, from an IgG1, IgG2, or IgG4 molecule (e.g., an upper and lower middle hinge sequence) and, in part, from an IgG3 molecule (e.g., an middle hinge sequence). In another example, an Fc region or moiety can comprise a chimeric hinge derived, in part, from an IgG1 molecule and, in part, from an IgG4 molecule. In a particular embodiment, the chimeric hinge can comprise upper and lower hinge domains from an IgG4 molecule and a middle hinge domain from an IgG1 molecule. Such a chimeric hinge can be made by introducing a proline substitution (Ser228Pro) at EU position 228 in the middle hinge domain of an IgG4 hinge region. In another embodiment, the chimeric hinge can comprise amino acids at EU positions 233-236 are from an IgG2 antibody and/or the Ser228Pro mutation, wherein the remaining amino acids of the hinge are from an IgG4 antibody (e.g., a chimeric hinge of the sequence ESKYGPPCPPCPAPPVAGP). Additional chimeric hinges are described in U.S. patent application Ser. No. 10/880,320, which is incorporated by reference herein in its entirety.

Specifically included within the definition of “Fc region” is an “aglycosylated Fc region”. By “aglycosylated Fc region” as used herein is Fc region that lacks a covalently linked oligosaccharide or glycan, e.g., at the N-glycosylation site at EU position 297, in one or more of the Fc moieties thereof. In certain embodiments the aglycosylated Fc region is fully aglycosylated, i.e., all of its Fc moieties lack carbohydrate. In other embodiments, the aglycosylation is partially aglycosylated (i.e., hemi-glycosylated). The aglycosylated Fc region may be a deglycosylated Fc region, that is an Fc region for which the Fc carbohydrate has been removed, for example chemically or enzymatically. Alternatively, the aglycosylated Fc region may be a nonglycosylated or unglycosylated, that is an antibody that was expressed without Fc carbohydrate, for example by mutation of one or residues that encode the glycosylation pattern, e.g., at the N-glycosylation site at EU position 297 or 299, by expression in an organism that does not naturally attach carbohydrates to proteins, (e.g., bacteria), or by expression in a host cell or organism whose glycosylation machinery has been rendered deficient by genetic manipulation or by the addition of glycosylation inhibitors (e.g., glycosyltransferase inhibitors). In alternative embodiments, the Fc region is a “glycosylated Fc region”, i.e., it is fully glycosylated at all available glycosylation sites.

The term “parental Fc polypeptide” includes a polypeptide containing an Fc region (e.g., an IgG antibody) for which stabilization is desired. Preferably the parental Fc polypeptide is an effector-less Fc polypeptide. Thus, the parental Fc polypeptide represents the original Fc polypeptide on which the methods of the instant invention are performed or which can be used a reference point for stability comparisons. The parental polypeptide may comprise a native (i.e. a naturally occurring) Fc region or moiety (e.g., a human IgG4 Fc region or moiety) or an Fc region with pre-existing amino acid sequence modifications (such as insertions, deletions and/or other alterations) of a naturally occurring sequence, but lacking one or more stabilizing amino acid.

The term “mutation” or “mutating” shall be understood to include physically making a mutation in a parental Fc polypeptide (e.g., by altering, e.g., by site-directed mutagenesis, a codon of a nucleic acid molecule encoding one amino acid to result in a codon encoding a different amino acid) or synthesizing a variant Fc region having an amino acid not found in the parental Fc region (e.g., by knowing the nucleotide sequence of a nucleic acid molecule encoding a parental Fc region and by designing the synthesis of a nucleic acid molecule comprising a nucleotide sequence encoding a variant of the parental Fc region without the need for mutating one or more nucleotides of a nucleic acid molecule which encodes a stabilized polypeptide of the invention).

In one exemplary embodiment, the parent Fc polypeptide comprises an Fc region from an effector-less Fc polypeptide. As used herein the term “effector-less Fc polypeptide” refers to an Fc polypeptide which has altered or reduced effector function as compared to a wild-type, aglycosylated antibody of the IgG1 isotype. Preferably, the effector function that is reduced or altered is an antibody-dependent effector function, e.g., ADCC and/or ADCP. In one embodiment, an effector-less Fc polypeptide has reduced effector function as a result of modified or reduced glycosylation in the Fc region of the Fc polypeptide, e.g., an aglycosylated Fc region. In another embodiment, the effector-less Fc polypeptide has reduced effector function due to the incorporation of an IgG4 Fc region or portion thereof (e.g., a CH2 and/or CH3 domain of an IgG4 antibody).

The terms “variant Fc polypeptide” or “Fc variant”, include an Fc polypeptide derived from a parental Fc polypeptide. The Fc variant differs from the parental Fc polypeptide in that it comprises stabilizing one or more stabilizing amino acid residues, e.g., due to the introduction of at least one Fc stabilizing mutation. In certain embodiments, the Fc variants of the invention comprise an Fc region (or Fc moiety) that is identical in sequence to that of a parental polypeptide but for the presence of one or more stabilizing Fc amino acids. In preferred embodiments, the Fc variant will have enhanced stability as compared to the parental Fc polypeptide and, optionally, equivalent or reduced effector function as compared to the parental Fc polypeptide.

A polypeptide or amino acid sequence “derived from” a designated polypeptide or protein refers to the origin of the polypeptide. Preferably, the polypeptide or amino acid sequence which is derived from a particular sequence has an amino acid sequence that is essentially identical to that sequence or a portion thereof, wherein the portion consists of at least 10-20 amino acids, preferably at least 20-30 amino acids, more preferably at least 30-50 amino acids, or which is otherwise identifiable to one of ordinary skill in the art as having its origin in the sequence. In the context of polypeptides, a “linear sequence” or a “sequence” is the order of amino acids in a polypeptide in an amino to carboxyl terminal direction in which residues that neighbor each other in the sequence are contiguous in the primary structure of the polypeptide.

Polypeptides (e.g., variant Fc polypeptides) derived from another polypeptide (e.g., a parental Fc polypeptide) may have one or more mutations relative to the starting or parent polypeptide, e.g., one or more amino acid residues which have been substituted with another amino acid residue or which has one or more amino acid residue insertions or deletions. Preferably, the polypeptide comprises an amino acid sequence which is not naturally occurring. Such variants necessarily have less than 100% sequence identity or similarity with the starting polypeptide. In a preferred embodiment, the variant will have an amino acid sequence from about 75% to less than 100% amino acid sequence identity or similarity with the amino acid sequence of the starting polypeptide, more preferably from about 80% to less than 100%, more preferably from about 85% to less than 100%, more preferably from about 90% to less than 100% (e.g., 91%, 92%, 93%, 94%, 95%, 96%, 97%, 98%, 99%) and most preferably from about 95% to less than 100%, e.g., over the entire length of the variant molecule or a portion thereof (e.g., an Fc region or Fc moiety). In one embodiment, there is one amino acid difference between a starting polypeptide sequence (e.g., the Fc region of a parental Fc polypeptide) and the sequence derived therefrom (e.g., the Fc region of a variant Fc polypeptide). In other embodiments, there are between two and ten amino acid differences between the starting polypeptide sequence and the variant polypeptide (e.g., about 2-20, about 2-15, about 2-10, about 5-20, about 5-15, about 5-10 amino acid differences). For example, there may be less than about 10 amino acid differences (e.g., two, three, four, five, six, seven, eight, nine, or ten amino acid differences). Identity or similarity with respect to this sequence is defined herein as the percentage of amino acid residues in the candidate sequence that are identical (i.e. same residue) with the starting amino acid residues, after aligning the sequences and introducing gaps, if necessary, to achieve the maximum percent sequence identity.

Preferred Fc polypeptides of the invention comprise an amino acid sequence (e.g., at least one Fc region or Fc moiety) derived from a human immunoglobulin sequence (e.g., an Fc region or Fc moiety from a human IgG molecule). However, polypeptides may comprise one or more amino acids from another mammalian species. For example, a primate Fc moiety or a primate binding site may be included in the subject polypeptides. Alternatively, one or more murine amino acids may be present in the Fc polypeptide. Preferred Fc polypeptides of the invention are not immunogenic.

It will also be understood by one of ordinary skill in the art that the Fc polypeptides of the invention may be altered such that they vary in amino acid sequence from the parental polypeptides from which they were derived, while retaining one or more desirable activities (e.g., reduced effector function) of the parental polypeptides. In particular embodiments, nucleotide or amino acid substitutions which stabilize the Fc polypeptide are made. In one embodiment, an isolated nucleic acid molecule encoding an Fc variant can be created by introducing one or more nucleotide substitutions, additions or deletions into the nucleotide sequence of the parental Fc polypeptide such that one or more amino acid substitutions, additions or deletions are introduced into the encoded protein. Mutations (e.g., stabilizing mutations) may be introduced by standard techniques, such as site-directed mutagenesis and PCR-mediated mutagenesis.

As used herein the term “protein stability” refers to an art-recognized measure of the maintenance of one or more physical properties of a protein in response to an environmental condition (e.g. an elevated or lowered temperature). In one embodiment, the physical property is the maintenance of the covalent structure of the protein (e.g. the absence of proteolytic cleavage, unwanted oxidation or deamidation). In another embodiment, the physical property is the presence of the protein in a properly folded state (e.g. the absence of soluble or insoluble aggregates or precipitates). In one embodiment, stability of a protein is measured by assaying a biophysical property of the protein, for example thermal stability, pH unfolding profile, stable removal of glycosylation, solubility, biochemical function (e.g., ability to bind to a protein (e.g., a ligand, a receptor, an antigen, etc.) or chemical moiety, etc.), and/or combinations thereof. In another embodiment, biochemical function is demonstrated by the binding affinity of an interaction. In one embodiment, a measure of protein stability is thermal stability, i.e., resistance to thermal challenge. Stability can be measured using methods known in the art and/or described herein. For example, the “Tm”, also referred to as the “transition temperature” may be measured. The Tm is the temperature at which 50% of a macromolecule, e.g., binding molecule, becomes denatured, and is considered to be the standard parameter for describing the thermal stability of a protein.

The term “amino acid” includes alanine (Ala or A); arginine (Arg or R); asparagine (Asn or N); aspartic acid (Asp or D); cysteine (Cys or C); glutamine (Gln or Q); glutamic acid (Glu or E); glycine (Gly or G); histidine (His or H); isoleucine (Ile or I): leucine (Leu or L); lysine (Lys or K); methionine (Met or M); phenylalanine (Phe or F); proline (Pro or P); serine (Ser or S); threonine (Thr or T); tryptophan (Trp or W); tyrosine (Tyr or Y); and valine (Val or V). Non-traditional amino acids are also within the scope of the invention and include norleucine, ornithine, norvaline, homoserine, and other amino acid residue analogues such as those described in Ellman et al. Meth. Enzym. 202:301-336 (1991). To generate such non-naturally occurring amino acid residues, the procedures of Noren et al. Science 244:182 (1989) and Ellman et al., supra, can be used. Briefly, these procedures involve chemically activating a suppressor tRNA with a non-naturally occurring amino acid residue followed by in vitro transcription and translation of the RNA. Introduction of the non-traditional amino acid can also be achieved using peptide chemistries known in the art. As used herein, the term “polar amino acid” includes amino acids that have net zero charge, but have non-zero partial charges in different portions of their side chains (e.g. M, F, W, S, Y, N, Q, C). These amino acids can participate in hydrophobic interactions and electrostatic interactions. As used herein, the term “charged amino acid” include amino acids that can have non-zero net charge on their side chains (e.g. R, K, H, E, D). These amino acids can participate in hydrophobic interactions and electrostatic interactions. As used herein the term “amino acids with sufficient steric bulk” includes those amino acids having side chains which occupy larger 3 dimensional space. Exemplary amino acids having side chain chemistries of sufficient steric bulk include tyrosine, tryptophan, arginine, lysine, histidine, glutamic acid, glutamine, and methionine, or analogs or mimetics thereof.

An “amino acid substitution” refers to the replacement of at least one existing amino acid residue in a predetermined amino acid sequence (an amino acid sequence of a starting polypeptide) with a second, different “replacement” amino acid residue. An “amino acid insertion” refers to the incorporation of at least one additional amino acid into a predetermined amino acid sequence. While the insertion will usually consist of the insertion of one or two amino acid residues, the present larger “peptide insertions”, can be made, e.g. insertion of about three to about five or even up to about ten, fifteen, or twenty amino acid residues. The inserted residue(s) may be naturally occurring or non-naturally occurring as disclosed above. An “amino acid deletion” refers to the removal of at least one amino acid residue from a predetermined amino acid sequence. As set forth above, these terms include actual changes to an existing physical nucleic acid molecule or changes made during a design process (e.g., on paper or on a computer) to an existing nucleic acid sequence.

In certain embodiments, the polypeptides of the invention are binding polypeptides. As used herein, the term “binding polypeptide” refers to polypeptides (e.g., Fc polypeptides) that comprise at least one target binding site or binding domain that specifically binds to a target molecule (such as an antigen or binding partner). For example, in one embodiment, a binding polypeptide of the invention comprises an immunoglobulin antigen binding site or the portion of a receptor molecule responsible for ligand binding or the portion of a ligand molecule that is responsible for receptor binding. The binding polypeptides of the invention comprise at least one binding site. In one embodiment, the binding polypeptides of the invention comprise at least two binding sites. In one embodiment, the binding polypeptides comprise two binding sites. In another embodiment, the binding polypeptides comprise three binding sites. In another embodiment, the binding polypeptides comprise four binding sites. In one embodiment, the binding sites are linked to each other in tandem. In other embodiments, the binding sites are located at different positions of the binding polypeptide, e.g., at one or more of the N- or C-terminal ends of the Fc region of an Fc polypeptide. For example, where the Fc region is a scFc region, a binding site may linked to N-terminal end, the C-terminal end, or both ends of the scFc region. Where the Fc region is a dimeric Fc region, binding sites may be linked to one or both N-terminal ends and/or one or both C-terminal ends.

The terms “binding domain”, “binding site” or “binding moiety”, as used herein, refers to the portion, region, or site of a binding polypeptide that has a biological activity (other than an Fc-mediated biological activity), e.g., which mediates specific binding with a target molecule (e.g. an antigen, ligand, receptor, substrate or inhibitor). Exemplary binding domains include biologically active proteins or moieties, an antigen binding site, a receptor binding domain of a ligand, a ligand binding domain of a receptor or an enzymatic domain. In another example, the term “binding moiety” refers to biologically active molecules or portions thereof which bind to components of a biological system (e.g., proteins in sera or on the surface of cells or in cellular matrix) and which binding results in a biological effect (e.g., as measured by a change in the active moiety and/or the component to which it binds (e.g., a cleavage of the active moiety and/or the component to which it binds, the transmission of a signal, or the augmentation or inhibition of a biological response in a cell or in a subject)).

The term “ligand binding domain” as used herein refers to a native receptor (e.g., cell surface receptor) or a region or derivative thereof retaining at least a qualitative ligand binding ability, and preferably the biological activity of the corresponding native receptor. The term “receptor binding domain” as used herein refers to a native ligand or region or derivative thereof retaining at least a qualitative receptor binding ability, and preferably the biological activity of the corresponding native ligand. In one embodiment, the binding polypeptides of the invention have at least one binding domain specific for a molecule targeted for reduction or elimination, e.g., a cell surface antigen or a soluble antigen. In preferred embodiments, the binding domain comprises or consists of an antigen binding site (e.g., comprising a variable heavy chain sequence and variable light chain sequence or six CDRs from an antibody placed into alternative framework regions (e.g., human framework regions optionally comprising one or more amino acid substitutions).

The term “binding affinity”, as used herein, includes the strength of a binding interaction and therefore includes both the actual binding affinity as well as the apparent binding affinity. The actual binding affinity is a ratio of the association rate over the disassociation rate. Therefore, conferring or optimizing binding affinity includes altering either or both of these components to achieve the desired level of binding affinity. The apparent affinity can include, for example, the avidity of the interaction.

The term “binding free energy” or “free energy of binding”, as used herein, includes its art-recognized meaning, and, in particular, as applied to binding site-ligand or Fc-FcR interactions in a solvent. Reductions in binding free energy enhance affinities, whereas increases in binding free energy reduce affinities.

The term “specificity” includes the number of potential binding sites which specifically bind (e.g., immunoreact with) a given target. A binding polypeptide may be monospecific and contain one or more binding sites which specifically bind the same target (e.g., the same epitope) or the binding polypeptide may be multispecific and contain two or more binding sites which specifically bind different regions of the same target (e.g., different epitopes) or different targets. In one embodiment, multispecific binding polypeptide (e.g., a bispecific polypeptide) having binding specificity for more than one target molecule (e.g., more than one antigen or more than one epitope on the same antigen) can be made. In another embodiment, the multispecific binding polypeptide has at least one binding domain specific for a molecule targeted for reduction or elimination and at least one binding domain specific for a target molecule on a cell. In another embodiment, the multispecific binding polypeptide has at least one binding domain specific for a molecule targeted for reduction or elimination and at least one binding domain specific for a drug. In yet another embodiment, the multispecific binding polypeptide has at least one binding domain specific for a molecule targeted for reduction or elimination and at least one binding domain specific for a prodrug. In yet another embodiment, the multispecific binding polypeptides are tetravalent antibodies that have two binding domains specific for one target molecule and two binding sites specific for the second target molecule.

As used herein the term “valency” refers to the number of potential binding domains in a binding polypeptide or protein. Each binding domain specifically binds one target molecule. When a binding polypeptide comprises more than one binding domain, each binding domain may specifically bind the same or different molecules (e.g., may bind to different ligands or different antigens, or different epitopes on the same antigen). In one embodiment, the binding polypeptides of the invention are monovalent. In another embodiment, the binding polypeptides of the invention are multivalent. In another embodiment, the binding polypeptides of the invention are bivalent. In another embodiment, the binding polypeptides of the invention are trivalent. In yet another embodiment, the binding polypeptides of the invention are tetravalent.

In certain aspects, the binding polypeptides of invention employ polypeptide linkers. As used herein, the term “polypeptide linkers” refers to a peptide or polypeptide sequence (e.g., a synthetic peptide or polypeptide sequence) which connects two domains in a linear amino acid sequence of a polypeptide chain. For example, polypeptide linkers may be used to connect a binding site to an Fc region (or Fc moiety) of an Fc polypeptide of the invention. Preferably, such polypeptide linkers provide flexibility to the polypeptide molecule. For example, in one embodiment, a VH domain or VL domain is fused or linked to a polypeptide linker and the N- or C-terminus of the polypeptide linker is attached to the C- or N-terminus of an Fc region (or Fc moiety) and the N-terminus of the polypeptide linker is attached to the N- or C-terminus of the VH or VL domain). In certain embodiments the polypeptide linker is used to connect (e.g., genetically fuse) two Fc moieties or domains of an scFc polypeptide. Such polypeptide linkers are also referred to herein as Fc connecting polypeptides. As used herein, the term “Fc connecting polypeptide” refers specifically to a linking polypeptide which connects (e.g., genetically fuses) two Fc moieties or domains. A binding molecule of the invention may comprise more than one peptide linker.

As used herein the term “properly folded polypeptide” includes polypeptides (e.g., binding polypeptides of the invention) in which all of the functional domains comprising the polypeptide are distinctly active. As used herein, the term “improperly folded polypeptide” includes polypeptides in which at least one of the functional domains of the polypeptide is not active. As used herein, a “properly folded Fc polypeptide” or “properly folded Fc region” comprises an Fc region (e.g., an scFc region) in which at least two component Fc moieties are properly folded such that the resulting Fc region comprises at least one effector function.

As used herein, the term “immunoglobulin” includes a polypeptide having a combination of two heavy and two light chains whether or not it possesses any relevant specific immunoreactivity. As used herein, the term “antibody” refers to such assemblies (e.g., intact antibody molecules, antibody fragments, or variants thereof) which have significant known specific immunoreactive activity to an antigen of interest (e.g. a tumor associated antigen). Antibodies and immunoglobulins comprise light and heavy chains, with or without an interchain covalent linkage between them. Basic immunoglobulin structures in vertebrate systems are relatively well understood.

As will be discussed in more detail below, the generic term “antibody” includes five distinct classes of antibody that can be distinguished biochemically. Fc moieties from each class of antibodies are clearly within the scope of the present invention, the following discussion will generally be directed to the IgG class of immunoglobulin molecules. With regard to IgG, immunoglobulins comprise two identical light polypeptide chains of molecular weight approximately 23,000 Daltons, and two identical heavy chains of molecular weight 53,000-70,000. The four chains are joined by disulfide bonds in a “Y” configuration wherein the light chains bracket the heavy chains starting at the mouth of the “Y” and continuing through the variable domain.

Light chains of an immunoglobulin are classified as either kappa or lambda (κ, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG₁, IgG₂, IgG₃, IgG₄, IgA₁, etc. are well characterized and are known to confer functional specialization. Modified versions of each of these classes and isotypes are readily discernable to the skilled artisan in view of the instant disclosure and, accordingly, are within the scope of the instant invention.

Both the light and heavy chains are divided into regions of structural and functional homology. The term “region” refers to a part or portion of a single immunoglobulin (as is the case with the term “Fc region”) or a single antibody chain and includes constant regions or variable regions, as well as more discrete parts or portions of said domains. For example, light chain variable domains include “complementarity determining regions” or “CDRs” interspersed among “framework regions” or “FRs”, as defined herein.

Certain regions of an immunoglobulin may be defined as “constant” (C) regions or “variable” (V) regions, based on the relative lack of sequence variation within the regions of various class members in the case of a “constant region”, or the significant variation within the regions of various class members in the case of a “variable regions”. The terms “constant region” and “variable region” may also be used functionally. In this regard, it will be appreciated that the variable regions of an immunoglobulin or antibody determine antigen recognition and specificity. Conversely, the constant regions of an immunoglobulin or antibody confer important effector functions such as secretion, transplacental mobility, Fc receptor binding, complement binding, and the like. The subunit structures and three dimensional configuration of the constant regions of the various immunoglobulin classes are well known.

The constant and variable regions of immunoglobulin heavy and light chains are folded into domains. The term “domain” refers to an independently folding, globular region of a heavy or light chain polypeptide comprising peptide loops (e.g., comprising 3 to 4 peptide loops) stabilized, for example, by β-pleated sheet and/or intrachain disulfide bond. Constant region domains on the light chain of an immunoglobulin are referred to interchangeably as “light chain constant region domains”, “CL regions” or “CL domains”. Constant domains on the heavy chain (e.g. hinge, CH1, CH2 or CH3 domains) are referred to interchangeably as “heavy chain constant region domains”, “CH” region domains or “CH domains”. Variable domains on the light chain are referred to interchangeably as “light chain variable region domains”, “VL region domains or “VL domains”. Variable domains on the heavy chain are referred to interchangeably as “heavy chain variable region domains”, “VH region domains” or “VH domains”.

By convention the numbering of the variable and constant region domains increases as they become more distal from the antigen binding site or amino-terminus of the immunoglobulin or antibody. The N-terminus of each heavy and light immunoglobulin chain is a variable region and at the C-terminus is a constant region; the CH3 and CL domains actually comprise the carboxy-terminus of the heavy and light chain, respectively. Accordingly, the domains of a light chain immunoglobulin are arranged in a VL-CL orientation, while the domains of the heavy chain are arranged in the VH-CH1-hinge-CH2-CH3 orientation.

Amino acid positions in a heavy chain constant region, including amino acid positions in the CH1, hinge, CH2, and CH3 domains, are numbered herein according to the EU index numbering system (see Kabat et al., in “Sequences of Proteins of Immunological Interest”, U.S. Dept. Health and Human Services, 5^{th} edition, 1991). In contrast, amino acid positions in a light chain constant region (e.g. CL domains) are numbered herein according to the Kabat index numbering system (see Kabat et al., ibid).

As used herein, the term “V_{H} domain” includes the amino terminal variable domain of an immunoglobulin heavy chain, and the term “V_{L} domain” includes the amino terminal variable domain of an immunoglobulin light chain according to the Kabat index numbering system.

As used herein, the term “CH1 domain” includes the first (most amino terminal) constant region domain of an immunoglobulin heavy chain that extends, e.g., from about EU positions 118-215. The CH1 domain is adjacent to the V_{H} domain and amino terminal to the hinge region of an immunoglobulin heavy chain molecule, and does not form a part of the Fc region of an immunoglobulin heavy chain. In one embodiment, a binding polypeptide of the invention comprises a CH1 domain derived from an immunoglobulin heavy chain molecule (e.g., a human IgG1 or IgG4 molecule).

As used herein, the term “hinge region” includes the portion of a heavy chain molecule that joins the CH1 domain to the CH2 domain. This hinge region comprises approximately 25 residues and is flexible, thus allowing the two N-terminal antigen binding regions to move independently. Hinge regions can be subdivided into three distinct domains: upper, middle, and lower hinge domains (Roux et al. J. Immunol. 1998, 161:4083).

As used herein, the term “CH2 domain” includes the portion of a heavy chain immunoglobulin molecule that extends, e.g., from about EU positions 231-340. The CH2 domain is unique in that it is not closely paired with another domain. Rather, two N-linked branched carbohydrate chains are interposed between the two CH2 domains of an intact native IgG molecule. In one embodiment, an binding polypeptide of the invention comprises a CH2 domain derived from an IgG1 molecule (e.g. a human IgG1 molecule). In another embodiment, an binding polypeptide of the invention comprises a CH2 domain derived from an IgG4 molecule (e.g., a human IgG4 molecule). In an exemplary embodiment, a polypeptide of the invention comprises a CH2 domain (EU positions 231-340), or a portion thereof.

As used herein, the term “CH3 domain” includes the portion of a heavy chain immunoglobulin molecule that extends approximately 110 residues from N-terminus of the CH2 domain, e.g., from about position 341-446b (EU numbering system). The CH3 domain typically forms the C-terminal portion of the antibody. In some immunoglobulins, however, additional domains may extend from CH3 domain to form the C-terminal portion of the molecule (e.g. the CH4 domain in the μ chain of IgM and the ξ chain of IgE). In one embodiment, an binding polypeptide of the invention comprises a CH3 domain derived from an IgG1 molecule (e.g., a human IgG1 molecule). In another embodiment, an binding polypeptide of the invention comprises a CH3 domain derived from an IgG4 molecule (e.g., a human IgG4 molecule).

As used herein, the term “CL domain” includes the first (most amino terminal) constant region domain of an immunoglobulin light chain that extends, e.g. from about Kabat position 107A-216. The CL domain is adjacent to the V_{L} domain. In one embodiment, an binding polypeptide of the invention comprises a CL domain derived from a kappa light chain (e.g., a human kappa light chain).

As indicated above, the variable regions of an antibody allow it to selectively recognize and specifically bind epitopes on antigens. That is, the V_{L} domain and V_{H} domain of an antibody combine to form the variable region (Fv) that defines a three dimensional antigen binding site. This quaternary antibody structure forms the antigen binding site present at the end of each arm of the Y. More specifically, the antigen binding site is defined by three complementary determining regions (CDRs) on each of the heavy and light chain variable regions.

As used herein, the term “antigen binding site” includes a site that specifically binds (immunoreacts with) an antigen such as a cell surface or soluble antigen). In one embodiment, the binding site includes an immunoglobulin heavy chain and light chain variable region and the binding site formed by these variable regions determines the specificity of the antibody. An antigen binding site is formed by variable regions that vary from one polypeptide to another. In one embodiment, a binding polypeptide of the invention comprises an antigen binding site comprising at least one heavy or light chain CDR of an antibody molecule (e.g., the sequence of which is known in the art or described herein). In another embodiment, a binding polypeptide of the invention comprises an antigen binding site comprising at least two CDRs from one or more antibody molecules. In another embodiment, a binding polypeptide of the invention comprises an antigen binding site comprising at least three CDRs from one or more antibody molecules. In another embodiment, a binding polypeptide of the invention comprises an antigen binding site comprising at least four CDRs from one or more antibody molecules. In another embodiment, a binding polypeptide of the invention comprises an antigen binding site comprising at least five CDRs from one or more antibody molecules. In another embodiment, a binding polypeptide of the invention comprises an antigen binding site comprising six CDRs from an antibody molecule. Exemplary antibody molecules comprising at least one CDR that can be included in the subject binding polypeptides are known in the art and exemplary molecules are described herein.

As used herein, the term “CDR” or “complementarity determining region” means the noncontiguous antigen combining sites found within the variable region of both heavy and light chain polypeptides. These particular regions have been described by Kabat et al., J. Biol. Chem. 252, 6609-6616 (1977) and Kabat et al., Sequences of protein of immunological interest. (1991), and by Chothia et al., J. Mol. Biol. 196:901-917 (1987) and by MacCallum et al., J. Mol. Biol. 262:732-745 (1996) where the definitions include overlapping or subsets of amino acid residues when compared against each other. The amino acid residues which encompass the CDRs as defined by each of the above cited references are set forth for comparison. Preferably, the term “CDR” is a CDR as defined by Kabat based on sequence comparisons.

CDR Definitions

| | Kabat¹ | Chothia² | MacCallum³ |
|---|---|---|---|
| V_{H} CDR1 | 31-35 | 26-32 | 30-35 |
| V_{H} CDR2 | 50-65 | 53-55 | 47-58 |
| V_{H} CDR3 | 95-102 | 96-101 | 93-101 |
| V_{L} CDR1 | 24-34 | 26-32 | 30-36 |
| V_{L} CDR2 | 50-56 | 50-52 | 46-55 |
| V_{L} CDR3 | 89-97 | 91-96 | 89-96 |

¹Residue numbering follows the nomenclature of Kabat et al., supra
²Residue numbering follows the nomenclature of Chothia et al., supra
³Residue numbering follows the nomenclature of MacCallum et al., supra

The term “framework region” or “FR region” as used herein, includes the amino acid residues that are part of the variable region, but are not part of the CDRs (e.g., using the Kabat definition of CDRs). Therefore, a variable region framework is between about 100-120 amino acids in length but includes only those amino acids outside of the CDRs. For the specific example of a heavy chain variable region and for the CDRs as defined by Kabat et al., framework region 1 corresponds to the domain of the variable region encompassing amino acids 1-30; framework region 2 corresponds to the domain of the variable region encompassing amino acids 36-49; framework region 3 corresponds to the domain of the variable region encompassing amino acids 66-94, and framework region 4 corresponds to the domain of the variable region from amino acids 103 to the end of the variable region. The framework regions for the light chain are similarly separated by each of the light chain variable region CDRs. Similarly, using the definition of CDRs by Chothia et al. or McCallum et al. the framework region boundaries are separated by the respective CDR termini as described above. In preferred embodiments, the CDRs are as defined by Kabat.

In naturally occurring antibodies, the six CDRs present on each monomeric antibody are short, non-contiguous sequences of amino acids that are specifically positioned to form the antigen binding site as the antibody assumes its three dimensional configuration in an aqueous environment. The remainder of the heavy and light variable domains show less inter-molecular variability in amino acid sequence and are termed the framework regions. The framework regions largely adopt a β-sheet conformation and the CDRs form loops which connect, and in some cases form part of, the β-sheet structure. Thus, these framework regions act to form a scaffold that provides for positioning the six CDRs in correct orientation by inter-chain, non-covalent interactions. The antigen binding site formed by the positioned CDRs defines a surface complementary to the epitope on the immunoreactive antigen. This complementary surface promotes the non-covalent binding of the antibody to the immunoreactive antigen epitope. The position of CDRs can be readily identified by one of ordinary skill in the art.

In certain embodiments, the binding polypeptides of the invention comprise at least two antigen binding domains (e.g., within the same binding polypeptide (e.g, at both the N- and C-terminus of a single polypeptide) or linked to each component binding polypeptide of a mutimeric binding protein of the invention) that provide for the association of the binding polypeptide with the selected antigen. The antigen binding domains need not be derived from the same immunoglobulin molecule. In this regard, the variable region may or may not be derived from any type of animal that can be induced to mount a humoral response and generate immunoglobulins against the desired antigen. As such, the variable region may be, for example, of mammalian origin e.g., may be human, murine, non-human primate (such as cynomolgus monkeys, macaques, etc.), lupine, camelid (e.g., from camels, llamas and related species).

The term “antibody variant” or “modified antibody” includes an antibody which does not occur in nature and which has an amino acid sequence or amino acid side chain chemistry which differs from that of a naturally-derived antibody by at least one amino acid or amino acid modification as described herein. As used herein, the term “antibody variant” includes synthetic forms of antibodies which are altered such that they are not naturally occurring, e.g., antibodies that comprise at least two heavy chain portions but not two complete heavy chains (such as, domain deleted antibodies or minibodies); multispecific forms of antibodies (e.g., bispecific, trispecific, etc.) altered to bind to two or more different antigens or to different epitopes on a single antigen); heavy chain molecules joined to scFv molecules; single-chain antibodies; diabodies; triabodies; and antibodies with altered effector function and the like.

As used herein the term “scFv molecule” includes binding molecules which consist of one light chain variable domain (VL) or portion thereof, and one heavy chain variable domain (VH) or portion thereof, wherein each variable domain (or portion thereof) is derived from the same or different antibodies. scFv molecules preferably comprise an scFv linker interposed between the VH domain and the VL domain. ScFv molecules are known in the art and are described, e.g., in U.S. Pat. No. 5,892,019, Ho et al. 1989. Gene 77:51; Bird et al. 1988 Science 242:423; Pantoliano et al. 1991. Biochemistry 30:10117; Milenic et al. 1991. Cancer Research 51:6363; Takkinen et al. 1991. Protein Engineering 4:837.

A “scFv linker” as used herein refers to a moiety interposed between the VL and VH domains of the scFv. scFv linkers preferably maintain the scFv molecule in a antigen binding conformation. In one embodiment, a scFv linker comprises or consists of an scFv linker peptide. In certain embodiments, a scFv linker peptide comprises or consists of a gly-ser polypeptide linker. In other embodiments, a scFv linker comprises a disulfide bond.

As used herein, the term “gly-ser polypeptide linker” refers to a peptide that consists of glycine and serine residues. An exemplary gly/ser polypeptide linker comprises the amino acid sequence (Gly₄ Ser)ₙ. In one embodiment, n=1. In one embodiment, n=2. In another embodiment, n=3, i.e., (Gly₄ Ser)₃. In another embodiment, n=4, i.e., (Gly₄ Ser)₄. In another embodiment, n=5. In yet another embodiment, n=6. In another embodiment, n=7. In yet another embodiment, n=8. In another embodiment, n=9. In yet another embodiment, n=10. Another exemplary gly/ser polypeptide linker comprises the amino acid sequence Ser(Gly₄Ser)ₙ. In one embodiment, n=1. In one embodiment, n=2. In a preferred embodiment, n=3. In another embodiment, n=4. In another embodiment, n=5. In yet another embodiment, n=6.

A used herein, the term “native cysteine” shall refer to a cysteine amino acid that occurs naturally at a particular amino acid position of a polypeptide and which has not been modified, introduced, or altered by the hand of man. The term “engineered cysteine residue or analog thereof” or “engineered cysteine or analog thereof” shall refer to a non-native cysteine residue or a cysteine analog (e.g. thiol-containing analogs such as thiazoline-4-carboxylic acid and thiazolidine-4 carboxylic acid (thioproline, Th)), which is introduced by synthetic means (e.g. by recombinant techniques, in vitro peptide synthesis, by enzymatic or chemical coupling of peptides or some combination of these techniques) into an amino acid position of a polypeptide that does not naturally contain a cysteine residue or analog thereof at that position.

As used herein the term “disulfide bond” includes the covalent bond formed between two sulfur atoms. The amino acid cysteine comprises a thiol group that can form a disulfide bond or bridge with a second thiol group. In most naturally occurring IgG molecules, the CH1 and CL regions are linked by native disulfide bonds and the two heavy chains are linked by two native disulfide bonds at positions corresponding to 239 and 242 using the Kabat numbering system (position 226 or 229, EU numbering system).

As used herein, the term “bonded cysteine” shall refer to a native or engineered cysteine residue within a polypeptide which forms a disulfide bond or other covalent bond with a second native or engineered cysteine or other residue present within the same or different polypeptide. An “intrachain bonded cysteine” shall refer to a bonded cysteine that is covalently bonded to a second cysteine present within the same polypeptide (ie. an intrachain disulfide bond). An “interchain bonded cysteine” shall refer to a bonded cysteine that is covalently bonded to a second cysteine present within a different polypeptide (ie. an interchain disulfide bond).

As used herein, the term “free cysteine” refers to a native or engineered cysteine amino acid residues within a polypeptide sequence (and analogs or mimetics thereof, e.g. thiazoline-4-carboxylic acid and thiazolidine-4 carboxylic acid (thioproline, Th)) that exists in a substantially reduced form. Free cysteines are preferably capable of being modified with an effector moiety of the invention.

The term “thiol modification reagent” shall refer to a chemical agent that is capable of selectively reacting with the thiol group of an engineered cysteine residue or analog thereof in a binding polypeptide (e.g., within an polypeptide linker of a binding polypeptide), and thereby providing means for site-specific chemical addition or crosslinking of effector moieties to the binding polypeptide, thereby forming a modified binding polypeptide. Preferably the thiol modification reagent exploits the thiol or sulfhydryl functional group which is present in a free cysteine residue. Exemplary thiol modification reagents include maleimides, alkyl and aryl halides, α-haloacyls, and pyridyl disulfides.

The term “functional moiety” includes moieties which, preferably, add a desirable function to the binding polypeptide. Preferably, the function is added without significantly altering an intrinsic desirable activity of the polypeptide, e.g., the antigen-binding activity of the molecule. A binding polypeptide of the invention may comprise one or more functional moieties, which may be the same or different. Examples of useful functional moieties include, but are not limited to, an effector moiety, an affinity moiety, and a blocking moiety.

Exemplary blocking moieties include moieties of sufficient steric bulk and/or charge such that reduced glycosylation occurs, for example, by blocking the ability of a glycosidase to glycosylate the polypeptide. The blocking moiety may additionally or alternatively, reduce effector function, for example, by inhibiting the ability of the Fc region to bind a receptor or complement protein. Preferred blocking moieties include cysteine adducts, cysteine, mixed disulfide adducts, and PEG moieties. Exemplary detectable moieties include fluorescent moieties, radioisotopic moieties, radiopaque moieties, and the like.

With respect to conjugation of chemical moieties, the term “linking moiety” includes moieties which are capable of linking a functional moiety to the remainder of the binding polypeptide. The linking moiety may be selected such that it is cleavable or non-cleavable. Uncleavable linking moieties generally have high systemic stability, but may also have unfavorable pharmacokinetics.

The term “spacer moiety” is a nonprotein moiety designed to introduce space into a molecule. In one embodiment a spacer moiety may be an optionally substituted chain of 0 to 100 atoms, selected from carbon, oxygen, nitrogen, sulfur, etc. In one embodiment, the spacer moiety is selected such that it is water soluble. In another embodiment, the spacer moiety is polyalkylene glycol, e.g., polyethylene glycol or polypropylene glycol.

The terms “PEGylation moiety” or “PEG moiety” includes a polyalkylene glycol compound or a derivative thereof, with or without coupling agents or derivitization with coupling or activating moieties (e.g., with thiol, triflate, tresylate, azirdine, oxirane, or preferably with a maleimide moiety, e.g., PEG-maleimide). Other appropriate polyalkylene glycol compounds include, maleimido monomethoxy PEG, activated PEG polypropylene glycol, but also charged or neutral polymers of the following types: dextran, colominic acids, or other carbohydrate based polymers, polymers of amino acids, and biotin derivatives.

As used herein, the term “effector moiety” (E) may comprise diagnostic and therapeutic agents (e.g. proteins, nucleic acids, lipids, drug moieties, and fragments thereof) with biological or other functional activity. For example, a binding polypeptide comprising an effector moiety conjugated to a binding polypeptide has at least one additional function or property as compared to the unconjugated polypeptide. For example, the conjugation of a cytotoxic drug moiety (e.g., an effector moiety) to a binding polypeptide (e.g., via its polypeptide linker) results in the formation of a modified polypeptide with drug cytotoxicity as second function (i.e. in addition to antigen binding). In another example, the conjugation of a second binding polypeptide to the first binding polypeptide may confer additional binding properties.

In one aspect, wherein the effector moiety is a genetically encoded therapeutic or diagnostic protein or nucleic acid, the effector moiety may be synthesized or expressed by either peptide synthesis or recombinant DNA methods that are well known in the art. In another aspect, wherein the effector is a non-genetically encoded peptide or a drug moiety, the effector moiety may be synthesized artificially or purified from a natural source.

As used herein, the term “drug moiety” includes anti-inflammatory, anticancer, anti-infective (e.g., anti-fungal, antibacterial, anti-parasitic, anti-viral, etc.), and anesthetic therapeutic agents. In a further embodiment, the drug moiety is an anticancer or cytotoxic agent. Compatible drug moieties may also comprise prodrugs.

As used herein, the term “prodrug” refers to a precursor or derivative form of a pharmaceutically active agent that is less active, reactive or prone to side effects as compared to the parent drug and is capable of being enzymatically activated or otherwise converted into a more active form in vivo. Prodrugs compatible with the invention include, but are not limited to, phosphate-containing prodrugs, amino acid-containing prodrugs, thiophosphate-containing prodrugs, sulfate containing prodrugs, peptide containing prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs that can be converted to the more active cytotoxic free drug. One skilled in the art may make chemical modifications to the desired drug moiety or its prodrug in order to make reactions of that compound more convenient for purposes of preparing modified binding proteins of the invention. The drug moieties also include derivatives, pharmaceutically acceptable salts, esters, amides, and ethers of the drug moieties described herein. Derivatives include modifications to drugs identified herein which may improve or not significantly reduce a particular drug's desired therapeutic activity.

As used herein, the term “anticancer agent” includes agents which are detrimental to the growth and/or proliferation of neoplastic or tumor cells and may act to reduce, inhibit or destroy malignancy. Examples of such agents include, but are not limited to, cytostatic agents, alkylating agents, antibiotics, cytotoxic nucleosides, tubulin binding agents, hormones and hormone antagonists, and the like. Any agent that acts to retard or slow the growth of immunoreactive cells or malignant cells is within the scope of the present invention.

An “affinity tag” or an “affinity moiety” is a chemical moiety that is attached to one or more of the binding polypeptide, polypeptide linker, or effector moiety in order to facilitate its separation from other components during a purification procedure. Exemplary affinity domains include the His tag, chitin binding domain, maltose binding domain, biotin, and the like.

An “affinity resin” is a chemical surface capable of binding the affinity domain with high affinity to facilitate separation of the protein bound to the affinity domain from the other components of a reaction mixture. Affinity resins can be coated on the surface of a solid support or a portion thereof. Alternatively, the affinity resin can comprise the solid support. Such solid supports can include a suitably modified chromatography column, microtiter plate, bead, or biochip (e.g. glass wafer). Exemplary affinity resins are comprised of nickel, chitin, amylase, and the like.

The term “vector” or “expression vector” is used herein to mean vectors used in accordance with the present invention as a vehicle for introducing into and expressing a desired polynucleotide in a cell. As known to those skilled in the art, such vectors may easily be selected from the group consisting of plasmids, phages, viruses and retroviruses. In general, vectors compatible with the instant invention will comprise a selection marker, appropriate restriction sites to facilitate cloning of the desired gene and the ability to enter and/or replicate in eukaryotic or prokaryotic cells.

For the purposes of this invention, numerous expression vector systems may be employed. For example, one class of vector utilizes DNA elements which are derived from animal viruses such as bovine papilloma virus, polyoma virus, adenovirus, vaccinia virus, baculovirus, retroviruses (RSV, MMTV or MOMLV) or SV40 virus. Others involve the use of polycistronic systems with internal ribosome binding sites. Exemplary vectors include those described in U.S. Pat. Nos. 6,159,730 and 6,413,777, and U.S. Patent Application No. 2003 0157641 A1. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation. In one embodiment, an inducible expression system can be employed. Additional elements may also be needed for optimal synthesis of mRNA. These elements may include signal sequences, splice signals, as well as transcriptional promoters, enhancers, and termination signals. In one embodiment, a secretion signal, e.g., any one of several well characterized bacterial leader peptides (e.g., pelB, phoA, or ompA), can be fused in-frame to the N terminus of a polypeptide of the invention to obtain optimal secretion of the polypeptide. (Lei et al. (1988), Nature, 331:543; Better et al. (1988) Science, 240:1041; Mullinax et al., (1990). PNAS, 87:8095).

The term “host cell” refers to a cell that has been transformed with a vector constructed using recombinant DNA techniques and encoding at least one heterologous gene. In descriptions of processes for isolation of proteins from recombinant hosts, the terms “cell” and “cell culture” are used interchangeably to denote the source of protein unless it is clearly specified otherwise. In other words, recovery of protein from the “cells” may mean either from spun down whole cells, or from the cell culture containing both the medium and the suspended cells. The host cell line used for protein expression is most preferably of mammalian origin; those skilled in the art are credited with ability to preferentially determine particular host cell lines which are best suited for the desired gene product to be expressed therein. Exemplary host cell lines include, but are not limited to, DG44 and DUXB11 (Chinese Hamster Ovary lines, DHFR minus), HELA (human cervical carcinoma), CVI (monkey kidney line), COS (a derivative of CVI with SV40 T antigen), R1610 (Chinese hamster fibroblast) BALBC/3T3 (mouse fibroblast), HAK (hamster kidney line), SP2/O (mouse myeloma), P3×63-Ag3.653 (mouse myeloma), BFA-1c1BPT (bovine endothelial cells), RAJI (human lymphocyte) and 293 (human kidney). CHO cells are particularly preferred. Host cell lines are typically available from commercial services, the American Tissue Culture Collection or from published literature. The polypeptides of the invention can also be expressed in non-mammalian cells such as bacteria or yeast or plant cells. In this regard it will be appreciated that various unicellular non-mammalian microorganisms such as bacteria can also be transformed; i.e. those capable of being grown in cultures or fermentation. Bacteria, which are susceptible to transformation, include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the polypeptides typically become part of inclusion bodies. The polypeptides must be isolated, purified and then assembled into functional molecules.

In addition to prokaryotes, eukaryotic microbes may also be used. Saccharomyces cerevisiae, or common baker's yeast, is the most commonly used among eukaryotic microorganisms although a number of other strains are commonly available including Pichia pastoris. For expression in Saccharomyces, the plasmid YRp7, for example, (Stinchcomb et al., (1979), Nature, 282:39; Kingsman et al., (1979), Gene, 7:141; Tschemper et al., (1980), Gene, 10:157) is commonly used. This plasmid already contains the TRP1 gene which provides a selection marker for a mutant strain of yeast lacking the ability to grow in tryptophan, for example ATCC No. 44076 or PEP4-1 (Jones, (1977), Genetics, 85:12). The presence of the trpl lesion as a characteristic of the yeast host cell genome then provides an effective environment for detecting transformation by growth in the absence of tryptophan.

In vitro production allows scale-up to give large amounts of the desired altered binding polypeptides of the invention. Techniques for mammalian cell cultivation under tissue culture conditions are known in the art and include homogeneous suspension culture, e.g. in an airlift reactor or in a continuous stirrer reactor, or immobilized or entrapped cell culture, e.g. in hollow fibers, microcapsules, on agarose microbeads or ceramic cartridges. If necessary and/or desired, the solutions of polypeptides can be purified by the customary chromatography methods, for example gel filtration, ion-exchange chromatography, hydrophobic interaction chromatography (HIC, chromatography over DEAE-cellulose or affinity chromatography.

As used herein, “tumor-associated antigens” means an antigen which is generally associated with tumor cells, i.e., occurring at the same or to a greater extent as compared with normal cells. More generally, tumor associated antigens comprise any antigen that provides for the localization of immunoreactive antibodies at a neoplastic cell irrespective of its expression on non-malignant cells. Such antigens may be relatively tumor specific and limited in their expression to the surface of malignant cells. Alternatively, such antigens may be found on both malignant and non-malignant cells. In certain embodiments, the binding polypeptides of the present invention preferably bind to tumor-associated antigens. Accordingly, the binding polypeptide of the invention may be derived, generated or fabricated from any one of a number of antibodies that react with tumor associated molecules.

As used herein, the term “malignancy” refers to a non-benign tumor or a cancer. As used herein, the term “cancer” includes a malignancy characterized by deregulated or uncontrolled cell growth. Exemplary cancers include: carcinomas, sarcomas, leukemias, and lymphomas. The term “cancer” includes primary malignant tumors (e.g., those whose cells have not migrated to sites in the subject's body other than the site of the original tumor) and secondary malignant tumors (e.g., those arising from metastasis, the migration of tumor cells to secondary sites that are different from the site of the original tumor).

As used herein, the phrase “subject that would benefit from administration of a binding polypeptide” includes subjects, such as mammalian subjects, that would benefit from administration of binding polypeptides used, e.g., for detection of an antigen recognized by a binding polypeptide of the invention (e.g., for a diagnostic procedure) and/or from treatment with a binding polypeptide to reduce or eliminate the target recognized by the binding polypeptide. For example, in one embodiment, the subject may benefit from reduction or elimination of a soluble or particulate molecule from the circulation or serum (e.g., a toxin or pathogen) or from reduction or elimination of a population of cells expressing the target (e.g., tumor cells). As discussed above, the binding polypeptide can be used in unconjugated form or can be conjugated, e.g., to a drug, prodrug, or an isotope, to form a modified binding polypeptide for administering to said subject.

The term “pegylation”, “polyethylene glycol”, or “PEG” includes a polyalkylene glycol compound or a derivative thereof, with or without coupling agents or derviatization with coupling or activating moieties (e.g., with thiol, triflate, tresylate, azirdine, oxirane, or preferably with a maleimide moiety, e.g., PEG-maleimide). Other appropriate polyalkylene glycol compounds include, but are not limited to, maleimido monomethoxy PEG, activated PEG polypropylene glycol, but also charged or neutral polymers of the following types: dextran, colominic acids, or other carbohydrate based polymers, polymers of amino acids, and biotin derivatives.

### (II) Parental Fc Polypeptides

The variant Fc polypeptides may be derived from parental or starting Fc polypeptide known in the art. In a preferred embodiment, the parental Fc polypeptide is as an antibody, and preferably IgG immunoglobulin, e.g., of the subtype IgG1, IgG2, IgG3, or IgG4, and preferably, of the subtype IgG1 or IgG4. The parental Fc polypeptide comprises an Fc region derived from an immunoglobulin, but may optionally further comprise a binding site which operably linked or fused to the Fc region. In a preferred embodiment, the forgoing polypeptide binds to an antigen such as a ligand, cytokine, receptor, cell surface antigen, or cancer cell antigen. Although the Examples herein employ an IgG antibody, it is understood that the method can be equally applied to an Fc region within any Fc polypeptide. When the Fc polypeptide is an antibody, the antibody can be synthetic, naturally-derived (e.g., from serum), produced by a cell line (e.g., a hybridoma), or produced in a transgenic organism.

In certain embodiments, the Fc polypeptides of the invention comprise a single Fc moiety of an Fc region. In other embodiments, the Fc polypeptide is a dcFc polypeptide. A dcFc polypeptide refers to a polypeptide comprising a dimeric Fc (or dcFc) region. In other embodiments, the Fc polypeptides of the invention are scFc polypeptides. As used herein, the term scFc polypeptide refers to a polypeptide comprising a single-chain Fc (scFc) region, e.g., a scFc polypeptide comprising at least two Fc moieties that are genetically fused, e.g., via a flexible polypeptide linker interposed between at least two of the Fc moieties. Exemplary scFc regions are disclosed in PCT Application No. PCT/US2008/006260, filed May 14, 2008, which is incorporated by reference herein.

In certain embodiments, the polypeptides of the invention may comprise a Fc region comprising Fc moieties of the same, or substantially the same, sequence composition (herein termed a “homomeric Fc region”). In other embodiments, the polypeptides of the invention may comprise a Fc region comprising at least two Fc moieties which are of different sequence composition (i.e., herein termed a “heteromeric Fc region”). In certain embodiments, the binding polypeptides of the invention comprise a Fc region comprising at least one insertion or amino acid substitution. In one exemplary embodiment, the heteromeric Fc region comprises an amino acid substitution in a first Fc moiety, but not in a second Fc moiety.

In one embodiment, the binding polypeptide of the invention may comprise a Fc region having two or more of its constituent Fc moieties independently selected from the Fc moieties described herein. In one embodiment, the Fc moieties are the same. In another embodiment, at least two of the Fc moieties are different. For example, the Fc moieties of the Fc polypeptides of the invention comprise the same number of amino acid residues or they may differ in length by one or more amino acid residues (e.g., by about 5 amino acid residues (e.g., 1, 2, 3, 4, or 5 amino acid residues), about 10 residues, about 15 residues, about 20 residues, about 30 residues, about 40 residues, or about 50 residues). In yet other embodiments, the Fc moieties may differ in sequence at or more amino acid positions. For example, at least two of the Fc moieties may differ at about 5 amino acid positions (e.g., 1, 2, 3, 4, or 5 amino acid positions), about 10 positions, about 15 positions, about 20 positions, about 30 positions, about 40 positions, or about 50 positions)

The parental Fc polypeptides may be assembled together or with other polypeptides to form multimeric Fc polypeptides or proteins (also, referred to herein as “multimers”). The multimeric Fc polypeptide or proteins of the invention comprise at least one parental Fc polypeptide of the invention. Accordingly, the parental polypeptide includes without limitation monomeric as well as multimeric (e.g., dimeric, trimeric, tetrameric, and hexameric) Fc polypeptides or proteins and the like. In certain embodiments, the constituent Fc polypeptides of said multimers are the same (ie. homomeric multimers, e.g. homodimers, homotrimers, homotetramers). In other embodiments, at least two constituent Fc polypeptides of the multimeric proteins of the invention are different (ie. heteromeric multimers, e.g. heterodimers, heterotrimers, heterotetramers). In certain embodiments, at least two of the Fc polypeptides are capable of forming a dimer.

In another embodiment, an Fc polypeptide of the invention comprises a dimeric Fc region (either a single chain polypeptide which forms a domer or a two chain polypeptide which forms a dimer) and is monomeric with respect to the biologically active moiety present in the molecule. For example, such an Fc construct can comprise one biologically active moiety only. One or two chain stabilized Fc monomeric constructs are desirable, e.g., when cross-linking of target molecules is not desired (for example, in the case of certain antibodies, e.g., anti-CD40 antibodies). In another embodiment, such an Fc construct can comprise two different biologically active moieties. In yet another embodiment, such an Fc construct can comprise two of the same biologically active moieties. In yet another embodiment, such an Fc construct can comprise more than two of the same biologically active moieties.

### A. Fc Moieties

Fc moieties useful for producing the parental Fc polypeptides of the present invention may be obtained from a number of different sources. In preferred embodiments, a Fc moiety of the binding polypeptide is derived from a human immunoglobulin. It is understood, however, that the Fc moiety may be derived from an immunoglobulin of another mammalian species, including for example, a rodent (e.g. a mouse, rat, rabbit, guinea pig) or non-human primate (e.g. chimpanzee, macaque) species. Moreover, the Fc may be derived from any immunoglobulin class, including IgM, IgG, IgD, IgA and IgE, and any immunoglobulin isotype, including IgG1, IgG2, IgG3 and IgG4. In a preferred embodiments, the human isotype IgG1 or IgG4 is used.

A variety of Fc moiety gene sequences (e.g. human constant region gene sequences) are available in the form of publicly accessible deposits. Constant region domains comprising an Fc moiety sequence can be selected having a particular effector function (or lacking a particular effector function) or with a particular modification to reduce immunogenicity. Many sequences of antibodies and antibody-encoding genes have been published and suitable Fc moiety sequences (e.g. hinge, CH2, and/or CH3 sequences, or portions thereof) can be derived from these sequences using art recognized techniques. The genetic material obtained using any of the foregoing methods may then be altered or synthesized to obtain Fc polypeptides of the present invention. It will further be appreciated that the scope of this invention encompasses alleles, variants and mutations of constant region DNA sequences.

Fc moiety sequences can be cloned, e.g., using the polymerase chain reaction and primers which are selected to amplify the domain of interest. To clone an Fc moiety sequence from an antibody, mRNA can be isolated from hybridoma, spleen, or lymph cells, reverse transcribed into DNA, and antibody genes amplified by PCR. PCR amplification methods are described in detail in U.S. Pat. Nos. 4,683,195; 4,683,202; 4,800,159; 4,965,188; and in, e.g., “PCR Protocols: A Guide to Methods and Applications” Innis et al. eds., Academic Press, San Diego, Calif. (1990); Ho et al. 1989. Gene 77:51; Horton et al. 1993. Methods Enzymol. 217:270). PCR may be initiated by consensus constant region primers or by more specific primers based on the published heavy and light chain DNA and amino acid sequences. As discussed above, PCR also may be used to isolate DNA clones encoding the antibody light and heavy chains. In this case the libraries may be screened by consensus primers or larger homologous probes, such as mouse constant region probes. Numerous primer sets suitable for amplification of antibody genes are known in the art (e.g., 5′ primers based on the N-terminal sequence of purified antibodies (Benhar and Pastan. 1994. Protein Engineering 7:1509); rapid amplification of cDNA ends (Ruberti, F. et al. 1994. J. Immunol. Methods 173:33); antibody leader sequences (Larrick et al. 1989 Biochem. Biophys. Res. Commun. 160:1250). The cloning of antibody sequences is further described in Newman et al., U.S. Pat. No. 5,658,570, filed Jan. 25, 1995, which is incorporated by reference herein.

The parental Fc polypeptides of the invention may comprise a single Fc moiety or multiple Fc moieties. Where there are two or more Fc moieties (e.g., 2, 3, 4, 5, 6, 7, 8, 9, 10, or more Fc moieties, at least two of the Fc moieties associate to form a properly folded Fc region (e.g., a dimeric Fc region or a single chain Fc region (scFc)). In one embodiment, the Fc moieties may be of different types. In one embodiment, at least one Fc moiety present in the parental Fc polypeptide comprises a hinge domain or portion thereof. In another embodiment, the parental Fc polypeptide comprises at least one Fc moiety which comprises at least one CH2 domain or portion thereof. In another embodiment, the parental Fc polypeptide comprises at least one Fc moiety which comprises at least one CH3 domain or portion thereof. In another embodiment, the parental Fc polypeptide comprises at least one Fc moiety which comprises at least one CH4 domain or portion thereof. In another embodiment, the parental Fc polypeptide comprises at least one Fc moiety which comprises at least one hinge domain or portion thereof and at least one CH2 domain or portion thereof (e.g, in the hinge-CH2 orientation). In another embodiment, the parental Fc polypeptide comprises at least one Fc moiety which comprises at least one CH2 domain or portion thereof and at least one CH3 domain or portion thereof (e.g, in the CH2-CH3 orientation). In another embodiment, the parental Fc polypeptide comprises at least one Fc moiety comprising at least one hinge domain or portion thereof, at least one CH2 domain or portion thereof, and least one CH3 domain or portion thereof, for example in the orientation hinge-CH2-CH3, hinge-CH3-CH2, or CH2-CH3-hinge.

In certain embodiments, the parental Fc polypeptide comprises at least one complete Fc region derived from one or more immunoglobulin heavy chains (e.g., an Fc moiety including hinge, CH2, and CH3 domains, although these need not be derived from the same antibody). In other embodiments, the parental Fc polypeptide comprises at least two complete Fc regions derived from one or more immunoglobulin heavy chains. In preferred embodiments, the complete Fc moiety is derived from a human IgG immunoglobulin heavy chain (e.g., human IgG1 or human IgG4).

In another embodiment, a parental Fc polypeptide comprises at least one Fc moiety comprising a complete CH3 domain (about amino acids 341-438 of an antibody Fc region according to EU numbering). In another embodiment, a parental Fc polypeptide comprises at least one Fc moiety comprising a complete CH2 domain (about amino acids 231-340 of an antibody Fc region according to EU numbering). In another embodiment, a parental Fc polypeptide comprises at least one Fc moiety comprising at least a CH3 domain, and at least one of a hinge region (about amino acids 216-230 of an antibody Fc region according to EU numbering), and a CH2 domain. In one embodiment, a parental Fc polypeptide comprises at least one Fc moiety comprising a hinge and a CH3 domain. In another embodiment, a parental Fc polypeptide comprises at least one Fc moiety comprising a hinge, a CH₂, and a CH₃ domain. In preferred embodiments, the Fc moiety is derived from a human IgG immunoglobulin heavy chain.

The constant region domains or portions thereof making up an Fc moiety may be derived from different immunoglobulin molecules. For example, a parental Fc polypeptide may comprise a hinge and/or CH2 domain or portion thereof derived from an IgG4 molecule and a CH3 region or portion thereof derived from an IgG1 molecule. In another embodiment, a parental Fc polypeptide can comprise a chimeric hinge domain. For example, the chimeric hinge can comprise a hinge domain derived, in part, from an IgG1 molecule and, in part, from an IgG3 molecule. In another embodiment, the chimeric hinge comprises a middle hinge domain from an IgG1 molecule and upper and lower hinge domains from an IgG4 molecule.

As set forth herein, it will be understood by one of ordinary skill in the art that a parental Fc moiety may be identical to the corresponding Fc moiety of naturally-occurring immunoglobulin or may be altered such that it varies in amino acid sequence. In certain embodiments, a parental Fc polypeptide is altered, e.g., by amino acid mutation (e.g., addition, deletion, or substitution). For example, the parental Fc polypeptide may be a Fc moiety having at least one amino acid substitution as compared to the wild-type Fc from which the Fc moiety is derived. For example, wherein the Fc moiety is derived from a human IgG1 antibody, a variant comprises at least one amino acid mutation (e.g., substitution) as compared to a wild type amino acid at the corresponding position of the human IgG1 Fc region.

The amino acid substitution(s) may be located at a position within the Fc moiety referred to as “corresponding” to the position number that that residue would be given in an Fc region in an antibody (as set forth using the EU numbering convention). One of skill in the art can readily generate alignments to determine what the EU number “corresponding” to a position in an Fc moiety would be.

In one embodiment, the substitution is at an amino acid position located in a hinge domain or portion thereof. In another embodiment, the substitution is at an amino acid position located in a CH2 domain or portion thereof. In another embodiment, the substitution is at an amino acid position located in a CH3 domain or portion thereof. In another embodiment, the substitution is at an amino acid position located in a CH4 domain or portion thereof.

In certain embodiments, the parental Fc polypeptide comprise more than one amino acid substitution. The parental Fc polypeptide may comprise, for example, 2, 3, 4, 5, 6, 7, 8, 9, 10 or more amino acid substitutions relative to a wild-type Fc region. Preferably, the amino acid substitutions are spatially positioned from each other by an interval of at least 1 amino acid position or more, for example, at least 2, 3, 4, 5, 6, 7, 8, 9, or 10 amino acid positions or more. More preferably, the engineered amino acids are spatially positioned apart from each other by an interval of at least 5, 10, 15, 20, or 25 amino acid positions or more.

In certain embodiments, the substitution confers an alteration of at least one effector function imparted by an Fc region comprising a wild-type Fc moiety (e.g., a reduction in the ability of the Fc region to bind to Fc receptors (e.g. FcγRI, FcγRII, or FcγRIII) or complement proteins (e.g. C1q), or to trigger antibody-dependent cell cytotoxicity (ADCC), phagocytosis, or complement-dependent cytotoxicity (CDC)).

The parental Fc polypeptides may employ art-recognized substitutions which are known to impart an alteration of effector function. Specifically, a parental Fc polypeptide of the invention may include, for example, a change (e.g., a substitution) at one or more of the amino acid positions disclosed in International PCT Publications WO88/07089A1, WO96/14339A1, WO98/05787A1, WO98/23289A1, WO99/51642A1, WO99/58572A1, WO00/09560A2, WO00/32767A1, WO00/42072A2, WO02/44215A2, WO02/060919A2, WO03/074569A2, WO04/016750A2, WO04/029207A2, WO04/035752A2, WO04/063351A2, WO04/074455A2, WO04/099249A2, WO05/040217A2, WO04/044859, WO05/070963A1, WO05/077981A2, WO05/092925A2, WO05/123780A2, WO06/019447A1, WO06/047350A2, and WO06/085967A2; US Patent Publication Nos. US2007/0231329, US2007/0231329, US2007/0237765, US2007/0237766, US2007/0237767, US2007/0243188, US20070248603, US20070286859, US20080057056; or U.S. Pat. Nos. 5,648,260; 5,739,277; 5,834,250; 5,869,046; 6,096,871; 6,121,022; 6,194,551; 6,242,195; 6,277,375; 6,528,624; 6,538,124; 6,737,056; 6,821,505; 6,998,253; 7,083,784; and 7,317,091, the portion of each of which pertaining to Fc mutations is incorporated by reference herein. In one embodiment, the specific change (e.g., the specific substitution of one or more amino acids disclosed in the art) may be made at one or more of the disclosed amino acid positions. In another embodiment, a different change at one or more of the disclosed amino acid positions (e.g., the different substitution of one or more amino acid position disclosed in the art) may be made.

In preferred embodiments, a parental Fc polypeptide may comprise an Fc moiety comprising an amino acid substitution at an amino acid position corresponding to EU amino acid position that is within the “15 Angstrom Contact Zone” of an Fc moiety. The 15 Angstrom Zone includes residues located at EU positions 243 to 261, 275 to 280, 282-293, 302 to 319, 336 to 348, 367, 369, 372 to 389, 391, 393, 408, and 424-440 of a full-length, wild-type Fc moiety.

In another embodiment, a parental Fc polypeptide comprises an Fc region comprising one or more truncated Fc moieties that are nonetheless sufficient to confer one or more functions to the Fc region. For example, the portion of an Fc moiety that binds to FcRn (i.e., the FcRn binding portion) comprises from about amino acids 282-438, EU numbering. Thus, an Fc moiety of a parental Fc polypeptide may comprise or consist of an FcRn binding portion. FcRn binding portions may be derived from heavy chains of any isotype, including IgG1, IgG2, IgG3 and IgG4. In one embodiment, an FcRn binding portion from an antibody of the human isotype IgG1 is used. In another embodiment, an FcRn binding portion from an antibody of the human isotype IgG4 is used. In certain embodiments, the FcRn binding portion is aglycosylated. In other embodiments, the FcRn binding portion is glycosylated.

In certain embodiments, a parental Fc polypeptide comprises an amino acid substitution to an Fc moiety which alters the antigen-independent effector functions of the antibody, in particular the circulating half-life of the antibody. Such polypeptides exhibit either increased or decreased binding to FcRn when compared to polypeptides lacking these substitutions and, therefore, have an increased or decreased half-life in serum, respectively. Parental Fc polypeptides with improved affinity for FcRn are anticipated to have longer serum half-lives, and such molecules have useful applications in methods of treating mammals where long half-life of the administered polypeptide is desired, e.g., to treat a chronic disease or disorder. In contrast, parental Fc polypeptides with decreased FcRn binding affinity are expected to have shorter half-lives, and such molecules are also useful, for example, for administration to a mammal where a shortened circulation time may be advantageous, e.g. for in vivo diagnostic imaging or in situations where the starting polypeptide has toxic side effects when present in the circulation for prolonged periods. Parental Fc polypeptides with decreased FcRn binding affinity are also less likely to cross the placenta and, thus, are also useful in the treatment of diseases or disorders in pregnant women. In addition, other applications in which reduced FcRn binding affinity may be desired include those applications in which localization the brain, kidney, and/or liver is desired. In one exemplary embodiment, the parental Fc polypeptides exhibit reduced transport across the epithelium of kidney glomeruli from the vasculature. In another embodiment, the binding polypeptides of the invention exhibit reduced transport across the blood brain barrier (BBB) from the brain, into the vascular space. In one embodiment, a parental Fc polypeptide with altered FcRn binding comprises at least one Fc moiety (e.g, one or two Fc moieties) having one or more amino acid substitutions within the “FcRn binding loop” of an Fc moiety. The FcRn binding loop is comprised of amino acid residues 280-299 (according to EU numbering) of a wild-type, full-length, Fc moiety. In other embodiments, a parental Fc polypeptide having altered FcRn binding affinity comprises at least one Fc moiety (e.g, one or two Fc moieties) having one or more amino acid substitutions within the 15 \acute{Å} FcRn “contact zone.”

As used herein, the term 15 \acute{Å} FcRn “contact zone” includes residues at the following positions of a wild-type, full-length Fc moiety: 243-261, 275-280, 282-293, 302-319, 336-348, 367, 369, 372-389, 391, 393, 408, 424, 425-440 (EU numbering). In preferred embodiments, a parental Fc polypeptide having altered FcRn binding affinity comprises at least one Fc moiety (e.g, one or two Fc moieties) having one or more amino acid substitutions at an amino acid position corresponding to any one of the following EU positions: 256, 277-281, 283-288, 303-309, 313, 338, 342, 376, 381, 384, 385, 387, 434 (e.g., N434A or N434K), and 438. Exemplary amino acid substitutions which altered FcRn binding activity are disclosed in International PCT Publication No. WO05/047327 which is incorporated by reference herein.

In other embodiments, a parental Fc polypeptide comprises at least one Fc moiety having engineered cysteine residue or analog thereof which is located at the solvent-exposed surface. Preferably the engineered cysteine residue or analog thereof does not interfere with an effector function conferred by the Fc region. In preferred embodiments, the Fc polypeptides comprise an Fc moiety comprising at least one engineered free cysteine residue or analog thereof that is substantially free of disulfide bonding with a second cysteine residue. In preferred embodiments, the Fc polypeptides may comprise an Fc moiety having engineered cysteine residues or analogs thereof at one or more of the following positions in the CH3 domain: 349-371, 390, 392, 394-423, 441-446, and 446b (EU numbering). In more preferred embodiments, the Fc polypeptides comprise an Fc variant having engineered cysteine residues or analogs thereof at any one of the following positions: 350, 355, 359, 360, 361, 389, 413, 415, 418, 422, 441, 443, and EU position 446b (EU numbering). Any of the above engineered cysteine residues or analogs thereof may subsequently be conjugated to a functional moiety using art-recognized techniques (e.g., conjugated with a thiol-reactive heterobifunctional linker).

### B. Effector-Less Fc Polypeptides

In certain embodiments, the parental Fc polypeptides are “effector-less” Fc polypeptides with altered or reduced effector function. Preferably, the effector function that is reduced or altered is an antigen-dependent effector function. For example, a parental Fc polypeptide may comprise a sequence variation (e.g., an amino acid substitution) which reduces the antigen-dependent effector functions of the polypeptide, in particular ADCC or complement activation, e.g., as compared to a wild type Fc polypeptide. Unfortunately, such parental Fc polypeptides often have reduced stability making them ideal candidates for stabilization according to the methods of the invention.

Fc polypeptides with decreased FcγR binding affinity are expected to reduce effector function, and such molecules are also useful, for example, for treatment of conditions in which target cell destruction is undesirable, e.g., where normal cells may express target molecules, or where chronic administration of the polypeptide might result in unwanted immune system activation. In one embodiment, the Fc polypeptide exhibits a reduction in at least one antigen-dependent effector function selected from the group consisting of opsonization, phagocytosis, complement dependent cytotoxicity, antibody-dependent cell cytotoxicity (ADCC), or effector cell modulation as compared to a Fc polypeptide comprising a wild type Fc region. In one embodiment the Fc polypeptide exhibits altered binding to an activating FcγR (e.g. FcγRI, FcγRIIa, or FcγRIIIa). In another embodiment, the Fc polypeptide exhibits altered binding affinity to an inhibitory FcγR (e.g. FcγRIIb). In other embodiments, an Fc polypeptide with decreased FcγR binding affinity (e.g. decreased FcγRI, FcγRII, or FcγRIIIa binding affinity) comprises at least one Fc moiety (e.g, one or two Fc moieties) having an amino acid substitution at an amino acid position corresponding to one or more of the following positions: 234, 236, 239, 241, 251, 252, 261, 265, 268, 293, 294, 296, 298, 299, 301, 326, 328, 332, 334, 338, 376, 378, and 435 (EU numbering). In other embodiments, an Fc polypeptide with decreased complement binding affinity (e.g. decreased C1q binding affinity) comprises an Fc moiety (e.g, one or two Fc moieties) having an amino acid substitution at an amino acid position corresponding to one or more of the following positions: 239, 294, 296, 301, 328, 333, and 376 (EU numbering). Exemplary amino acid substitutions which altered FcγR or complement binding activity are disclosed in International PCT Publication No. WO05/063815 which is incorporated by reference herein. In certain preferred embodiments, binding polypeptide of the invention may comprise one or more of the following specific substitutions: S239D, S239E, M252T, H268D, H268E, 1332D, 1332E, N434A, and N434K (i.e., one or more of these substitutions at an amino acid position corresponding to one or more of these EU numbered position in an antibody Fc region).

In certain exemplary embodiments, the effector function of the parental ‘effector-less’ polypeptide may be altered or reduced due to an aglycosylated Fc region within the parental Fc polypeptide. In certain embodiments, the aglycosylated Fc region is generated by an amino acid substitution which alters the glycosylation of the Fc region. For example, the asparagine at EU position 297 within the Fc region may altered (e.g., by substitution, insertion, deletion, or by chemical modification) to inhibit its glycosylation. In another exemplary embodiment, the amino acid residue at EU position 299 (e.g., Threonine (T)) is substituted with (e.g., with Alanine (A)) to reduce glycosylation at the adjacent residue 297. Exemplary amino acid substitutions which reduce or alter glycosylation are disclosed in International PCT Publication No. WO05/018572 and US Patent Publication No. 2007/0111281, which are incorporated by reference herein. In other embodiments, the aglycosylated Fc region is generated by enzymatic or chemical removal of oligosaccharide or expression of the Fc polypeptide in a host cell that is unable to glycosylate the Fc region (e.g., a bacterial host cell or a mammalian host cell with impaired glycosylation machinery).

In certain embodiments, the aglycosylated Fc region is partially aglycosylated or hemi-glycosylated. For example, the Fc region may comprise a first, glycosylated, Fc moiety (e.g., a glycosylated CH2 region) and a second, aglycosylated, Fc moiety (e.g., an aglycosylated CH2 region). In other embodiments, the Fc region may be fully aglycosylated, i.e., none of its Fc moieties are glycosylated.

The aglycosylated Fc region of an “effector-less” polypeptide may be of any IgG isotype (e.g., IgG1, IgG2, IgG3, or IgG4). In one exemplary embodiment, the parental Fc polypeptide may comprises the aglycosylated Fc region of an IgG4 antibody such as “agly IgG4.P”. Agly IgG4.P is an engineered form of an IgG4 antibody that includes a proline substitution (Ser228Pro) in the hinge region and a Thr299Ala mutation in the CH2 domain to produce an aglycosylated Fc region (EU numbering). Agly IgG4.P has been shown to have no measurable immune effector function in vitro. In another exemplary embodiment, the parental Fc polypeptide comprises the aglycosylated Fc region of an IgG1 antibody, such as “agly IgG1”. Agly IgG1 is an aglycosylated form of the IgG immunoglobulin IgG1 with a Thr299Ala mutation (EU numbering) that confers a low effector function profile. Both agly IgG4.P and agly IgG1 antibodies represent an important class of therapeutic reagents where immune effector function is not desired.

In certain exemplary embodiments, the “effector-less” parental Fc polypeptide comprises a Fc region which is derived from an IgG4 antibody. The IgG4 Fc region may be identical to the wild-type Fc region or it may have one or more modifications to the wild-type IgG4 sequence. Such IgG4-like Fc polypeptides have reduced effector function as a result of the inherently reduced ability of an IgG4 antibody to bind to complement and/or Fc receptors. Parental Fc polypeptides of the IgG4 isotype may be either glycosylated or aglycosylated. Furthermore, the Fc region of an IgG4-like Fc polypeptide may comprise the complete Fc moiety of an IgG4 antibody or it may comprise a chimeric Fc moiety wherein a portion of the Fc moiety is from an IgG4 antibody and the remainder is from an antibody of another isotype. In one exemplary embodiment, the chimeric Fc moiety comprises a CH3 domain from an IgG1 antibody and CH2 domain from an IgG4 antibody. In another embodiment, the IgG4 antibody comprises a chimeric hinge, wherein the upper and lower hinge domains are from an IgG4 antibody but the middle hinge domain is from an IgG1 antibody as a result of a proline substitution (Ser228Pro) in the hinge region. In yet another embodiment, the parental chimeric chimeric IgG4 antibody comprises a chimeric hinge, wherein the upper and lower hinge domains are from an IgG4 antibody but the middle hinge domain is from an IgG1 antibody as a result of a proline substitution (Ser228Pro) in the hinge region, a CH1 domain from an IgG1 or IgG4 antibody, a CH2 domain (or positions 292-340, EU numbering) from an IgG4 antibody, and a CH1CH3 domain from an IgG1 antibody.

In certain embodiments, the reduced effector function of an “effector-less” Fc polypeptide is reduced binding to an Fc receptor (FcR), such as the FcγRI, FcγRII, FcγRIII, and/or FcγRIIIb receptor or a complement protein, for example, the complement protein C1q. This change in binding can be by a factor of about 1 fold or more, e.g., by about 2, 3, 4, 5, 6, 7, 8, 9, 10, 15, 50, or 100-fold or more, or by any interval or range thereof. These decreases in effector function, e.g., Fc binding to an Fc receptor or complement protein, are readily calculated based on, e.g., the percent reductions in binding activity determined using the assays described herein or assays known in the art.

In one embodiment of the invention an stabilized Fc polypeptide comprises a single chain Fc region. Such single chain Fc regions are known in the art (see, e.g., WO200801243, WO2008131242; WO2008153954) and can be made using known methods. Stabilizing amino acids as taught herein may be incorporated into one or more Fc moieties of such constructs using methods known to those of skill in the art. Such single chain Fc regions or genetically-fused Fc regions are synthetic Fc region comprised of Fc domains (or Fc moieties) genetically linked within a single polypeptide chain (i.e., encoded in a single contiguous genetic sequence). Accordingly, a genetically-fused Fc region (i.e., a scFc region) is monomeric in that they comprise one polypeptide chain, yet the appropriate portions of the molecule dimerize to form n Fc region. It will be understood that the teachings herein with respect to Fc moieties are applicable to both two chain Fc dimers and single chain Fc dimers. For example, either type of Fc region construct may be derived from, e.g., an IgG1 or IgG4 antibody or may be chimeric (e.g., comprising a chimeric hinge and/or comprising a CH2 domain from an IgG4 antibody and a CH3 domain from an IgG1 antibody.

(III). Variant Fc Polypeptides with Stabilized Fc Regions

In certain aspects, the invention provides variant Fc polypeptides which comprise amino acid sequences which are variants of any one of the parental Fc polypeptides described supra. In particular, the variant Fc polypeptides of the invention comprise an Fc region (or Fc moiety) with an amino acid sequence which is derived from the Fc region (or Fc moiety) of a parental Fc polypeptide. Preferably, the variant Fc polypeptide differs from the parental Fc polypeptide by the presence of at least one of the stabilizing Fc mutations described herein. In certain embodiments, the Fc variant may comprise additional amino acid sequence alterations. In preferred embodiments, the Fc variant will have enhanced stability as compared to the parent Fc polypeptide and, optionally, altered effector function as compared to the parental Fc polypeptide. For example, the variant Fc polypeptide may have an antigen-dependent effector function that is equivalent to or lower than the antigen-dependent effector function (e.g., ADCC and/or CDC) of the parental Fc polypeptide. Additionally or alternatively, the variant Fc polypeptide may have an antigen-independent effector function (e.g., extended half-life) relative to the parental Fc polypeptide.

In certain embodiments, the variant Fc polypeptide comprises an Fc region (or Fc moiety) that is essentially identical to the Fc region of a parental Fc polypeptide (Fc moiety) but for about one or more mutations (e.g., about 1 to about 20, about 1 to about 15, about 1 to about 10, about 1 to about 5, about 1 to about 4, about 1 to about 3, about 2 to about 20, about 2 to about 15, about 2 to about 10, about 5 to about 20, or about 5 to about 10) mutations relative to the starting or parent polypeptide, e.g., one or more amino acid residues which have been substituted with another amino acid residue or which has one or more amino acid residue insertions or deletions. In certain embodiments, the variant Fc polypeptide has 1, 2, 3, 4, 5, 6, 7, 8, 9, 10, 11, 12, 13, 14, 15, 16, 17, 18, 19 or 20 mutations relative to the starting polypeptide. Preferably, the variant polypeptide comprises an amino acid sequence which is not naturally occurring.

Such variants necessarily have less than 100% sequence identity or similarity with the starting polypeptide. In a preferred embodiment, the variant will have an amino acid sequence from about 75% to less than 100% amino acid sequence identity or similarity with the amino acid sequence of the starting polypeptide, more preferably from about 80% to less than 100%, more preferably from about 85% to less than 100%, more preferably from about 90% to less than 100% (e.g., 91-99%, 92-99%, 93-99%, 94-99%, 95-99%, 96-99%, 97-99%, 98-99%, or 99%) and most preferably from about 95% to less than 100%, e.g., over the entire length of the variant molecule or a portion thereof (e.g., an Fc region or Fc moiety). In one embodiment, there is one amino acid difference between a starting polypeptide sequence (e.g., the Fc region of a parental Fc polypeptide) and the sequence derived therefrom (e.g., the Fc region of a variant Fc polypeptide).

In certain embodiments, the variant Fc polypeptides of the invention are stabilized Fc polypeptides. That is, the stabilized polypeptides comprise at least one sequence variation or mutation that is stabilizing Fc mutation. As used herein, the term “stabilizing Fc mutation” includes a mutation within an Fc region of a variant Fc polypeptide which confers enhanced protein stability (e.g. thermal stability) variant Fc polypeptide as compared to the parental Fc polypeptide from which it is derived. Preferably, the stabilizing mutation comprises the substitution of a destabilizing amino acid in an Fc region with a replacement amino acid that confers enhanced protein stability (herein a “stabilizing amino acid”) to the Fc region. In one embodiment, a stabilized Fc polypeptide of the invention comprises one or more amino acid stabilizing Fc mutations (e.g., 1, 2, 3, 4, 5, 6, 7, 8, 9, 10, 11, 12, 13, 14, 15, 16, 17, 18, 19 or 20 stabilizing mutations). Stabilizing Fc mutations are preferably introduced into a CH2 domain, a CH3 domain, or both CH2 and CH3 domains of an Fc region.

In certain exemplary embodiments, a variant Fc polypeptide of the invention is a stabilized variant of an “effector-less” parental Fc polypeptide described supra. That is, the stabilized variant has enhanced stability relative to the “effector-less parent Fc polypeptide”. In one exemplary embodiment, the variant Fc polypeptide is a stabilized variant of a parental Fc polypeptide comprising the aglycosylated Fc region of an IgG1 antibody, e.g., an aglycosylated IgG1 Fc region comprising a T299A mutation (EU numbering). In another exemplary embodiment, the variant Fc polypeptide is a stabilized variant of a parental Fc polypeptide comprising the Fc region of a glycosylated or aglycosylated IgG4 antibody. For example, the variant Fc polypeptide may comprise a stabilizing mutation in an Fc region derived from an “agly IgG4.P” antibody.

Preferably, the stabilized Fc polypeptides of the invention exhibit enhanced stability when compared to the variant Fc polypeptide under identical measurement conditions. It will be recognized, however, that the degree to which the stability of Fc variant polypeptide is enhanced relative to its parent Fc polypeptide may vary under the chosen measurement conditions. For example, the enhancement of stability may be observed at a particular pH, e.g., an acidic, neutral or basic pH. In one embodiment, the enhanced stability is observed at an acidic pH of less than about 6.0 (e.g., about 6.0, about 5.5, about 5.0, about 4.5, or about 4.0). In another embodiment, the enhanced stability is observed at a neutral pH of about 6.0 to about 8.0 (e.g., about 6.0, about 6.5, about 7.0, about 7.5, about 8.0). In another embodiment, the enhanced stability is observed at a basic pH of about 8.0 to about 10.0 (e.g., about 8.0, about 8.5, about 9.0, about 9.5, about 10.0).

The enhanced thermal stability of the variant Fc polypeptide can be evaluated, e.g., using any of the methods described below. In certain embodiments, the stabilized Fc polypeptides have Fc regions (or Fc moieties) with a thermal stability (e.g., a melting temperature or Tm) that is greater than about 0.1, about 0.25, about 0.5, about 0.75, about 1, about 1.25, about 1.5, about 1.75, about 2, about 3, about 4, about 5, about 6, about 7, about 8, about 9, about 10, about 11, about 12, about 13, about 14, about 15, about 16, about 17, about 18, about 19, about 20, about 25, about 30, about 40 or about 50 degrees Celsius higher than that of the parental polypeptide from which it is derived.

In certain embodiments, stabilized Fc polypeptide variants of the invention are expressed as a monomeric, soluble protein of which is no more than 25% in dimeric, tetrameric, or otherwise aggregated form (e.g., less than about 25%, about 20%, about 15%, about 10%, or about 5%).

In another embodiment, stabilized Fc polypeptides have a T50 of greater than 40° C. (e.g., 40, 41, 42, 43, 44, 45, 46, 47, 48, 49° C., or more) in a thermal challenge assay (see U.S. patent application Ser. No. 11/725,970, which is incorporated by reference herein, as well as Example 2 infra). In more preferred embodiments, stabilized Fc molecules of the invention have a T50 of greater than 50° C. (e.g., 50, 51, 52, 53, 54, 55, 56, 57, 58° C. or more). In more preferred embodiments, stabilized Fc molecules of the invention have a T50 of greater than 60° C. (e.g., 60, 61, 62, 63, 64, 65° C., or more). In yet more preferred embodiments, stabilized Fc molecules of the invention have a T50 of greater than 65° C. (e.g., 65, 66, 67, 68, 69, 70° C., or more). In still more preferred embodiments, stabilized Fc molecules of the invention have a T50 of greater than 70° C. (e.g., 70, 71, 73, 74, 75° C., or more).

In certain embodiments, stabilized Fc molecules of the invention have CH2 domains with Tm values greater than about 60° C. (e.g., about 61, 62, 63, 64, 65° C. or higher), greater than 65° C. (e.g., 65, 66, 67, 68, 69° C. or higher), or greater than about 70° C. (e.g., 71, 72, 73, 74, 75° C. or higher). In other embodiments, stabilized Fc molecules of the invention have CH3 domains with Tm values greater than about 70° C. (e.g., 71, 72, 73, 74, 75° C. or higher), greater than about 75° C. (e.g., 76, 77, 78, 79, 80° C. or higher), or greater than 80° C. (e.g., 81, 82, 83, 84, 85° C. or higher). In particular embodiments, said stabilized Fc polypeptides are variants of a parental Fc polypeptide comprising an aglycosylated or glycosylation Fc region of an IgG4 antibody (e.g., agly IgG4.P). In other embodiments, said stabilized Fc polypeptides are variants of a parental Fc polypeptide comprising an aglycosylated Fc region of an IgG1 antibody (e.g., agly IgG1). In yet other embodiments, the stabilized Fc molecule of the invention has a Fc region or Fc moiety (e.g., a CH2 and/or CH3 domain) with a thermal stability that is substantial the same or greater than that of a glycosylated IgG1 antibody.

In certain embodiments, variant Fc polypeptides of the invention result in reduced aggregation as compared to the parental Fc polypeptides from which they are derived. In one embodiment, a stabilized Fc molecule produced by the methods of the invention has a decrease in aggregation of at least 1% relative to the parental Fc molecule. In other embodiments, the stabilized Fc polypeptide has a decrease in aggregation of at least 2%, at least 5%, at least 10%, at least 20%, at least 30%, at least 50%, at least 75%, or at least 100%, relative to the parental molecule.

In other embodiments, stabilized Fc polypeptides of the invention result in increased long-term stability or shelf-life as compared to parental Fc polypeptides from which they are derived. In one embodiment, a stabilized Fc molecule produced by the methods of the invention has an increase in shelf life of at least 1 day relative to the unstabilized binding molecule. This means that a preparation of stabilized Fc polypeptides has substantially the same amount of biologically active variant Fc polypeptides as present on the previous day, and the preparation does not have any appreciable aggregation or decomposition of the variant polypeptide. In other embodiments, the stabilized Fc molecule has an increase in shelf life of at least 2 days, at least 5 days, at least 1 week, at least 2 weeks, at least 1 month, at least 2 months, at least 6 months, or at least 1 year, relative to the unstabilized Fc molecule.

In certain embodiments, stabilized Fc polypeptides of the invention are expressed at increased yield as compared to their parental Fc polypeptides. In one embodiment, a stabilized Fc polypeptide of the invention has an increase in yield of at least 1% relative to the parent Fc molecule. In other embodiments, the stabilized Fc polypeptide has an increase in yield of at least 2%, at least 5%, at least 10%, at least 20%, at least 30%, at least 50%, at least 75%, at least 80%, at least 90%, at least 95%, at least 98% or at least 100%, relative to the parental Fc molecule.

In exemplary embodiments, stabilized Fc polypeptides of the invention are expressed at increased yields (as compared to their parental Fc polypeptides) in a host cell, e.g., a bacterial or eukaryotic (e.g., yeast or mammalian) host cell. Exemplary mammalian host cells which can be used to express a nucleic acid molecule encoding a stabilized Fc polypeptide of the invention include Chinese Hamster Ovary (CHO) cells, HELA (human cervical carcinoma) cells, CVI (monkey kidney line) cells, COS (a derivative of CVI with SV40 T antigen) cells, R1610 (Chinese hamster fibroblast) cells, BALBC/3T3 (mouse fibroblast) cells, HAK (hamster kidney line) cells, SP2/O (mouse myeloma) cells, BFA-1c1BPT cells (bovine endothelial cells), RAJI (human lymphocyte) cells, PER.C6® (human retina-derived cell line, Crucell, The Netherlands) and 293 cells (human kidney).

In other embodiments, the stabilized Fc polypeptides of the invention are expressed at increased yields (relative to an their parental Fc polypeptides) in a host cell under large-scale (e.g., commercial scale) conditions. In exemplary embodiments, the stabilized Fc molecule have increased yield when expressed in at least 10 liters of culture media. In other embodiments, a stabilized Fc binding molecule has an increase in yield when expressed from a host cell in at least 20 liters, at least 50 liters, at least 75 liters, at least 100 liters, at least 200 liters, at least 500 liters, at least 1000 liters, at least 2000 liters, at least 5,000 liters, or at least 10,000 liters of culture media. In an exemplary embodiment, at least 10 mg (e.g., 10 mg, 20 mg, 50 mg, or 100 mg) of a stabilized Fc molecule are produced for every liter of culture media.

### (a) Stabilizing Fc Amino Acids

In certain embodiments, the stabilized Fc molecules of the invention comprise one or more of the following stabilizing Fc amino acids at the indicated positions (e.g., 1, 2, 3, 4, 5, 6, 7, 8, or more stabilizing Fc mutations) which are independently selected from the group consisting of:
a) a substitution of an amino acid at EU position 240, e.g., with phenylalanine (240F),
b) substitution of an amino acid (e.g., valine) at EU position 262, e.g., with leucine (262L);
c) substitution of an amino acid (e.g., valine) at EU position 266, e.g., with phenylalanine (266F);
d) substitution of an amino acid (e.g., threonine) at EU position 299, e.g., with lysine (299K);
e) substitution of an amino acid (e.g., threonine) at EU position 307, e.g., with proline (307P);
f) substitution of an amino acid (e.g., leucine) at EU position 309, e.g., with lysine (309K), methionine (309M), or proline (309P);
g) a substitution of an amino acid (e.g., valine) at EU position 323, e.g., with phenylalanine (323F);
h) a substitution of an amino acid (e.g., aspartic acid) at EU position 399, e.g., with serine (399S);
i) a substitution of an amino acid (e.g., arginine) at EU position 409, e.g., with lysine (409K) or methionine (409L); and
j) a substitution of an amino acid (e.g., valine) at EU position 427, e.g., with phenylalanine (427F).

In one exemplary embodiment, the stabilized Fc polypeptide comprises stabilizing Fc mutation (a). In another exemplary embodiment, the stabilized Fc polypeptide comprises stabilizing Fc mutation (b). In another exemplary embodiment, the stabilized Fc polypeptide comprises stabilizing Fc mutation (c). In another exemplary embodiment, the stabilized Fc polypeptide comprises stabilizing Fc mutation (d). In another exemplary embodiment, the stabilized Fc polypeptide comprises stabilizing Fc mutation (e). In another exemplary embodiment, the stabilized Fc polypeptide comprises stabilizing Fc mutation (f). In another exemplary embodiment, the stabilized Fc polypeptide comprises stabilizing Fc mutation (g). In another exemplary embodiment, the stabilized Fc polypeptide comprises stabilizing Fc mutation (h). In another exemplary embodiment, the stabilized Fc polypeptide comprises stabilizing Fc mutation (i). In another exemplary embodiment, the stabilized Fc polypeptide comprises stabilizing Fc mutation (j).

In one exemplary embodiment, a stabilized Fc polypeptide of the invention comprises two or more (e.g., 2, 3, 4, or 5) of stabilizing mutations (a)-(j) above. In certain embodiments, two or more of stabilizing mutations (d)-(j) or (d)-(h). For example, a stabilized Fc polypeptide of the invention may comprise any one of the following combinations of stabilizing mutations: (d) and (e), (d) and (f), (d) and (g), (d) and (h), (d) and (i), (d) and (j), (e) and (f), (e) and (g), (e) and (h), (e) and (i), (e) and (j), (f) and (g), (f) and (h), (f) and (i), (f) and (j), (h) and (i), (h) and (j), (i) and (j). In another exemplary embodiment, a stabilized Fc polypeptide of the invention comprises mutations (d), (e), and (f). In another exemplary embodiment, a stabilized Fc polypeptide of the invention comprises mutations (d), (e), and (g). In another exemplary embodiment, a stabilized Fc polypeptide of the invention comprises mutations (d), (e), and (h). In another exemplary embodiment, a stabilized Fc polypeptide of the invention comprises mutations (d), (f), and (g). In another exemplary embodiment, a stabilized Fc polypeptide of the invention comprises mutations (d), (g), and (h). In another exemplary embodiment, a stabilized Fc polypeptide of the invention comprises mutations (e), (f), and (g). In another exemplary embodiment, a stabilized Fc polypeptide of the invention comprises mutations (e), (g), and (h). In another exemplary embodiment, a stabilized Fc polypeptide of the invention comprises mutations (f), (g), and (h). In another exemplary embodiment, a stabilized Fc polypeptide of the invention comprises mutations (e), (f), (g), and (h).

In another embodiment, a stabilized Fc polypeptide of the invention comprises a CH2 domain (or amino acids 292-340 thereof) of an IgG4 molecule and a CH3 domain from an IgG1 molecule, having a Gln (Q) residue at position 297. In another embodiment, a stabilized Fc polypeptide of the invention comprises a CH2 and CH3 domain of an IgG1 molecule and a Lys (K) residue at position 299, either alone or in combination with an Asp (D) residue at position 297.

### (b) Exemplary Stabilized Fc Moieties

Exemplary stabilized Fc moieties of the invention can be found throughout the application, Examples, and sequence listing.

In certain exemplary embodiments, a stabilized Fc polypeptide of the invention comprises an stabilized IgG4 Fc region comprising one, two or more of the Fc moiety amino acid sequences set forth in Table 1 below. Stabilizing Fc mutations are underlined in bold italics.

TABLE 1
Stabilized IgG4 Fc moieties

| Fc Moiety | | |
|---|---|---|
| (Fc mutation(s), | | |
| glycosylation | | |
| state) | Sequence | |
| pCN579: | ESKYGPPCPPCPAPEFLGGPSVFLFPPKPKDTLMISR | SEQ ID |
| (T299K, | TPEVTCVVVDVSQEDPEVQFNWYVDGVEVHNAK | NO: 1 |
| aglycosylated) | TKPREEQFNS [symbol] YRVVSVLTVLHQDWLNGKEYKCK | |
| | VSNKGLPSSIEKTISKAKGQPREPQVYTLPPSQEEM | |
| | TKNQVSLTCLVKGFYPSDIAVEWESNGQPENNYK | |
| | TTPPVLDSDGSFFLYSRLTVDKSRWQEGNVFSCSV | |
| | MHEALHNHYTQKSLSLSLG | |
| EC301 (T299K, | ESKYGPPCPPCPAPEFLGGPSVFLFPPKPKDTLMISR | SEQ ID |
| V427F | TPEVTCVVVDVSQEDPEVQFNWYVDGVEVHNAK | NO: 2 |
| aglycosylated) | TKPREEQFNS [symbol] YRVVSVLTVLHQDWLNGKEYKCK | |
| | VSNKGLPSSIEKTISKAKGQPREPQVYTLPPSQEEM | |
| | TKNQVSLTCLVKGFYPSDIAVEWESNGQPENNYK | |
| | TTPPVLDSDGSFFLYSRLTVDKSRWQEGNVFSCS [symbol] | |
| | MHEALHNHYTQKSLSLSLG | |
| EC302 (T299K, | E S K Y G P P C P P C P A P E F L G G P S V F L F P | SEQ ID |
| D399S, | P K P K D T L M I S R T P E V T C V V V D V S Q E | NO: 3 |
| aglycosylated) | D P E V Q F N W Y V D G V E V H N A K T K P R E | |
| | E Q F N S [symbol] Y R V V S V L T V L H Q D W L N G K E | |
| | Y K C K V S N K G L P S S I E K T I S K A K G Q P R | |
| | E P Q V Y T L P P S Q E E M T K N Q V S L T C L V | |
| | K G F Y P S D I A V E W E S N G Q P E N N Y K T T | |
| | P P V L [symbol] S D G S F F L Y S R L T V D K S R W Q E G | |
| | N V F S C S V M H E A L H N H Y T Q K S L S L S L | |
| | G | |
| EC303 (T307P, | E S K Y G P P C P P C P A P E F L G G P S V F L F P | SEQ ID |
| V427F | P K P K D T L M I S R T P E V T C V V V D V S Q E | NO: 4 |
| glycosylated) | D P E V Q F N W Y V D G V E V H N A K T K P R E | |
| | E Q F N S T Y R V V S V L [symbol] V L H Q D W L N G K E | |
| | Y K C K V S N K G L P S S I E K T I S K A K G Q P R | |
| | E P Q V Y T L P P S Q E E M T K N Q V S L T C L V | |
| | K G F Y P S D I A V E W E S N G Q P E N N Y K T T | |
| | P P V L D S D G S F F L Y S R L T V D K S R W Q E | |
| | G N V F S C S [symbol] M H E A L H N H Y T Q K S L S L S | |
| | L G | |
| EC304 (T307P, | E S K Y G P P C P P C P A P E F L G G P S V F L F P | SEQ ID |
| D399S, | P K P K D T L M I S R T P E V T C V V V D V S Q E | NO: 5 |
| glycosylated) | D P E V Q F N W Y V D G V E V H N A K T K P R E | |
| | E Q F N S T Y R V V S V L [symbol] V L H Q D W L N G K E | |
| | Y K C K V S N K G L P S S I E K T I S K A K G Q P R | |
| | E P Q V Y T L P P S Q E E M T K N Q V S L T C L V | |
| | K G F Y P S D I A V E W E S N G Q P E N N Y K T T | |
| | P P V L [symbol] S D G S F F L Y S R L T V D K S R W Q E G | |
| | N V F S C S V M H E A L H N H Y T Q K S L S L S L | |
| | G | |
| EC305 (T299K, | E S K Y G P P C P P C P A P E F L G G P S V F L F P | SEQ ID |
| D399S, V427F | P K P K D T L M I S R T P E V T C V V V D V S Q E | NO: 6 |
| aglycosylated) | D P E V Q F N W Y V D G V E V H N A K T K P R E | |
| | E Q F N S [symbol] Y R V V S V L T V L H Q D W L N G K E | |
| | Y K C K V S N K G L P S S I E K T I S K A K G Q P R | |
| | E P Q V Y T L P P S Q E E M T K N Q V S L T C L V | |
| | K G F Y P S D I A V E W E S N G Q P E N N Y K T T | |
| | P P V L [symbol] S D G S F F L Y S R L T V D K S R W Q E G | |
| | N V F S C S [symbol] M H E A L H N H Y T Q K S L S L S L | |
| | G | |
| EC306: (T307P, | E S K Y G P P C P P C P A P E F L G G P S V F L F P | SEQ ID |
| D399S, V427F | P K P K D T L M I S R T P E V T C V V V D V S Q E | NO: 7 |
| glycosylated) | D P E V Q F N W Y V D G V E V H N A K T K P R E | |
| | E Q F N S T Y R V V S V L [symbol] V L H Q D W L N G K E | |
| | Y K C K V S N K G L P S S I E K T I S K A K G Q P R | |
| | E P Q V Y T L P P S Q E E M T K N Q V S L T C L V | |
| | K G F Y P S D I A V E W E S N G Q P E N N Y K T T | |
| | P P V L [symbol] S D G S F F L Y S R L T V D K S R W Q E G | |
| | N V F S C S [symbol] M H E A L H N H Y T Q K S L S L S L | |
| | G | |
| EC307 (T299K, | E S K Y G P P C P P C P A P E F L G G P S V F L F P | SEQ ID |
| V348F, V427F | P K P K D T L M I S R T P E V T C V V V D V S Q E | NO: 8 |
| aglycosylated) | D P E V Q F N W Y V D G V E V H N A K T K P R E | |
| | E Q F N S [symbol] Y R V V S V L T V L H Q D W L N G K E | |
| | Y K C K V S N K G L P S S I E K T I S K A K G Q P R | |
| | E P Q [symbol] Y T L P P S Q E E M T K N Q V S L T C L V | |
| | K G F Y P S D I A V E W E S N G Q P E N N Y K T T | |
| | P P V L D S D G S F F L Y S R L T V D K S R W Q E | |
| | G N V F S C S [symbol] M H E A L H N H Y T Q K S L S L S | |
| | L G | |
| EC308 (T307P, | E S K Y G P P C P P C P A P E F L G G P S V F L F P | SEQ ID |
| V323F | P K P K D T L M I S R T P E V T C V V V D V S Q E | NO: 9 |
| glycosylated) | D P E V Q F N W Y V D G V E V H N A K T K P R E | |
| | E Q F N S T Y R V V S V L [symbol] V L H Q D W L N G K E | |
| | Y K C K [symbol] S N K G L P S S I E K T I S K A K G Q P R | |
| | E P Q V Y T L P P S Q E E M T K N Q V S L T C L V | |
| | K G F Y P S D I A V E W E S N G Q P E N N Y K T T | |
| | P P V L D S D G S F F L Y S R L T V D K S R W Q E | |
| | G N V F S C S V M H E A L H N H Y T Q K S L S L S | |
| | L G | |
| EC309 (V240F | E S K Y G P P C P P C P A P E F L G G P S [symbol] F L F P P | SEQ ID |
| glycosylated) | K P K D T L M I S R T P E V T C V V V D V S Q E D | NO: 10 |
| | P E V Q F N W Y V D G V E V H N A K T K P R E E | |
| | Q F N S T Y R V V S V L T V L H Q D W L N G K E Y | |
| | K C K V S N K G L P S S I E K T I S K A K G Q P R E | |
| | P Q V Y T L P P S Q E E M T K N Q V S L T C L V K | |
| | G F Y P S D I A V E W E S N G Q P E N N Y K T T P | |
| | P V L D S D G S F F L Y S R L T V D K S R W Q E G | |
| | N V F S C S V M H E A L H N H Y T Q K S L S L S L | |
| | G | |
| EC300 (T307P | E S K Y G P P C P P C P A P E F L G G P S V F L F P | SEQ ID |
| glycosylated) | P K P K D T L M I S R T P E V T C V V V D V S Q E | NO: 11 |
| | D P E V Q F N W Y V D G V E V H N A K T K P R E | |
| | E Q F N S T Y R V V S V L [symbol] V L H Q D W L N G K E | |
| | Y K C K V S N K G L P S S I E K T I S K A K G Q P R | |
| | E P Q V Y T L P P S Q E E M T K N Q V S L T C L V | |
| | K G F Y P S D I A V E W E S N G Q P E N N Y K T T | |
| | P P V L D S D G S F F L Y S R L T V D K S R W Q E | |
| | G N V F S C S V M H E A L H N H Y T Q K S L S L S | |
| | L G | |
| EC321 (L309P, | E S K Y G P P C P P C P A P E F L G G P S V F L F P | SEQ ID |
| D399S | P K P K D T L M I S R T P E V T C V V V D V S Q E | NO: 12 |
| glycosylated) | D P E V Q F N W Y V D G V E V H N A K T K P R E | |
| | E Q F N S T Y R V V S V L T V [symbol] H Q D W L N G K E | |
| | Y K C K V S N K G L P S S I E K T I S K A K G Q P R | |
| | E P Q V Y T L P P S Q E E M T K N Q V S L T C L V | |
| | K G F Y P S D I A V E W E S N G Q P E N N Y K T T | |
| | P P V L [symbol] S D G S F F L Y S R L T V D K S R W Q E G | |
| | N V F S C S V M H E A L H N H Y T Q K S L S L S L | |
| | G | |
| EC322 (L309M, | E S K Y G P P C P P C P A P E F L G G P S V F L F P | SEQ ID |
| D399S | P K P K D T L M I S R T P E V T C V V V D V S Q E | NO: 13 |
| glycosylated) | D P E V Q F N W Y V D G V E V H N A K T K P R E | |
| | E Q F N S T Y R V V S V L T V [symbol] H Q D W L N G K | |
| | E Y K C K V S N K G L P S S I E K T I S K A K G Q P | |
| | R E P Q V Y T L P P S Q E E M T K N Q V S L T C L | |
| | V K G F Y P S D I A V E W E S N G Q P E N N Y K T | |
| | T P P V L [symbol] S D G S F F L Y S R L T V D K S R W Q E | |
| | G N V F S C S V M H E A L H N H Y T Q K S L S L S | |
| | L G | |
| EC323 (L309K, | E S K Y G P P C P P C P A P E F L G G P S V F L F P | SEQ ID |
| D399S | P K P K D T L M I S R T P E V T C V V V D V S Q E | NO: 14 |
| glycosylated) | D P E V Q F N W Y V D G V E V H N A K T K P R E | |
| | E Q F N S T Y R V V S V L T V [symbol] H Q D W L N G K E | |
| | Y K C K V S N K G L P S S I E K T I S K A K G Q P R | |
| | E P Q V Y T L P P S Q E E M T K N Q V S L T C L V | |
| | K G F Y P S D I A V E W E S N G Q P E N N Y K T T | |
| | P P V L [symbol] S D G S F F L Y S R L T V D K S R W Q E G | |
| | N V F S C S V M H E A L H N H Y T Q K S L S L S L | |
| | G | |
| EC324 (T307P, | E S K Y G P P C P P C P A P E F L G G P S V F L F P | SEQ ID |
| L309P, D399S | P K P K D T L M I S R T P E V T C V V V D V S Q E | NO: 15 |
| glycosylated) | D P E V Q F N W Y V D G V E V H N A K T K P R E | |
| | E Q F N S T Y R V V S V L [symbol] V [symbol] H Q D W L N G K E | |
| | Y K C K V S N K G L P S S I E K T I S K A K G Q P R | |
| | E P Q V Y T L P P S Q E E M T K N Q V S L T C L V | |
| | K G F Y P S D I A V E W E S N G Q P E N N Y K T T | |
| | P P V L [symbol] S D G S F F L Y S R L T V D K S R W Q E G | |
| | N V F S C S V M H E A L H N H Y T Q K S L S L S L | |
| | G | |
| EC325 (T307P, | E S K Y G P P C P P C P A P E F L G G P S V F L F P | SEQ ID |
| L309M, D399S | P K P K D T L M I S R T P E V T C V V V D V S Q E | NO: 16 |
| glycosylated) | D P E V Q F N W Y V D G V E V H N A K T K P R E | |
| | E Q F N S T Y R V V S V L [symbol] V [symbol] H Q D W L N G K | |
| | E Y K C K V S N K G L P S S I E K T I S K A K G Q P | |
| | R E P Q V Y T L P P S Q E E M T K N Q V S L T C L | |
| | V K G F Y P S D I A V E W E S N G Q P E N N Y K T | |
| | T P P V L [symbol] S D G S F F L Y S R L T V D K S R W Q E | |
| | G N V F S C S V M H E A L H N H Y T Q K S L S L S | |
| | L G | |
| EC326 (T307P, | E S K Y G P P C P P C P A P E F L G G P S V F L F P | SEQ ID |
| L309K, D399S | P K P K D T L M I S R T P E V T C V V V D V S Q E | NO: 17 |
| glycosylated) | D P E V Q F N W Y V D G V E V H N A K T K P R E | |
| | E Q F N S T Y R V V S V L [symbol] V [symbol] H Q D W L N G K E | |
| | Y K C K V S N K G L P S S I E K T I S K A K G Q P R | |
| | E P Q V Y T L P P S Q E E M T K N Q V S L T C L V | |
| | K G F Y P S D I A V E W E S N G Q P E N N Y K T T | |
| | P P V L [symbol] S D G S F F L Y S R L T V D K S R W Q E G | |
| | N V F S C S V M H E A L H N H Y T Q K S L S L S L | |
| | G | |
| YC401 | ESKYGPPCPPCPAPEFLGGPSVFLFPPKPKDTLMISR | SEQ ID |
| (T299A, T307P, | TPEVTCVVVDVSQEDPEVQFNWYVDGVEVHNAK | NO: 18 |
| D399S | TKPREEQFNS [symbol] YRVVSVL [symbol] VLHQDWLNGKEYKCK | |
| aglycosylated) | VSNKGLPSSIEKTISKAKGQPREPQVYTLPPSQEEM | |
| | TKNQVSLTCLVKGFYPSDIAVEWESNGQPENNYK | |
| | TTPPVL [symbol] SDGSFFLYSRLTVDKSRWQEGNVFSCSV | |
| | MHEALHNHYTQKSLSLSLG | |
| YC402 (T299A, | ESKYGPPCPPCPAPEFLGGPSVFLFPPKPKDTLMISR | SEQ ID |
| L309K, D399S | TPEVTCVVVDVSQEDPEVQFNWYVDGVEVHNAK | NO: 19 |
| aglycosylated) | TKPREEQFNS [symbol] YRVVSVLTV [symbol] HQDWLNGKEYKCK | |
| | VSNKGLPSSIEKTISKAKGQPREPQVYTLPPSQEEM | |
| | TKNQVSLTCLVKGFYPSDIAVEWESNGQPENNYK | |
| | TTPPVL [symbol] SDGSFFLYSRLTVDKSRWQEGNVFSCSV | |
| | MHEALHNHYTQKSLSLSLG | |
| YC403 (T299A, | ESKYGPPCPPCPAPEFLGGPSVFLFPPKPKDTLMISR | SEQ ID |
| T307P, L309K, | TPEVTCVVVDVSQEDPEVQFNWYVDGVEVHNAK | NO: 20 |
| D399S | TKPREEQFNS [symbol] YRVVSVL [symbol] V [symbol] HQDWLNGKEYKCK | |
| aglycosylated) | VSNKGLPSSIEKTISKAKGQPREPQVYTLPPSQEEM | |
| | TKNQVSLTCLVKGFYPSDIAVEWESNGQPENNYK | |
| | TTPPVL [symbol] SDGSFFLYSRLTVDKSRWQEGNVFSCSV | |
| | MHEALHNHYTQKSLSLSLG | |
| YC404 (T299K, | ESKYGPPCPPCPAPEFLGGPSVFLFPPKPKDTLMISR | SEQ ID |
| T307P, D399S | TPEVTCVVVDVSQEDPEVQFNWYVDGVEVHNAK | NO: 21 |
| aglycosylated) | TKPREEQFNS [symbol] YRVVSVL [symbol] VLHQDWLNGKEYKCK | |
| | VSNKGLPSSIEKTISKAKGQPREPQVYTLPPSQEEM | |
| | TKNQVSLTCLVKGFYPSDIAVEWESNGQPENNYK | |
| | TTPPVL [symbol] SDGSFFLYSRLTVDKSRWQEGNVFSCSV | |
| | MHEALHNHYTQKSLSLSLG | |
| YC405 (T299K, | ESKYGPPCPPCPAPEFLGGPSVFLFPPKPKDTLMISR | SEQ ID |
| L309K, D399S | TPEVTCVVVDVSQEDPEVQFNWYVDGVEVHNAK | NO: 22 |
| aglycosylated) | TKPREEQFNS [symbol] YRVVSVLTV [symbol] HQDWLNGKEYKCK | |
| | VSNKGLPSSIEKTISKAKGQPREPQVYTLPPSQEEM | |
| | TKNQVSLTCLVKGFYPSDIAVEWESNGQPENNYK | |
| | TTPPVL [symbol] SDGSFFLYSRLTVDKSRWQEGNVFSCSV | |
| | MHEALHNHYTQKSLSLSLG | |
| YC406 (T299K, | ESKYGPPCPPCPAPEFLGGPSVFLFPPKPKDTLMISR | SEQ ID |
| T307P, L309K, | TPEVTCVVVDVSQEDPEVQFNWYVDGVEVHNAK | NO: 23 |
| D399S | TKPREEQFNS [symbol] YRVVSVL [symbol] V [symbol] HQDWLNGKEYKCK | |
| aglycosylated) | VSNKGLPSSIEKTISKAKGQPREPQVYTLPPSQEEM | |
| | TKNQVSLTCLVKGFYPSDIAVEWESNGQPENNYK | |
| | TTPPVL [symbol] SDGSFFLYSRLTVDKSRWQEGNVFSCSV | |
| | MHEALHNHYTQKSLSLSLG | |

In other exemplary embodiments, a stabilized Fc polypeptide of the invention comprises an stabilized chimeric Fc region with one, two or more of the chimeric Fc moiety amino acid sequences set forth in Table 2 below.

TABLE 2
Stabilized Chimeric Fc moieties

| Fc Moiety | | |
|---|---|---|
| (Fc mutation(s), | | |
| glycosylation | | |
| state) | Sequence | |
| EAG2296 | ESKYGPPCPPCPAPEFLGGPSVFLFPPKPKDTLMI | SEQ ID |
| (T299A, IgG4 | SRTPEVTCVVVDVSQEDPEVQFNWYVDGVEVH | NO: 24 |
| CH2/IgG1 CH3 | NAKTKPREEQFNS [symbol] YRVVSVLTVLHQDWLNGK | |
| chimera) | EYKCKVSNKGLPSSIEKTISKAKGQPREPQVYTL | |
| | PPSRDELTKNQVSLTCLVKGFYPSDIAVEWESN | |
| | GQPENNYKTTPPVLDSDGSFFLYSKLTVDKSRW | |
| | QQGNVFSCSVMHEALHNHYTQKSLSLSPG | |
| EAG2287 | ESKYGPPCPPCPAPEFLGGPSVFLFPPKPKDTLMI | SEQ ID |
| (T299K, IgG4 | SRTPEVTCVVVDVSQEDPEVQFNWYVDGVEVH | NO: 25 |
| CH2/IgG1 CH3 | NAKTKPREEQFNS [symbol] YRVVSVLTVLHQDWLNGK | |
| chimera) | EYKCKVSNKGLPSSIEKTISKAKGQPREPQVYTL | |
| | PPSRDELTKNQVSLTCLVKGFYPSDIAVEWESN | |
| | GQPENNYKTTPPVLDSDGSFFLYSKLTVDKSRW | |
| | QQGNVFSCSVMHEALHNHYTQKSLSLSPG | |
| EC330 (T299A, | ESKYGPPCPPCPAPEFLGGPSVFLFPPKPKDTLMI | SEQ ID |
| T307P IgG4 | SRTPEVTCVVVDVSQEDPEVQFNWYVDGVEVH | NO: 26 |
| CH2/IgG1 CH3 | NAKTKPREEQFNS [symbol] YRVVSVL [symbol] VLHQDWLNGK | |
| chimera) | EYKCKVSNKGLPSSIEKTISKAKGQPREPQVYTL | |
| | PPSRDELTKNQVSLTCLVKGFYPSDIAVEWESN | |
| | GQPENNYKTTPPVLDSDGSFFLYSKLTVDKSRW | |
| | QQGNVFSCSVMHEALHNHYTQKSLSLSPG | |
| EC331 (T299K, | ESKYGPPCPPCPAPEFLGGPSVFLFPPKPKDTLMI | SEQ ID |
| T307P IgG4 | SRTPEVTCVVVDVSQEDPEVQFNWYVDGVEVH | NO: 27 |
| CH2/IgG1 CH3 | NAKTKPREEQFNS [symbol] YRVVSVL [symbol] VLHQDWLNGK | |
| chimera) | EYKCKVSNKGLPSSIEKTISKAKGQPREPQVYTL | |
| | PPSRDELTKNQVSLTCLVKGFYPSDIAVEWESN | |
| | GQPENNYKTTPPVLDSDGSFFLYSKLTVDKSRW | |
| | QQGNVFSCSVMHEALHNHYTQKSLSLSPG | |
| pEAG2300 | ESKYGPPCPPCPAPPVAGPSVFLFPPKPKDTLMIS | SEQ ID |
| (IgG4 chimeric | RTPEVTCVVVDVSQEDPEVQFNWYVDGVEVHN | NO: 28 |
| hinge + IgG1 | AKTKPREEQFNSTYRVVSVLTVLHQDWLNGKE | |
| CH3) | YKCKVSNKGLPSSIEKTISKAKGQPREPQVYTLP | |
| | PSRDELTKNQVSLTCLVKGFYPSDIAVEWESNG | |
| | QPENNYKTTPPVLDSDGSFFLYSKLTVDKSRWQ | |
| | QGNVFSCSVMHEALHNHYTQKSLSLSPG | |
| (N297Q, IgG4 | ESKYGPPCPPCPAPPVAGPSVFLFPPKPKDTLMIS | SEQ ID |
| CH2/IgG1 CH3 | RTPEVTCVVVDVSQEDPEVQFNWYVDGVEVHN | NO: 59 |
| chimera) | AKTKPREEQF [symbol] STYRVVSVLTVLHQDWLNGKE | |
| | YKCKVSNKGLPSSIEKTISKAKGQPREPQVYTLP | |
| | PSRDELTKNQVSLTCLVKGFYPSDIAVEWESNG | |
| | QPENNYKTTPPVLDSDGSFFLYSKLTVDKSRWQ | |
| | QGNVFSCSVMHEALHNHYTQKSLSLSPG | |

In other exemplary embodiments, a stabilized Fc polypeptide of the invention comprises an stabilized aglycosylated IgG1 Fc region with one, two or more of the IgG1 Fc moiety amino acid sequences set forth in Table 3 below.

TABLE 3
Stabilized Aglycosylated IgG1 Fc moieties

| Fc Moiety | | |
|---|---|---|
| (Fc | | |
| mutation(s), | | |
| glycosylation | | |
| state) | Sequence | |
| SDE1 | EPKSCDKTHTCPPCPAPELLGGPSVFLFPPKPKDT | SEQ ID |
| (T299K, | LMISRTPEVTC [symbol] VVDVSHEDPEVKFNWYVDGVE | NO: 29 |
| V262L | VHNAKTKPREEQYNS [symbol] YRVVSVLTVLHQDWLN | |
| aglycosylated) | GKEYKCKVSNKALPAPIEKTISKAKGQPREPQVY | |
| | TLPPSRDELTKNQVSLTCLVKGFYPSDIAVEWES | |
| | NGQPENNYKTTPPVLDSDGSFFLYSKLTVDKSR | |
| | WQQGNVFSCSVMHEALHNHYTQKSLSLSPG | |
| SDE2 | EPKSCDKTHTCPPCPAPELLGGPSVFLFPPKPKDT | SEQ ID |
| (T299K, | LMISRTPEVTCVV [symbol] DVSHEDPEVKFNWYVDGVE | NO: 30 |
| V264T, | VHNAKTKPREEQYNS [symbol] YRVVSVLTVLHQDWLN | |
| aglycosylated) | GKEYKCKVSNKALPAPIEKTISKAKGQPREPQVY | |
| | TLPPSRDELTKNQVSLTCLVKGFYPSDIAVEWES | |
| | NGQPENNYKTTPPVLDSDGSFFLYSKLTVDKSR | |
| | WQQGNVFSCSVMHEALHNHYTQKSLSLSPG | |
| SDE3 | EPKSCDKTHTCPPCPAPELLGGPSVFLFPPKPKDT | SEQ ID |
| (T299K, | LMISRTPEVTCVVVD [symbol] SHEDPEVKFNWYVDGVE | NO: 31 |
| V266F, | VHNAKTKPREEQYNS [symbol] YRVVSVLTVLHQDWLN | |
| aglycosylated) | GKEYKCKVSNKALPAPIEKTISKAKGQPREPQVY | |
| | TLPPSRDELTKNQVSLTCLVKGFYPSDIAVEWES | |
| | NGQPENNYKTTPPVLDSDGSFFLYSKLTVDKSR | |
| | WQQGNVFSCSVMHEALHNHYTQKSLSLSPG | |
| SDE4 | EPKSCDKTHTCPPCPAPELLGGPSVFLFPPKPKDT | SEQ ID |
| (T299K, | LMISRTPEVTC [symbol] V [symbol] DVSHEDPEVKFNWYVDGVE | NO: 32 |
| V262L, | VHNAKTKPREEQYNS [symbol] YRVVSVLTVLHQDWLN | |
| V264T, | GKEYKCKVSNKALPAPIEKTISKAKGQPREPQVY | |
| aglycosylated) | TLPPSRDELTKNQVSLTCLVKGFYPSDIAVEWES | |
| | NGQPENNYKTTPPVLDSDGSFFLYSKLTVDKSR | |
| | WQQGNVFSCSVMHEALHNHYTQKSLSLSPG | |
| SDE5 | EPKSCDKTHTCPPCPAPELLGGPSVFLFPPKPKDT | SEQ ID |
| (T299K, | LMISRTPEVTCVV [symbol] D [symbol] SHEDPEVKFNWYVDGVE | NO: 33 |
| V264T, | VHNAKTKPREEQYNS [symbol] YRVVSVLTVLHQDWLN | |
| V266F, | GKEYKCKVSNKALPAPIEKTISKAKGQPREPQVY | |
| aglycosylated) | TLPPSRDELTKNQVSLTCLVKGFYPSDIAVEWES | |
| | NGQPENNYKTTPPVLDSDGSFFLYSKLTVDKSR | |
| | WQQGNVFSCSVMHEALHNHYTQKSLSLSPG | |
| SDE6 | EPKSCDKTHTCPPCPAPELLGGPSVFLFPPKPKDT | SEQ ID |
| (T299K, Loop | LMISRTPEVTCVVVDVS__PDP_VKFNWYVDGVE | NO: 34 |
| replacement, | VHNAKTKPREEQYNS [symbol] YRVVSVLTVLHQDWLN | |
| aglycosylated) | GKEYKCKVSNKALPAPIEKTISKAKGQPREPQVY | |
| | TLPPSRDELTKNQVSLTCLVKGFYPSDIAVEWES | |
| | NGQPENNYKTTPPVLDSDGSFFLYSKLTVDKSR | |
| | WQQGNVFSCSVMHEALHNHYTQKSLSLSPG | |
| SDE7 | EPKSCDKTHTCPPCPAPELLGGPSVFLFPPKPKDT | SEQ ID |
| (T299K, Loop | LMISRTPEVTC [symbol] V [symbol] DVS__PDP_VKFNWYVDGVE | NO: 35 |
| replacement, | VHNAKTKPREEQYNS [symbol] YRVVSVLTVLHQDWLN | |
| V262L/V264T, | GKEYKCKVSNKALPAPIEKTISKAKGQPREPQVY | |
| aglycosylated) | TLPPSRDELTKNQVSLTCLVKGFYPSDIAVEWES | |
| | NGQPENNYKTTPPVLDSDGSFFLYSKLTVDKSR | |
| | WQQGNVFSCSVMHEALHNHYTQKSLSLSPG | |
| SDE8 | EPKSCDKTHTCPPCPAPELLGGPSVFLFPPKPKDT | SEQ ID |
| (T299K, | LMISRTPEVTC [symbol] V [symbol] D [symbol] SHEDPEVKFNWYVDGVE | NO: 36 |
| V262L, | VHNAKTKPREEQYNS [symbol] YRVVSVLTVLHQDWLN | |
| V264T, | GKEYKCKVSNKALPAPIEKTISKAKGQPREPQVY | |
| V266F, | TLPPSRDELTKNQVSLTCLVKGFYPSDIAVEWES | |
| aglycosylated | NGQPENNYKTTPPVLDSDGSFFLYSKLTVDKSR | |
| | WQQGNVFSCSVMHEALHNHYTQKSLSLSPG | |
| SDE9 (T299K, | EPKSCDKTHTCPPCPAPELLGGPSVFLFPPKPKDT | SEQ ID |
| Loop | LMISRTPEVTC [symbol] V [symbol] D [symbol] S__PDP_VKFNWYVDGVE | NO: 37 |
| replacement, | VHNAKTKPREEQYNS [symbol] YRVVSVLTVLHQDWLN | |
| V262L/V264T/ | GKEYKCKVSNKALPAPIEKTISKAKGQPREPQVY | |
| V266F, | TLPPSRDELTKNQVSLTCLVKGFYPSDIAVEWES | |
| aglycosylated) | NGQPENNYKTTPPVLDSDGSFFLYSKLTVDKSR | |
| | WQQGNVFSCSVMHEALHNHYTQKSLSLSPG | |
| CN578 | EPKSCDKTHTCPPCPAPELLGGPSVFLFPPKPKDT | SEQ ID |
| (T299K) | LMISRTPEVTCVVVDVSHEDPEVKFNWYVDGVE | NO: 60 |
| | VHNAKTKPREEQYNS [symbol] YRVVSVLTVLHQDWLN | |
| | GKEYKCKVSNKALPAPIEKTISKAKGQPREPQVY | |
| | TLPPSRDELTKNQVSLTCLVKGFYPSDIAVEWES | |
| | NGQPENNYKTTPPVLDSDGSFFLYSKLTVDKSR | |
| | WQQGNVFSCSVMHEALHNHYTQKSLSLSPG | |
| CN647 | EPKSCDKTHTCPPCPAPELLGGPSVFLFPPKPKDT | SEQ ID |
| (T299K + | LMISRTPEVTCVVVDVSHEDPEVKFNWYVDGVE | NO: 61 |
| N297D) | VHNAKTKPREEQY [symbol] S [symbol] YRVVSVLTVLHQDWLN | |
| | GKEYKCKVSNKALPAPIEKTISKAKGQPREPQVY | |
| | TLPPSRDELTKNQVSLTCLVKGFYPSDIAVEWES | |
| | NGQPENNYKTTPPVLDSDGSFFLYSKLTVDKSR | |
| | WQQGNVFSCSVMHEALHNHYTQKSLSLSPG | |
| CN646 | EPKSCDKTHTCPPCPAPELLGGPSVFLFPPKPKDT | SEQ ID |
| (T299K + | LMISRTPEVTCVVVDVSHEDPEVKFNWYVDGVE | NO: 62 |
| N297S) | VHNAKTKPREEQY [symbol] S [symbol] YRVVSVLTVLHQDWLN | |
| | GKEYKCKVSNKALPAPIEKTISKAKGQPREPQVY | |
| | TLPPSRDELTKNQVSLTCLVKGFYPSDIAVEWES | |
| | NGQPENNYKTTPPVLDSDGSFFLYSKLTVDKSR | |
| | WQQGNVFSCSVMHEALHNHYTQKSLSLSPG | |
| CN645 | EPKSCDKTHTCPPCPAPELLGGPSVFLFPPKPKDT | SEQ ID |
| (T299K + | LMISRTPEVTCVVVDVSHEDPEVKFNWYVDGVE | NO: 63 |
| N297P) | VHNAKTKPREEQY [symbol] S [symbol] YRVVSVLTVLHQDWLN | |
| | GKEYKCKVSNKALPAPIEKTISKAKGQPREPQVY | |
| | TLPPSRDELTKNQVSLTCLVKGFYPSDIAVEWES | |
| | NGQPENNYKTTPPVLDSDGSFFLYSKLTVDKSR | |
| | WQQGNVFSCSVMHEALHNHYTQKSLSLSPG | |

### (IV). Methods for Stabilizing Variant Fc Polypeptides

In certain aspects, the invention pertains to a method of stabilizing a polypeptide comprising an Fc region (e.g., an aglycosylated Fc region), the method comprising: (a) selecting one or more amino acid positions within at least one Fc moiety of a starting Fc region for mutation; and (b) mutating the one or more amino acid positions selected for mutation, thereby stabilizing the polypeptide.

In one embodiment, the starting Fc region is an IgG1 Fc region. In another embodiment, the starting Fc region is an IgG4 Fc region. In another embodiment, the starting Fc region is a chimeric Fc region. In one embodiment, the starting Fc region is an aglycosylated IgG1 Fc region. In another embodiment, the starting Fc region is an aglycosylated IgG4 Fc region.

In one embodiment, an amino acid position selected for mutation is in an extended loop in the Fc region of a starting IgG molecule (e.g., an IgG4 molecule). In another embodiment, the amino acid position selected for mutation resides in the interface between CH3 domains. In another embodiment, an amino acid position selected for mutation is near a contact site with the carbohydrate in the 1hzh crystal structure (e.g., V264, R292 or V303). In other embodiments, the amino acid position may be near the CH3/CH2 interface, or near the CH3/CH2 interface (e.g., H310). In another embodiment, one or more mutations that alter the overall surface charge of the Fc region, e.g., in one or more of a set of surface exposed glutamine residues (Q268, Q274 or Q355) may be made. In another embodiment, the amino acid positions are valine residues found in the “valine core” of CH2 and CH3. The “valine core” in CH2 is five valine residues (V240, V255, V263, V302 and V323) that all are orientated into the same proximal interior core of the CH2 domain. A similar “valine core” is observed for CH3 (V348, V369, V379, V397, V412 and V427). In another embodiment, an amino acid position selected for mutation is at a position that is predicted to interact with or contact the N-linked carbohydrate at amino acid 297. Such amino acid positions can be identified by examining a crystal structure of the Fc region bound to a cognate Fc receptor (e.g., FcγRIIIa). Exemplary amino acids which form interactions with N297 include a loop formed by residues 262-270.

Exemplary amino acid positions include amino acid positions 240, 255, 262-266, 267-271, 292-299, 302-309, 379, 397-399, 409, 412 and 427 according to the EU numbering convention. In certain embodiments, the one or more amino acid positions selected for mutation are one or more amino acid positions selected from the group consisting of: 240, 255, 262, 263, 264, 266, 268, 274, 292, 299, 302, 303, 307, 309, 323, 348, 355, 369, 379, 397, 399, 409, 412 and 427. In certain embodiments, the one or more amino acid positions selected for mutation are one or more amino acid positions selected from the group consisting of: 240, 262, 264, 266, 297, 299, 307, 309, 399, 409 and 427. In another embodiment, the one or more amino acid positions are one or more amino acid positions selected from the group consisting of: 297, 299, 307, 309, 409 and 427. In another embodiment, the one or more amino acid positions are selected from amino acid residues 240, 262, 264, and 266. In another embodiment, at least one of the amino acid positions is at EU position 297. In another embodiment, at least one of the amino acid positions is at EU position 299. In another embodiment, at least one of the amino acid positions is at EU position 307. In another embodiment, at least one of the amino acid positions is at EU position 309. In another embodiment, at least one of the amino acid positions is at EU position 399. In another embodiment, at least one of the amino acid positions is at EU position 409. In another embodiment, at least one of the amino acid positions is at EU position 427.

In certain embodiments, the Fc region is an IgG1 Fc region. In certain embodiments, wherein the Fc region is an IgG1 Fc region, the one or more amino acid positions are selected from amino acid residues 240, 262, 264, 299, 297, and 266. In other embodiments, wherein the Fc region is an IgG4 Fc region, the one or more amino acid positions are selected from amino acid residues 297, 299, 307, 309, 399, 409 and 427.

In one embodiment, the mutation reduces the size of the amino acid side chain at the amino acid position (e.g., a substitution with an alanine (A), a serine (S) or threonine (T)). In another embodiment, the mutation is a substitution with an amino acid having a non-polar side chain (e.g., a substitution with a glycine (G), an alanine (A), a valine (V), a leucine (L), an isoleucine (I), a methionine (M), a proline (P), a phenylalanine (F), and a tryptophan (W)). In another embodiment, a mutation adds hydrophobicity to the CH3 interface, e.g., to increase the association between the two interacting domains (e.g., Y349F, T350V and T394V) or increase bulk in the side chains of the interface (e.g., F405Y). In another embodiment, one or more amino acids of the “valine core” are substituted with isoleucines or phenylalanines in order to increase their stability. In another embodiment, amino acids (e.g., L351 and/or L368) are mutated to higher branched hydrophobic sidechains.

In one embodiment, the mutation is a substitution with an alanine (A). In one embodiment, the mutation is a substitution with a phenylalanine (F). In another embodiment, the mutation is a substitution with a leucine (L). In one embodiment, the mutation is a substitution with a threonine (T). In another embodiment, the mutation is a substitution with a lysine (K). In one embodiment, the mutation is a substitution with a proline (P). In one embodiment, the mutation is a substitution with a phenylalanine (F).

In one embodiment, the mutating comprises one or more of the mutations or substitutions set forth in Table 1.1, Table 1.2, Table 1.3, and/or Table 1.4 infra.

In certain embodiments, the mutating comprises one or more substitutions selected from the group consisting of: 240F, 262L, 264T, 266F, 297Q, 297S, 297D, 299A, 299K, 307P, 309K, 309M, 309P, 323F, 399S, 409M and 427F (EU Numbering Convention). In another embodiment, the mutating comprises one or more substitutions selected from the group consisting of: 299A, 299K, 307P, 309K, 309M, 309P, 323F, 399E, 399S, 409K, 409M and 427F. In another embodiment, the one or more amino acid positions are selected from amino acid residues 240F, 262L, 264T, and 266F. In another embodiment, at least one of the substitutions is 299A. In another embodiment, at least one of the substitutions is 299K. In another embodiment, at least one of the substitutions is 307P. In another embodiment, at least one of the substitutions is 309K.

In another embodiment, at least one of the substitutions is 309M. In another embodiment, at least one of the substitutions is 309P. In another embodiment, at least one of the substitutions is 323F. In another embodiment, at least one of the substitutions is 399S. In another embodiment, at least one of the substitutions is 399E. In another embodiment, at least one of the substitutions is 409K. In another embodiment, at least one of the substitutions is 409M. In another embodiment, at least one of the substitutions is 427F.

In another embodiment, the mutating comprises two or more substitutions (e.g., 2, 3, 4, or 5). In another embodiment, the mutating comprises three or more substitutions (e.g., 3, 4, 5, or 6). In yet another embodiment, the stabilized Fc region comprises four or more substitutions (e.g., 4, 5, 6, or 7).

In another aspect, the invention pertains to a method of making a stabilized binding molecule comprising a stabilized Fc region, the method comprising genetically fusing a polypeptide comprising stabilized Fc region of the invention to the amino terminus or the carboxy terminus of a binding moiety. In certain embodiments, the stabilized Fc region is stabilized according to the methods of the invention.

### V. Methods of Evaluating Protein Stability

The stability properties of the compositions of the invention can be analyzed using methods known in the art. Stability parameters acceptable to those in the art may be employed. Exemplary parameters are described in more detail below. In exemplary embodiments, thermal stability is evaluated. In proffered embodiments, the expression levels (e.g., as measured by % yield) of the compositions of the invention are evaluated. In other preferred embodiments, the aggregation levels of the compositions of the invention are evaluated.

In certain embodiments, the stability properties of an Fc polypeptide are compared with that of a suitable control. Exemplary controls include a parental Fc polypeptide such as a wild-type Fc polypeptide, wild-type (glycosylated) IgG1 or IgG4 antibody. Another exemplary control is an aglycosylated Fc polypeptide, an aglycosylated IgG1 or IgG4 antibody.

In one embodiment, one or more parameters described below are measured. In one embodiment, one or more of these parameters is measured following expression in a mammalian cell. In one embodiment, one or more parameters described below are measured under large scale manufacturing conditions (e.g., expression of Fc polypeptide or molecules comprising Fc polypeptide in a bioreactor).

### a) Thermal Stability

The thermal stability of the compositions of the invention may be analyzed using a number of non-limiting biophysical or biochemical techniques known in the art. In certain embodiments, thermal stability is evaluated by analytical spectroscopy.

An exemplary analytical spectroscopy method is Differential Scanning calorimetry (DSC). DSC employs a calorimeter which is sensitive to the heat absorbances that accompany the unfolding of most proteins or protein domains (see, e.g. Sanchez-Ruiz, et al., Biochemistry, 27: 1648-52, 1988). To determine the thermal stability of a protein, a sample of the protein is inserted into the calorimeter and the temperature is raised until the Fc polypeptide (or a CH2 or CH3 domain thereof) unfolds. The temperature at which the protein unfolds is indicative of overall protein stability.

Another exemplary analytical spectroscopy method is Circular Dichroism (CD) spectroscopy. CD spectrometry measures the optical activity of a composition as a function of increasing temperature. Circular dichroism (CD) spectroscopy measures differences in the absorption of left-handed polarized light versus right-handed polarized light which arise due to structural asymmetry. A disordered or unfolded structure results in a CD spectrum very different from that of an ordered or folded structure. The CD spectrum reflects the sensitivity of the proteins to the denaturing effects of increasing temperature and is therefore indicative of a protein's thermal stability (see van Mierlo and Steemsma, J. Biotechnol., 79(3):281-98, 2000).

Another exemplary analytical spectroscopy method for measuring thermal stability is Fluorescence Emission Spectroscopy (see van Mierlo and Steemsma, supra). Yet another exemplary analytical spectroscopy method for measuring thermal stability is Nuclear Magnetic Resonance (NMR) spectroscopy (see, e.g. van Mierlo and Steemsma, supra).

In other embodiments, the thermal stability of a composition of the invention is measured biochemically. An exemplary biochemical method for assessing thermal stability is a thermal challenge assay. In a “thermal challenge assay”, a composition of the invention is subjected to a range of elevated temperatures for a set period of time. For example, in one embodiment, test Fc polypeptide comprising Fc regions are subject to an range of increasing temperatures, e.g., for 1-1.5 hours. The ability of the Fc region to bind an Fc receptor (e.g., an FcγR, Protein A, or Protein G) is then assayed by a relevant biochemical assay (e.g, ELISA or DELFIA). An exemplary thermal challenge assay is described in Example 2 infra.

In one embodiment, such an assay may be done in a high-throughput format. In another embodiment, a library of Fc variants may be created using methods known in the art. Fc expression may be induced and Fc may be subjected to thermal challenge. The challenged test samples may be assayed for binding and those Fc polypeptides which are stable may be scaled up and further characterized.

In certain embodiments, thermal stability is evaluated by measuring the melting temperature (Tm) of a composition of the invention using any of the above techniques (e.g. analytical spectroscopy techniques). The melting temperature is the temperature at the midpoint of a thermal transition curve wherein 50% of molecules of a composition are in a folded state.

In other embodiments, thermal stability is evaluated by measuring the specific heat or heat capacity (Cp) of a composition of the invention using an analytical calorimetric technique (e.g. DSC). The specific heat of a composition is the energy (e.g. in kcal/mol) required to raise by 1° C., the temperature of 1 mol of water. As large Cp is a hallmark of a denatured or inactive protein composition. In certain embodiments, the change in heat capacity (ΔCp) of a composition is measured by determining the specific heat of a composition before and after its thermal transition. In other embodiments, thermal stability may be evaluated by measuring or determining other parameters of thermodynamic stability including Gibbs free energy of unfolding (AG), enthalpy of unfolding (ΔH), or entropy of unfolding (ΔS).

In other embodiments, one or more of the above biochemical assays (e.g. a thermal challenge assay) is used to determine the temperature (ie. the T_{C} value) at which 50% of the composition retains its activity (e.g. binding activity).

### b) % Aggregation

In certain embodiments, the stability of a composition of the invention is determined by measuring its propensity to aggregate. Aggregation can be measured by a number of non-limiting biochemical or biophysical techniques. For example, the aggregation of a composition of the invention may be evaluated using chromatography, e.g. Size-Exclusion Chromatography (SEC). SEC separates molecules on the basis of size. A column is filled with semi-solid beads of a polymeric gel that will admit ions and small molecules into their interior but not large ones. When a protein composition is applied to the top of the column, the compact folded proteins (ie. non-aggregated proteins) are distributed through a larger volume of solvent than is available to the large protein aggregates. Consequently, the large aggregates move more rapidly through the column, and in this way the mixture can be separated or fractionated into its components. Each fraction can be separately quantified (e.g. by light scattering) as it elutes from the gel. Accordingly, the % aggregation of a composition of the invention can be determined by comparing the concentration of a fraction with the total concentration of protein applied to the gel. Stable compositions elute from the column as essentially a single fraction and appear as essentially a single peak in the elution profile or chromatogram.

In preferred embodiments, SEC is used in conjunction with in-line light scattering (e.g. classical or dynamic light scattering) to determine the % aggregation of a composition. In certain preferred embodiments, static light scattering is employed to measure the mass of each fraction or peak, independent of the molecular shape or elution position. In other preferred embodiments, dynamic light scattering is employed to measure the hydrodynamic size of a composition. Other exemplary methods for evaluating protein stability include High-Speed SEC (see e.g. Corbett et al., Biochemistry. 23(8):1888-94, 1984).

In a preferred embodiment, the % aggregation is determined by measuring the fraction of protein aggregates within the protein sample. In a preferred embodiment, the % aggregation of a composition is measured by determining the fraction of folded protein within the protein sample.

### c) % Yield

In other embodiments, the stability of a composition of the invention is evaluated by measuring the amount of protein that is recovered (herein the “% yield”) following expression (e.g. recombinant expression) of the protein. For example, the % yield can be measured by determining milligrams of protein recovered for every ml of host culture media (ie. mg/ml of protein). In a preferred embodiment the % yield is evaluated following expression in a mammalian host cell (e.g. a CHO cell).

### d) % Loss

In yet other embodiments, the stability of a composition of the invention is evaluated by monitoring the loss of protein at a range of temperatures (e.g. from −80 to 25° C.) following storage for a defined time period. The amount or concentration of recovered protein can be determined using any protein quantification method known in the art, and compared with the initial concentration of protein. Exemplary protein quantification methods include SDS-PAGE analysis or the Bradford assay for (Bradford, et al., Anal. Biochem. 72, 248, (1976)). A preferred method for evaluating % loss employs any of the analytical SEC methods described supra. It will be appreciated that % Loss measurements can be determined under any desired storage condition or storage formulation, including, for example, lyophilized protein preparations.

### e) % Proteolysis

In still other embodiments, the stability of a composition of the invention is evaluated by determining the amount of protein that is proteolyzed following storage under standard conditions. In an exemplary embodiment, proteolysis is determined by SDS-PAGE a sample of the protein wherein the amount of intact protein is compared with the amount of low-molecular weight fragments which appear on the SDS-PAGE gel. In another exemplary embodiment, proteolysis is determined by Mass Spectrometry (MS), wherein the amount of protein of the expected molecular weight is compared with the amount of low-molecular weight protein fragments within the sample.

### f) Binding Affinity

In still other embodiments, the stability of a composition of the invention may be assessed by determining its target binding affinity. A wide variety of methods for determining binding affinity are known in the art. An exemplary method for determining binding affinity employs surface plasmon resonance. Surface plasmon resonance is an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Jönsson, U., et al. (1993) Ann. Biol. Clin. 51:19-26; Jönsson, U., et al. (1991) Biotechniques 11:620-627; Johnsson, B., et al. (1995) J. Mol. Recognit. 8:125-131; and Johnnson, B., et al. (1991) Anal. Biochem. 198:268-277.

### g) Other Binding Studies

In yet other embodiments, the stability of a composition of the invention may be assessed by quantifying the binding of a labeled compound to denatured or unfolded portions of a binding molecule. Such molecules are preferably hydrophobic, as they preferably bind or interact with large hydrophobic patches of amino acids that are normally buried in the interior of the native protein, but which are exposed in a denatured or unfolded binding molecule. An exemplary labeled compound is the hydrophobic fluorescent dye, 1-anilino-8-naphthaline sulfonate (ANS).

### (VI) Stabilized Binding Polypeptides Comprising Stabilized Fc Regions

In certain aspects, the invention provides stabilized binding polypeptides comprising the stabilized Fc polypeptides of the invention. As described above, variant Fc polypeptides of the invention (and/or the parental Fc polypeptides from which the are derived) may further comprise a binding site to form a stabilized binding polypeptide. A variety of binding polypeptides of alternative designs are within the scope of the invention. For example, one or more binding sites can be fused to, linked with, or incorporated within (e.g., veneered onto) a Fc region of the Fc polypeptide in multiple orientations. In one exemplary embodiment, a binding polypeptide comprises a binding site fused to an N-terminus of the Fc region. In another exemplary embodiment, a binding polypeptide comprises a binding site at a C-terminus of the Fc region. The binding polypeptide of the invention may comprise binding sites at both an C-terminus and an N-terminus of a Fc region. In yet other embodiments, the binding polypeptide may comprise a binding site in an N-terminal and/or C-terminal interdomain region of an Fc region (e.g., between the CH2 and CH3 domains of an Fc moiety). Alternatively, the binding site may be incorporated in an interdomain region between the hinge and CH2 domains of an Fc moiety. In other embodiments, wherein the Fc region of the Fc polypeptide is a scFc region, a binding polypeptide may comprise one or more binding sites within a linker polypeptide which links two or more Fc moieties of a scFc region as a single contiguous sequence.

In still further embodiments, the stabilized binding polypeptide of the invention comprises a binding site which is introduced into an Fc moiety of a stabilized Fc region. For example, a binding site may be veneered into an N-terminal CH2 domain, an N-terminal CH3 domain, a C-terminal CH2 domain, and/or a C-terminal CH3 domain. In one embodiment, the CDR loops of an antibody are veneered into one or both CH3 domains scFc region. Methods for veneering CDR loops and other binding moieties into the CH2 and/or CH3 domains of an Fc region are disclosed, for example, in International PCT Publication No. WO 08/003,116, which is incorporated by reference herein.

It is recognized by those skilled in the art that an stabilized binding polypeptide may comprise two or more binding sites (e.g., 2, 3, 4, or more binding sites) which are linked, fused, or integrated (e.g., veneered) into a stabilized Fc region of an Fc polypeptide of the invention using any combination of the orientations.

In certain embodiments, the binding polypeptides of the invention comprise two binding sites and at least one stabilized Fc region. For example, binding sites may be operably linked to both the N-terminus and C-terminus of a stabilized Fc region. In other exemplary embodiments, binding sites may be operably linked to both the N- and C-terminal ends of multiple stabilized Fc regions. Where the stabilized Fc region is a scFc region, two or more scFc regions may be linked together in series to form a tandem array of stabilized Fc regions.

In other embodiments, two or more binding sites are linked to each other (e.g., via a polypeptide linker) in series, and the tandem array of binding sites is operably linked (e.g., chemically conjugated or genetically fused (e.g., either directly or via a polypeptide linker)) to either the C-terminus or the N-terminus of a stabilized Fc region or a tandem array of stabilized Fc regions (i.e., tandem stabilized scFc regions). In other embodiments, the tandem array of binding sites is operably linked to both the C-terminus and the N-terminus of a single stabilized Fc region or a tandem array of stabilized Fc regions.

In other embodiments, a stabilized binding polypeptide of the invention is a trivalent binding polypeptide comprising three binding sites. An exemplary trivalent binding polypeptide of the invention is bispecific or trispecific. For example, a trivalent binding polypeptide may be bivalent (i.e., have two binding sites) for one specificity and monovalent for a second specificity.

In yet other embodiments, a stabilized binding polypeptide of the invention is a tetravalent binding polypeptide comprising four binding sites. An exemplary tetravalent binding polypeptide of the invention is bispecific. For example, a tetravalent binding polypeptide may be bivalent (i.e., have two binding sites) for each specificity.

As mentioned above, in other embodiments, one or more binding sites may be inserted between two Fc moieties of a stabilized scFc region. For example, one or more binding sites may form all or part of a polypeptide linker of a binding polypeptide of the invention.

Preferred binding polypeptides of the invention comprise at least one of an antigen binding site (e.g., an antigen binding site of an antibody, antibody variant, or antibody fragment), a receptor binding portion of ligand, or a ligand binding portion of a receptor.

In other embodiments, the binding polypeptides of the invention comprise at least one binding site comprising one or more of any one of the biologically-relevant peptides discussed supra.

In certain embodiments, the binding polypeptides of the invention have at least one binding site specific for a target molecule which mediates a biological effect. In one embodiment, the binding site modulates cellular activation or inhibition (e.g., by binding to a cell surface receptor and resulting in transmission of an activating or inhibitory signal). In one embodiment, the binding site is capable of initiating transduction of a signal which results in death of the cell (e.g., by a cell signal induced pathway, by complement fixation or exposure to a payload (e.g., a toxic payload) present on the binding molecule), or which modulates a disease or disorder in a subject (e.g., by mediating or promoting cell killing, by promoting lysis of a fibrin clot or promoting clot formation, or by modulating the amount of a substance which is bioavailable (e.g., by enhancing or reducing the amount of a ligand such as TNFα in the subject)). In another embodiment, the binding polypeptides of the invention have at least one binding site specific for an antigen targeted for reduction or elimination, e.g., a cell surface antigen or a soluble antigen, together with at least one genetically-fused Fc region (i.e., scFc region).

In another embodiment, binding of the binding polypeptides of the invention to a target molecule (e.g. antigen) results in the reduction or elimination of the target molecule, e.g., from a tissue or from circulation. In another embodiment, the binding polypeptide has at least one binding site specific for a target molecule that can be used to detect the presence of the target molecule (e.g., to detect a contaminant or diagnose a condition or disorder). In yet another embodiment, a binding polypeptide of the invention comprises at least one binding site that targets the molecule to a specific site in a subject (e.g., to a tumor cell, an immune cell, or blood clot).

In certain embodiments, the binding polypeptides of the invention may comprise two or more binding sites. In one embodiment, the binding sites are identical. In another embodiment, the binding sites are different.

In other embodiments, the binding polypeptides of the invention may be assembled together or with other polypeptides to form binding proteins having two or more polypeptides (“binding proteins” or “multimers”), wherein at least one polypeptide of the multimer is a binding polypeptide of the invention. Exemplary multimeric forms include dimeric, trimeric, tetrameric, and hexameric altered binding proteins and the like. In one embodiment, the polypeptides of the binding protein are the same (ie. homomeric altered binding proteins, e.g. homodimers, homotetramers). In another embodiment, the polypeptides of the binding protein are different (e.g. heteromeric).

In one embodiment, an polypeptide of the invention a CH1 domain from an IgG4 antibody, a CH2 domain from an IgG4 antibody and a CH3 domain from an IgG1 antibody. In one embodiment, the polypeptide further comprises a Ser228Pro substitution. The polypeptide may further comprise a mutation at amino acid 297 and/or 299, e.g., 297Q and/or 299K or 297S and/or 299K. The polypeptide may also comprise a CH1 domain from an IgG1 or an IgG4 antibody, a CH2 domain from an IgG4 antibody and a CH3 domain from an IgG1 antibody; which polypeptide may comprise one or more of a Ser228Pro, 297Q or 299K substitutions. The amino acid sequence of an Fc region consisting of a CH1 domain from an IgG4 molecule (with an Ser228Pro substitution), a CH2 domain from an IgG4 antibody and a CH3 domain from an IgG1 antibody is provided in SEQ ID NO: 28. In one embodiment, a stabilized Fc polypeptide of the invention comprises the amino acid sequence set forth in SEQ ID NO:25. In one embodiment, a stabilized Fc polypeptide of the invention comprises the amino acid sequence set forth in SEQ ID NO:59. In one embodiment, a stabilized Fc polypeptide of the invention comprises the amino acid sequence set forth in SEQ ID NO:60. In one embodiment, a stabilized Fc polypeptide of the invention comprises the amino acid sequence set forth in SEQ ID NO:61. In one embodiment, a stabilized Fc polypeptide of the invention comprises the amino acid sequence set forth in SEQ ID NO:62.

In one embodiment, the Fc region of a polypeptide of the invention is a single chain (scFc). In one embodiment, a molecule comprising an Fc region described in this paragraph is monovalent. In one embodiment, the molecule comprising an Fc region described in this paragraph is monovalent and the Fc region is a scFc. Molecules comprising an Fc region described herein may also comprise an scFv.

i. Antigen Binding Sites

### (a) Antibodies

In certain embodiments, a binding polypeptide of the invention comprises at least one antigen binding site of an antibody. Binding polypeptides of the invention may comprise a variable region or portion thereof (e.g. a VL and/or VH domain) derived from an antibody using art recognized protocols. For example, the variable domain may be derived from antibody produced in a non-human mammal, e.g., murine, guinea pig, primate, rabbit or rat, by immunizing the mammal with the antigen or a fragment thereof. See Harlow & Lane, supra, incorporated by reference for all purposes. The immunoglobulin may be generated by multiple subcutaneous or intraperitoneal injections of the relevant antigen (e.g., purified tumor associated antigens or cells or cellular extracts comprising such antigens) and an adjuvant. This immunization typically elicits an immune response that comprises production of antigen-reactive antibodies from activated splenocytes or lymphocytes.

While the variable region may be derived from polyclonal antibodies harvested from the serum of an immunized mammal, it is often desirable to isolate individual lymphocytes from the spleen, lymph nodes or peripheral blood to provide homogenous preparations of monoclonal antibodies (MAbs) from which the desired variable region is derived. Rabbits or guinea pigs are typically used for making polyclonal antibodies. Mice are typically used for making monoclonal antibodies. Monoclonal antibodies can be prepared against a fragment by injecting an antigen fragment into a mouse, preparing “hybridomas” and screening the hybridomas for an antibody that specifically binds to the antigen. In this well known process (Kohler et al., (1975), Nature, 256:495) the relatively short-lived, or mortal, lymphocytes from the mouse which has been injected with the antigen are fused with an immortal tumor cell line (e.g. a myeloma cell line), thus, producing hybrid cells or “hybridomas” which are both immortal and capable of producing the antibody genetically encoded by the B cell. The resulting hybrids are segregated into single genetic strains by selection, dilution, and regrowth with each individual strain comprising specific genes for the formation of a single antibody. They produce antibodies which are homogeneous against a desired antigen and, in reference to their pure genetic parentage, are termed “monoclonal”.

Hybridoma cells thus prepared are seeded and grown in a suitable culture medium that preferably contains one or more substances that inhibit the growth or survival of the unfused, parental myeloma cells. Those skilled in the art will appreciate that reagents, cell lines and media for the formation, selection and growth of hybridomas are commercially available from a number of sources and standardized protocols are well established. Generally, culture medium in which the hybridoma cells are growing is assayed for production of monoclonal antibodies against the desired antigen. Preferably, the binding specificity of the monoclonal antibodies produced by hybridoma cells is determined by immunoprecipitation or by an in vitro assay, such as a radioimmunoassay (RIA) or enzyme-linked immunosorbent assay (ELISA). After hybridoma cells are identified that produce antibodies of the desired specificity, affinity and/or activity, the clones may be subcloned by limiting dilution procedures and grown by standard methods (Goding, Monoclonal Antibodies: Principles and Practice, pp 59-103 (Academic Press, 1986)). It will further be appreciated that the monoclonal antibodies secreted by the subclones may be separated from culture medium, ascites fluid or serum by conventional purification procedures such as, for example, affinity chromatography (e.g., protein-A, protein-G, or protein-L affinity chromatography), hydroxylapatite chromatography, gel electrophoresis, or dialysis.

Optionally, antibodies may be screened for binding to a specific region or desired fragment of the antigen without binding to other nonoverlapping fragments of the antigen. The latter screening can be accomplished by determining binding of an antibody to a collection of deletion mutants of the antigen and determining which deletion mutants bind to the antibody. Binding can be assessed, for example, by Western blot or ELISA. The smallest fragment to show specific binding to the antibody defines the epitope of the antibody. Alternatively, epitope specificity can be determined by a competition assay is which a test and reference antibody compete for binding to the antigen. If the test and reference antibodies compete, then they bind to the same epitope or epitopes sufficiently proximal such that binding of one antibody interferes with binding of the other.

DNA encoding the desired monoclonal antibody may be readily isolated and sequenced using any of the conventional procedures described supra for the isolation of constant region domain sequences (e.g., by using oligonucleotide probes that are capable of binding specifically to genes encoding the heavy and light chains of murine antibodies). The isolated and subcloned hybridoma cells serve as a preferred source of such DNA. More particularly, the isolated DNA (which may be synthetic as described herein) may be used to clone the desired variable region sequences for incorporation in the binding polypeptides of the invention.

In other embodiments, the binding site is derived from a fully human antibody. Human or substantially human antibodies may be generated in transgenic animals (e.g., mice) that are incapable of endogenous immunoglobulin production (see e.g., U.S. Pat. Nos. 6,075,181, 5,939,598, 5,591,669 and 5,589,369, each of which is incorporated herein by reference). For example, it has been described that the homozygous deletion of the antibody heavy-chain joining region in chimeric and germ-line mutant mice results in complete inhibition of endogenous antibody production. Transfer of a human immunoglobulin gene array to such germ line mutant mice will result in the production of human antibodies upon antigen challenge. Another preferred means of generating human antibodies using SCID mice is disclosed in U.S. Pat. No. 5,811,524 which is incorporated herein by reference. It will be appreciated that the genetic material associated with these human antibodies may also be isolated and manipulated as described herein.

Yet another highly efficient means for generating recombinant antibodies is disclosed by Newman, Biotechnology, 10: 1455-1460 (1992). Specifically, this technique results in the generation of primatized antibodies that contain monkey variable domains and human constant sequences. This reference is incorporated by reference in its entirety herein. Moreover, this technique is also described in commonly assigned U.S. Pat. Nos. 5,658,570, 5,693,780 and 5,756,096 each of which is incorporated herein by reference.

In another embodiment, lymphocytes can be selected by micromanipulation and the variable genes isolated. For example, peripheral blood mononuclear cells can be isolated from an immunized mammal and cultured for about 7 days in vitro. The cultures can be screened for specific IgGs that meet the screening criteria. Cells from positive wells can be isolated. Individual Ig-producing B cells can be isolated by FACS or by identifying them in a complement-mediated hemolytic plaque assay. Ig-producing B cells can be micromanipulated into a tube and the VH and VL genes can be amplified using, e.g., RT-PCR. The VH and VL genes can be cloned into an antibody expression vector and transfected into cells (e.g., eukaryotic or prokaryotic cells) for expression.

Alternatively, variable (V) domains can be obtained from libraries of variable gene sequences from an animal of choice. Libraries expressing random combinations of domains, e.g., V_{H} and V_{L} domains, can be screened with a desired antigen to identify elements which have desired binding characteristics. Methods of such screening are well known in the art. For example, antibody gene repertoires can be cloned into a λ bacteriophage expression vector (Huse, W D et al. (1989). Science, 2476:1275). In addition, cells (Francisco et al. (1994), PNAS, 90:10444; Georgiou et al. (1997), Nat. Biotech., 15:29; Boder and Wittrup (1997) Nat. Biotechnol. 15:553; Boder et al., (2000), PNAS, 97:10701; Daugtherty, P. et al. (2000) J. Immunol. Methods. 243:211) or viruses (e.g., Hoogenboom, H R. (1998), Immunotechnology 4:1; Winter et al. (1994). Annu. Rev. Immunol. 12:433; Griffiths, A D. (1998). Curr. Opin. Biotechnol. 9:102) expressing antibodies on their surface can be screened.

Those skilled in the art will also appreciate that DNA encoding antibody variable domains may also be derived from antibody libraries expressed in phage, yeast, or bacteria using methods known in the art. Exemplary methods are set forth, for example, in EP 368 684 B1; U.S. Pat. No. 5,969,108; Hoogenboom et al., (2000) Immunol. Today 21:371; Nagy et al. (2002) Nat. Med. 8:801; Huie et al. (2001), PNAS, 98:2682; Lui et al. (2002), J. Mol. Biol. 315:1063, each of which is incorporated herein by reference. Several publications (e.g., Marks et al. (1992), Bio/Technology 10:779-783) have described the production of high affinity human antibodies by chain shuffling, as well as combinatorial infection and in vivo recombination as a strategy for constructing large phage libraries. In another embodiment, ribosomal display can be used to replace bacteriophage as the display platform (see, e.g., Hanes, et al. (1998), PNAS 95:14130; Hanes and Pluckthun. (1999), Curr. Top. Microbiol. Immunol. 243:107; He and Taussig. (1997), Nuc. Acids Res., 25:5132; Hanes et al. (2000), Nat. Biotechnol. 18:1287; Wilson et al. (2001), PNAS, 98:3750; or Irving et al. (2001) J. Immunol. Methods 248:31).

Preferred libraries for screening are human variable gene libraries. V_{L} and V_{H} domains from a non-human source may also be used. Libraries can be naïve, from immunized subjects, or semi-synthetic (Hoogenboom and Winter. (1992). J. Mol. Biol. 227:381; Griffiths et al. (1995) EMBO J. 13:3245; de Kruif et al. (1995). J. Mol. Biol. 248:97; Barbas et al. (1992), PNAS, 89:4457). In one embodiment, mutations can be made to immunoglobulin domains to create a library of nucleic acid molecules having greater heterogeneity (Thompson et al. (1996), J. Mol. Biol. 256:77; Lamminmaki et al. (1999), J. Mol. Biol. 291:589; Caldwell and Joyce. (1992), PCR Methods Appl. 2:28; Caldwell and Joyce. (1994), PCR Methods Appl. 3:S136). Standard screening procedures can be used to select high affinity variants. In another embodiment, changes to V_{H} and V_{L} sequences can be made to increase antibody avidity, e.g., using information obtained from crystal structures using techniques known in the art.

Moreover, variable region sequences useful for producing the binding polypeptides of the present invention may be obtained from a number of different sources. For example, as discussed above, a variety of human gene sequences are available in the form of publicly accessible deposits. Many sequences of antibodies and antibody-encoding genes have been published and suitable variable region sequences (e.g. VL and VH sequences) can be chemically synthesized from these sequences using art recognized techniques.

In another embodiment, at least one variable region domain present in a binding polypeptide of the invention is catalytic (Shokat and Schultz. (1990). Annu. Rev. Immunol. 8:335). Variable region domains with catalytic binding specificities can be made using art recognized techniques (see, e.g., U.S. Pat. No. 6,590,080, U.S. Pat. No. 5,658,753). Catalytic binding specificities can work by a number of basic mechanisms similar to those identified for enzymes to stabilize the transition state, thereby reducing the free energy of activation. For example, general acid and base residues can be optimally positioned for participation in catalysis within catalytic active sites; covalent enzyme-substrate intermediates can be formed; catalytic antibodies can also be in proper orientation for reaction and increase the effective concentration of reactants by at least seven orders of magnitude (Fersht et al., (1968), J. Am. Chem. Soc. 90:5833) and thereby greatly reduce the entropy of a chemical reaction. Finally, catalytic antibodies can convert the energy obtained upon substrate binding and/or subsequent stabilization of the transition state intermediate to drive the reaction.

Acid or base residues can be brought into the antigen binding site by using a complementary charged molecule as an immunogen. This technique has proved successful for elicitation of antibodies with a hapten containing a positively-charged ammonium ion (Shokat, et al., (1988), Chem. Int. Ed. Engl. 27:269-271). In another approach, antibodies can be elicited to stable compounds that resemble the size, shape, and charge of the transition state intermediate of a desired reaction (i.e., transition state analogs). See U.S. Pat. No. 4,792,446 and U.S. Pat. No. 4,963,355 which describe the use of transition state analogs to immunize animals and the production of catalytic antibodies. Both of these patents are hereby incorporated by reference. Such molecules can be administered as part of an immunoconjugate, e.g., with an immunogenic carrier molecule, such as KLH.

In another embodiment, a variable region domain of an altered antibody of the invention consists of a V_{H} domain, e.g., derived from camelids, which is stable in the absence of a V_{L} chain (Hamers-Casterman et al. (1993). Nature, 363:446; Desmyter et al. (1996). Nat. Struct. Biol. 3: 803; Decanniere et al. (1999). Structure, 7:361; Davies et al. (1996). Protein Eng., 9:531; Kortt et al. (1995). J. Protein Chem., 14:167).

Further, a binding polypeptide of the invention may comprise a variable domain or CDR derived from a fully murine, fully human, chimeric, humanized, non-human primate or primatized antibody. Non-human antibodies, or fragments or domains thereof, can be altered to reduce their immunogenicity using art recognized techniques. Humanized antibodies are antibodies derived from non-human antibodies, that have been modified to retain or substantially retain the binding properties of the parent antibody, but which are less immunogenic in humans that the parent, non-human antibodies. In the case of humanized target antibodies, this may be achieved by various methods, including (a) grafting the entire non-human variable domains onto human constant regions to generate chimeric target antibodies; (b) grafting at least a part of one or more of the non-human complementarity determining regions (CDRs) into a human framework and constant regions with or without retention of critical framework residues; (c) transplanting the entire non-human variable domains, but “cloaking” them with a human-like section by replacement of surface residues. Such methods are disclosed in Morrison et al., (1984), PNAS. 81: 6851-5; Morrison et al., (1988), Adv. Immunol. 44: 65-92; Verhoeyen et al., (1988), Science 239: 1534-1536; Padlan, (1991), Molec. Immun. 28: 489-498; Padlan, (1994), Molec. Immun. 31: 169-217; and U.S. Pat. Nos. 5,585,089, 5,693,761 and 5,693,762 all of which are hereby incorporated by reference in their entirety.

De-immunization can also be used to decrease the immunogenicity of a binding polypeptide of the invention. As used herein, the term “de-immunization” includes modification of T cell epitopes (see, e.g., WO9852976A1, WO0034317A2). For example, VH and VL sequences are analyzed and a human T cell epitope “map” from each V region showing the location of epitopes in relation to complementarity-determining regions (CDRs) and other key residues within the sequence is generated. Individual T cell epitopes from the T cell epitope map are analyzed in order to identify alternative amino acid substitutions with a low risk of altering the activity of the final antibody. A range of alternative VH and VL sequences are designed comprising combinations of amino acid substitutions and these sequences are subsequently incorporated into a range of polypeptides of the invention that are tested for function. Typically, between 12 and 24 variant antibodies are generated and tested. Complete heavy and light chain genes comprising modified V and human C regions are then cloned into expression vectors and the subsequent plasmids introduced into cell lines for the production of whole antibody. The antibodies are then compared in appropriate biochemical and biological assays, and the optimal variant is identified.

In one embodiment, the variable domains employed in a binding polypeptide of the invention are altered by at least partial replacement of one or more CDRs. In another embodiment, variable domains can optionally be altered, e.g., by partial framework region replacement and sequence changing. In making a humanized variable region the CDRs may be derived from an antibody of the same class or even subclass as the antibody from which the framework regions are derived, however, it is envisaged that the CDRs will be derived from an antibody of different class and preferably from an antibody from a different species. It may not be necessary to replace all of the CDRs with the complete CDRs from the donor variable region to transfer the antigen binding capacity of one variable domain to another. Rather, it may only be necessary to transfer those residues that are necessary to maintain the activity of the binding domain. Given the explanations set forth in U.S. Pat. Nos. 5,585,089, 5,693,761 and 5,693,762, it will be well within the competence of those skilled in the art, either by carrying out routine experimentation or by trial and error testing to obtain a functional antigen binding site with reduced immunogenicity.

In one embodiment, a binding polypeptide of the invention comprises at least one CDR from an antibody that recognizes a desired target. In another embodiment, an altered antibody of the present invention comprises at least two CDRs from an antibody that recognizes a desired target. In another embodiment, an altered antibody of the present invention comprises at least three CDRs from an antibody that recognizes a desired target. In another embodiment, an altered antibody of the present invention comprises at least four CDRs from an antibody that recognizes a desired target. In another embodiment, an altered antibody of the present invention comprises at least five CDRs from an antibody that recognizes a desired target. In another embodiment, an altered antibody of the present invention comprises all six CDRs from an antibody that recognizes a desired target.

In one embodiment, antigen binding sites employed in the binding polypeptides of the present invention may be immunoreactive with one or more tumor-associated antigens. For example, for treating a cancer or neoplasia an antigen binding domain of a binding polypeptide preferably binds to a selected tumor associated antigen. Given the number of reported antigens associated with neoplasias, and the number of related antibodies, those skilled in the art will appreciate that a binding polypeptide of the invention may comprise a variable region sequence or portion thereof derived from any one of a number of whole antibodies. More generally, such a variable region sequence may be obtained or derived from any antibody (including those previously reported in the literature) that reacts with an antigen or marker associated with the selected condition. Exemplary tumor-associated antigens bound by the binding polypeptides of the invention include for example, pan B antigens (e.g. CD20 found on the surface of both malignant and non-malignant B cells such as those in non-Hodgkin's lymphoma) and pan T cell antigens (e.g. CD2, CD3, CD5, CD6, CD7). Other exemplary tumor associated antigens comprise but are not limited to MAGE-1, MAGE-3, MUC-1, HPV 16, HPV E6 & E7, TAG-72, CEA, α-Lewis^{y}, L6-Antigen, CD19, CD22, CD23, CD25, CD30, CD33, CD37, CD44, CD52, CD56, CD80, mesothelin, PSMA, HLA-DR, EGF Receptor, VEGF, VEGF Receptor, Cripto antigen, and HER2Receptor.

In other embodiments, the binding polypeptide of the invention may comprise the complete antigen binding site (or variable regions or CDR sequences thereof) from antibodies that have previously been reported to react with tumor-associated antigens. Exemplary antibodies capable of reacting with tumor-associated antigens include: 2B8, Lym 1, Lym 2, LL2, Her2, B1, BR96, MB1, BH3, B4, B72.3, 5E8, B3F6, 5E10, α-CD33, α-CanAg, α-CD56, α-CD44v6, α-Lewis, and α-CD30. More specifically, these exemplary antibodies include, but are not limited to 2B8 and C2B8 (Zevalin® and Rituxan®, Biogen Idec, Cambridge), Lym 1 and Lym 2 (Techniclone), LL2 (Immunomedics Corp., New Jersey), Trastuzumab (Herceptin®, Genentech Inc., South San Francisco), Tositumomab (Bexxar®, Coulter Pharm., San Francisco), Alemtzumab (Campath®, Millennium Pharmaceuticals, Cambridge), Gemtuzumab ozogamicin (Mylotarg®, Wyeth-Ayerst, Philadelphia), Abagovomab (Menarini, Italy), CEA-Scan™ (Immunomedics, Morris Plains, N.J.), Capromab (Prostascint®, Cytogen Corp.), Edrecolomab (Panorex®, Johnson & Johnson, New Brunswick, N.J.), Igovomab (CIS Bio Intl., France), Mitumomab (BEC2, Imclone Systems, Somerville, N.J.), Nofetumomab (Verluma®, Boehringer Ingleheim, Ridgefield, Conn.), OvaRex (Altarex Corp., Waltham, Mass.), Satumomab (Onoscint®, Cytogen Corp.), Apolizumab (REMITOGEN™, Protein Design Labs, Fremont, Calif.), Labetuzumab (CEACIDE™, Immunomedics Inc., Morris Plains, N.J.), Pertuzumab (OMNITARG™, Genentech Inc., S. San Francisco, Calif.), Panitumumab (Vectibix®, Amgen, Thousand Oaks, Calif.), Cetuximab (Erbitux®, Imclone Systems, New York), Bevacizumab (Avastin®, Genentech Inc., South San Francisco), BR96, BL22, LMB9, LMB2, MB1, BH3, B4, B72.3 (Cytogen Corp.), SS1 (NeoPharm), CC49 (National Cancer Institute), Cantuzumab mertansine (ImmunoGen, Cambridge), MNL 2704 (Milleneum Pharmaceuticals, Cambridge), Bivatuzumab mertansine (Boehringer Ingelheim, Germany), Trastuzumab-DM1 (Genentech, South San Francisco), My9-6-DM1 (ImmunoGen, Cabridge), SGN-10, -15, -25, and -35 (Seattle Genetics, Seattle), and 5E10 (University of Iowa). In yet other embodiments, the binding polypeptides may comprise the binding site of an anti-CD23 antibody (e.g., Lumiliximab), an anti-CD80 antibody (e.g., Galiximab), or an anti-VL5/α5β1-integrin antibody (e.g., Volociximab). In other embodiments, the binding polypeptides of the present invention will bind to the same tumor-associated antigens as the antibodies enumerated immediately above. In particularly preferred embodiments, the polypeptides will be derived from or bind the same antigens as Y2B8, C2B8, CC49 and C5E10.

Other binding sites that can be incorporated into the subject binding molecules include those found in: Orthoclone OKT3 (anti-CD3) (Johnson&Johnson, Brunswick, N.J.), ReoPro® (anti-GpIIb/gIIa)(Centocor, Horsham, Pa.), Zenapax® (anti-CD25)(Roche, Basel, Switzerland), Remicade® (anti-TNFα)(Centocor, Horsham, Pa.), Simulect® (anti-CD25)(Novartis, Basel, Switzerland), Synagis® (anti-RSV)(Medimmune, Gaithersburg, Md.), Humira® (anti-TNFα) (Abbott, Abbott Park, Ill.), Xolair® (anti-IgE) (Genentech, South San Francisco, Calif.), Raptiva® (anti-CD11a) (Genentech), Tysabri® (Biogenldec, Cambridge, Mass.), Lucentis® (anti-VEGF) (Genentech), and Soliris® (Alexion Pharmaceuticals, Cheshire, Conn.).

In one embodiment, a binding molecule of the invention may have one or more binding sites derived from one or more of the following antibodies. tositumomab (BEXXAR®), muromonab (ORTHOCLONE®) and ibritumomab (ZEVALIN®), cetuximab (ERBITUX™), rituximab (MABTHERA®/RITUXAN®), infliximab (REMICADE®), abciximab (REOPRO®) and basiliximab (SIMULECT®), efalizumab (RAPTIVA®, bevacizumab (AVASTIN®), alemtuzumab (CAMPATH®), trastuzumab (HERCEPTIN®), gemtuzumab (MYLOTARG®), palivizumab (SYNAGIS®), omalizumab (XOLAIR®), daclizumab (ZENAPAX®), natalizumab (TYSABRI®) and ranibizumab (LUVENTIS®), adalimumab (HUMIRA®) and panitumumab (VECTIBIX®).

In one embodiment, the binding polypeptide will bind to the same antigen as Rituxan®. Rituxan® (also known as, rituximab, IDEC-C2B8 and C2B8) was the first FDA-approved monoclonal antibody for treatment of human B-cell lymphoma (see U.S. Pat. Nos. 5,843,439; 5,776,456 and 5,736,137 each of which is incorporated herein by reference). Y2B8 (90Y labeled 2B8; Zevalin®; ibritumomab tiuxetan) is the murine, parent antibody of C2B8. Rituxan® is a chimeric, anti-CD20 monoclonal antibody which is growth inhibitory and reportedly sensitizes certain lymphoma cell lines for apoptosis by chemotherapeutic agents in vitro. The antibody efficiently binds human complement, has strong FcR binding, and can effectively kill human lymphocytes in vitro via both complement dependent (CDC) and antibody-dependent (ADCC) mechanisms (Reff et al., Blood 83: 435-445 (1994)). Those skilled in the art will appreciate that binding polypeptide of the invention may comprises variable regions or CDRs of C2B8 or 2B8, in order to provide binding polypeptide that are even more effective in treating patients presenting with CD20+ malignancies.

In other embodiments of the present invention, the binding polypeptide of the invention will bind to the same tumor-associated antigen as CC49. CC49 binds human tumor-associated antigen TAG-72 which is associated with the surface of certain tumor cells of human origin, specifically the LS174T tumor cell line. LS174T is a variant of the LS180 colon adenocarcinoma line.

Binding polypeptides of the invention may comprise antigen binding sites derived from numerous murine monoclonal antibodies that have been developed and which have binding specificity for TAG-72. One of these monoclonal antibodies, designated B72.3, is a murine IgG1 produced by hybridoma B72.3. B72.3 is a first generation monoclonal antibody developed using a human breast carcinoma extract as the immunogen (see Colcher et al., Proc. Natl. Acad. Sci. (USA), 78:3199-3203 (1981); and U.S. Pat. Nos. 4,522,918 and 4,612,282, each of which is incorporated herein by reference). Other monoclonal antibodies directed against TAG-72 are designated “CC” (for colon cancer). As described by Schlom et al. (U.S. Pat. No. 5,512,443 which is incorporated herein by reference) CC monoclonal antibodies are a family of second generation murine monoclonal antibodies that were prepared using TAG-72 purified with B72.3. Because of their relatively good binding affinities to TAG-72, the following CC antibodies are preferred: CC49, CC 83, CC46, CC92, CC30, CC11, and CC15. Schlom et al. have also produced variants of a humanized CC49 antibody as disclosed in PCT/US99/25552 and single chain Fv (scFv) constructs as disclosed in U.S. Pat. No. 5,892,019, each of which is also incorporated herein by reference. Those skilled in the art will appreciate that each of the foregoing antibodies, constructs or recombinants, and variations thereof, may be synthetic and used to provide binding sites for the production of binding polypeptides in accordance with the present invention.

In addition to the anti-TAG-72 antibodies discussed above, various groups have also reported the construction and partial characterization of domain-deleted CC49 and B72.3 antibodies (e.g., Calvo et al. Cancer Biotherapy, 8(1):95-109 (1993), Slavin-Chiorini et al. Int. J. Cancer 53:97-103 (1993) and Slavin-Chiorini et al. Cancer. Res. 55:5957-5967 (1995). Accordingly, binding polypeptides may comprise antigen binding sites, variable region, or CDRs derived from these antibodies as well.

In one embodiment, a binding polypeptide of the invention comprises an antigen binding site that binds to the CD23 antigen (U.S. Pat. No. 6,011,138). In a preferred embodiment, a binding polypeptide of the invention binds to the same epitope as the 5E8 antibody. In another embodiment, a binding polypeptide of the invention comprises at least one CDR (e.g., 1, 2, 3, 4, 5, or 6 CDRs) from an anti-CD23 antibody, e.g., the 5E8 antibody (e.g., Lumiliximab).

In one embodiment, a binding polypeptide of the invention binds to the CRIPTO-I antigen (WO02/088170A2 or WO03/083041A2). In a more preferred embodiment, a binding polypeptide of the invention binds to the same epitope as the B3F6 antibody. In still another embodiment, an altered antibody of the invention comprises at least one CDR (e.g., 1, 2, 3, 4, 5, or 6 CDRs) or variable region from an anti-CRIPTO-I antibody, e.g., the B3F6 antibody.

In another embodiment, a binding polypeptide of the invention binds to antigen which is a member of the TNF superfamily of receptors (“TNFRs”). In another embodiment, the binding molecules of the invention bind at least one target that transduces a signal to a cell, e.g., by binding to a cell surface receptor, such as a TNF family receptor. By “transduces a signal” it is meant that by binding to the cell, the binding molecule converts the extracellular influence on the cell surface receptor into a cellular response, e.g., by modulating a signal transduction pathway. The term “TNF receptor” or “TNF receptor family member” refers to any receptor belonging to the Tumor Necrosis Factor (“TNF”) superfamily of receptors. Members of the TNF Receptor Superfamily (“TNFRSF”) are characterized by an extracellular region with two or more cysteine-rich domains (˜40 amino acids each) arranged as cysteine knots (see Dempsey et al., Cytokine Growth Factor Rev. (2003). 14(3-4):193-209). Upon binding their cognate TNF ligands, TNF receptors transduce signals by interacting directly or indirectly with cytoplasmic adapter proteins known as TRAFs (TNF receptor associate factors). TRAFs can induce the activation of several kinase cascades that ultimately lead to the activation of signal transduction pathways such as NF-KappaB, JNK, ERK, p38 and PI3K, which in turn regulate cellular processes ranging from immune function and tissue differentiation to apoptosis. The nucleotide and amino acid sequences of several TNF receptors family members are known in the art and include at least 29 human genes: TNFRSF1A (TNFR1, also known as DR1, CD120a, TNF-R-I p55, TNF-R, TNFR1, TNFAR, TNF-R55, p55TNFR, p55R, or TNFR60, GenBank G1 No. 4507575; see also U.S. Pat. No. 5,395,760)), TNFRSF1B (CD120b, also known as p75, TNF-R, TNF-R-II, TNFR80, TNFR2, TNF-R75, TNFBR, or p75TNFR; GenBank G1 No. 4507577), TNFRSF3 (Lymphotoxin Beta Receptor (LTβR), also known as TNFR2-RP, CD18, TNFR-RP, TNFCR, or TNF-R-III; G1 Nos. 4505038 and 20072212), TNFRSF4 (OX40, also known as ACT35, TXGP1L, or CD134 antigen; G1 Nos. 4507579 and 8926702), TNFRSF5 (CD40, also known as p50 or Bp50; G1 Nos. 4507581 and 23312371), TNFRSF6 (FAS, also known as FAS-R, DcR-2, DR2, CD95, APO-1, or APT1; GenBank G1 Nos. 4507583, 23510421, 23510423, 23510425, 23510427, 23510429, 23510431, and 23510434)), TNFRSF6B (DcR3, DR3; GenBank G1 Nos. 4507569, 23200021, 23200023, 23200025, 23200027, 23200029, 23200031, 23200033, 23200035, 23200037, and 23200039), TNFRSF7 (CD27, also known as Tp55 or S152; GenBank G1 No. 4507587), TNFRSF8 (CD30, also known as Ki-1, or D1S166E; GenBank G1 Nos. 4507589 and 23510437), TNFRSF9 (4-1-BB, also known as CD137 or ILA; G1 Nos. 5730095 and 728738), TNFRSF10A (TRAIL-R1, also known as DR4 or Apo2; GenBank G1 No. 21361086), TNFRSF10B (TRAIL-R2, also known as DR5, KILLER, TRICK2A, or TRICKB; GenBank G1 Nos. 22547116 and 22547119), TNFRSF10C (TRAIL-R3, also known as DcR1, LIT, or TRID; GenBank G1 No. 22547121), TNFRSF10D (TRAIL-R4, also known as DcR2 or TRUNDD), TNFRSF11A (RANK; GenBank G1 No. 4507565; see U.S. Pat. Nos. 6,562,948; 6,537,763; 6,528,482; 6,479,635; 6,271,349; 6,017,729), TNFRSF11B (Osteoprotegerin (OPG), also known as OCIF or TR1; G1 Nos. 38530116, 22547122 and 33878056), TNFRSF12 (Translocating chain-Association Membrane Protein (TRAMP), also known as DR3, WSL-1, LARD, WSL-LR, DDR3, TR3, APO-3, Fn14, or TWEAKR; GenBank G1 No. 7706186; US Patent Application Publication No. 2004/0033225A1), TNFRSF12L (DR3L), TNFRSF13B (TACI; G1 No. 6912694), TNFRSF13C (BAFFR; G1 No. 16445027), TNFRSF14 (Herpes Virus Entry Mediator (HVEM), also known as ATAR, TR2, LIGHTR, or HVEA; GenBank G1 Nos. 23200041, 12803895, and 3878821), TNFRSF16 (Low-Affinity Nerve Growth Factor Receptor (LNGFR), also known as Neurotrophin Receptor or p75(NTR); GenBank G1 Nos. 128156 and 4505393), TNFRSF17 (BCM, also known as BCMA; G1 No. 23238192), TNFRSF18 (ATTR, also known as GITR; GenBank G1 Nos. 4759246, 23238194 and 23238197), TNFRSF19 (Troy/Trade, also known as TAJ; GenBank G1 Nos. 23238202 and 23238204), TNFRSF20 (RELT, also known as F1114993; G1 Nos. 21361873 and 23238200), TNFRSF21 (DR6), TNFRSF22 (SOBa, also known as Tnfrh2 or 2810028K06Rik), and TNFRSF23 (mSOB, also known as Tnfrh1). Other TNF family members include EDAR1 (Ectodysplasin A Receptor, also known as Downless (DL), ED3, ED5, ED1R, EDA3, EDA1R, EDA-A1R; GenBank G1 No. 11641231; U.S. Pat. No. 6,355,782), XEDAR (also known as EDA-A2R; GenBank G1 No. 11140823); and CD39 (G1 Nos. 2135580 and 765256). In another embodiment, an altered antibody of the invention binds to a TNF receptor family member lacking a death domain. In one embodiment, the TNF receptor lacking a death domain is involved in tissue differentiation. In a more specific embodiment, the TNF receptor involved in tissue differentiation is selected from the group consisting of LTβR, RANK, EDAR1, XEDAR, Fn14, Troy/Trade, and NGFR. In another embodiment, the TNF receptor lacking a death domain is involved in immune regulation. In a more specific embodiment, TNF receptor family member involved in immune regulation is selected from the group consisting of TNFR2, HVEM, CD27, CD30, CD40, 4-1BB, OX40, and GITR. Exemplary antibodies which can provide binding sites specific for these, as well as other targets described herein are known in the art. For example, Exemplary anti-CD40 antibody sequences can be found, e.g., in U.S. Pat. Nos. 6,051,228 and 6,312,693.

In another embodiment, a binding polypeptide of the invention binds to a TNF ligand belonging to the TNF ligand superfamily. TNF ligands bind to distinct receptors of the TNF receptor superfamily and exhibit 15-25% amino acid sequence homology with each other (Gaur et al., Biochem. Pharmacol. (2003), 66(8):1403-8). The nucleotide and amino acid sequences of several TNF Receptor (Ligand) Superfamily (“TNFSF”) members are known in the art and include at least 16 human genes: TNFSF1 (also known as Lymphotoxin-α (LTA), TNFβ or LT, G1 No.: 34444 and 6806893), TNFSF2 (also known as TNF, TNFα, or DIF; G1 No. 25952111), TNFSF3 (also known as Lymphotoxin-β (LTB), TNFC, or p33), TNFSF4 (also known as OX-40L, gp34, CD134L, or tax-transcriptionally activated glycoprotein 1, 34 kD (TXGP1); G1 No. 4507603), TNFSF5 (also known as CD40LG, IMD3, HIGM1, CD40L, hCD40L, TRAP, CD154, or gp39; G1 No. 4557433), TNFSF6 (also known as FasL or APT1LG1; GenBank G1 No. 4557329), TNFSF7 (also known as CD70, CD27L, or CD27LG; G1 No. 4507605), TNFSF8 (also known as CD30LG, CD30L, or CD153; G1 No. 4507607), TNFSF9 (also known as 4-1BB-L or ILA ligand; G1 No. 4507609), TNFSF10 (also known as TRAIL, Apo-2L, or TL2; G1 No. 4507593), TNFSF11 (also known as TRANCE, RANKL, OPGL, or ODF; G1 Nos. 4507595 and 14790152), TNFSF12 (also known as Fn14L, TWEAK, DR3LG, or APO3L; G1 Nos. 4507597 and 23510441), TNFSF13 (also known as APRIL), TNFSF14 (also known as LIGHT, LTg, or HVEM-L; G1 Nos. 25952144 and 25952147), TNFSF15 (also known as TL1 or VEGI), or TNFSF16 (also known as AITRL, TL6, hGITRL, or GITRL; G1 No. 4827034). Other TNF ligand family members include EDAR1 & XEDAR ligand (ED1; G1 No. 4503449; Monreal et al. (1998) Am J Hum Genet. 63:380), Troy/Trade ligand, BAFF (also known as TALL1; G1 No. 5730097), and NGF ligands (e.g. NGF-(3 (G1 No. 4505391), NGF-2/NTF3; G1 No. 4505469), NTF5 (G1 No. 5453808)), BDNF (G1 Nos. 25306267, 25306235, 25306253, 25306257, 25306261, 25306264; IFRD1 (G1 No. 4504607)). In a more specific embodiment, the TNF ligand is involved in immune regulation (e.g., CD40L or TWEAK).

In still other embodiments, a binding polypeptide of the invention binds to a molecule which is useful in treating an autoimmune or inflammatory disease or disorder. For example, a binding polypeptide may bind to an antigen present on an immune cell (e.g., a B or T cell) or an autoantigen responsible for an autoimmune disease or disorder. The antigen associated with an autoimmune or inflammatory disorder may be a tumor-associated antigen described supra. Thus, a tumor associated antigen may also be an autoimmune or inflammatory associated disorder. As used herein, the term “autoimmune disease or disorder” refers to disorders or conditions in a subject wherein the immune system attacks the body's own cells, causing tissue destruction. Autoimmune diseases include general autoimmune diseases, i.e., in which the autoimmune reaction takes place simultaneously in a number of tissues, or organ specific autoimmune diseases, i.e., in which the autoimmune reaction targets a single organ. Examples of autoimmune diseases that can be diagnosed, prevented or treated by the methods and compositions of the present invention include, but are not limited to, Crohn's disease; Inflammatory bowel disease (IBD); systemic lupus erythematosus; ulcerative colitis; rheumatoid arthritis; Goodpasture's syndrome; Grave's disease; Hashimoto's thyroiditis; pemphigus vulgaris; myasthenia gravis; scleroderma; autoimmune hemolytic anemia; autoimmune thrombocytopenic purpura; polymyositis and dermatomyositis; pernicious anemia; Sjögren's syndrome; ankylosing spondylitis; vasculitis; type I diabetes mellitus; neurological disorders, multiple sclerosis, and secondary diseases caused as a result of autoimmune diseases.

In other embodiments, the binding polypeptides of the invention bind to a target molecule associated with an inflammatory disease or disorder. As used herein the term “inflammatory disease or disorder” includes diseases or disorders which are caused, at least in part, or exacerbated by inflammation, e.g., increased blood flow, edema, activation of immune cells (e.g., proliferation, cytokine production, or enhanced phagocytosis). For example, a binding polypeptide of the invention may bind to an inflammatory factor (e.g., a matrix metalloproteinase (MMP), TNFα, an interleukin, a plasma protein, a cytokine, a lipid metabolite, a protease, a toxic radical, a mitochondrial protein, an apoptotic protein, an adhesion molecule, etc.) involved or present in an area in aberrant amounts, e.g., in amounts which may be advantageous to alter, e.g., to benefit the subject. The inflammatory process is the response of living tissue to damage. The cause of inflammation may be due to physical damage, chemical substances, micro-organisms, tissue necrosis, cancer or other agents. Acute inflammation is short-lasting, e.g., lasting only a few days. If it is longer lasting however, then it may be referred to as chronic inflammation.

Inflammatory disorders include acute inflammatory disorders, chronic inflammatory disorders, and recurrent inflammatory disorders. Acute inflammatory disorders are generally of relatively short duration, and last for from about a few minutes to about one to two days, although they may last several weeks. The main characteristics of acute inflammatory disorders include increased blood flow, exudation of fluid and plasma proteins (edema) and emigration of leukocytes, such as neutrophils. Chronic inflammatory disorders, generally, are of longer duration, e.g., weeks to months to years or even longer, and are associated histologically with the presence of lymphocytes and macrophages and with proliferation of blood vessels and connective tissue. Recurrent inflammatory disorders include disorders which recur after a period of time or which have periodic episodes. Examples of recurrent inflammatory disorders include asthma and multiple sclerosis. Some disorders may fall within one or more categories. Inflammatory disorders are generally characterized by heat, redness, swelling, pain and loss of function. Examples of causes of inflammatory disorders include, but are not limited to, microbial infections (e.g., bacterial, viral and fungal infections), physical agents (e.g., burns, radiation, and trauma), chemical agents (e.g., toxins and caustic substances), tissue necrosis and various types of immunologic reactions. Examples of inflammatory disorders include, but are not limited to, osteoarthritis, rheumatoid arthritis, acute and chronic infections (bacterial, viral and fungal); acute and chronic bronchitis, sinusitis, and other respiratory infections, including the common cold; acute and chronic gastroenteritis and colitis; acute and chronic cystitis and urethritis; acute respiratory distress syndrome; cystic fibrosis; acute and chronic dermatitis; acute and chronic conjunctivitis; acute and chronic serositis (pericarditis, peritonitis, synovitis, pleuritis and tendinitis); uremic pericarditis; acute and chronic cholecystis; acute and chronic vaginitis; acute and chronic uveitis; drug reactions; and burns (thermal, chemical, and electrical).

In one preferred embodiment, a binding polypeptide of the invention binds to

CD40L antibody (e.g., to the same epitope as (i.e., competes with) a 5C8 antibody). In still another embodiment, a polypeptide of the invention comprises at least one antigen binding site, one or more CDRs (e.g., 1, 2, 3, 4, 5, or 6 CDRs), or one or more variable regions (VH or VL) from an anti-CD40L antibody (e.g. a 5C8 antibody). CD40L (CD154, gp39), a transmembrane protein, is expressed on activated CD4⁺ T cells, mast cells, basophils, eosinophils, natural killer (NK) cells, and activated platelets. CD40L is important for T-cell-dependent B-cell responses. A prominent function of CD40L, isotype switching, is demonstrated by the hyper-immunoglobulin M (IgM) syndrome in which CD40L is congenitally deficient. The interaction of CD40L-CD40 (on antigen-presenting cells such as dendritic cells) is essential for T-cell priming and the T-cell-dependent humoral immune response. Therefore, interruption of the CD40-CD40L interaction with an anti-CD40L monoclonal antibody (mAb) has been considered to be a possible therapeutic strategy in human autoimmune disease, based upon the above information and on studies in animals. Exemplary anti-CD40L antibodies from which the binding polypeptides of the invention may be derived include the mouse antibody 5C8, disclosed in U.S. Pat. No. 5,474,771, which is incorporated by reference herein, as well as humanized versions thereof, e.g., the Hu5C8 antibody disclosed in the Examples. Other anti-CD40L antibodies are known in the art (see e.g., U.S. Pat. No. 5,961,974 and International Publication No. WO 96/23071). In particular embodiments, an anti-CD40L binding polypeptide of the invention comprises a VH and/or VL sequence of the 5C8 antibody.

In yet other embodiments, a binding polypeptide of the invention binds to a molecule which is useful in treating a neurological disease or disorder. For example, a binding polypeptide may bind to an antigen present on a neural cell (e.g., a neuron, a glial cell, or a). In certain embodiments, the antigen associated with a neurological disorder may be an autoimmune or inflammatory disorder described supra. As used herein, the term “neurological disease or disorder” includes disorders or conditions in a subject wherein the nervous system either degenerates (e.g., neurodegenerative disorders, as well as disorders where the nervous system fails to develop properly or fails to regenerate following injury, e.g., spinal cord injury. Examples of neurological disorders that can be diagnosed, prevented or treated by the methods and compositions of the present invention include, but are not limited to, Multiple Sclerosis, Huntington's Disease, Alzheimer's Disease, Parkinson's Disease, neuropathic pain, traumatic brain injury, Guillain-Barré syndrome and chronic inflammatory demyelinating polyneuropathy (CIDP).

Exemplary molecules that are useful in treating a neurological disease or disorder, and against which binding polypeptides of the invention can be targeted, include a LINGO protein, e.g., LINGO-1 and LINGO-4; a semaphorin protein, e.g., semaphorin-6A; a Death Receptor (DR) protein, e.g., DR6, a TRAIN (or TAJ) protein; TRKA, TRKB; and a NOGO protein.

### (b) Antigen Binding Fragments

In other embodiments, a binding site of a binding polypeptide of the invention may comprise an antigen binding fragment. The term “antigen-binding fragment” refers to a polypeptide fragment of an immunoglobulin, antibody, or antibody variant which binds antigen or competes with intact antibody (i.e., with the intact antibody from which they were derived) for antigen binding (i.e., specific binding). For example, said antigen binding fragments can be derived from any of the antibodies or antibody variants described supra. Antigen binding fragments can be produced by recombinant or biochemical methods that are well known in the art. Exemplary antigen-binding fragments include single domain antibody, Fv, scFv, Fab, Fab′, and (Fab′)₂.

In exemplary embodiments, a binding polypeptide of the invention comprises at least one antigen binding fragment that is operably linked (e.g., chemically conjugated or genetically-fused (e.g., directly fused or fused via a polypeptide linker)) to the C-terminus and/or N-terminus of a stabilized Fc region of an variant Fc polypeptide. In one exemplary embodiment, a binding polypeptide of the invention comprises an antigen binding fragment (e.g, a Fab) which is operably linked to the N-terminus (or C-terminus) of at least one stabilized Fc region via a hinge domain or portion thereof (e.g., an IgG1 hinge or portion thereof, e.g., a human IgG1 hinge). An exemplary hinge domain portion comprises the sequence DKTHTCPPCPAPELLGG.

### (c) Single Chain Binding Molecules

In other embodiments, a binding molecule of the invention may comprise a binding site from single chain binding molecule (e.g., a singe chain variable region or scFv). Techniques described for the production of single chain antibodies (U.S. Pat. No. 4,694,778; Bird, Science 242:423-442 (1988); Huston et al., Proc. Natl. Acad. Sci. USA 85:5879-5883 (1988); and Ward et al., Nature 334:544-554 (1989)) can be adapted to produce single chain binding molecules. Single chain antibodies are formed by linking the heavy and light chain fragments of the Fv region via an amino acid bridge, resulting in a single chain antibody. Techniques for the assembly of functional Fv fragments in E. coli may also be used (Skerra et al., Science 242:1038-1041 (1988)).

In certain embodiments, a binding polypeptide of the invention comprises one or more binding sites or regions comprising or consisting of a single chain variable region sequence (scFv). Single chain variable region sequences comprise a single polypeptide having one or more antigen binding sites, e.g., a V_{L} domain linked by a flexible linker to a V_{H} domain. The VL and/or VH domains may be derived from any of the antibodies or antibody variants described supra. ScFv molecules can be constructed in a V_{H}-linker-V_{L} orientation or V_{L}-linker-V_{H} orientation. The flexible linker that links the V_{L} and V_{H} domains that make up the antigen binding site preferably comprises from about 10 to about 50 amino acid residues. In one embodiment, the polypeptide linker is a gly-ser polypeptide linker. An exemplary gly/ser polypeptide linker is of the formula (Gly4Ser)n, wherein n is a positive integer (e.g., 1, 2, 3, 4, 5, or 6). Other polypeptide linkers are known in the art. Antibodies having single chain variable region sequences (e.g. single chain Fv antibodies) and methods of making said single chain antibodies are well-known in the art (see e.g., Ho et al. 1989. Gene 77:51; Bird et al. 1988 Science 242:423; Pantoliano et al. 1991. Biochemistry 30:10117; Milenic et al. 1991. Cancer Research 51:6363; Takkinen et al. 1991. Protein Engineering 4:837).

In certain embodiments, a scFv molecule employed in a binding polypeptide of the invention is a stabilized scFv molecule. In one embodiment, the stabilized scFv molecule may comprise a scFv linker interposed between a V_{H} domain and a V_{L} domain, wherein the V_{H} and V_{L} domains are linked by a disulfide bond between an amino acid in the V_{H} and an amino acid in the V_{L} domain. In other embodiments, the stabilized scFv molecule may comprise a scFv linker having an optimized length or composition. In yet other embodiments, the stabilized scFv molecule may comprise a V_{H} or V_{L} domain having at least one stabilizing amino acid substitution(s). In yet another embodiment, a stabilized scFv molecule may have at least two of the above listed stabilizing features. Stabilized scFv molecules have improved protein stability or impart improved protein stability to the binding polypeptide to which it is operably linked. Preferred scFv linkers of the invention improve the thermal stability of a binding polypeptide of the invention by at least about 2° C. or 3° C. as compared to a conventional binding polypeptide. Comparisons can be made, for example, between the scFv molecules of the invention. In certain preferred embodiments, the stabilized scFv molecule comprises a (Gly₄Ser)₄ scFv linker and a disulfide bond which links V_{H} amino acid 44 and V_{L} amino acid 100. Other exemplary stabilized scFv molecules which may be employed in the binding polypeptides of the invention are described in U.S. Provisional Patent Application No. 60/873,996, filed on Dec. 8, 2006 or U.S. patent application Ser. No. 11/725,970, filed on Mar. 19, 2007, each of which is incorporated herein by reference in its entirety.

In certain exemplary embodiments, the binding polypeptides of the invention comprise at least one scFv molecule that is operably linked (e.g., chemically conjugated or genetically-fused (e.g., directly fused or fused via a polypeptide linker) to the C-terminus and/or N-terminus of a genetically-fused Fc region (i.e., a scFc region). In one exemplary embodiment, a binding polypeptide of the invention comprises at least one scFv molecule (e.g, one or more stabilized scFv molecules) which are operably linked to the N-terminus (or C-terminus) of at least one genetically-fused Fc region via a hinge domain or portion thereof (e.g., an IgG1 hinge or portion thereof, e.g., a human IgG1 hinge). An exemplary hinge domain portion comprises the sequence DKTHTCPPCPAPELLGG.

In certain embodiments, a binding polypeptide of the invention comprises a tetravalent binding site or region formed by fusing two or more scFv molecules in series. For example, in one embodiment, scFv molecules are combined such that a first scFv molecule is operably linked at its N-terminus (e.g., via a polypeptide linker (e.g., a gly/ser polypeptide linker)) to at least one additional scFv molecule having the same or different binding specificity. Tandem arrays of scFv molecules are operably linked to the N-terminus and/or C-terminus of at least one genetically-fused Fc region (i.e., a scFc region) to form a binding polypeptide of the invention.

In another embodiment, a binding polypeptide of the invention comprises a tetravalent binding site or region which is formed by operably linking a scFv molecule (e.g. via a polypeptide linker) to an antigen biding fragment (e.g., a Fab fragment). Said tetravalent binding site or region is operably linked to the N-terminus and/or C-terminus of at least one genetically-fused Fc region (i.e., a scFc region) to form a binding polypeptide of the invention.

### (d) Modified Antibodies

In other aspects, the binding polypeptides of the invention may comprise antigen binding sites, or portions thereof, derived from modified forms of antibodies. Exemplary such forms include, e.g., minibodies, diabodies, triabodies, nanobodies, camelids, Dabs, tetravalent antibodies, intradiabodies (e.g., Jendreyko et al. 2003. J. Biol. Chem. 278:47813), fusion proteins (e.g., antibody cytokine fusion proteins, proteins fused to at least a portion of an Fc receptor), and bispecific antibodies. Other modified antibodies are described, for example in U.S. Pat. No. 4,745,055; EP 256,654; Faulkner et al., Nature 298:286 (1982); EP 120,694; EP 125,023; Morrison, J. Immun. 123:793 (1979); Kohler et al., Proc. Natl. Acad. Sci. USA 77:2197 (1980); Raso et al., Cancer Res. 41:2073 (1981); Morrison et al., Ann. Rev. Immunol. 2:239 (1984); Morrison, Science 229:1202 (1985); Morrison et al., Proc. Natl. Acad. Sci. USA 81:6851 (1984); EP 255,694; EP 266,663; and WO 88/03559. Reassorted immunoglobulin chains also are known. See, for example, U.S. Pat. No. 4,444,878; WO 88/03565; and EP 68,763 and references cited therein.

In one embodiment, a binding polypeptide of the invention comprises an antigen binding site or region which is a minibody or an antigen binding site derived therefrom. Minibodies are dimeric molecules made up of two polypeptide chains each comprising a scFv molecule which is fused to a CH3 domain or portion thereof via a polypeptide linker. Minibodies can be made by linking a scFv component and polypeptide linker-CH3 component using methods described in the art (see, e.g., U.S. Pat. No. 5,837,821 or WO 94/09817A1). These components can be isolated from separate plasmids as restriction fragments and then ligated and recloned into an appropriate vector (e.g., an expression vector). Appropriate assembly (e.g., of the open reading frame (ORF) encoding the monomeric minibody polypeptide chain) can be verified by restriction digestion and DNA sequence analysis. In one embodiment, a binding polypeptide of the invention comprises the scFv component of a minibody which is operably linked to at least one stabilized Fc region of a variant Fc polypeptide. In another embodiment, a binding polypeptide of the invention comprises a tetravalent minibody as a binding site or region. Tetravalent minibodies can be constructed in the same manner as minibodies, except that two scFv molecules are linked using a polypeptide linker. The linked scFv-scFv construct is then operably linked to a stabilized Fc region to form a binding polypeptide of the invention.

In another embodiment, a binding polypeptide of the invention comprises an antigen binding site or region which is a diabody or an antigen binding site derived therefrom. Diabodies are dimeric, tetravalent molecules each having a polypeptide similar to scFv molecules, but usually having a short (e.g., less than 10 and preferably 1-5) amino acid residue linker connecting both variable domains, such that the V_{L} and V_{H} domains on the same polypeptide chain cannot interact. Instead, the V_{L} and V_{H} domain of one polypeptide chain interact with the V_{H} and V_{L} domain (respectively) on a second polypeptide chain (see, for example, WO 02/02781). In one embodiment, a binding polypeptide of the invention comprises a diabody which is operably linked to the N-terminus and/or C-terminus of at least one stabilized Fc region of an Fc polypeptide of the invention.

In certain embodiments, the binding molecule comprises a single domain binding molecule (e.g. a single domain antibody) linked to an stabilized Fc region. Exemplary single domain molecules include an isolated heavy chain variable domain (V_{H}) of an antibody, i.e., a heavy chain variable domain, without a light chain variable domain, and an isolated light chain variable domain (V_{L}) of an antibody, i.e., a light chain variable domain, without a heavy chain variable domain. Exemplary single-domain antibodies employed in the binding molecules of the invention include, for example, the Camelid heavy chain variable domain (about 118 to 136 amino acid residues) as described in Hamers-Casterman, et al., Nature 363:446-448 (1993), and Dumoulin, et al., Protein Science 11:500-515 (2002). Other exemplary single domain antibodies include single VH or VL domains, also known as Dabs® (Domantis Ltd., Cambridge, UK). Yet other single domain antibodies include shark antibodies (e.g., shark Ig-NARs). Shark Ig-NARs comprise a homodimer of one variable domain (V-NAR) and five C-like constant domains (C-NAR), wherein diversity is concentrated in an elongated CDR3 region varying from 5 to 23 residues in length. In camelid species (e.g., llamas), the heavy chain variable region, referred to as VHH, forms the entire antigen-binding domain. The main differences between camelid VHH variable regions and those derived from conventional antibodies (VH) include (a) more hydrophobic amino acids in the light chain contact surface of VH as compared to the corresponding region in VHH, (b) a longer CDR3 in VHH, and (c) the frequent occurrence of a disulfide bond between CDR1 and CDR3 in VHH. Methods for making single domain binding molecules are described in U.S. Pat. Nos. 6,005,079 and 6,765,087, both of which are incorporated herein by reference. Exemplary single domain antibodies comprising VHH domains include Nanobodies® (Ablynx NV, Ghent, Belgium).

### (e) Non-Immunoglobulin Binding Molecules

In certain other embodiments, the binding polypeptides of the invention comprise one or more binding sites derived from a non-immunoglobulin binding molecule. As used herein, the term “non-immunoglobulin binding molecules” are binding molecules whose binding sites comprise a portion (e.g., a scaffold or framework) which is derived from a polypeptide other than an immunoglobulin, but which may be engineered (e.g., mutagenized) to confer a desired binding specificity.

Other examples of binding molecules comprising binding sites not derived from antibody molecules include receptor binding sites and ligand binding sites which are discussed in more detail infra.

Non-immunoglobulin binding molecules can comprise binding site portions that are derived from a member of the immunoglobulin superfamily that is not an immunoglobulin (e.g. a T-cell receptor or a cell-adhesion protein (e.g., CTLA-4, N-CAM, telokin)). Such binding molecules comprise a binding site portion which retains the conformation of an immunoglobulin fold and is capable of specifically binding a target molecule. In other embodiments, non-immunoglobulin binding molecules of the invention also comprise a binding site with a protein topology that is not based on the immunoglobulin fold (e.g. such as ankyrin repeat proteins or fibronectins) but which nonetheless are capable of specifically binding to a target.

Non-immunoglobulin binding molecules may be identified by selection or isolation of a target-binding variant from a library of binding molecules having artificially diversified binding sites. Diversified libraries can be generated using completely random approaches (e.g., error-prone PCR, exon shuffling, or directed evolution) or aided by art-recognized design strategies. For example, amino acid positions that are usually involved when the binding site interacts with its cognate target molecule can be randomized by insertion of degenerate codons, trinucleotides, random peptides, or entire loops at corresponding positions within the nucleic acid which encodes the binding site (see e.g., U.S. Pub. No. 20040132028). The location of the amino acid positions can be identified by investigation of the crystal structure of the binding site in complex with the target molecule. Candidate positions for randomization include loops, flat surfaces, helices, and binding cavities of the binding site. In certain embodiments, amino acids within the binding site that are likely candidates for diversification can be identified by their homology with the immunoglobulin fold. For example, residues within the CDR-like loops of fibronectin may be randomized to generate a library of fibronectin binding molecules (see, e.g., Koide et al., J. Mol. Biol., 284: 1141-1151 (1998)). Other portions of the binding site which may be randomized include flat surfaces. Following randomization, the diversified library may then be subjected to a selection or screening procedure to obtain binding molecules with desired binding characteristics. For example, selection can be achieved by art-recognized methods such as phage display, yeast display, or ribosome display.

In one embodiment, a binding molecule of the invention comprises a binding site from a fibronectin binding molecule. Fibronectin binding molecules (e.g., molecules comprising the Fibronectin type I, II, or III domains) display CDR-like loops which, in contrast to immunoglobulins, do not rely on intra-chain disulfide bonds. Methods for making fibronectin binding polypeptides are described, for example, in WO 01/64942 and in U.S. Pat. Nos. 6,673,901, 6,703,199, 7,078,490, and 7,119,171, which are incorporated herein by reference. In one exemplary embodiment, the fibronectin binding polypeptide is as AdNectin® (Adnexus Therpaeutics, Waltham, Mass.).

In another embodiment, a binding molecule of the invention comprises a binding site from an Affibody® (Abcam, Cambridge, Mass.). Affibodies are derived from the immunoglobulin binding domains of staphylococcal Protein A (SPA) (see e.g., Nord et al., Nat. Biotechnol., 15: 772-777 (1997)). Affibody binding sites employed in the invention may be synthesized by mutagenizing an SPA-related protein (e.g., Protein Z) derived from a domain of SPA (e.g., domain B) and selecting for mutant SPA-related polypeptides having a desired binding affinity. Other methods for making affibody binding sites are described in U.S. Pat. Nos. 6,740,734 and 6,602,977 and in WO 00/63243, each of which is incorporated herein by reference.

In another embodiment, a binding molecule of the invention comprises a binding site from an Anticalin® (Pieris AG, Friesing, Germany). Anticalins (also known as lipocalins) are members of a diverse β-barrel protein family whose function is to bind target molecules in their barrel/loop region. Lipocalin binding sites may be engineered to bind a desired target by randomizing loop sequences connecting the strands of the barrel (see e.g., Schlehuber et al., Drug Discov. Today, 10: 23-33 (2005); Beste et al., PNAS, 96: 1898-1903 (1999). Anticalin binding sites employed in the binding molecules of the invention may be obtainable starting from polypeptides of the lipocalin family which are mutated in four segments that correspond to the sequence positions of the linear polypeptide sequence comprising amino acid positions 28 to 45, 58 to 69, 86 to 99 and 114 to 129 of the Bilin-binding protein (BBP) of Pieris brassica. Other methods for making anticalin binding sites are described in WO99/16873 and WO 05/019254, each of which is incorporated herein by reference.

In another embodiment, a binding molecule of the invention comprises a binding site from a cysteine-rich polypeptide. Cysteine-rich domains employed in the practice of the present invention typically do not form a α-helix, a β sheet, or a β-barrel structure. Typically, the disulfide bonds promote folding of the domain into a three-dimensional structure. Usually, cysteine-rich domains have at least two disulfide bonds, more typically at least three disulfide bonds. An exemplary cysteine-rich polypeptide is an A domain protein. A-domains (sometimes called “complement-type repeats”) contain about 30-50 or 30-65 amino acids. In some embodiments, the domains comprise about 35-45 amino acids and in some cases about 40 amino acids. Within the 30-50 amino acids, there are about 6 cysteine residues. Of the six cysteines, disulfide bonds typically are found between the following cysteines: C1 and C3, C2 and C5, C4 and C6. The A domain constitutes a ligand binding moiety. The cysteine residues of the domain are disulfide linked to form a compact, stable, functionally independent moiety. Clusters of these repeats make up a ligand binding domain, and differential clustering can impart specificity with respect to the ligand binding. Exemplary proteins containing A-domains include, e.g., complement components (e.g., C6, C7, C8, C9, and Factor I), serine proteases (e.g., enteropeptidase, matriptase, and corin), transmembrane proteins (e.g., ST7, LRP3, LRP5 and LRP6) and endocytic receptors (e.g., Sortilin-related receptor, LDL-receptor, VLDLR, LRP1, LRP2, and ApoER2). Methods for making A domain proteins of a desired binding specificity are disclosed, for example, in WO 02/088171 and WO 04/044011, each of which is incorporated herein by reference.

In other embodiments, a binding molecule of the invention comprises a binding site from a repeat protein. Repeat proteins are proteins that contain consecutive copies of small (e.g., about 20 to about 40 amino acid residues) structural units or repeats that stack together to form contiguous domains. Repeat proteins can be modified to suit a particular target binding site by adjusting the number of repeats in the protein. Exemplary repeat proteins include Designed Ankyrin Repeat Proteins (i.e., a DARPins®, Molecular Partners, Zurich, Switzerland) (see e.g., Binz et al., Nat. Biotechnol., 22: 575-582 (2004)) or leucine-rich repeat proteins (ie., LRRPs) (see e.g., Pancer et al., Nature, 430: 174-180 (2004)). All so far determined tertiary structures of ankyrin repeat units share a characteristic composed of a β-hairpin followed by two antiparallel α-helices and ending with a loop connecting the repeat unit with the next one. Domains built of ankyrin repeat units are formed by stacking the repeat units to an extended and curved structure. LRRP binding sites from part of the adaptive immune system of sea lampreys and other jawless fishes and resemble antibodies in that they are formed by recombination of a suite of leucine-rich repeat genes during lymphocyte maturation. Methods for making DARpin or LRRP binding sites are described in WO 02/20565 and WO 06/083275, each of which is incorporated herein by reference.

Other non-immunoglobulin binding sites which may be employed in binding molecules of the invention include binding sites derived from Src homology domains (e.g. SH2 or SH3 domains), PDZ domains, beta-lactamase, high affinity protease inhibitors, or small disulfide binding protein scaffolds such as scorpion toxins. Methods for making binding sites derived from these molecules have been disclosed in the art, see e.g., Silverman et al., Nat. Biotechnol., 23(12): 1493-4 (2005); Panni et al, J. Biol. Chem., 277: 21666-21674 (2002), Schneider et al., Nat. Biotechnol., 17: 170-175 (1999); Legendre et al., Protein Sci., 11:1506-1518 (2002); Stoop et al., Nat. Biotechnol., 21: 1063-1068 (2003); and Vita et al., PNAS, 92: 6404-6408 (1995). Yet other binding sites may be derived from a binding domain selected from the group consisting of an EGF-like domain, a Kringle-domain, a PAN domain, a Gla domain, a SRCR domain, a Kunitz/Bovine pancreatic trypsin Inhibitor domain, a Kazal-type serine protease inhibitor domain, a Trefoil (P-type) domain, a von Willebrand factor type C domain, an Anaphylatoxin-like domain, a CUB domain, a thyroglobulin type I repeat, LDL-receptor class A domain, a Sushi domain, a Link domain, a Thrombospondin type I domain, an Immunoglobulin-like domain, a C-type lectin domain, a MAM domain, a von Willebrand factor type A domain, a Somatomedin B domain, a WAP-type four disulfide core domain, a F5/8 type C domain, a Hemopexin domain, a Laminin-type EGF-like domain, a C2 domain, a CTLA-4 domain, and other such domains known to those of ordinary skill in the art, as well as derivatives and/or variants thereof. Additional non-immunoglobulin binding polypeptides include Avimers® (Avidia, Inc., Mountain View, Calif.—see International PCT Publication No. WO 06/055689 and US Patent Pub 2006/0234299), Telobodies® (Biotech Studio, Cambridge, Mass.), Evibodies® (Evogenix, Sydney, Australia—see U.S. Pat. No. 7,166,697), and Microbodies® (Nascacell Technologies, Munich, Germany).

ii. Binding Portions of Receptors and Ligands

In other aspects, the binding polypeptides of the invention comprise a ligand binding site of a receptor and/or a receptor binding portion of a ligand which is operably linked to a stabilized Fc region.

In certain embodiments, the binding polypeptide is a fusion of a ligand binding portion of a receptor and/or a receptor binding portion of a ligand with a stabilized Fc region. Any transmembrane regions or lipid or phospholipid anchor recognition sequences of the ligand binding receptor are preferably inactivated or deleted prior to fusion. DNA encoding the ligand or ligand binding partner is cleaved by a restriction enzyme at or proximal to the 5′ and 3′ ends of the DNA encoding the desired ORF segment. The resultant DNA fragment is then readily inserted (e.g., ligated in-frame) into DNA encoding a genetically-fused Fc region. The precise site at which the fusion is made may be selected empirically to optimize the secretion or binding characteristics of the soluble fusion protein. DNA encoding the fusion protein is then subcloned into an appropriate expression vector than can be transfected into a host cell for expression.

In one embodiment, a binding polypeptide of the invention combines the binding site(s) of the ligand or receptor (e.g. the extracellular domain (ECD) of a receptor) with a stabilized Fc region. In one embodiment, the binding domain of the ligand or receptor domain will be operably linked (e.g. fused via a polypeptide linker) to the C-terminus of a stabilized Fc region. N-terminal fusions are also possible. In exemplary embodiments, fusions are made to the C-terminus of the stabilized Fc region, or immediately N-terminal to the hinge domain a stabilized Fc region.

In certain embodiments, the binding site or domain of the ligand-binding portion of a receptor may be derived from a receptor bound by an antibody or antibody variant described supra. In other embodiments, the ligand binding portion of a receptor is derived from a receptor selected from the group consisting of a receptor of the Immunoglobulin (Ig) superfamily (e.g., a soluble T-cell receptor, e.g., mTCR®(Medigene AG, Munich, Germany), a receptor of the TNF receptor superfamily described supra (e.g., a soluble TNFα receptor of an immunoadhesin, e.g., Enbrel® (Wyeth, Madison, N.J.)), a receptor of the Glial Cell-Derived Neurotrophic Factor (GDNF) receptor family (e.g., GFRα3), a receptor of the G-protein coupled receptor (GPCR) superfamily, a receptor of the Tyrosine Kinase (TK) receptor superfamily, a receptor of the Ligand-Gated (LG) superfamily, a receptor of the chemokine receptor superfamily, IL-1/Toll-like Receptor (TLR) superfamily, and a cytokine receptor superfamily.

In other embodiments, the binding site or domain of the receptor-binding portion of a ligand may be derived from a ligand bound by an antibody or antibody variant described supra. For example, the ligand may bind a receptor selected from the group consisting of a receptor of the Immunoglobulin (Ig) superfamily, a receptor of the TNF receptor superfamily, a receptor of the G-protein coupled receptor (GPCR) superfamily, a receptor of the Tyrosine Kinase (TK) receptor superfamily, a receptor of the Ligand-Gated (LG) superfamily, a receptor of the chemokine receptor superfamily, IL-1/Toll-like Receptor (TLR) superfamily, and a cytokine receptor superfamily. In one exemplary embodiment, the binding site of the receptor-binding portion of a ligand is derived from a ligand belonging to the TNF ligand superfamily described supra (e.g., CD40L). In another embodiment, an exemplary target molecule is CD200 or CD200R

In other exemplary embodiments, a binding polypeptide of the invention may comprise one or more ligand binding domains or receptor binding domains derived from one or more of the following proteins:

1. Cytokines and Cytokine Receptors

Cytokines have pleiotropic effects on the proliferation, differentiation, and functional activation of lymphocytes. Various cytokines, or receptor binding portions thereof, can be utilized in the fusion proteins of the invention. Exemplary cytokines include the interleukins (e.g. IL-1, IL-2, IL-3, IL-4, IL-5, IL-6, IL-7, IL-8, IL-10, IL-11, IL-12, IL-13, and IL-18), the colony stimulating factors (CSFs) (e.g. granulocyte CSF (G-CSF), granulocyte-macrophage CSF (GM-CSF), and monocyte macrophage CSF (M-CSF)), tumor necrosis factor (TNF) alpha and beta, cytotoxic T lymphocyte antigen 4 (CTLA-4), and interferons such as interferon-α, β, or γ (U.S. Pat. Nos. 4,925,793 and 4,929,554).

Cytokine receptors typically consist of a ligand-specific alpha chain and a common beta chain. Exemplary cytokine receptors include those for GM-CSF, IL-3 (U.S. Pat. No. 5,639,605), IL-4 (U.S. Pat. No. 5,599,905), IL-5 (U.S. Pat. No. 5,453,491), IL10 receptor, IFNγ (EP0240975), and the TNF family of receptors (e.g., TNFα (e.g. TNFR-1 (EP 417, 563), TNFR-2 (EP 417,014) lymphotoxin beta receptor).

2. Adhesion Proteins

Adhesion molecules are membrane-bound proteins that allow cells to interact with one another. Various adhesion proteins, including leukocyte homing receptors and cellular adhesion molecules, or receptor binding portions thereof, can be incorporated in a fusion protein of the invention. Leucocyte homing receptors are expressed on leucocyte cell surfaces during inflammation and include the β-1 integrins (e.g. VLA-1, 2, 3, 4, 5, and 6) which mediate binding to extracellular matrix components, and the 132-integrins (e.g. LFA-1, LPAM-1, CR3, and CR4) which bind cellular adhesion molecules (CAMs) on vascular endothelium. Exemplary CAMs include ICAM-1, ICAM-2, VCAM-1, and MAdCAM-1. Other CAMs include those of the selectin family including E-selectin, L-selectin, and P-selectin.

3. Chemokines

Chemokines, chemotactic proteins which stimulate the migration of leucocytes towards a site of infection, can also be incorporated into a fusion protein of the invention. Exemplary chemokines include Macrophage inflammatory proteins (MIP-1-α and MIP-1-β), neutrophil chemotactic factor, and RANTES (regulated on activation normally T-cell expressed and secreted).

4. Growth Factors and Growth Factor Receptors

Growth factors or their receptors (or receptor binding or ligand binding portions thereof) may be incorporated in the fusion proteins of the invention. Exemplary growth factors include Vascular Endothelial Growth Factor (VEGF) and its isoforms (U.S. Pat. No. 5,194,596); Fibroblastic Growth Factors (FGF), including aFGF and bFGF; atrial natriuretic factor (ANF); hepatic growth factors (HGFs; U.S. Pat. Nos. 5,227,158 and 6,099,841), neurotrophic factors such as bone-derived neurotrophic factor (BDNF), glial cell derived neurotrophic factor ligands (e.g., GDNF, neuturin, artemin, and persephin), neurotrophin-3, -4, -5, or -6 (NT-3, NT-4, NT-5, or NT-6), or a nerve growth factor such as NGF-β platelet-derived growth factor (PDGF) (U.S. Pat. Nos. 4,889,919, 4,845,075, 5,910,574, and 5,877,016); transforming growth factors (TGF) such as TGF-alpha and TGF-beta (WO 90/14359), osteoinductive factors including bone morphogenetic protein (BMP); insulin-like growth factors-I and -II (IGF-I and IGF-II; U.S. Pat. Nos. 6,403,764 and 6,506,874); Erythropoietin (EPO); Thrombopoeitin (TPO; stem-cell factor (SCF), thrombopoietin (TPO, c-Mpl ligand), and the Wnt polypeptides (U.S. Pat. No. 6,159,462).

Exemplary growth factor receptors which may be used as targeting receptor domains of the invention include EGF receptors; VEGF receptors (e.g. Flt1 or Flk1/KDR), PDGF receptors (WO 90/14425); HGF receptors (U.S. Pat. Nos. 5,648,273, and 5,686,292), and neurotrophic receptors including the low affinity receptor (LNGFR), also termed as p75^{NTR} or p75 which binds NGF, BDNF, and NT-3, and high affinity receptors that are members of the trk family of the receptor tyrosine kinases (e.g. trkA, trkB (EP 455,460), trkC (EP 522,530)).

5. Hormones

Exemplary growth hormones for use as targeting agents in the fusion proteins of the invention include renin, human growth hormone (HGH; U.S. Pat. No. 5,834,598), N-methionyl human growth hormone; bovine growth hormone; growth hormone releasing factor; parathyroid hormone (PTH); thyroid stimulating hormone (TSH); thyroxine; proinsulin and insulin (U.S. Pat. Nos. 5,157,021 and 6,576,608); follicle stimulating hormone (FSH); calcitonin, luteinizing hormone (LH), leptin, glucagons; bombesin; somatropin; mullerian-inhibiting substance; relaxin and prorelaxin; gonadotropin-associated peptide; prolactin; placental lactogen; OB protein; or mullerian-inhibiting substance.

6. Clotting Factors

Exemplary blood coagulation factors for use as targeting agents in the fusion proteins of the invention include the clotting factors (e.g., factors V, VII, VIII, IX, X, XI, XII and XIII, von Willebrand factor); tissue factor (U.S. Pat. Nos. 5,346,991, 5,349,991, 5,726,147, and 6,596,84); thrombin and prothrombin; fibrin and fibrinogen; plasmin and plasminogen; plasminogen activators, such as urokinase or human urine or tissue-type plasminogen activator (t-PA).

### III. Multispecific Binding Polypeptides

In certain particular aspects, a binding polypeptide of the invention is multispecific, i.e., has at least one binding site that binds to a first molecule or epitope of a molecule and at least one second binding site that binds to a second molecule or to a second epitope of the first molecule. Multispecific binding molecules of the invention may comprise at least two binding sites, wherein at least one of the binding sites is derived from or comprises a binding site from one of binding molecules described supra. In certain embodiments, at least one binding site of a multispecific binding molecule of the invention is an antigen binding region of an antibody or an antigen binding fragment thereof (e.g. an antibody or antigen binding fragment described supra).

(a) Bispecific Molecules

In one embodiment, a binding polypeptide of the invention is bispecific. Bispecific binding polypeptides can bind to two different target sites, e.g., on the same target molecule or on different target molecules. For example, in the case of the binding polypeptides of the invention, a bispecific variant thereof can bind to two different epitopes, e.g., on the same antigen or on two different antigens. Bispecific binding polypeptides can be used, e.g., in diagnostic and therapeutic applications. For example, they can be used to immobilize enzymes for use in immunoassays. They can also be used in diagnosis and treatment of cancer, e.g., by binding both to a tumor associated molecule and a detectable marker (e.g., a chelator which tightly binds a radionuclide). Bispecific binding polypeptide can also be used for human therapy, e.g., by directing cytotoxicity to a specific target (for example by binding to a pathogen or tumor cell and to a cytotoxic trigger molecule, such as the T cell receptor or the Fcγ receptor). Bispecific binding polypeptides can also be used, e.g., as fibrinolytic agents or vaccine adjuvants.

Examples of bispecific binding polypeptides include those with at least two arms directed against different tumor cell antigens; bispecific altered binding proteins with at least one arm directed against a tumor cell antigen and at least one arm directed against a cytotoxic trigger molecule (such as anti-Fc.gamma.RI/anti-CD15, anti-p185.sup.HER2/Fc.gamma.RIII (CD16), anti-CD3/anti-malignant B-cell (1D10), anti-CD3/anti-p185.sup.HER2, anti-CD3/anti-p97, anti-CD3/anti-renal cell carcinoma, anti-CD3/anti-OVCAR-3, anti-CD3/L-D1 (anti-colon carcinoma), anti-CD3/anti-melanocyte stimulating hormone analog, anti-EGF receptor/anti-CD3, anti-CD3/anti-CAMA1, anti-CD3/anti-CD19, anti-CD3/MoV18, anti-neural cell adhesion molecule (NCAM)/anti-CD3, anti-folate binding protein (FBP)/anti-CD3, anti-pan carcinoma associated antigen (AMOC-31)/anti-CD3); bispecific binding polypeptides with at least one arm which binds specifically to a tumor antigen and at least one arm which binds to a toxin (such as anti-saporin/anti-Id-1, anti-CD22/anti-saporin, anti-CD7/anti-saporin, anti-CD38/anti-saporin, anti-CEA/anti-ricin A chain, anti-interferon-.alpha.(IFN-.alpha.)/anti-hybridoma idiotype, anti-CEA/anti-vinca alkaloid); bispecific binding polypeptides for converting enzyme activated prodrugs (such as anti-CD30/anti-alkaline phosphatase (which catalyzes conversion of mitomycin phosphate prodrug to mitomycin alcohol)); bispecific binding polypeptides which can be used as fibrinolytic agents (such as anti-fibrin/anti-tissue plasminogen activator (tPA), anti-fibrin/anti-urokinase-type plasminogen activator (uPA)); bispecific binding polypeptides for targeting immune complexes to cell surface receptors (such as anti-low density lipoprotein (LDL)/anti-Fc receptor (e.g. Fc.gamma.RI, Fc.gamma.RII or Fc.gamma.RIII)); bispecific binding polypeptides for use in therapy of infectious diseases (such as anti-CD3/anti-herpes simplex virus (HSV), anti-T-cell receptor:CD3 complex/anti-influenza, anti-Fc.gamma.R/anti-HIV; bispecific binding polypeptides for tumor detection in vitro or in vivo such as anti-CEA/anti-EOTUBE, anti-CEA/anti-DPTA, anti-p185HER2/anti-hapten); bispecific binding polypeptides as vaccine adjuvants (see Fanger et al., supra); and bispecific binding polypeptides as diagnostic tools (such as anti-rabbit IgG/anti-ferritin, anti-horse radish peroxidase (HRP)/anti-hormone, anti-somatostatin/anti-substance P, anti-HRP/anti-FITC, anti-CEA/anti-.beta.-galactosidase (see Nolan et al., supra)). Examples of trispecific polypeptides include anti-CD3/anti-CD4/anti-CD37, anti-CD3/anti-CD5/anti-CD37 and anti-CD3/anti-CD8/anti-CD37.

In a preferred embodiment, a bispecific binding polypeptide of the invention has one arm which binds to CRIPTO-I. In another preferred embodiment, a bispecific binding polypeptide of the invention has one arm which binds to LTβR. In another preferred embodiment, a bispecific binding polypeptide of the invention has one arm which binds to TRAIL-R2. In another preferred embodiment, a bispecific binding polypeptide of the invention has one arm which binds to LTβR and one arm which binds to TRAIL-R2.

Multispecific binding polypeptide of the invention may be monovalent for each specificity or be multivalent for each specificity. For example, binding polypeptides of the invention may comprise one binding site that reacts with a first target molecule and one binding site that reacts with a second target molecule or it may comprise two binding sites that react with a first target molecule and two binding sites that react with a second target molecule.

Binding polypeptides of the invention may have at least two binding specificities from two or more binding domains of a ligand or receptor). They can be assembled as heterodimers, heterotrimers or heterotetramers, essentially as disclosed in WO 89/02922 (published Apr. 6, 1989), in EP 314, 317 (published May 3, 1989), and in U.S. Pat. No. 5,116,964 issued May 2, 1992. Examples include CD4-IgG/TNFreceptor-IgG and CD4-IgG/L-selectin-IgG. The last mentioned molecule combines the lymph node binding function of the lymphocyte homing receptor (LHR, L-selectin), and the HIV binding function of CD4, and finds potential application in the prevention or treatment of HIV infection, related conditions, or as a diagnostic.

(b) scFv-Containing Multispecific Binding Molecules

In one embodiment, the multispecific binding molecules of the invention are multispecific binding molecules comprising at least one scFv molecule, e.g. an scFv molecule described supra. In other embodiments, the multispecific binding molecules of the invention comprise two scFv molecules, e.g. a bispecific scFv (Bis-scFv). In certain embodiments, the scFv molecule is a conventional scFv molecule. In other embodiments, the scFv molecule is a stabilized scFv molecule described supra. In certain embodiments, a multispecific binding molecule may be created by linking a scFv molecule (e.g., a stabilized scFv molecule) with a binding molecule scaffold comprising an scFc molecule. In one embodiment, the starting molecule is selected from the binding molecules described supra, and the scFv molecule and the starting binding molecule have different binding sites. For example, a binding molecule of the invention may comprise a scFv molecule with a first binding specificity linked to a second scFv molecule or a non-scFv binding molecule, that imparts second binding specificity. In one embodiment, a binding molecule of the invention is a naturally occurring antibody to which a stabilized scFv molecule has been fused.

When a stabilized scFv is linked to a parent binding molecule, linkage of the stabilized scFv molecule preferably improves the thermal stability of the binding molecule by at least about 2° C. or 3° C. In one embodiment, the scFv-containing binding molecule of the invention has a 1° C. improved thermal stability as compared to a conventional binding molecule. In another embodiment, a binding molecule of the invention has a 2° C. improved thermal stability as compared to a conventional binding molecule. In another embodiment, a binding molecule of the invention has a 4, 5, 6° C. improved thermal stability as compared to a conventional binding molecule.

In one embodiment, the multispecific binding molecules of the invention comprise at least one scFv (e.g. 2, 3, or 4 scFvs, e.g., stabilized scFvs). Further details regarding scFv molecules can be found in U.S. Ser. No. 11/725,970, incorporated by reference herein.

In one embodiment, the binding molecules of the invention are multispecific multivalent binding molecules having at least one scFv fragment with a first binding specificity and at least one scFv with a second binding specificity. In preferred embodiments, at least one of the scFv molecules is stabilized.

In another embodiment, the binding molecules of the invention are scFv tetravalent binding molecules. In preferred embodiments at least one of the scFv molecules is stabilized.

### (c) Multispecific Binding Molecule Fragments

In certain embodiments, binding polypeptide of the invention may comprise a binding site from a multispecific binding molecule fragment. Multispecific binding molecule fragments include bispecific Fab2 or multispecific (e.g. trispecific) Fab3 molecules. For example, a multispecific binding molecule fragment may comprise chemically conjugated multimers (e.g. dimers, trimers, or tetramers) of Fab or scFv molecules having different specificities.

### (d) Tandem Variable Domain Binding Molecules

In other embodiments, the multispecific binding molecule of the invention may comprise a binding molecule comprising tandem antigen binding sites. For example, a variable domain may comprise an antibody heavy chain that is engineered to include at least two (e.g., two, three, four, or more) variable heavy domains (VH domains) that are directly fused or linked in series, and an antibody light chain that is engineered to include at least two (e.g., two, three, four, or more) variable light domains (VL domains) that are direct fused or linked in series. The VH domains interact with corresponding VL domains to forms a series of antigen binding sites wherein at least two of the binding sites bind different epitopes. Tandem variable domain binding molecules may comprise two or more of heavy or light chains and are of higher order valency (e.g., bivalent or tetravalent). Methods for making tandem variable domain binding molecules are known in the art, see e.g. WO 2007/024715.

### (e) Dual Specificity Binding Molecules

In other embodiments, the multispecific binding molecule of the invention may comprise a single binding site having dual binding specificity. For example, a dual specificity binding molecule of the invention may comprise a binding site which cross-reacts with two epitopes. Art-recognized methods for producing dual specificity binding molecules are known in the art. For example, dual specificity binding molecules can be isolated by screening for binding molecules which bind both a first epitope and counter-screening the isolated binding molecules for the ability to bind to a second epitope.

### (f) Multispecific Fusion Proteins

In another embodiment, a multispecific binding molecule of the invention is a multispecific fusion protein. As used herein the phrase “multispecific fusion protein” designates fusion proteins (as hereinabove defined) having at least two binding specificities and further comprising an scFc. Multispecific fusion proteins can be assembled, e.g., as heterodimers, heterotrimers or heterotetramers, essentially as disclosed in WO 89/02922 (published Apr. 6, 1989), in EP 314, 317 (published May 3, 1989), and in U.S. Pat. No. 5,116,964 issued May 2, 1992. Preferred multispecific fusion proteins are bispecific. In certain embodiments, at least of the binding specificities of the multispecific fusion protein comprises an scFv, e.g., a stabilized scFv.

A variety of other multivalent antibody constructs may be developed by one of skill in the art using routine recombinant DNA techniques, for example as described in PCT International Application No. PCT/US86/02269; European Patent Application No. 184,187; European Patent Application No. 171,496; European Patent Application No. 173,494; PCT International Publication No. WO 86/01533; U.S. Pat. No. 4,816,567; European Patent Application No. 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; Shaw et al. (1988) J. Natl. Cancer Inst. 80:1553-1559); Morrison (1985) Science 229:1202-1207; Oi et al. (1986) BioTechniques 4:214; U.S. Pat. No. 5,225,539; Jones et al. (1986) Nature 321:552-525; Verhoeyan et al. (1988) Science 239:1534; Beidler et al. (1988) J. Immunol. 141:4053-4060; and Winter and Milstein, Nature, 349, pp. 293-99 (1991)). Preferably non-human antibodies are “humanized” by linking the non-human antigen binding domain with a human constant domain (e.g. Cabilly et al., U.S. Pat. No. 4,816,567; Morrison et al., Proc. Natl. Acad. Sci. U.S.A., 81, pp. 6851-55 (1984)).

Other methods which may be used to prepare multivalent antibody constructs are described in the following publications: Ghetie, Maria-Ana et al. (2001) Blood 97:1392-1398; Wolff, Edith A. et al. (1993) Cancer Research 53:2560-2565; Ghetie, Maria-Ana et al. (1997) Proc. Natl. Acad. Sci. 94:7509-7514; Kim, J. C. et al. (2002) Int. J. Cancer 97(4):542-547; Todorovska, Aneta et al. (2001) Journal of Immunological Methods 248:47-66; Coloma M. J. et al. (1997) Nature Biotechnology 15:159-163; Zuo, Zhuang et al. (2000) Protein Engineering (Suppl.) 13(5):361-367; Santos A. D., et al. (1999) Clinical Cancer Research 5:3118s-3123s; Presta, Leonard G. (2002) Current Pharmaceutical Biotechnology 3:237-256; van Spriel, Annemiek et al., (2000) Review Immunology Today 21(8) 391-397.

### (VII). Production of Stabilized Fc Polypeptides

The stabilized Fc polypeptides of the invention can be synthesized or expressed in cells which express nucleic acid molecules encoding the amino acid sequence of the polypeptide. Coding sequences can be selected using the genetic code and, optionally, optimized for the expression system selected.

For example, having selected a variant Fc polypeptide with enhanced stability, for example, a chimeric, human, humanized, or synthetic IgG antibody, a variety of methods are available for producing such polypeptides. Because of the degeneracy of the code, a variety of nucleic acid sequences will encode each amino acid sequence of the polypeptide. The desired nucleic acid sequences can be produced by de novo solid-phase DNA synthesis or by PCR mutagenesis of an earlier prepared polynucleotide encoding the Fc polypeptide. Oligonucleotide-mediated mutagenesis is one method for substituting the codon encoding an amino acid of a polypeptide with a stabilizing mutation. For example, the target polypeptide DNA is altered by hybridizing an oligonucleotide encoding the desired mutation to a single-stranded DNA template. After hybridization, a DNA polymerase is used to synthesize an entire second complementary strand of the template that incorporates the oligonucleotide primer, and encodes the selected alteration in the variant polypeptide DNA. In one embodiment, genetic engineering, e.g., primer-based PCR mutagenesis, is sufficient to alter the first amino acid, as defined herein, for producing a polynucleotide encoding a polypeptide that, when expressed in a eukaryotic cell, will now have a stabilized Fc region, for example, stabilized aglycosylated Fc region.

The variant Fc polypeptides of the invention typically comprise at least a portion of an antibody constant region (Fc), typically that of a human immunoglobulin. Ordinarily, the antibody will contain both light chain and heavy chain constant regions. The heavy chain constant region usually includes CH1, hinge, CH2, and CH3 regions whether derived from antibodies of the same or different isotypes. It is understood, however, that the antibodies described herein include antibodies having all types of constant regions, including IgM, IgG, IgD, and IgE, and any isotype, including IgG1, IgG2, IgG3, and IgG4. In one embodiment, the human isotype IgG1 is used. In another embodiment, the human isotype IgG4 is used. In one embodiment, a chimeric Fc region is used. Light chain constant regions can be lambda or kappa. The humanized antibody may comprise sequences from more than one class or isotype. Antibodies can be expressed as tetramers containing two light and two heavy chains, as separate heavy chains, light chains, as Fab, Fab′ F(ab′)2, and Fv, or as single chain Fv antibodies (scFv) in which heavy and light chain variable domains are linked through a spacer.

Methods for determining the effector function of a polypeptide comprising an Fc region, for example, an antibody, are described herein and include cell-based bridging assays to determine changes in the ability of a modified Fc region to bind to an Fc receptor. Other binding assays may be used to determine the ability of an Fc region to bind to a complement protein, for example, the C1q complement protein. Additional techniques for determining the effector function of a modified Fc region are described in the art.

### VIII. Stabilized Fc-Containing Polypeptides Comprising Functional Moieties

The variant Fc-containing polypeptides of the invention may be further modified to provide a desired effect. For example, the Fc region of the variant Fc-polypeptide may be linked, for example, covalently linked, to an additional moiety, i.e., a functional moiety such as, for example, a blocking moiety, a detectable moiety, a diagnostic moiety, and/or a therapeutic moiety. Exemplary functional moieties are first described below followed by useful chemistries for linking such functional moieties to the different amino acid side chain chemistries.

Examples of useful functional moieties include, but are not limited to, a blocking moiety, a detectable moiety, a diagnostic moiety, and a therapeutic moiety.

Exemplary blocking moieties include moieties of sufficient steric bulk and/or charge such that effector function is reduced, for example, by inhibiting the ability of the Fc region to bind a receptor or complement protein. Preferred blocking moieties include a polyalkylene glycol moiety, for example, a PEG moiety and preferably a PEG-maleimide moiety. Preferred pegylation moieties (or related polymers) can be, for example, polyethylene glycol (“PEG”), polypropylene glycol (“PPG”), polyoxyethylated glycerol (“POG”) and other polyoxyethylated polyols, polyvinyl alcohol (“PVA) and other polyalkylene oxides, polyoxyethylated sorbitol, or polyoxyethylated glucose. The polymer can be a homopolymer, a random or block copolymer, a terpolymer based on the monomers listed above, straight chain or branched, substituted or unsubstituted as long as it has at least one active sulfone moiety. The polymeric portion can be of any length or molecular weight but these characteristics can affect the biological properties. Polymer average molecular weights particularly useful for decreasing clearance rates in pharmaceutical applications are in the range of 2,000 to 35,000 daltons. In addition, if two groups are linked to the polymer, one at each end, the length of the polymer can impact upon the effective distance, and other spatial relationships, between the two groups. Thus, one skilled in the art can vary the length of the polymer to optimize or confer the desired biological activity. PEG is useful in biological applications for several reasons. PEG typically is clear, colorless, odorless, soluble in water, stable to heat, inert to many chemical agents, does not hydrolyze, and is nontoxic. Pegylation can improve pharmacokinetic performance of a molecule by increasing the molecule's apparent molecular weight. The increased apparent molecular weight reduces the rate of clearance from the body following subcutaneous or systemic administration. In many cases, pegylation can decrease antigenicity and immunogenicity. In addition, pegylation can increase the solubility of a biologically-active molecule.

Pegylated antibodies and antibody fragments may generally be used to treat conditions that may be alleviated or modulated by administration of the antibodies and antibody fragments described herein. Generally the pegylated aglycosylated antibodies and antibody fragments have increased half-life, as compared to the nonpegylated aglycosylated antibodies and antibody fragments. The pegylated aglycosylated antibodies and antibody fragments may be employed alone, together, or in combination with other pharmaceutical compositions.

Examples of detectable moieties which are useful in the methods and polypeptides of the invention include fluorescent moieties, radioisotopic moieties, radiopaque moieties, and the like, e.g. detectable labels such as biotin, fluorophores, chromophores, spin resonance probes, or radiolabels. Exemplary fluorophores include fluorescent dyes (e.g. fluorescein, rhodamine, and the like) and other luminescent molecules (e.g. luminal). A fluorophore may be environmentally-sensitive such that its fluorescence changes if it is located close to one or more residues in the modified protein that undergo structural changes upon binding a substrate (e.g. dansyl probes). Exemplary radiolabels include small molecules containing atoms with one or more low sensitivity nuclei (¹³C, ¹⁵N, ²H, ¹²⁵I, ¹²³I, ⁹⁹Tc, ⁴³K, ⁵²Fe, ⁶⁷Ga, ⁶⁸Ga, ¹¹¹In and the like). Other useful moieties are known in the art.

Examples of diagnostic moieties which are useful in the methods and polypeptides of the invention include detectable moieties suitable for revealing the presence of a disease or disorder. Typically a diagnostic moiety allows for determining the presence, absence, or level of a molecule, for example, a target peptide, protein, or proteins, that is associated with a disease or disorder. Such diagnostics are also suitable for prognosing and/or diagnosing a disease or disorder and its progression.

Examples of therapeutic moieties which are useful in the methods and polypeptides of the invention include, for example, anti-inflammatory agents, anti-cancer agents, anti-neurodegenerative agents, and anti-infective agents. The functional moiety may also have one or more of the above-mentioned functions.

Exemplary therapeutics include radionuclides with high-energy ionizing radiation that are capable of causing multiple strand breaks in nuclear DNA, and therefore suitable for inducing cell death (e.g., of a cancer). Exemplary high-energy radionuclides include: ⁹⁰Y, ¹²⁵I, ¹³¹I, ¹²³I, ¹¹¹In, ¹⁰⁵Rh, ¹⁵³Sm, ⁶⁷Cu, ⁶⁷Ga, ¹⁶⁶Ho, ¹⁷⁷Lu, ¹⁸⁶Re and ¹⁸⁸Re. These isotopes typically produce high energy α- or β-particles which have a short path length. Such radionuclides kill cells to which they are in close proximity, for example neoplastic cells to which the conjugate has attached or has entered. They have little or no effect on non-localized cells and are essentially non-immunogenic.

Exemplary therapeutics also include cytotoxic agents such as cytostatics (e.g. alkylating agents, DNA synthesis inhibitors, DNA-intercalators or cross-linkers, or DNA-RNA transcription regulators), enzyme inhibitors, gene regulators, cytotoxic nucleosides, tubulin binding agents, hormones and hormone antagonists, anti-angiogenesis agents, and the like.

Exemplary therapeutics also include alkylating agents such as the anthracycline family of drugs (e.g. adriamycin, caminomycin, cyclosporin-A, chloroquine, methopterin, mithramycin, porfiromycin, streptonigrin, porfiromycin, anthracenediones, and aziridines). In another embodiment, the chemotherapeutic moiety is a cytostatic agent such as a DNA synthesis inhibitor. Examples of DNA synthesis inhibitors include, but are not limited to, methotrexate and dichloromethotrexate, 3-amino-1,2,4-benzotriazine 1,4-dioxide, aminopterin, cytosine β-D-arabinofuranoside, 5-fluoro-5′-deoxyuridine, 5-fluorouracil, ganciclovir, hydroxyurea, actinomycin-D, and mitomycin C. Exemplary DNA-intercalators or cross-linkers include, but are not limited to, bleomycin, carboplatin, carmustine, chlorambucil, cyclophosphamide, cis-diammineplatinum(II) dichloride (cisplatin), melphalan, mitoxantrone, and oxaliplatin.

Exemplary therapeutics also include transcription regulators such as actinomycin D, daunorubicin, doxorubicin, homoharringtonine, and idarubicin. Other exemplary cytostatic agents that are compatible with the present invention include ansamycin benzoquinones, quinonoid derivatives (e.g. quinolones, genistein, bactacyclin), busulfan, ifosfamide, mechlorethamine, triaziquone, diaziquone, carbazilquinone, indoloquinone EO9, diaziridinyl-benzoquinone methyl DZQ, triethylenephosphoramide, and nitrosourea compounds (e.g. carmustine, lomustine, semustine).

Exemplary therapeutics also include cytotoxic nucleosides such as, for example, adenosine arabinoside, cytarabine, cytosine arabinoside, 5-fluorouracil, fludarabine, floxuridine, ftorafur, and 6-mercaptopurine; tubulin binding agents such as taxoids (e.g. paclitaxel, docetaxel, taxane), nocodazole, rhizoxin, dolastatins (e.g. Dolastatin-10, -11, or -15), colchicine and colchicinoids (e.g. ZD6126), combretastatins (e.g. Combretastatin A-4, AVE-6032), and vinca alkaloids (e.g. vinblastine, vincristine, vindesine, and vinorelbine (navelbine)); anti-angiogenesis compounds such as Angiostatin K1-3, DL-α-difluoromethyl-ornithine, endostatin, fumagillin, genistein, minocycline, staurosporine, and (±)-thalidomide.

Exemplary therapeutics also include hormones and hormone antagonists, such as corticosteroids (e.g. prednisone), progestins (e.g. hydroxyprogesterone or medroprogesterone), estrogens, (e.g. diethylstilbestrol), antiestrogens (e.g. tamoxifen), androgens (e.g. testosterone), aromatase inhibitors (e.g. aminogluthetimide), 17-(allylamino)-17-demethoxygeldanamycin, 4-amino-1,8-naphthalimide, apigenin, brefeldin A, cimetidine, dichloromethylene-diphosphonic acid, leuprolide (leuprorelin), luteinizing hormone-releasing hormone, pifithrin-α, rapamycin, sex hormone-binding globulin, and thapsigargin.

Exemplary therapeutics also include enzyme inhibitors such as, S(+)-camptothecin, curcumin, (−)-deguelin, 5,6-dichlorobenz-imidazole 1-β-D-ribofuranoside, etoposide, formestane, fostriecin, hispidin, 2-imino-1-imidazolidineacetic acid (cyclocreatine), mevinolin, trichostatin A, tyrphostin AG 34, and tyrphostin AG 879.

Exemplary therapeutics also include gene regulators such as 5-aza-2′-deoxycytidine, 5-azacytidine, cholecalciferol (vitamin D₃), 4-hydroxytamoxifen, melatonin, mifepristone, raloxifene, trans-retinal (vitamin A aldehydes), retinoic acid, vitamin A acid, 9-cis-retinoic acid, 13-cis-retinoic acid, retinol (vitamin A), tamoxifen, and troglitazone.

Exemplary therapeutics also include cytotoxic agents such as, for example, the pteridine family of drugs, diynenes, and the podophyllotoxins. Particularly useful members of those classes include, for example, methopterin, podophyllotoxin, or podophyllotoxin derivatives such as etoposide or etoposide phosphate, leurosidine, vindesine, leurosine and the like.

Still other cytotoxins that are compatible with the teachings herein include auristatins (e.g. auristatin E and monomethylauristan E), calicheamicin, gramicidin D, maytansanoids (e.g. maytansine), neocarzinostatin, topotecan, taxanes, cytochalasin B, ethidium bromide, emetine, tenoposide, colchicin, dihydroxy anthracindione, mitoxantrone, procaine, tetracaine, lidocaine, propranolol, puromycin, and analogs or homologs thereof.

Other types of functional moieties are known in the art and can be readily used in the methods and compositions of the present invention based on the teachings contained herein.

Chemistries for linking the foregoing functional moieties be they small molecules, nucleic acids, polymers, peptides, proteins, chemotherapeutics, or other types of molecules to particular amino acid side chains are known in the art (for a detailed review of specific linkers see, for example, Hermanson, G. T., Bioconjugate Techniques, Academic Press (1996)).

### IX. Expression of Stabilized Fc Polypeptides

The variant Fc polypeptides of the invention are preferably produced by recombinant expression of nucleic acid molecules encoding the polypeptides of the invention. In one embodiment, a nucleic acid molecule endocing a stabilized Fc polypeptide of the invention is present in a vector. In the case of antibodies, nucleic acids encoding light and heavy chain variable regions, optionally linked to constant regions, are inserted into expression vectors. The light and heavy chains can be cloned in the same or different expression vectors. The DNA segments encoding immunoglobulin chains are operably linked to control sequences in the expression vector(s) that ensure the expression of immunoglobulin polypeptides. Expression control sequences include, but are not limited to, promoters (e.g., naturally-associated or heterologous promoters), signal sequences, enhancer elements, and transcription termination sequences. Preferably, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences, and the collection and purification of the crossreacting antibodies.

These expression vectors are typically replicable in the host organisms either as episomes or as an integral part of the host chromosomal DNA. Commonly, expression vectors contain selection markers (e.g., ampicillin-resistance, hygromycin-resistance, tetracycline resistance or neomycin resistance) to permit detection of those cells transformed with the desired DNA sequences (see, e.g., Itakura et al., U.S. Pat. No. 4,704,362).

E. coli is one prokaryotic host particularly useful for cloning the polynucleotides (e.g., DNA sequences) of the present invention. Other microbial hosts suitable for use include bacilli, such as Bacillus subtilus, and other enterobacteriaceae, such as Salmonella, Serratia, and various Pseudomonas species.

Other microbes, such as yeast, are also useful for expression. Saccharomyces and Pichia are exemplary yeast hosts, with suitable vectors having expression control sequences (e.g., promoters), an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for methanol, maltose, and galactose utilization.

In addition to microorganisms, mammalian tissue culture may also be used to express and produce the polypeptides of the present invention (e.g., polynucleotides encoding immunoglobulins or fragments thereof). See Winnacker, From Genes to Clones, VCH Publishers, N.Y., N.Y. (1987). Eukaryotic cells are actually preferred, because a number of suitable host cell lines capable of secreting heterologous proteins (e.g., intact immunoglobulins) have been developed in the art, and include CHO cell lines, various COS cell lines, HeLa cells, 293 cells, myeloma cell lines, transformed B-cells, and hybridomas. Expression vectors for these cells can include expression control sequences, such as an origin of replication, a promoter, and an enhancer (Queen et al., Immunol. Rev. 89:49 (1986)), and necessary processing information sites, such as ribosome binding sites, RNA splice sites, polyadenylation sites, and transcriptional terminator sequences. Preferred expression control sequences are promoters derived from immunoglobulin genes, SV40, adenovirus, bovine papilloma virus, cytomegalovirus and the like. See Co et al., J. Immunol. 148:1149 (1992). In preferred embodiments, it will be understood that a polypeptide of the invention is a mature polypeptide, i.e., that it lacks a signal sequence.

Alternatively, sequences encoding variant Fc polypeptides of the invention can be incorporated in transgenes for introduction into the genome of a transgenic animal and subsequent expression in the milk of the transgenic animal (see, e.g., Deboer et al., U.S. Pat. No. 5,741,957, Rosen, U.S. Pat. No. 5,304,489, and Meade et al., U.S. Pat. No. 5,849,992). Suitable transgenes include coding sequences for light and/or heavy chains in operable linkage with a promoter and enhancer from a mammary gland specific gene, such as casein or beta lactoglobulin.

The vectors containing the polynucleotide sequences of interest (e.g., the heavy and light chain encoding sequences and expression control sequences) can be transferred into the host cell by well-known methods, which vary depending on the type of cellular host. For example, calcium chloride transfection is commonly utilized for prokaryotic cells, whereas calcium phosphate treatment, electroporation, lipofection, biolistics or viral-based transfection may be used for other cellular hosts. (See generally Sambrook et al., Molecular Cloning: A Laboratory Manual (Cold Spring Harbor Press, 2nd ed., 1989). Other methods used to transform mammalian cells include the use of polybrene, protoplast fusion, liposomes, electroporation, and microinjection (see generally, Sambrook et al., supra). For production of transgenic animals, transgenes can be microinjected into fertilized oocytes, or can be incorporated into the genome of embryonic stem cells, and the nuclei of such cells transferred into enucleated oocytes.

The polypeptides of the invention can be expressed using a single vector or two vectors. For example, when the antibody heavy and light chains are cloned on separate expression vectors, the vectors are co-transfected to obtain expression and assembly of intact immunoglobulins. Once expressed, the whole antibodies, their dimers, individual light and heavy chains, or other immunoglobulin forms of the present invention can be purified according to standard procedures of the art, including ammonium sulfate precipitation, affinity columns, column chromatography, HPLC purification, gel electrophoresis and the like (see generally Scopes, Protein Purification (Springer-Verlag, N.Y., (1982)). Substantially pure immunoglobulins of at least about 90 to 95% homogeneity are preferred, and 98 to 99% or more homogeneity most preferred, for pharmaceutical uses.

The stabilized Fc molecules of the invention are particularly suited to large scale production as they are resistant to agitation that occurs when production is scaled up. In addition, these molecules are stable during shipping and storage.

In one embodiment, the invention pertains to a method for large scale manufacture of a polypeptide comprising a stabilized Fc fusion protein, the method comprising:

(a) genetically fusing at least one stabilized Fc moiety to a polypeptide to form a stabilized fusion protein;

(b) transfecting a mammalian host cell with a nucleic acid molecule encoding the stabilized fusion protein,

(c) culturing the host cell of step (b) in 10 L or more of culture medium under conditions such that the stabilized fusion protein is expressed;

such that the stabilized fusion protein is produced.

In another embodiment, the method comprises: culturing a host cell expressing a nucleic acid molecule encoding the stabilized fusion protein in 10 L or more of culture medium under conditions such that the stabilized fusion protein is expressed and recovering the stabilized fusion protein from the culture medium. Optionally, one or more purification steps can be employed to obtain a composition of the desired purity (e.g. in which contamination from irrelevant proteins, aggregates, inactive forms of molecules is reduced).

### X. Prophylactic, Diagnostic, and Therapeutic Methods

The present invention is also directed inter alia to use of stabilized Fc polypeptides suitable for the prognosis, diagnosis, or treatment of diseases, including, for example, disorders where it is desirable to bind an antigen using a therapeutic antibody but refrain from triggering effector function.

Accordingly, in certain embodiments, the variant Fc polypeptides of the present invention are useful in the prevention or treatment of immune disorders including, for example, glomerulonephritis, scleroderma, cirrhosis, multiple sclerosis, lupus nephritis, atherosclerosis, inflammatory bowel diseases or rheumatoid arthritis. In another embodiment, the variant Fc polypeptides of the invention can be used to treat or prevent inflammatory disorders, including, but not limited to, Alzheimer's, severe asthma, atopic dermatitis, cachexia, CHF-ischemia, coronary restinosis, Crohn's disease, diabetic nephropathy, lymphoma, psoriasis, fibrosis/radiation-induced, juvenile arthritis, stroke, inflammation of the brain or central nervous system caused by trauma, and ulcerative colitis.

Other inflammatory disorders which can be prevented or treated with the variant Fc polypeptides of the invention include inflammation due to corneal transplantation, chronic obstructive pulmonary disease, hepatitis C, multiple myeloma, and osteoarthritis.

In another embodiment, the variant Fc polypeptides of the invention can be used to prevent or treat neoplasia, including, but not limited to bladder cancer, breast cancer, head and neck cancer, Kaposi's sarcoma, melanoma, ovarian cancer, small cell lung cancer, stomach cancer, leukemia/lymphoma, and multiple myeloma. Additional neoplasia conditions include, cervical cancer, colo-rectal cancer, endometrial cancer, kidney cancer, non-squamous cell lung cancer, and prostate cancer.

In another embodiment, the variant Fc polypeptides of the invention can be used to prevent or treat neurodegenerative disorders, including, but not limited to Alzheimer's, stroke, and traumatic brain or central nervous system injuries. Additional neurodegenerative disorders include ALS/motor neuron disease, diabetic peripheral neuropathy, diabetic retinopathy, Huntington's disease, macular degeneration, and Parkinson's disease.

In still another embodiment, the variant Fc polypeptides of the invention an be used to prevent or treat an infection caused by a pathogen, for example, a virus, prokaryotic organism, or eukaryotic organism.

In clinical applications, a subject is identified as having or at risk of developing one of the above-mentioned conditions by exhibiting at least one sign or symptom of the disease or disorder. At least one variant Fc polypeptide of the invention or compositions comprising at least one variant Fc polypeptide is administered in a sufficient amount to treat at least one symptom of a disease or disorder, for example, as mentioned above. In one embodiment, a subject is identified as exhibiting at least one sign or symptom of a disease or disorder associated with detrimental CD154 activity (also known as CD40 ligand or CD40L; see, e.g., Yamada et al., Transplantation, 73:S36-9 (2002); Schonbeck et al., Cell. Mol. Life. Sci. 42:4-43 (2001); Kirk et al., Philos. Trans. R. Soc. Lond. B. Sci. 356:691-702 (2001); Fiumara et al., Br. J. Haematol. 113:265-74 (2001); and Biancone et al., Int. J. Mol. Med. 3(4):343-53 (1999)).

Accordingly, a variant Fc polypeptide of the invention is suitable for administration as a therapeutic immunological reagent to a subject under conditions that generate a beneficial therapeutic response in a subject, for example, for the prevention or treatment of a disease or disorder, as for example, described herein.

Therapeutic agents of the invention are typically substantially pure from undesired contaminant. This means that an agent is typically at least about 50% w/w (weight/weight) purity, as well as being substantially free from interfering proteins and contaminants. Sometimes the agents are at least about 80% w/w and, more preferably at least 90 or about 95% w/w purity. However, using conventional protein purification techniques, for example as described herein, homogeneous peptides of at least 99% w/w can be obtained.

The methods can be used on both asymptomatic subjects and those currently showing symptoms of disease.

In another aspect, the invention features administering a variant Fc polypeptide with a pharmaceutical carrier as a pharmaceutical composition. Alternatively, the variant Fc polypeptide can be administered to a subject by administering a polynucleotide encoding the polypeptide. Where the Fc polypeptide is an antibody, the polynucleotide may be expressed to produce one or both of the heavy and light chains of the antibody. In certain embodiments, the polynucleotide is expressed to produce the heavy and light chains in the subject. In exemplary embodiments, the subject is monitored for the level of administered antibody in the blood of the subject.

The invention thus fulfills a longstanding need for therapeutic regimes for preventing or ameliorating immune conditions, for example, CD154-associated immune conditions.

It is also understood the antibodies of the invention are suitable for diagnostic or research applications, especially, for example, an diagnostic or research application comprising a cell-based assay where reduced effector function is desirable.

### XI. Animal Models for Testing the Efficacy of Fc Polypeptide

An antibody of the invention can be administered to a non-human mammal in need of, for example, an Fc polypeptide therapy, either for veterinary purposes or as an animal model of human disease, e.g., an immune disease or condition stated above. Regarding the latter, such animal models may be useful for evaluating the therapeutic efficacy of antibodies of the invention (e.g., testing of effector function, dosages, and time courses of administration).

Examples of animal models which can be used for evaluating the therapeutic efficacy of Fc polypeptides of the invention for preventing or treating rheumatoid arthritis (RA) include adjuvant-induced RA, collagen-induced RA, and collagen mAb-induced RA (Holmdahl et al., (2001) Immunol. Rev. 184:184; Holmdahl et al., (2002) Ageing Res. Rev. 1:135; Van den Berg (2002) Curr. Rheumatol. Rep. 4:232).

Examples of animal models which can be used for evaluating the therapeutic efficacy of antibodies or antigen-binding fragments of the invention for preventing or treating inflammatory bowel disease (IBD) include TNBS-induced IBD, DSS-induced IBD, and (Padol et al. (2000) Eur. J. Gastrolenterol. Hepatol. 12:257; Murthy et al. (1993) Dig. Dis. Sci. 38:1722).

Examples of animal models which can be used for evaluating the therapeutic efficacy of antibodies or antigen-binding fragments of the invention for preventing or treating glomerulonephritis include anti-GBM-induced glomerulonephritis (Wada et al. (1996) Kidney Int. 49:761-767) and anti-thy1-induced glomerulonephritis (Schneider et al. (1999) Kidney Int. 56:135-144).

Examples of animal models which can be used for evaluating the therapeutic efficacy of variant Fc polypeptides of the invention for preventing or treating multiple sclerosis include experimental autoimmune encephalomyelitis (EAE) (Link and Xiao (2001) Immunol. Rev. 184:117-128).

Animal models can also be used for evaluating the therapeutic efficacy of variant Fc polypeptides of the invention for preventing or treating CD154-related conditions, such as systemic erythematosus lupus (SLE), for example using the MRL-Fas^{lpr} mice (Schneider, supra; Tesch et al. (1999) J. Exp. Med. 190).

### XII. Treatment Regimes and Dosages

In prophylactic applications, pharmaceutical compositions or medicaments are administered to a subject suffering from a disorder treatable with a polypeptide having an Fc region, for example, an immune system disorder, in an amount sufficient to eliminate or reduce the risk, lessen the severity, or delay the outset of the disorder, including biochemical, histologic and/or behavioral symptoms of the disorder, its complications and intermediate pathological phenotypes presenting during development of the disorder. In therapeutic applications, compositions or medicaments are administered to a subject suspected of, or already suffering from such a disorder in an amount sufficient to cure, or at least partially arrest, the symptoms of the disorder (biochemical, histologic and/or behavioral), including its complications and intermediate pathological phenotypes in development of the disorder. The polypeptides of the invention are particularly useful for modulating the biological activity of a cell surface antigen that resides in the blood, where the disease being treated or prevented is caused at least in part by abnormally high or low biological activity of the antigen.

In some methods, administration of agent reduces or eliminates the immune disorder, for example, inflammation, such as associated with CD154 activity. An amount adequate to accomplish therapeutic or prophylactic treatment is defined as a therapeutically- or prophylactically-effective dose. In both prophylactic and therapeutic regimes, agents are usually administered in several dosages until a sufficient immune response has been achieved.

Effective doses of the compositions of the present invention, for the treatment of the above described conditions vary depending upon many different factors, including means of administration, target site, physiological state of the subject, whether the subject is human or an animal, other medications administered, and whether treatment is prophylactic or therapeutic. Usually, the subject is a human but non-human mammals including transgenic mammals can also be treated.

For passive immunization with a variant Fc polypeptide, the dosage ranges from about 0.0001 to 100 mg/kg, and more usually 0.01 to 20 mg/kg, of the host body weight. For example dosages can be 1 mg/kg body weight or 10 mg/kg body weight or within the range of 1-10 mg/kg, preferably at least 1 mg/kg. Subjects can be administered such doses daily, on alternative days, weekly or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months. Exemplary dosage schedules include 1-10 mg/kg or 15 mg/kg on consecutive days, 30 mg/kg on alternate days or 60 mg/kg weekly. In some methods, two or more monoclonal antibodies with different binding specificities are administered simultaneously, in which case the dosage of each antibody administered falls within the ranges indicated.

Polypeptides are usually administered on multiple occasions. Intervals between single dosages can be weekly, monthly or yearly. In some methods, dosage is adjusted to achieve a plasma antibody concentration of 1-1000 μg/ml and in some methods 25-300 μg/ml. Alternatively, polypeptides can be administered as a sustained release formulation, in which case less frequent administration is required. Dosage and frequency vary depending on the half-life of the antibody in the subject. In general, human antibodies show the longest half-life, followed by humanized antibodies, chimeric antibodies, and nonhuman antibodies.

The dosage and frequency of administration can vary depending on whether the treatment is prophylactic or therapeutic. In prophylactic applications, compositions containing the present antibodies or a cocktail thereof are administered to a subject not already in the disease state to enhance the subject's resistance. Such an amount is defined to be a “prophylactic effective dose.” In this use, the precise amounts again depend upon the subject's state of health and general immunity, but generally range from 0.1 to 25 mg per dose, especially 0.5 to 2.5 mg per dose. A relatively low dosage is administered at relatively infrequent intervals over a long period of time. Some subjects continue to receive treatment for the rest of their lives.

In therapeutic applications, a relatively high dosage (e.g., from about 1 to 200 mg of antibody per dose, with dosages of from 5 to 25 mg being more commonly used) at relatively short intervals is sometimes required until progression of the disease is reduced or terminated, and preferably until the subject shows partial or complete amelioration of symptoms of disease. Thereafter, the patent can be administered a prophylactic regime.

Doses for nucleic acids encoding antibodies range from about 10 ng to 1 g, 100 ng to 100 mg, 1 μg to 10 mg, or 30-300 μg DNA per subject. Doses for infectious viral vectors vary from 10-100, or more, virions per dose.

Therapeutic agents can be administered by parenteral, topical, intravenous, oral, subcutaneous, intraarterial, intracranial, intraperitoneal, intranasal or intramuscular means for prophylactic and/or therapeutic treatment. The most typical route of administration of a protein drug is intravascular, subcutaneous, or intramuscular, although other routes can be effective. In some methods, agents are injected directly into a particular tissue where deposits have accumulated, for example intracranial injection. In some methods, antibodies are administered as a sustained release composition or device, such as a Medipad™ device. The protein drug can also be administered via the respiratory tract, e.g., using a dry powder inhalation device.

Agents of the invention can optionally be administered in combination with other agents that are at least partly effective in treatment of immune disorders.

### XIII. Pharmaceutical Compositions

The therapeutic compositions of the invention include at least one stabilized Fc polypeptide of the invention in a pharmaceutically acceptable carrier. A “pharmaceutically acceptable carrier” refers to at least one component of a pharmaceutical preparation that is normally used for administration of active ingredients. As such, a carrier may contain any pharmaceutical excipient used in the art and any form of vehicle for administration. The compositions may be, for example, injectable solutions, aqueous suspensions or solutions, non-aqueous suspensions or solutions, solid and liquid oral formulations, salves, gels, ointments, intradermal patches, creams, lotions, tablets, capsules, sustained release formulations, and the like. Additional excipients may include, for example, colorants, taste-masking agents, solubility aids, suspension agents, compressing agents, enteric coatings, sustained release aids, and the like.

Agents of the invention are often administered as pharmaceutical compositions comprising an active therapeutic agent, i.e., and a variety of other pharmaceutically acceptable components. See Remington's Pharmaceutical Science (15th ed., Mack Publishing Company, Easton, Pa. (1980)). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

Variant Fc polypeptides can be administered in the form of a depot injection or implant preparation, which can be formulated in such a manner as to permit a sustained release of the active ingredient. An exemplary composition comprises monoclonal antibody at 5 mg/mL, formulated in aqueous buffer consisting of 50 mM L-histidine, 150 mM NaCl, adjusted to pH 6.0 with HCl.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above (see Langer, Science 249: 1527 (1990) and Hanes, Advanced Drug Delivery Reviews 28:97 (1997)).

The following examples are included for purposes of illustration and should not be construed as limiting the invention.

### EXAMPLES

Throughout the examples, the following materials and methods were used unless otherwise stated.

### Materials and Methods

In general, the practice of the present invention employs, unless otherwise indicated, conventional techniques of chemistry, molecular biology, recombinant DNA technology, immunology (especially, e.g., antibody technology), and standard techniques in electrophoresis. See, e.g., Sambrook, Fritsch and Maniatis, Molecular Cloning: Cold Spring Harbor Laboratory Press (1989); Antibody Engineering Protocols (Methods in Molecular Biology), 510, Paul, S., Humana Pr (1996); Antibody Engineering: A Practical Approach (Practical Approach Series, 169), McCafferty, Ed., Irl Pr (1996); Antibodies: A Laboratory Manual, Harlow et al., C.S.H.L. Press, Pub. (1999); and Current Protocols in Molecular Biology, eds. Ausubel et al., John Wiley & Sons (1992).

### Parental Antibodies

For producing the stabilized antibodies of the invention, polynucleotides encoding either a model human antibody (e.g., hu5c8), variant antibodies thereof, or corresponding Fc regions, were introduced into standard expression vectors. The human antibody hu5c8 and variants thereof are described in, e.g., U.S. Pat. Nos. 5,474,771 and 6,331,615. The amino acid sequences are provided below for, respectively, the hu5c8 IgG4 heavy chain (SEQ ID NO: 37), hu5c8 light chain (SEQ ID NO: 38), hu5c8 Fab (SEQ ID NO:39), complete Fc moiety from parental IgG4 antibody (SEQ ID NO:40), parental IgG4 Fc moiety with S228P mutation (SEQ ID NO:41), and parental aglycosylated IgG4 Fc moiety with S228P/T299A mutations (SEQ ID NO:42). The leader sequence for the heavy chain was MDWTWRVFCLLAVAPGAHS. Also provided is the heavy chain (SEQ ID NO: 43) and Fc moiety (SEQ ID NO:44) sequences of a parental IgG1 aglycosylated hu5c8 antibody.

Hu 5c8 IgG4 heavy chain (EAG1807)
(SEQ ID NO: 37)
Q V Q L V Q S G A E V V K P G A S V K L S C K A S G Y I F T S Y Y M Y W V K
Q A P G Q G L E W I G E I N P S N G D T N F N E K F K S K A T L T V D K S A S
T A Y M E L S S L R S E D T A V Y Y C T R S D G R N D M D S W G Q G T L V T
V S S A S T K G P S V F P L A P C S R S T S E S T A A L G C L V K D Y F P E P V
T V S W N S G A L T S G V H T F P A V L Q S S G L Y S L S S V V T V P S S S L G
T K T Y T C N V D H K P S N T K V D K R V E S K Y G P P C P P C P A P E F L G
G P S V F L F P P K P K D T L M I S R T P E V T C V V V D V S Q E D P E V Q F N
W Y V D G V E V H N A K T K P R E E Q F N S T Y R V V S V L T V L H Q D W L
N G K E Y K C K V S N K G L P S S I E K T I S K A K G Q P R E P Q V Y T L P P S
Q E E M T K N Q V S L T C L V K G F Y P S D I A V E W E S N G Q P E N N Y K T
T P P V L D S D G S F F L Y S R L T V D K S R W Q E G N V F S C S V M H E A L
H N H Y T Q K S L S L S L G
Hu 5c8 light chain
(SEQ ID NO: 38)
DIVLTQSPATLSVSPGERATISCRASQRVSSSTYSYMHWYQQKPGQPPKLLIKYA
SNLESGVPARFSGSGSGTDFTLTISSVEPEDFATYYCQHSWEIPPTFGGGTKLEIK
RTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGEC
Hu 5c8 VH/CH1 domains
(SEQ ID NO: 39)
Q V Q L V Q S G A E V V K P G A S V K L S C K A S G Y I F T S Y Y M Y W V K
Q A P G Q G L E W I G E I N P S N G D T N F N E K F K S K A T L T V D K S A S
T A Y M E L S S L R S E D T A V Y Y C T R S D G R N D M D S W G Q G T L V T
V S S A S T K G P S V F P L A P C S R S T S E S T A A L G C L V K D Y F P E P V
T V S W N S G A L T S G V H T F P A V L Q S S G L Y S L S S V V T V P S S S L G
T K T Y T C N V D H K P S N T K V D K R V
Parental IgG4 Fc moiety
(SEQ ID NO: 40)
E S K Y G P P C P S C P A P E F L G G P S V F L F P P K P K D T L M I S R T P E V
T C V V V D V S Q E D P E V Q F N W Y V D G V E V H N A K T K P R E E Q F N
S T Y R V V S V L T V L H Q D W L N G K E Y K C K V S N K G L P S S I E K T I
S K A K G Q P R E P Q V Y T L P P S Q E E M T K N Q V S L T C L V K G F Y P S
D I A V E W E S N G Q P E N N Y K T T P P V L D S D G S F F L Y S R L T V D K
S R W Q E G N V F S C S V M H E A L H N H Y T Q K S L S L S L G
Parental IgG4 Fc moiety with S228P mutation
(SEQ ID NO: 41)
E S K Y G P P C P P C P A P E F L G G P S V F L F P P K P K D T L M I S R T P E V
T C V V V D V S Q E D P E V Q F N W Y V D G V E V H N A K T K P R E E Q F N
S T Y R V V S V L T V L H Q D W L N G K E Y K C K V S N K G L P S S I E K T I
S K A K G Q P R E P Q V Y T L P P S Q E E M T K N Q V S L T C L V K G F Y P S
D I A V E W E S N G Q P E N N Y K T T P P V L D S D G S F F L Y S R L T V D K
S R W Q E G N V F S C S V M H E A L H N H Y T Q K S L S L S L G
Parental IgG4 Aglycosylated Fc moiety with S228P/T299A mutations (YC407)
(SEQ ID NO: 42)
ESKYGPPCPPCPAPEFLGGPSVFLFPPKPKDTLMISRTPEVTCVVVDVSQEDPEVQ
FNWYVDGVEVHNAKTKPREEQFNSAYRVVSVLTVLHQDWLNGKEYKCKVSN
KGLPSSIEKTISKAKGQPREPQVYTLPPSQEEMTKNQVSLTCLVKGFYPSDIAVE
WESNGQPENNYKTTPPVLDSDGSFFLYSRLTVDKSRWQEGNVFSCSVMHEALH
NHYTQKSLSLSLG
Parental IgG1 Aglycosylated Fc moiety
(SEQ ID NO: 43)
EPKSCDKTHTCPPCPAPELLGGPSVFLFPPKPKDTLMISRTPEVTCVVVDVSHED
PEVKFNWYVDGVEVHNAKTKPREEQYNSAYRVVSVLTVLHQDWLNGKEYKC
KVSNKALPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSVMHE
ALHNHYTQKSLSLSPG
Parental IgG4 Aglycosylated Fc with S228P/N297Q mutations (EAG2412)
(SEQ ID NO: 44)
ESKYGPPCPPCPAPEFLGGPSVFLFPPKPKDTLMISRTPEVTCVVVDVSQEDPEVQ
FNWYVDGVEVHNAKTKPREEQFQSTYRVVSVLTVLHQDWLNGKEYKCKVSN
KGLPSSIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALH
NHYTQKSLSLSP
Parental IgG1
(SEQ ID NO: 45)
EPKSCDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKC
KVSNKALPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSVMHE
ALHNHYTQKSLSLSPG

### Example 1

### Rational Design of Gain-in-Stability IgG Fc Mutations

Aglycosylated antibodies represent an important class of therapeutic reagents where immune effector function is not desired. However, it is well established that removal of the CH2 associated oligosaccharides in IgG1 and IgG4 affects antibody conformation and stability. Loss of antibody stability can present process development challenges adversely impacting program timelines and resources. Here we detail a number of methods utilized to design a library of amino acid positions in CH2 and CH3 to generate increased stability for IgG Fc.

### A. Covariation and Residue Frequency Designs for Effector-Less IgGs: IgG4 CH2 Domain

Covariation analyses with the diverse C1-class Ig-fold sequence database were performed as described previously (Glaser et al., 2007; Wang et al., 2008). Compilation and structure/HMM-based alignment of C1-class Ig-fold sequences was also performed as described previously (Glaser et al., 2007). The covariation analyses consist of a dataset of correlation coefficients, φ-values, relating how a pair of amino acids is or is not found to be co-conserved within particular protein sequences. φ-values range from −1.0 to 1.0. A φ-value of 1.0 indicates that when an amino acid is found at one position within a subset of sequences, another amino acid at a different residue position is also always found to be present in that subset. A φ-value of −1.0 indicates that when an amino acid is found at one position within a subset of sequences, another amino acid at a different residue position is never present in that sequence subset. Absolute φ-values greater than 0.2 were found to be statistically significant for the dataset that was analysed (Glaser et al., 2007; Wang et al., 2008). Based on experience with the dataset, φ-values >0.25 were deemed to be meaningful (i.e., there is likely to be a physical reason for the co-existence of the amino acid pair), while φ-values >0.5 were deemed to be very strong and likely co-conserved for important functional or structural reasons.

For this study, the CH2 sequence from IgG4 was used as a query sequence and a φ-values >0.3 was used as a cut-off to identify mutations by covariation. The residues identified from the covariation analysis are listed in Table 1.1 (all subsequent residues detailed throughout the rest of Example 1 are listed in Table 1.1). In Table 1.1, each residue gives reference to desired amino acid substitutions at that position according to the EU numbering system. “Rationale” refers to the design method employed. Covariation and Residue Frequency are described in detail in U.S. patent application Ser. No. 11/725,970. The number of additional covariation links refers to the additional covariation relationships formed by mutation to the listed amino acid type at a given position minus the number of covariation relationships lost by making this substitution. The number of additional covariation links is meant to be an additional measure of the quality of the suggested covariation mutation. In the case where multiple amino acid substitutions are suggested with no predominant associated additional number of covariation links, a library approach was used at this position in which all 20 amino acids were screened using the Delphia thermal challenge assay (detailed in Example 2). Using this methodology, six amino acid positions were identified with specific covariation mutations suggested: L242P (meaning L at position 242 changed to a P), Q268D, N286T, T307P, Y319F and S330A. In addition, five residue positions were identified to have multiple preferred (positive additional covariation links) substitutions, and a library approach was utilized. These positions are: D270, P271, E294, A299, and N315.

The methods for improving stability based on residue frequency analysis at individual positions within a protein fold has been successfully used (Steipe, 2004; Demarest et al., 2006)—and described previously in the patent application BGNA242-1 “STABILIZED POLYPEPTIDES AND METHODS FOR EVALUATING AND INCREASING THE STABILITY OF SAME” for identification of library positions within the anti-LTβR antibody BHA10 V_{H} and V_{L}-domains. Residue frequency analysis was used to identify five residue positions for gain-in-stability mutations: N276S, K288R, V308I, S324N, and G327A. In addition, two residues were generated by PCR error in the production of the covariation and residue frequency mutations: L309 and N325.

TABLE 1.1
Residues for Gain-in-Stability Mutations and Rationale

| | IgG4 | Most Frequent | Residue | Covari- | # of additional | Mutantions | |
|---|---|---|---|---|---|---|---|
| EU# | Residue | Residue | frequncy | ation 0.3 | covariation links | made | Rationale |
| 242 | L | I | 0.27 | P | 1 | P | Covariation |
| 268 | Q | Q | 1.00 | D | 1 | D | Covariation |
| 270 | D | D | 1.00 | * | library | nnk | Covariation |
| 271 | P | P | 1.00 | * | library | nnk | Covariation |
| 286 | N | T | 0.17 | T | 10 | T | Covariation |
| 294 | E | E | 1.00 | * | library | nnk | Covariation |
| 299 | A | T | 1.00 | * | library | K, Y, L | Covariation |
| 307 | T | P | 0.35 | P | 5 | P | Covariation |
| 315 | N | N | 1.00 | * | library | nnk | Covariation |
| 319 | Y | F | 0.27 | F | 1 | F | Covariation |
| 330 | S | A | 1.00 | A | 10 | A | Covariation |
| 276 | N | S | 0.70 | | | S | Residue |
| | | | | | | | Frequency |
| 288 | K | R | 0.91 | | | R | Residue |
| | | | | | | | Frequency |
| 308 | V | I | 0.35 | | | I | Residue |
| | | | | | | | Frequency |
| 324 | S | N | 0.21 | | | H, N | Residue |
| | | | | | | | Frequency |
| 327 | G | A | 1.00 | | | A | Residue |
| | | | | | | | Frequency |
| 309 | L | | | | | M, K, P | Screening |
| 325 | N | N | | | | H | Screening |
| 269 | E | | | | | library | Structural |
| | | | | | | | Analysis: In |
| | | | | | | | extended loop |
| 349 | Y | | | | | F | Structural |
| | | | | | | | Analysis: |
| | | | | | | | Interface |
| 350 | T | | | | | V | Structural |
| | | | | | | | Analysis: |
| | | | | | | | Interface |
| 394 | T | | | | | V | Structural |
| | | | | | | | Analysis: |
| | | | | | | | Interface |
| 399 | D | | | | | E, S | Structural |
| | | | | | | | Analysis: |
| | | | | | | | Interface |
| 405 | F | | | | | Y | Structural |
| | | | | | | | Analysis: |
| | | | | | | | Interface |
| 409 | R | | | | | K, M, I | Structural |
| | | | | | | | Analysis: |
| | | | | | | | Interface |
| 266 | V | | | | | F, Y | Structural |
| | | | | | | | Analysis: |
| | | | | | | | Interior bulk |
| 264 | V | | | | | K, T, N | Structural |
| | | | | | | | Analysis: Near |
| | | | | | | | carbohydrate |
| 292 | R | | | | | S, F | Structural |
| | | | | | | | Analysis: Near |
| | | | | | | | carbohydrate |
| 303 | V | | | | | S | Structural |
| | | | | | | | Analysis: Near |
| | | | | | | | carbohydrate |
| 310 | H | | | | | K, S, A | Structural |
| | | | | | | | Analysis: |
| | | | | | | | Near CH3 |
| 268 | Q | | | | | H | Structural |
| | | | | | | | Analysis: |
| | | | | | | | Residue Charge |
| 274 | Q | | | | | H, R | Structural |
| | | | | | | | Analysis: |
| | | | | | | | Residue Charge |
| 355 | Q | | | | | R, H | Structural |
| | | | | | | | Analysis: |
| | | | | | | | Residue Charge |
| 419 | E | | | | | Q, K | Structural |
| | | | | | | | Analysis: |
| | | | | | | | Residue Charge |
| 240 | V | | | | | I | Structural |
| | | | | | | | Analysis: |
| | | | | | | | Thermostable |
| 255 | V | | | | | I | Structural |
| | | | | | | | Analysis: |
| | | | | | | | Thermostable |
| 263 | V | | | | | I | Structural |
| | | | | | | | Analysis: |
| | | | | | | | Thermostable |
| 302 | V | | | | | I | Structural |
| | | | | | | | Analysis: |
| | | | | | | | Thermostable |
| 323 | V | | | | | I | Structural |
| | | | | | | | Analysis: |
| | | | | | | | Thermostable |
| 348 | V | | | | | I | Structural |
| | | | | | | | Analysis: |
| | | | | | | | Thermostable |
| 351 | L | | | | | I | Structural |
| | | | | | | | Analysis: |
| | | | | | | | Thermostable |
| 363 | V | | | | | I | Structural |
| | | | | | | | Analysis: |
| | | | | | | | Thermostable |
| 368 | L | | | | | I | Structural |
| | | | | | | | Analysis: |
| | | | | | | | Thermostable |
| 369 | V | | | | | I | Structural |
| | | | | | | | Analysis: |
| | | | | | | | Thermostable |
| 379 | V | | | | | I | Structural |
| | | | | | | | Analysis: |
| | | | | | | | Thermostable |
| 397 | V | | | | | I | Structural |
| | | | | | | | Analysis: |
| | | | | | | | Thermostable |
| 412 | V | | | | | I | Structural |
| | | | | | | | Analysis: |
| | | | | | | | Thermostable |
| 427 | V | | | | | I, F | Structural |
| | | | | | | | Analysis: |
| | | | | | | | Thermostable |

### B. Structural Analysis Designs for Effector-Less IgGs

In additional to the design of mutations by covariation and residue frequency analysis, structure analysis of the published crystal structure of intact human IgG b.12 antibody (pdb code: 1hzh; ref: Saphire, E. O., et al. (2001) Crystal structure of a neutralizing human IGG against HIV-1: a template for vaccine design. Science 293:1155-1159). The structural analysis identified specific structural qualities that could be modified to improve the stability of IgG molecules. In order to shift the stability of an IgG4 molecule closer to the stability of an IgG1, a number of mutations were made to compensate for the structural differences in between IgG1 and IgG4 molecules. One such mutation is located in an extended loop in IgG4, E269. A library approach was used to screen for residues that might compensate for the additional length of this loop. This loop was also the subject to additional changes as detailed in part C. of this Example.

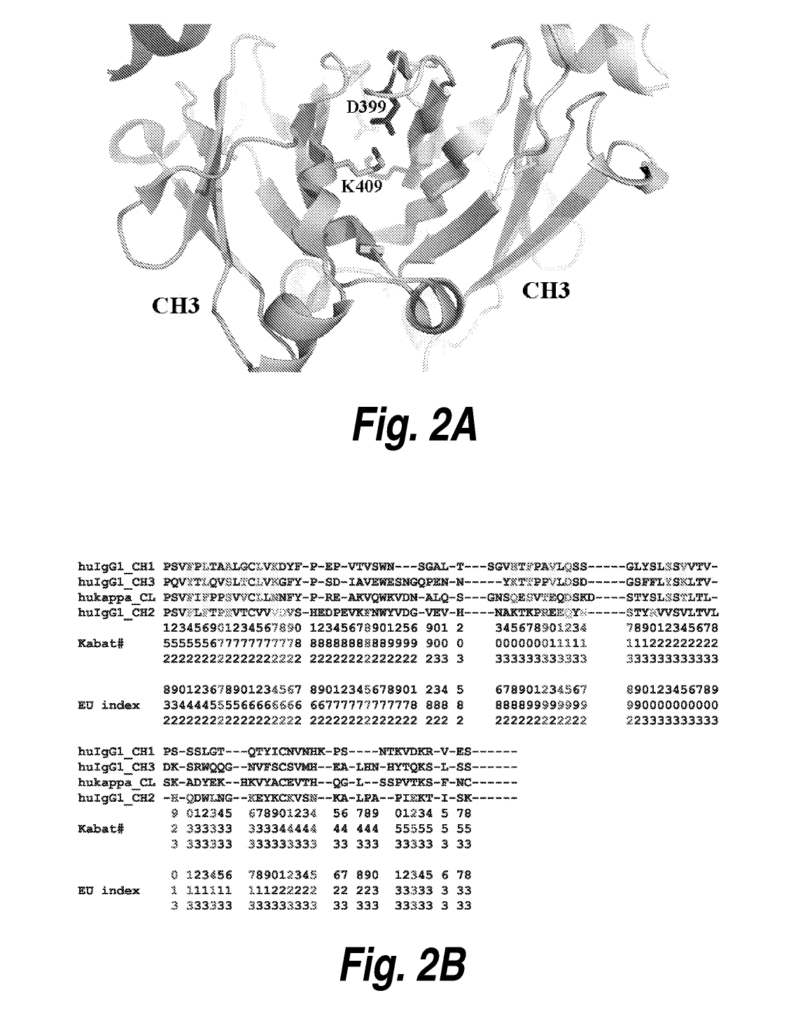

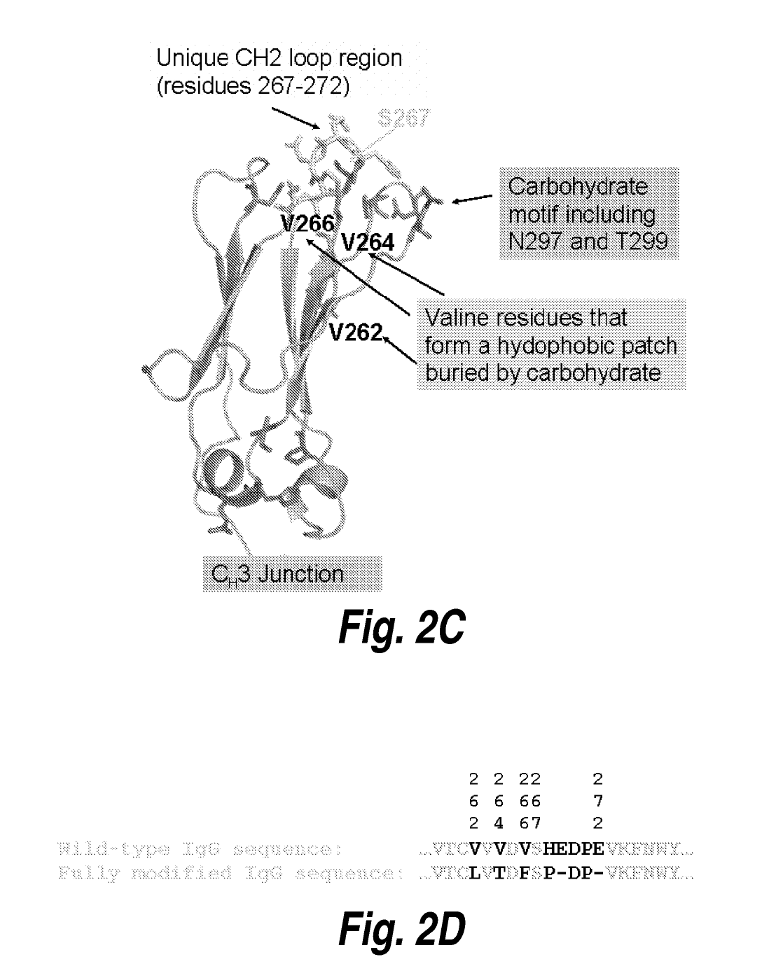

The interface between the CH3 domains constitutes the largest protein-protein contact area in the Fc domain of IgG molecules. A single substitutional difference in this interface between IgG1 and IgG4 is located at residue 409. In IgG1, a lysine is located at position 409 and in IgG4 molecules an arginine is located at position 409. Substitution of R409 in IgG4 to the IgG1 K409 was designed to introduce the superior stability qualities observed for the IgG1 CH3. R409M and R4091 were also designed to test this theory. To better accommodate the added bulk of the arginine in the IgG4 CH3 interface, a number of mutations were made at the contacting residue D399 from the opposite CH3 domain: D399E and D399S (FIG. 2A). By substituting a smaller side chain at this position, the opposite CH3 domain could better accommodate the added bulk of the arginine and increase the overall stability of the CH3 domain. Another approach was used in designing mutations that added hydrophobicity to the CH3 interface to increase the association between the two interacting domains (Y349F, T350V and T394V) as well as increase bulk in the side chains of the interface (F405Y). Mutations were also designed to test for stabilization in residues that were located near contact sites with the carbohydrate in the 1hzh crystal structure (V264, R292, V303) as well as H310 near the CH3/CH2 interface. A set of surface exposed glutamine residues (Q268, Q274 and Q355) were also the focus of a number of mutations to alter the overall surface charge. The same approach was used for E419.

Finally, one of the most common mechanisms used to explain the increased thermostability of thermophilic proteins involves tighter packing of the interior core of the protein (ref: Jaenicke, R. and Zavodszky, P. 1990. Proteins under extreme physical conditions. FEBS Lett. 268: 344-349). To recapitulate this phenomenon, valine residues found in the “valine core” of CH2 and CH3 were substituted with isoleucines or phenylalanines. Increase in stability was predicted from the additional branched side chains and greater associated bulk. The “valine core” in CH2 is five valine residues (V240, V255, V263, V302 and V323) that all are orientated into the same proximal interior core of the CH2 domain. A similar “valine core” is observed for CH3 (V348, V369, V379, V397, V412 and V427). In addition, L351 and L368 were mutated to higher branched hydrophobic sidechains.

### C. Covariation Designs for Effector-Less IgGs: Concerted Mutations Near the CH2 Glycosylation Site Based on Covariation Patterns Observed in Other C-Class Ig-Domains

The IgG C_{H}2 domain co-conserves many residues to maintain interactions with both the N-linked carbohydrate at EU position N297 and interactions with the various FcγR forms of CD16, CD32, and CD64. Removal of the carbohydrate leads to a dramatic reduction in FcγR-binding by IgG-Fcs (Taylor and Garber, 2005). For the designs described here, the co-mutability of residues near the N-linked carbohydrate within the IgG-Fc was investigated by substituting with amino acids found to be co-conserved in other C-class Ig-fold domains. The affect these co-mutations would have on FcγR-binding and on the stability of the C_{H}2 domain in the presence and absence of the N-linked carbohydrate was investigated, as it was possible these modifications might be both particularly well tolerated within an aglycosly-Fc and may reduce the interactions with FcγRs in both aglycosyl and glycosylated Fc moieties.

Residues important for potentially interacting with the N-linked carbohydrate were the focus of this study. IgG1-C_{H}2 residues that make direct contact with the carbohydrate at N297 were identified using a published crystal structure of IgG1-Fc bound to FcγRIIIa and the program MOLMOL (Sondermann, P., Huber, R., Oosthuizen, V., Jacob, U. (2000) The 3.2 Å crystal structure of the human IgG1 Fc fragment-FcgRIII complex. Nature, 406: 267-273; Koradi, R., Billeter, M. & Wuthrich, K. (1996) MOLMOL: a program for display and analysis of macromolecular structures. J. Mol. Graph. 14: 51-55). It was these amino acids that were the focus of the covariation analyses and designs.

Compilation and structure/HMM-based alignment of C1-class Ig-fold sequences was performed as described previously (Glaser et al., 2007). Covariation analyses with the diverse C1-class Ig-fold sequence database were also performed as described previously (Glaser et al., 2007; Wang et al., 2008). The covariation analyses consist of a dataset of correlation coefficients, φ-values, relating how a pair of amino acids is or is not found to be co-conserved within particular protein sequences. O-values range from −1.0 to 1.0. A φ-value of 1.0 indicates that when an amino acid is found at one position within a subset of sequences, another amino acid at a different residue position is also always found to be present in that subset. A φ-value of −1.0 indicates that when an amino acid is found at one position within a subset of sequences, another amino acid at a different residue position is never present in that sequence subset. Absolute φ-values greater than 0.2 were found to be statistically significant for the dataset that was analysed (Glaser et al., 2007; Wang et al., 2008). Based on experience with the dataset, φ-values >0.25 were deemed to be meaningful (i.e., there is likely to be a physical reason for the co-existence of the amino acid pair), while φ-values >0.5 were deemed to be very strong and likely co-conserved for important functional or structural reasons.

Based on structural analyses, it was found that hydrophobic residues V262 and V264 form a hydrophobic patch on the surface of the C_{H}2 domain that is sequestered from solvent by the N-linked carbohydrate. Additionally, V266 is a residue in the proximity of V262 and V264 and is unique to C_{H}2 domains, although it exists in a loop and buries itself into the interior of the domain. V262, V264, and V266 were found to be highly co-conserved within the IgG-C_{H}2 domain with highly significant correlation coefficients between one another (φ-values: V262-V264=0.44; V262-V26=0.40; V264-V266=0.54). The residues are highlighted in our structure-based sequence alignment of the IgG constant domains (FIG. 2B).

The three valine residues (262, 264, and 266) also have strong correlation coefficients with residues that form a unique loop structure in C_{H}2 domains (residues 267-271). This loop is two amino acids longer than the consensus loops formed by the other IgG constant domains C_{L}, C_{H}1, and C_{H}3. The specific correlations are between V262 and E269 and D270 (φ-values=0.38 and 0.31, respectively), V264 and 5267, D268, and E269 (φ-values=0.27, 0.44, and 0.52, respectively), and V266 and S267 (φ-value=0.30). Based on these correlations, we surmised that this loop may be important for positioning the loop containing N297 and its carbohydrate as well as positioning the loop containing residues 325-330 that is known to be important for interactions with FcγRs (Sondermann et al., 2000; Shields et al., 2001).

Based on these observations, we generated designs to investigate the tolerability (i.e., impact on the folding and stability of the C_{H}2 domain) of other amino acid types at these positions, particularly in aglycosyl-IgG. Another aspect we wished to observe was the affect modification at these sites might have on the FcγR-binding properties of an IgG. The amino acid changes that were made within the C_{H}2 domain based on these observations are listed in Table 1.2 and are shown on the structure of IgG-Fc in FIG. 2C (Sondermann, P., Huber, R., Oosthuizen, V., Jacob, U. (2000) The 3.2 Å crystal structure of the human IgG1 Fc fragment-FcgRIII complex. Nature, 406: 267-273). An alignment of the native sequence against the sequence (SDE9) containing all the mutations is shown in FIG. 2D.

TABLE 1.2
Mutations to aglycosyl-IgG1 CH2 domain.

| Construct | Native Amino Acid(s)/EU#/Mutant Amino Acid |
|---|---|
| SD401 | A299K^{a}, V262L |
| SD402 | A299K^{a}, V264T |
| SD403 | A299K^{a}, V266F |
| SD404 | A299K^{a}, V262L, V264T |
| SD405 | A299K^{a}, V264T, V266F |
| SDE8 | A299K^{a}, V262L, V264T, V266F |
| SD407 | A299K^{a}, Loop Replace (6 a. acids)-267SHEDPE272 with |
| | (4 a. acids)-PDPV |
| SDE7 | A299K^{a}, V262L, V264T, Loop Replace (6 a. acids)- |
| | 267SHEDPE272 with (4 a. acids)-PDPV |
| SDE9^{b} | A299K^{a}, V262L, V264T, V266F, Loop Replace |
| | (6 a. acids)-267SHEDPE272 with (4 a. acids)-PDPV |

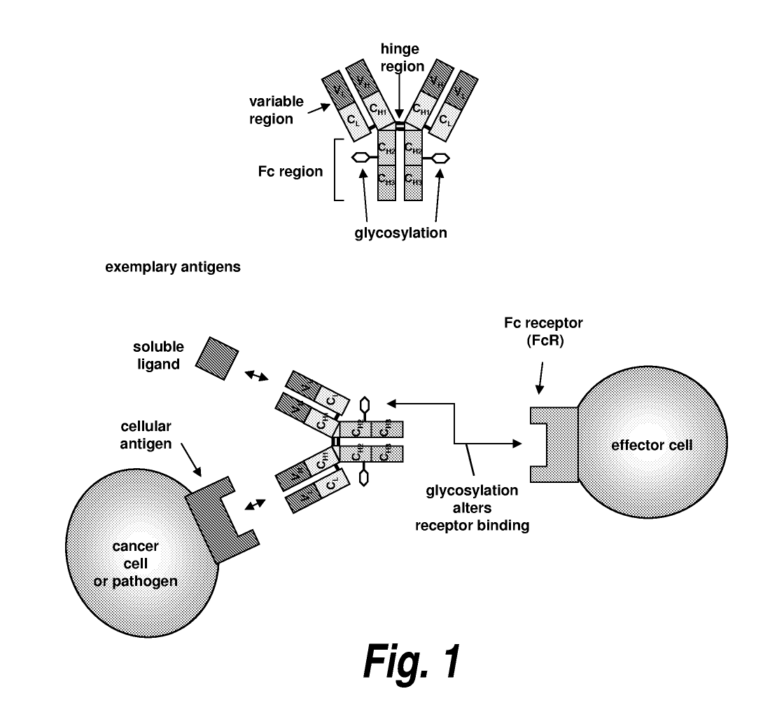

^{a}A299K mutation was made to interrupt the N-linked glycosylation motif resulting in an aglycosyl-IgG.
^{b}An alignment of the native sequence against the fully modified sequence is shown in FIG. 1D.

### D. Supporting Mutations

In order to test the specificity of a particular type of mutation at a given residue position, we have designed a series of additional mutations. These include testing different amino acid types (polar, hydrophobic, and charged) at residue positions that were shown to increase stability. We will also test the application of all gain-in-stability mutations to various IgG isotypes and glycosylation states. These mutations are listed in Table 1.3.

TABLE 1.3
Supporting mutations

| | | | Already |
|---|---|---|---|
| Number | Format | Constructs | Made |
| Positional | | | |
| 1 | IgG4.P agly | T299D | |
| 2 | | T299R | |
| 3 | | T299F | |
| 4 | | T299E | |
| 5 | | T299P | |
| 6 | | T299Q | |
| 7 | | T299N | |
| 8 | | T299S | |
| 9 | | T307V | |
| 10 | | T307D | |
| 11 | | T307K | |
| 12 | | T307S | |
| 13 | | L309I | |
| 14 | | L309D | |
| 15 | | L309R | |
| 16 | | L309T | |
| 17 | | D399A | |
| | | [symbol] | EC311 |
| 18 | | D399K | |
| | | [symbol] | EC310 |
| 19 | | T307P, L309K, T299K, R409K | |
| 20 | | T307P, L309K, T299K, R409M | |
| 21 | | T307P, L309K, T299K, R409M, | |
| | | D399N | |
| 22 | | T307P, L309K, T299K, R409M, | |
| | | D399E | |
| Isotype | | | |
| 23 | IgG1 agly | T307P | |
| 24 | | L309K | |
| 25 | | T307P, L309K | |
| 26 | | T307P, L309K, T299K | |
| 27 | IgG1 | T307P | |
| 28 | | L309K | |
| 29 | | T307P, L309K | |
| Variable | | | |
| 30 | BIIB022 | EC326 | |
| 31 | | EC331 | |
| 32 | | pEAG2300 | |

### E. Additional Multiple Mutation Constructs

In order to reduce potential T-cell epitopes generated from peptides with stability mutation T299K and to utilize the T307P and D399S stability mutations in combination with other mutations that result in an aglycosylated IgG1 and IgG4, we will also generate the following constructs (Table 1.4).

TABLE 1.4
Additional multiple mutation constructs

| | | |
|---|---|---|
| Number | Format | Constructs |

T-cell epitope

| | | |
|---|---|---|
| 1 | IgG1 agly | N297P, T299K |
| 2 | IgG1 agly | N297D, T299K |
| 3 | IgG1 agly | N297S, T299K |

Additional effectorless, stability engineered

| | | |
|---|---|---|
| 4 | IgG4.P agly | N297Q, T307P, D399S |
| 5 | IgG4.P agly/IgG1 | N297Q, T307P/IgG1 CH3 |
| | Chimeric | |

### Example 2

### Thermal Stability Screening of IgG Fc Antibody Domains Produced in E. coli

A modified thermal challenge assay described in U.S. patent application Ser. No. 11/725,970 was employed as a stability screen to determine the amount of soluble IgG Fc protein at 40° C. retained following a thermal challenge event at pH 4.5.

E. coli strain W3110 (ATCC, Manassas, Va. Cat. #27325) was transformed with plasmids encoding pBRM012 (IgG1) and pBRM013 (IgG4 with S228P, T299A mutations) Fc's plus C-terminal Histidine tag under the control of an inducible ara C promoter. Transformants were grown overnight in expression media consisting of SB (Teknova, Half Moon Bay, Ca. Cat. # S0140) supplemented with 0.6% glycine, 0.6% Triton X100, 0.02% arabinose, and 50 μg/ml carbenicillin at 30° C. Bacteria was pelleted by centrifugation and supernatants harvested for further treatment.

After thermal challenge, the aggregated material was removed by centrifugation and soluble Fc samples remaining in the treated, cleared supernatant were assayed for binding to Protein A (Sigma P7837) by DELFIA assay. Two 96-well plates (MaxiSorp, Nalge Nunc, Rochester, N.Y., Cat. #437111) were coated for one hour at 37° C. with Protein A at 0.5 μg/ml in PBS, and then blocked with DELFIA assay buffer (DAB, 10 mM Tris HCl, 150 mM NaCl, 20 μM EDTA, 0.5% BSA, 0.02% Tween 20, 0.01% NaN₃, pH 7.4) for one hour with shaking at room temperature. The plate was washed 3 times with DAB without BSA (Wash buffer), and 10 μl of supernatant were added to 90 of DAB to achieve a final volume of 100 μl (reference plate). 10 μA of 10% HOAc was next added to each supernatant in a polypropylene plate to achieve a sample pH of 4.5. The plate was incubated for 90 minutes at 40° C. and denatured proteins were removed by centrifugation at 1400×g. 10 μl of acid and heat treated supernatant were added to in another DELFIA plate containing 90 μl of DAB supplemented with 100 mM Tris, pH 8.0 (challenge plate). The DELFIA plates were incubated at room temperature with shaking for one hour, and washed 3 times as before. Bound Fc was detected by addition of 100 μl per well of DAB containing 250 ng/ml of Eu-labeled anti-His₆ antibody (Perkin Elmer, Boston, Mass., Cat. # AD0109) and incubated at room temperature with shaking for one hour. The plate was washed 3 times with Wash buffer, and 100 μl of DELFIA enhancement solution (Perkin Elmer, Boston, Mass., Cat. #4001-0010) was added per well. Following incubation for 15 minutes, the plate was read using the Europium method on a Victor 2 (Perkin Elmer, Boston, Mass.). Data was analyzed by ranking the ratio of Eu-fluorescence between the reference and challenge plates for the various constructs at 40° C. Fluorescence values greater than the value for pBRM013 were interpreted as an increase in stability over the target construct (IgG4.P agly). Data is shown in Table 2.1.

TABLE 2.1
Delphia Thermal Challenge Assay Results

| | IgG4 | | | |
|---|---|---|---|---|
| EU# | Residue | Mutant | Rationale | normAvgF (T = 40° C.) |
| 242 | L | P | Covariation | <4.33 |
| 242 | L | I | Residue | <4.33 |
| | | | Frequency | |
| 268 | Q | D | Covariation | <4.33 |
| 268 | Q | H | Residue Charge | <4.33 |
| 270 | D | nnk | Covariation | <4.33 |
| 271 | P | nnk | Covariation | <4.33 |
| 274 | Q | H | Residue Charge | <4.33 |
| 274 | Q | R | Residue Charge | <4.33 |
| 276 | N | S | Residue | <4.33 |
| | | | Frequency | |
| 286 | N | T | Covariation | <4.33 |
| 288 | K | R | Residue | <4.33 |
| | | | Frequency | |
| 294 | E | nnk | Covariation | <4.33 |
| 299 | A | K | Covariation | 4.83 |
| 299 | A | Y | Covariation | 4.71 |
| 299 | A | L | Covariation | <4.33 |
| 307 | T | P | Covariation | 5.43 |
| 308 | V | I | Residue | <4.33 |
| | | | Frequency | |
| 309 | L | M | | 5.17 |
| 309 | L | K | | <4.33 |
| 309 | L | P | | <4.33 |
| 315 | N | nnk | Covariation | <4.33 |
| 319 | Y | F | Covariation | <4.33 |
| 324 | S | H | Residue | <4.33 |
| | | | Frequency | |
| 324 | S | N | Residue | <4.33 |
| | | | Frequency | |
| 327 | G | A | Residue | <4.33 |
| | | | Frequency | |
| 330 | S | A | Residue | <4.33 |
| | | | Frequency | |
| 355 | Q | R | Residue Charge | <4.33 |
| 355 | Q | H | Residue Charge | <4.33 |
| 419 | E | Q | Residue Charge | <4.33 |
| 419 | E | K | Residue Charge | <4.33 |

| | |
|---|---|
| wt IgG1 agly | 5.45 |
| wt IgG4.P agly | 4.33 |

Combinations

| | | | |
|---|---|---|---|
| 276 | N | S | 5.49 |
| 307 | T | P | |
| 286 | N | T | 5.31 |
| 307 | T | P | |
| 276 | N | S | 5.25 |
| 286 | N | T | |
| 307 | T | P | |
| 308 | V | I | 4.99 |
| 309 | L | K | |

### Example 3

### Production of Stabilized IgG Fc Antibodies

A. Mutagenesis, Transient Expression of Stabilized IgG Fc Moieties in E. coli and Purification

Stability mutations were incorporated into an the BRM13 construct previously detailed in Example 2, by Site-Directed mutagenesis using a Stratagene Quik-Change Lightning mutagenesis kit. Primers were designed between 36-40 bases in length with the mutation in the middle with 10-15 bases of correct sequence on both sides, at least 40% GC content, starting and terminating in one or more C/G bases. All mutant constructs are listed in Table 3.1 below.

TABLE 3.1
IgG-Fc constructs Expressed and Purified from E. coli

| | Final AA Substitution |
|---|---|
| BRM013 | IgG4.P S228P, T299A |
| BRM023 | S228P, T299A, T307P |
| BRM030 | S228P, T299K |
| CR103 | S228P, T299A, R409K |
| CR104 | S228P, T299A, R409M |
| CR105 | S228P, T299A, R409L |
| CR106 | S228P, T299A, R409I |
| CR107 | S228P, T299A, D399S |
| CR108 | S228P, T299A, D399N |
| CR109 | S228P, T299A, D399E |
| CR110 | S228P, T299A, V369I |
| CR111 | S228P, T299A, V379I |
| CR112 | S228P, T299A, V397I |
| CR113 | S228P, T299A, V427I |
| CR114 | S228P, T299A, V427F |
| CR115 | S228P, T299A, V240I |
| CR116 | S228P, T299A, V263I |
| CR117 | S228P, T299A, V273I |
| CR118 | S228P, T299A, V302I |
| CR119 | S228P, T299A, V323I |

Following the PCR using the primers that would introduce the mutation, each mutagenesis was digested with a Dpn I restriction enzyme at 37° C. for 5 minutes in order to completely digest the parental plasmid. The mutagenesis reactions were then transformed into XL1-Blue E. Coli ultracompetent cells. Ampicillin resistant colonies were screened and DNA sequencing was used to confirm the right sequence from the mutagenesis reaction.

Sequence confirmed DNA was transform the into 3110 cells by electroporation using the EC3 program. Unique colonies were picked and grown in a starter culture in 10 ml LB-amp overnight. This preculture was transferred to 1 L expression media [SB+0.02% arabinose+amp/carb 50 mg/L] and grown overnight at 32° C. Cells were spun down in a centrifuge and resuspended completely in the 100 ml of spheroplast buffer (20% sucrose, 1 mM EDTA, 10 mM Tris-HCl pH 8.0, and lysozyme (0.01% w/v)). Cells were spun down and resultant protein was in supernatant.

The IgG-Fc constructs were purified by batch-purification using Protein A Sepharose FF (GE Healthcare). The Fc molecule was eluted from the Protein A Sepharose using 0.1 M glycine at pH 3.0, neutralized with Tris base, and finally dialyzed into PBS using the Pierce 10 ml dialysis cassettes (10,000 MWCO cutoff).

### B. Mutagenesis, Transient Expression of Stabilized Antibodies in CHO Cells, Antibody Purification, and Characterization

Stability mutations were incorporated into an IgG4.P antibody (a VH construct already containing a proline hinge mutation at amino acid 228) by Site-Directed mutagenesis using a Stratagene Quik-Change Lightning mutagenesis kit. The antigen recognizing Fab was from the anti-CD40 antibody 5c8. Primers were designed between 36-40 bases in length with the mutation in the middle with 10-15 bases of correct sequence on both sides, at least 40% GC content, starting and terminating in one or more C/G bases. All glycosylated and aglycosylated mutant constructs are listed in Table 3.2.

TABLE 3.2
Protein yield from 1 L culture and % Monomer as measured by
Analytical Size-Exclusion Chromatography (IgG1 constructs in italics)

| | | |
|---|---|---|
| | yield | |
| Final AA Substitution | (mg) | % monomer |

I. Glycosylated

| | | | |
|---|---|---|---|
| EC301 | S228P, A299K, V427F | 2.2 | 53% |
| EC302 | S228P, A299K, D399S | 4.3 | 98.60% |
| EC303 | S228P, T307P, V427F | 1.7 | 98.20% |
| EC304 | S228P, T307P, D399S | 2.9 | 99.00% |
| EC305 | S228P, A299K, V427F, | 5 | 99.10% |
| | D399S | | |
| EC306 | S228P, T307P, V427F, | 15.3 | 28% |
| | D399S | | |
| EC307 | S228P, A299K, V427F, | 0 | — |
| | V348F | | |
| EC308 | S228P, T307P, V323F | 9 | 99.50% |
| EC309 | S228P, V240F | 15.75 | 98.10% |
| EC321 | S228P, D399S, L309P | 13.3 | 97.80% |
| EC322 | S228P, D399S, L309M | 13.3 | 97.50% |
| EC323 | S228P, D399S, L309K | 13.41 | 98.40% |
| EC324 | S228P, T307P, D399S, L309P | 15.66 | 97% |
| EC325 | S228P, T307P, D399S, | 8.1 | 97.80% |
| | L309M | | |
| EC326 | S228P, T307P, D399S, | 21.1 | 98.60% |
| | L309K | | |
| EC300 | S228P, T307P | 16 | 98.30% |

II. Aglycosylated

| | | | |
|---|---|---|---|
| EC330 | S228P/T299A/T307/IgG1- | 21.42 | 98.10% |
| | CH3 | | |
| EC331 | S228P/T299K/T307/IgG1- | 7 | 98.70% |
| | CH3 | | |
| YC401 | S228P, T299A, T307P, | 3 | 96% |
| | D399S | | |
| YC402 | S228P, T299A, L309K, | 3 | 95% |
| | D399S | | |
| YC403 | S228P, T299A, T307P, | 4 | 95.10% |
| | D399S, L309K | | |
| YC404 | S228P, T299K, T307P, | 5 | 97.22% |
| | D399S | | |
| YC405 | S228P, T299K, L309K, | 4.5 | 95% |
| | D399S | | |
| YC406 | S228P, T299K, T307P, | 3.5 | 96% |
| | D399S, L309K | | |
| YC407 | S228P, T299A | 4.07 | 96.90% |
| CN578 | T299K (IgG1) | 9.38 | 100% |
| CN579 | S228P, T299K | 11.55 | 90% |
| pEAG2296 | S228P/T299A/IgG1-CH3 | 7.24 | 98% |
| pEAG2287 | S228P/T299K/IgG1-CH3 | 14.2 | 100% |
| SDE1 | A299K, V262L | 4.91 | 100% |
| SDE2 | A299K, V264T | 2.8 | 100% |
| SDE3 | A299K, V266F | 8.96 | 95.15% |
| SDE4 | A299K, V262L, V264T | 2.6 | 95.20% |
| SDE5 | A299K, V264T, V266F | 3.93 | 95.40% |
| SDE6 | A299K, Loop Replacement | 2.11 | 95.95% |
| SDE7 | A299K, Loop + V262L/V264T | 8.54 | 99.10% |
| SDE8 | A299K, V262L, V264T, | 6.83 | 98.90% |
| | V266F | | |
| SDE9 | A299K, Loop + | 6.46 | 99.20% |
| | V262L/V264T/V266F | | |

Following the PCR using the primers that would introduce the mutation, each mutagenesis was digested with a Dpn I restriction enzyme at 37° C. for 5 minutes in order to completely digest the parental plasmid. The mutagenesis reactions were then transformed into XL10-Gold E. Coli ultracompetent cells. Ampicillin resistant colonies were screened and DNA sequencing was used to confirm the right sequence from the mutagenesis reaction.

DNA from confirmed sequences were scaled up and transformed into TOP10 E. coli competent cells (Invitrogen Corporation, Carlsbad, Calif.). E. coli colonies transformed to ampicillin drug resistance were screened for presence of inserts. Colonies were then cultured into large scale culture of 250 ml. A Qiagen HiSpeed Maxiprep kit was used to extract and purify the DNA from the bacterial culture for transient transfection. The DNA was quantified using an 8280 to measure DNA concentration to be used for transfection.

The mutant plasmids along with an equal amount of 5c8 VL plasmid were then used to co-transfect CHO-S cells for transient expression of antibody protein. The amount of DNA to be used for the transfection was 0.5 mg/L of the VH and 0.5 mg/L of the VL. The transfection media (CHO-S-SFMII from Invitrogen with LONG R4IGF-1 from SAFC) was prepared at 5% of the transfection volume with 1 mg/ml of PEI (Polysciences Cat. #23966) in a ratio of 3 mg of PEI to 1 mg of DNA. DNA was added to the transfection media/PEI solution and swirled then sat at room temperature for 5 minutes. The mixture was then added to 500 ml of CHO-S cells at 1e6 cells/ml. After 4 hours at 37° C. at 5% CO₂, 1× volume of expansion media (CHOM37+20 g/l PDSF+Penstrep/amphostericin) was added for a final culture volume of 1 L. On day 1, 10 ml of cotton hydrolysate at 200 g/L was added and the temperature was dropped to 28° C. Culture viability was monitored until the viability dropped below 70% (8-12 days). Titers for protein expression were also checked at this point using the Octet (ForteBio) in measuring binding to anti-IgG tips. The cells were harvested by spinning down the culture sat 2400 rpm for 10 minutes, and then the supernatant filtered through 0.2 um ultrafilters.

The 5C8 antibody was captured from the supernatant using Protein A Sepharose FF (GE Healthcare) on AKTA (Amersham Biosciences). The antibody molecule was eluted from the Protein A using 0.1 M glycine at pH 3.0, neutralized with Tris base, dialyzed into PBS using the Pierce 10 ml dialysis cassettes (10,000 MWCO cutoff), concentrated to 1 ml final volume, and the further purified using preparative size exclusion chromatography (TOSOHASS, TOSOH Biosciences). The 5C8 molecule was dialyzed into a 20 mmol citrate, 150 mmol NaCl solution at pH 6.0. Purity and percentage of monomer antibody product was assessed by 4-20% Tris-glycine SDS-PAGE and analytical size-exclusion HPLC, respectively.

### B. Confirmation of Protein Sequences and Post-Translational Modifications of Stability Engineering Antibodies Using Mass Spectral Analysis

The samples were analyzed under reducing conditions. Reduction took place in 100 mM DTT in the presence of 4M guanidine HCl for 1 hour at 37° C. Prior to injection, the samples were diluted 1:1 with PBS. Glacial acetic acid was added to the mix to a final concentration of 2% (v/v). 5 μg of each sample was injected onto a phenyl column and analyzed by ESI-TOF. A bind and elute method was used. Buffer A contains 0.03% TFA in water and buffer B contains 0.025% TFA in acetonitrile. Flow rate was kept constant at 100 μl per minute. Spectra were obtained from the Analyst software and deconvoluted using MaxEnt1. After reduced analysis, 3 of the samples were detected as glycoforms, therefore, deglycosylation was performed on the 3 samples: EC323, EC326 and EAG2300. Deglycosylation was performed under reducing condition: 1 mU of N-glycanase/2 μg of protein in the presence of 20 mM DTT, 10 mM Tris pH 7.0. The samples were deglycosylated at 37° C. After 2 hours, an additional 30 mM of DTT was added to the samples in the presence of 2.7M guanidine HCl and incubated at 37° C. for an additional 30 minutes. 5 μg of each reduced, deglycosylated sample were injected onto a phenyl column and analyzed as detailed above.

Results confirmed the identities of all 13 samples with conversion of the N-terminal glutamine (Q) of the heavy chain to pyroglutamic acid (PE). Table 3.3 lists the masses obtained for all samples, glycosylated and deglycosylated. All light chains and heavy chains contained low levels of glycation of 1% or less. Masses corresponding to the unmodified N-terminal glutamine were observed in each of the samples at a relative intensity of ˜20-40%. All light chain deconvoluted spectra were identical as expected.

TABLE 3.3
Masses Detected

| | | Detected | Theoretical |
|---|---|---|---|
| Sample ID | Probable Assignment | Mass | Mass |
| YC401 | LC 1-218 | 23857 | 23858 |
| | HC 1-444 Q→PE | 48640 | 48641 |
| YC402 | LC 1-218 | 23857 | 23858 |
| | HC 1-444 Q→PE | 48659 | 48660 |
| YC403 | LC 1-218 | 23857 | 23858 |
| | HC 1-444 Q→PE | 48655 | 48656 |
| YC404 | LC 1-218 | 23857 | 23858 |
| | HC 1-444 Q→PE | 48697 | 48698 |
| YC405 | LC 1-218 | 23857 | 23858 |
| | HC 1-444 Q→PE | 48716 | 48717 |
| YC406 | LC 1-218 | 23857 | 23858 |
| | HC 1-444 Q→PE | 48712 | 48713 |
| YC407 | LC 1-218 | 23857 | 23858 |
| | HC 1-444 Q→PE | 48672 | 48673 |
| EC323 | LC 1-218 | 23857 | 23858 |
| | HC 1-444 Q→PE, G0F | 50134 | 50135 |
| | HC 1-444 Q→PE, G1F | 50297 | 50297 |
| | HC 1-444 Q→PE, G2F | 50459 | 50459 |
| | HC 1-444 Q→PE, G0 (Minus | 49988 | 49989 |
| | fucose) | | |
| EC323 | LC 1-218 | 23857 | 23858 |
| Deglycosylated | HC 1-444 Q→PE | 48690 | 48690 |
| EC326 | LC 1-218 | 23857 | 23858 |
| | HC 1-444 Q→PE, G0F | 50130 | 50131 |
| | HC 1-444 Q→PE, G1F | 50293 | 50293 |
| | HC 1-444 Q→PE, G2F | 50454 | 50455 |
| | HC 1-444 Q→PE, G0 (Minus | 49984 | 49985 |
| | fucose) | | |
| EC326 | LC 1-218 | 23857 | 23858 |
| Deglycosylated | HC 1-444 Q→PE | 48685 | 48686 |
| EC331 | LC 1-218 | 23857 | 23858 |
| | HC 1-444 Q→PE | 48676 | 48677 |
| EAG2300 | LC 1-218 | 23857 | 23858 |
| | HC 1-443 Q→PE, G0F | 49919 | 49920 |
| | HC 1-443 Q→PE, G1F | 50081 | 50082 |
| | HC 1-443 Q→PE, G2F | 50243 | 50244 |
| EAG2300 | LC 1-218 | 23857 | 23858 |
| Deglycosylated | HC 1-443 Q→PE | 48473 | 48475 |
| CN578 | LC 1-218 | 23857 | 23858 |
| | HC 1-447 Q→PE | 48885 | 48885 |
| CN579 | LC 1-218 | 23857 | 23858 |
| | HC 1-444 Q→PE | 48729 | 48730 |

Samples EC323, EC326 and EAG2300 contained the usual G0F, G1F, G2F biantennary glycans with the G0F as the most abundant specie followed by G1F then G2F. Samples EC323 and EC326 contained a peak at −146Da from the G0F peak which corresponds to a G0F glycan missing a core fucose (G0). For EC323, the relative percentage intensity of G0 (minus fucose) was 2% while that of the EC326 sample was 23%. All 3 glycosylated samples contained low levels (<1%) of sialic acid on the G2F glycan.

All sample chains contained a −18Da peak which has been shown to be an instrument artifact related to elevated gas temperature of the ESI-TOF. A temperature of 350° C. was used to eliminate TFA adducts.

### Example 4

### Thermal Stability of Aglycosylated IgG Fc Antibodies

Protein stability is a central issue for the development and scale up of protein therapeutics. Insufficient stability may lead to a number of development issues ranging from unsuitability for scale-up production in bioreactors, difficulties in protein purification, and unsuitability for pharmaceutical preparation and use. In order to generate an effector-function deficient Fc backbone, mutations were introduced into agly IgG4.P(S228P) to increase the overall stability of the CH2 and CH3 domains. The goal of this study was to investigate whether the designed mutations increase thermal stability. Therefore, the thermostability of each construct was assessed using differential scanning calorimetry (DSC). Both the E. coli produced Fc-domain constructs and full length antibody constructs were assessed by DSC. The expression and purification methods for the E. coli produced Fc-domain constructs and the full length antibody constructs are detailed in Example 3.

The antibodies were dialyzed against a 25 mM sodium citrate, 150 mM NaCl buffer at pH 6.0. Antibodies were concentration to 1 mg/mL and measured by UV absorbance. Scans were performed using an automated capillary DSC (MicroCal, LLC, Northampton, Mass.). Two buffer scans were performed for baseline subtraction. Scans ran from 20-105° C. at 1° C./min using the medium feedback mode. Scans were then analyzed using the software Origin (MicroCal LLC, Northampton, Mass.). Nonzero baselines were corrected using a third-order polynomial and the unfolding transitions of each antibody were fit using the non-two-state unfolding model. To further assess the stability of these constructs, the full length antibodies were dialyzed against a 25 mM sodium phosphate, 25 mM sodium citrate, 150 nM NaCl buffer at pH 4.5. The same DSC protocol was used as detailed above.

E. coli expressed Fc-domain constructs lacking the Fab domain were used to test stability enhancement of the mutations identified in the Delphia thermal challenge assay as detailed in Example 2. The constructs BRM023, BRM030 and CR103-119 are listed along with their melting temperatures in Table 4.1.

TABLE 4.1
Melting Temperatures of E. coli Expressed
IgG Fc constructs as measured by DSC.

| | | Tm (° C.) | | | |
|---|---|---|---|---|---|
| DSC | Final AA Substitution | CH2 | CH3 | Fab | Source |
| BRM012 | IgG1 (agly b/c | 65.9 | 82.6 | n/a | E. coli |
| | expressed in E. coli) | | | | |
| BRM013 | IgG4.P S228P, T299A | 62.3 | 71.15 | n/a | E. coli |
| BRM023 | S228P, T299A, T307P | 66.2 | 69.9 | n/a | E. coli |
| BRM030 | S228P, T299K | 65.7 | 70 | n/a | E. coli |
| CR103 | S228P, T299A, R409K | 58.3 | 83.2 | n/a | E. coli |
| CR104 | S228P, T299A, R409M | 60.9 | 77.7 | n/a | E. coli |
| CR105 | S228P, T299A, R409L | — | — | n/a | E. coli |
| CR106 | S228P, T299A, R409I | X | X | n/a | E. coli |
| CR107 | S228P, T299A, D399S | 58.4 | 74.9 | n/a | E. coli |
| CR108 | S228P, T299A, D399N | 57.2 | 70.4 | n/a | E. coli |
| CR109 | S228P, T299A, D399E | 58.4 | 66.9 | n/a | E. coli |
| CR109 2 | S228P, T299A, D399E | 57.1 | 68.1 | n/a | E. coli |
| CR110 | S228P, T299A, V369I | 60.5 | 65.6 | n/a | E. coli |
| CR111 | S228P, T299A, V379I | 57.7 | 66.8 | n/a | E. coli |
| CR112 | S228P, T299A, V397I | 59.7 | 72 | n/a | E. coli |
| CR113 | S228P, T299A, V427I | X | X | n/a | E. coli |
| CR114 | S228P, T299A, V427F | 61.6 | 75.3 | n/a | E. coli |
| CR115 | S228P, T299A, V240I | X | X | n/a | E. coli |
| CR116 | S228P, T299A, V263I | X | X | n/a | E. coli |
| CR117 | S228P, T299A, V273I | X | X | n/a | E. coli |
| CR118 | S228P, T299A, V302I | 59.7 | 71.7 | n/a | E. coli |
| CR119 | S228P, T299A, V323I | 59.1 | 59.1 | n/a | E. coli |

As depicted in Table 4.1, the agly IgG1 and IgG4.P(S228P, T299A) Fc moiety controls had melting temperatures of 65.9° C. and 62.3° C. respectively for CH2 and 82.6° C. and 71.2° C., respectively for CH3. Of the single site mutations, BRM023 (T307P) and BRM030 (T299K) showed a 3.4-3.9° C. increase in CH2 melting temperature over the agly IgG4.P (S228P, T299A) control. Substitution at position R409 with Lysine or Methionine, showed an increase of 12 and 6.6° C. in the CH3 melting temperature. Substitution to smaller, hydrophobic side chains (Leu and Ile) did not confer increased stability for CH3. This position represents the single difference in the CH3 interface between IgG1 and IgG4. Mutations at position D399 were made to compensate for the added bulk of the Arginine side chain at position 409 in the IgG4 CH3 interface (as detailed in Example 1). A substitution of a smaller side chain (Ser) facilitated an increase in melting temperature of ˜4° C. Substitution to either a side chain with same size but lacking charge (Asp) or to a larger side chain with same charge (Glu) both showed no increase in stability. Substitutions in the hydrophobic valine core as detailed in Example 1, showed either no effect or a decrease in melting temperature with the exception of V427F which showed an increase in CH3 melting temperature of ˜4° C.

To evaluate single and combinations of multiple mutations, full length IgG molecules were utilized. Mutations were incorporated into full length 5c8 antibodies as detailed in Example 3. The effects of the mutations on the melting temperatures of the CH2 and CH3 domains as measured by DSC at pH 6.0 and pH 4.5 are summarized in Table 4.2 below.

TABLE 4.2
Melting Temperatures of Full Length IgG constructs as measured by DSC

| | | Tm (° C.) | | | | | | |
|---|---|---|---|---|---|---|---|---|
| | | pH 6.0 | | | pH 4.5 | | | |
| DSC | Final AA Substitution | CH2 | CH3 | Fab | CH2 | CH3 | Fab | Source |
| | IgG4.P agly (S228P, | 53.8 | 70 | 76.67 | 38.5 | 60.2 | 69 | CHO |
| | T299A) | | | | | | | |
| | IgG4.P (S228P) | 64.14 | 73.66 | 77.2 | 51.04 | 63.23 | 68.84 | CHO |
| | IgG1 agly (T299A) | 58.8 | 85.3 | 77.2 | | | | CHO |
| | IgG1 | 71.5 | 84.9 | 77.5 | 60 | 75.5 | 69 | CHO |
| EC301 | S228P, T299K, V427F | 44.8 | 54.77 | 76.26 | | | | CHO |
| EC302 | S228P, T299K, D399S | 60.4 | 74.4 | 77 | 42.8 | 66.37 | 69.61 | CHO |
| EC303 | S228P, T307P, V427F | 63 | 75 | 76.6 | | | | CHO |
| EC304 | S228P, T307P, D399S | 67.4 | 75.4 | 77.6 | 54.46 | 66.74 | 69.85 | CHO |
| EC305 | S228P, T299K, V427F, | 47.1 | 74.81 | 77.1 | | | | CHO |
| | D399S | | | | | | | |
| EC306 | S228P, T307P, V427F, | 52.8 | 75 | 77.4 | | | | CHO |
| | D399S | | | | | | | |
| EC307 | S228P, T299K, V427F, | | | | | | | CHO |
| | V348F | | | | | | | |
| EC308 | S228P, T307P, V323F | 63.47 | 73.71 | 77.15 | | | | CHO |
| EC309 | S228P, V240F | 50.1 | 73.5 | 77.3 | | | | CHO |
| EC321 | S228P, D399S, L309P | 60.2 | 75.1 | 77.5 | | | | CHO |
| EC322 | S228P, D399S, L309M | 62.1 | 74.8 | 77.4 | | | | CHO |
| EC323 | S228P, D399S, L309K | 64.7 | 74.8 | 77.5 | 53.11 | 66.6 | 69.82 | CHO |
| EC324 | S228P, T307P, D399S, | 62.7 | 74.8 | 77.5 | | | | CHO |
| | L309P | | | | | | | |
| EC325 | S228P, T307P, D399S, | 65.21 | 74.98 | 77.5 | | | | CHO |
| | L309M | | | | | | | |
| EC326 | S228P, T307P, D399S, | 67.5 | 75.23 | 77.6 | 56.48 | 66.73 | 69.95 | CHO |
| | L309K | | | | | | | |
| EC300 | S228P, T307P | 62.5 | 74.8 | 77.4 | | | | CHO |
| EC330 | S228P/T299A/T307/IgG | 60.5 | 84.5 | 76.8 | 43 | 77.36 | 68.75 | CHO |
| | 1-CH3 | | | | | | | |
| EC331 | S228P/T299K/T307/IgG | 65.5 | 84.77 | 76.6 | 47.4 | 77.1 | 68.2 | CHO |
| | 1-CH3 | | | | | | | |
| YC401 | S228P, T299A, T307P, | 61.55 | 75 | 77.15 | 46.35 | 71.31 | 68.04 | CHO |
| | D399S | | | | | | | |
| YC402 | S228P, T299A, L309K, | 59.95 | 74.52 | 77.02 | 47.14 | 72.54 | 69.32 | CHO |
| | D399S | | | | | | | |
| YC403 | S228P, T299A, T307P, | 62.21 | 74.77 | 77.1 | 51.64 | 73.53 | 70.44 | CHO |
| | D399S, L309K | | | | | | | |
| YC404 | S228P, T299K, T307P, | 63.44 | 75.14 | 77.24 | 50.8 | 71.93 | 68.76 | CHO |
| | D399S | | | | | | | |
| YC405 | S228P, T299K, L309K, | 63.16 | 74.81 | 77.16 | 49.4 | 71.92 | 68.66 | CHO |
| | D399S | | | | | | | |
| YC406 | S228P, T299K, T307P, | 66.2 | 74.1 | 77.23 | 53.53 | 72.3 | 69.25 | CHO |
| | D399S, L309K | | | | | | | |
| YC407 | S228P, T299A | 55.8 | 73.05 | 76.78 | 41.52 | 72.52 | 67.53 | CHO |
| CN578 | T299K (IgG1) | 65.4 | 85.2 | 77.7 | 47.6 | 72.2 | 67.8 | CHO |
| CN579 | S228P, T299K | 60.9 | 73.7 | 77.2 | 42.1 | 61.1 | 68.6 | CHO |
| pEAG2296 | S228P/T299/IgG1-CH3 | 54.6 | 85.2 | 76.4 | 35.1 | 77.5 | 68.1 | CHO |
| pEAG2287 | S228P/T299K/IgG1-CH3 | 60 | 85.2 | 76.4 | 41.4 | 77.4 | 68.1 | CHO |
| SDE1 | T299K, V262L | | | | | | | CHO |
| SDE2 | T299K, V264T | 64.81 | 85.12 | 77.32 | 50.61 | 73.72 | 70.01 | CHO |
| SDE3 | T299K, V266F | 58.03 | 85.25 | 77.3 | | | | CHO |
| SDE4 | T299K, V262L, V264T | 63.24 | 85.11 | 77.22 | | | | CHO |
| SDE5 | T299K, V264T, V266F | 58.3 | 84.95 | 77.35 | | | | CHO |
| SDE6 | T299K, Loop | 61.68 | 85.16 | 77.16 | | | | CHO |
| | Replacement | | | | | | | |
| SDE7 | T299K, Loop + | 59.2 | 84.89 | 76.97 | | | | CHO |
| | V13L/V15T | | | | | | | |
| SDE8 | T299K, V262L, V264T, | 56.98 | 85.21 | 77.13 | | | | CHO |
| | V266F | | | | | | | |
| SDE9 | T299K, Loop + | 53.45 | 85.04 | 77.03 | | | | CHO |
| | V13L/V15T/V17F | | | | | | | |

As depicted in Table 4.2, the D399S mutation increased the thermal stability of the CH3 domain in agly IgG4.P on average by 2° C. at pH 6.0 and by as much as 10° C. at pH 4.5. The mutant T299K is used to generate an aglycosylated CH2. The lysine substitution at position 299 increases the melting temperature by 5° C. at pH 6.0 and by 11° C. at pH 4.5 in the IgG4.P molecule over a substitution of alanine in this position. The T299K mutation also increases the Tm for IgG1 CH2 by 6° C. at pH 6.0. The T307P mutation showed an increase of 4° C. for the glycosylated IgG4.P CH2 domain when used in combination with D399S. By itself, T307P did not increase the melting temperature in the glycosylated IgG4.P form. In the aglycosylated form, the T307P mutation increased the CH2 Tm by 6° C. When combined with the T299K mutation, the Tm for CH2 increased by 8° C. The L309K mutation conferred a 1° C. increase in stability for the aglycosylated IgG4.P when in combination with T307P and T299A. However, in combination of T307P and T299K, the L309K mutation conferred an increase of 3° C. In the glycosylated form of IgG4.P, the L309K mutation increases the Tm for CH2 by 2° C. The L309K mutation conferred a 1° C. increase in stability for the aglycosylated IgG4.P when in combination with T307P and T299A. However, in combination of T307P and T299K, the L309K mutation conferred an increase of 3° C. at pH 4.5. The V323F mutation in CH2 showed no effect on the melting temperature of the CH2 domain while a V240F mutation decreased the melting temperature by 13° C. In addition, the V427F mutation also showed a decrease in the Tm of 13° C. for CH2.

The most dramatic increase in melting temperatures is observed in the combination of T299K, T307P, L309K and D399S in IgG4.P. This construct shows an increase in the Tm for CH2 of 11° C. (pH 6.0) and 12° C. (pH 4.5) when compared to T299A IgG4.P. In fact the T299K mutation increases the Tm by 2-3° C. when in combination with T307P, L309K and D399S over the T299A mutation. Additionally, the introduction of T299K into the IgG4.P CH2 in combination with the conversion of the CH3 of the IgG4.P isotype to the CH3 from IgG1 resulted in an increase of 6° C. and 15° C., for the CH2 and CH3 domains respectively over the agly IgG4.P

Mutations identified in covariation studies of CH2 glycosylation show none to little effect on the Tm for IgG1 CH2 (V262L, and V264T in combination with V262L, Loop replacement), or a decreased effect of 7° C. (V266F, V264T & V266F, Loop & V264T & V266F). A large decrease in melting Tm of 10-12° C. was observed for the combination of V262L, V264T and V266F.

In summary, T299K, T307P, L309K showed the ability to increase the thermal stability of the CH2 domain either as single mutations or in combinations with each other. D399S conferred stability to the CH3 domain of IgG4.P.

### Example 5

### Agitation and pH Hold Step Studies of IgG Fc Antibodies

It is highly desirable for a protein therapeutic to have a long shelf life, with minimal changes to the physical or chemical properties of the protein during manufacturing production and storage. Evaluating related stresses is an important part of formulation development and two types of associated stress were evaluated for the IgG Fc mutants.

### A. Agitation Stress

Agitation mimics stresses encountered during manufacturing and processing as well as simulates the stress during actual shipping (i.e. shipment of the drug product vials to test site). Therefore, agitation stability was analyzed over the course of 48 hours, and protein aggregation or precipitation was monitored using analytical size exclusion chromatography (SEC) and turbidity was measured by monitoring absorbance at 320 nM. Turbidity is a measure of light scattering due to aggregation and precipitant formation that makes the protein/buffer solution cloudy or even opaque in extreme cases. The following method was used consistently in each set of experiments: 1 ml of each sample at 0.5 mg/ml was shaken in a 3 ml formulation tube at 650 rpm, sealed with a rubber stopper, and sealed again with parafilm. 100 μl of sample were extracted at the necessary time points (0, 6, 24, and 48 hours) and spun down at 14,000 rpm for 5 minutes to spin down aggregates or precipitants formed. The samples were then run and analyzed on an analytical SEC column. Aggregated protein elutes at shorter retention times and protein degradation products elute at longer retention times in the SEC elution profile. Therefore the percentage of monomer species was used to monitor the overall stability of the protein at a given time point.

Constructs with the highest thermal stabilization (see Example 4) were chosen for the agitations studies. For the aglycosylated IgG4 molecules, YC401 through YC403 (all aglycosylated IgG4.P T299A and D399S [plus T307P, L309K, and T307P/L309K respectively], YC404 through YC406 (all aglycosylated IgG4.P T299K and D399S [plus T307P, L309K, and T307P/L309K respectively], YC407 as the wild-type IgG4.P aglycosylated (T299A) control, CN578 (aglycosylated IgG1 A299K), EC331 (which is the aglycosylated IgG4.P T299K and T307P with an IgG1 CH3 domain), an aglycosylated IgG1 (T299A), aglycosylated IgG4.P (T299A) and an aglycosylated IgG1 (T299A) were selected for study. For the glycosylated molecules, EC304 (glycosylated IgG4.P T307P, D399S), EC323 (glycosylated IgG4.P D399S, L309K), EC326 (glycosylated IgG4.P T307P, D399S, L309K), glycosylated IgG4.P T299A, and glycosylated IgG1 were selected for study.

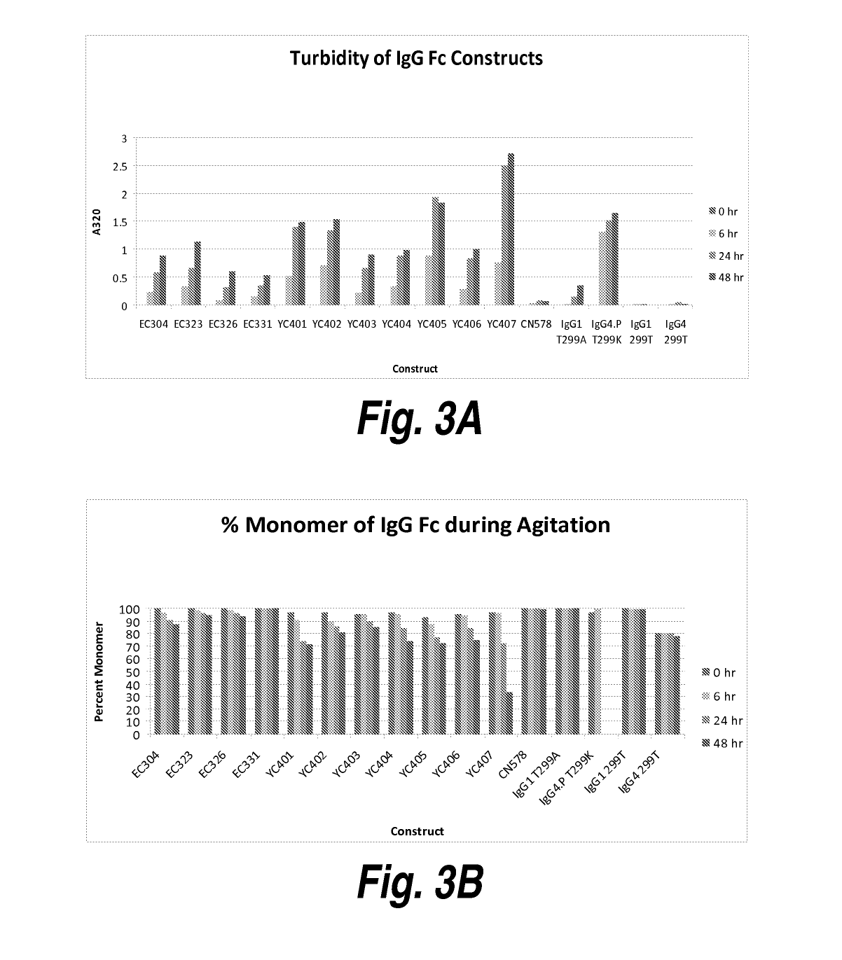

Comparing the aglycosylated mutants in terms of turbidity (see Table 5.1 below and FIG. 3A), YC403 (aglycosylated IgG4.P T299A, T307P, L309K, and D399S) and YC406 (aglycosylated IgG4.P T299K, T307P, L309K, and D399S) showed the lowest amount of turbidity compared to the wild type YC407 (aglycosylated IgG4.P T299A). Both constructs consistently show one-third of the turbidity compared to the wild-type at each time point. The only difference between the two constructs is T299A (YC403) and T299K (YC406).

TABLE 5.1
Turbidity of Constructs at Time Points During Agitation

Time

| | 0 hr | 6 hr | 24 hr | 48 hr |
|---|---|---|---|---|
| EC304 | 0 | 0.232 | 0.584 | 0.89 |
| EC323 | 0 | 0.333 | 0.672 | 1.139 |
| EC326 | 0 | 0.088 | 0.316 | 0.595 |
| EC331 | 0 | 0.157 | 0.343 | 0.54 |
| YC401 | 0 | 0.51 | 1.406 | 1.49 |
| YC402 | 0 | 0.717 | 1.331 | 1.54 |
| YC403 | 0 | 0.221 | 0.675 | 0.892 |
| YC404 | 0 | 0.334 | 0.884 | 0.977 |
| YC405 | 0 | 0.885 | 1.94 | 1.841 |
| YC406 | 0 | 0.29 | 0.838 | 0.993 |
| YC407 | 0 | 0.772 | 2.5 | 2.709 |
| CN578 | 0 | 0.029 | 0.078 | 0.072 |
| IgG1 | 0 | 0.019 | 0.144 | 0.348 |
| T299A | | | | |
| IgG4.P | 0 | 1.322 | 1.51 | 1.657 |
| T299K | | | | |
| IgG1 | 0 | 0.009 | 0.01 | 0.008 |
| 299T | | | | |
| IgG4 | 0 | 0.009 | 0.0465 | 0.0185 |
| 299T | | | | |

In comparing the % monomer (see Table 5.2 below and FIG. 3B, all of the mutant construct showed reduced aggregation over time. At the 24 hour time point, all the mutant constructs were better than the wild-type, and at the 48 hour time point, most constructs were at least 2 fold better. However, the construct that retained the highest % monomer was YC403, followed by YC402 (aglycosylated IgG4.P T299A, L309K and D399S), and then YC406 (aglycosylated IgG4.P T299K, T307P, L309K, and D399S). These constructs showed the least gradual loss in % monomer over time. The common mutation seen amongst the best ranking constructs is the L309K mutation. This data demonstrates that the mutations chosen improve the overall stability in a mechanical stress context. Comparing both agitation measurements for the aglycosylated IgG4.P constructs, the YC403 (aglycosylated IgG4.P T299A, T307P, L309K, and D399S) and YC406 (aglycosylated IgG4.P T299K, T307P, L309K, and D399S) constructs best resist the mechanical stress over time. Both molecules show additive mutations (T307P/L309K) that enable the thermal and structural stability to improve.

TABLE 5.2
% Monomer of constructs at time points during agitation

Time

| | 0 hr | 6 hr | 24 hr | 48 hr |
|---|---|---|---|---|
| EC304 | 100 | 96.5 | 90.52 | 87.22 |
| EC323 | 100 | 98.16 | 96.29 | 94.37 |
| EC326 | 100 | 98.11 | 95.89 | 93.35 |
| EC331 | 100 | 100 | 100 | 100 |
| YC401 | 96.5 | 90.17 | 73.63 | 71.07 |
| YC402 | 96.6 | 89.45 | 85.49 | 80.88 |
| YC403 | 95 | 95.382 | 90.03 | 84.75 |
| YC404 | 97 | 95.45 | 83.78 | 73.5 |
| YC405 | 92.7 | 87.46 | 76.72 | 72.1 |
| YC406 | 95.5 | 94.12 | 83.89 | 74.8 |
| YC407 | 97 | 95.88 | 72.3 | 33.4 |
| CN578 | 99.73 | 100 | 100 | 99.3 |
| IgG1 | 100 | 100 | 100 | 100 |
| T299A | | | | |
| IgG4.P | 96.7 | 100 | 0 | 0 |
| T299K | | | | |
| IgG1 | 100 | 99.01 | 98.85 | 99 |
| 299T | | | | |
| IgG4 | 80.51 | 80.2 | 80.35 | 78.06 |
| 299T | | | | |

For IgG1 aglycosylated molecules, CN578 (aglycosylated IgG1 A299K) showed minimal turbidity and it also showed essentially no aggregation throughout the entire experiment. CN578 performs better than the IgG1 T299A and also the wild-type IgG1 299T molecule, thus showing that the A299K mutation has minimal effect on agitation for an aglycosylated IgG1 molecule. CN578 is 5-fold better in the turbidity study than the IgG1 T299A. The CN578 molecule also shows no aggregation over a 48 hour time span, which is the same result as both aglycosylated IgG1 T299A and glycosylated IgG1 299T. EC331 (which is the aglycosylated IgG4.P T299K and T307P with an IgG1 CH3 domain) performed very well compared to the other constructs, as it also maintained 100% monomer throughout the agitation study. It showed a 2-fold improvement in turbidity compared to the IgG4.P agly constructs (YC series). This data suggests that the IgG1 CH3 portion greatly aids in both the thermal and structural stability of the molecule.

Among the glycosylated molecules, EC304 (glycosylated IgG4.P T307P, D399S), EC323 (glycosylated IgG4.P D399S, L309K), EC326 (glycosylated IgG4.P T307P, D399S, L309K), there is an improvement in % monomer over the course of the aggregation study compared to the wild-type glycosylated IgG4.P molecule (See Table 5.2 and FIG. 3B. Yet the turbidity greatly increases at every time point even up to 75-fold. Consistently, each molecule contains a D399S, so it may be possible that this mutation destabilizes the structural stability as the data shows.

### B. Low pH Hold Step Studies

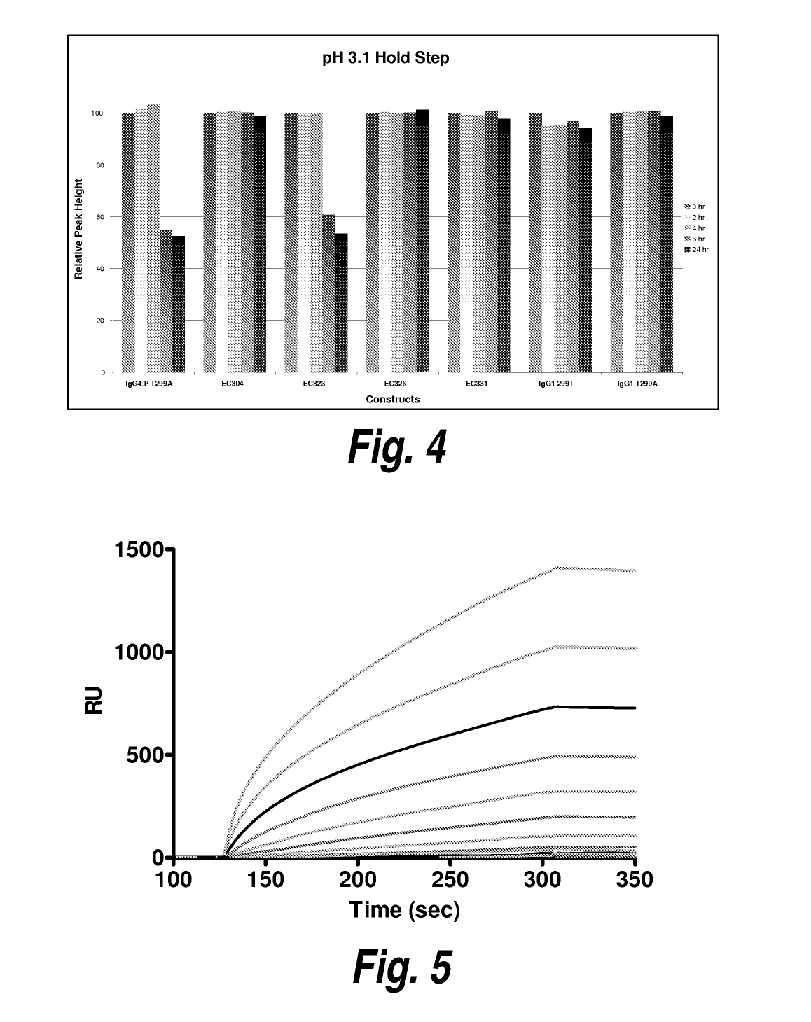

It is highly desirable for a protein therapeutic to have manufacturability and scalability. Performing a pH hold step study is essential for process development. A pH hold study mimics the process development during the production and purification stages of the protein. For the production stage, reproducibility and consistency in the protein are essential for quality assurance. This method can be used to measure stability of a protein at either a high or low pH. For the study, 1 mg of protein was loaded onto a protein-A column using an AKTA (Pharmacia Biotech, now GE Healthcare) and eluted with acetate buffer at pH 3.1. The protein was held at the low pH for 2 hour intervals up to 6 hours. A 100 μl aliquot was taken and then run on analytical SEC to measure loss of protein due to degradation and aggregation. The results are summarized in Table 5.3 (see below) and FIG. 4.

TABLE 5.3
Relative Peak Height over time of IgG Fc's at low pH hold

| | Time | | | | |
|---|---|---|---|---|---|
| | 0 hr | 2 hr | 4 hr | 6 hr | 24 hr |
| EC304 | 100 | 100.74 | 100.53 | 100.18 | 98.82 |
| EC323 | 100 | 99.66 | 99.99 | 60.8 | 53.47 |
| EC326 | 100 | 100.83 | 99.8 | 100.16 | 101.27 |
| EC331 | 100 | 99.22 | 99.09 | 100.82 | 97.79 |
| IgG1 | 100 | 95.15 | 95.12 | 96.76 | 94.08 |
| 299T | | | | | |
| IgG1 | 100 | 100.39 | 100.5 | 100.91 | 99.01 |
| T299A | | | | | |
| IgG4.P | 100 | 101.68 | 103.33 | 54.84 | 52.53 |
| T299A | | | | | |

For this study, EC304 (glycosylated IgG4.P T307P, D399S), EC323 (glycosylated IgG4.P D399S, L309K), EC326 (glycosylated IgG4.P T307P, D399S, L309K), glycosylated IgG4.P T299A, EC331 (which is the aglycosylated IgG4.P T299K and T307P with an IgG1 CH3 domain), an aglycosylated IgG1 (T299A), aglycosylated IgG4.P (T299A) and glycosylated IgG1 were selected for study. From the data, EC331 is shown to be able to withstand the low pH hold for at least 6 hours without losing much yield. This is an improvement compared to the aglycosylated IgG4 wild type control that was run. It is predicted that the other aglycosylated constructs will not lose any protein due to degradation as this construct was able to withstand the low pH hold. With both being glycosylated, EC304 and EC326 also maintain their yields, which is also comparable to the glycosylated IgG1 wild-type. EC323, which is also glycosylated, did not fair so well over time. It is hypothesized that the L309K mutation alone needs to be stabilized together with a T307P mutation, which is seen in the more stabilized EC326 construct.

### Example 6

### Fc Receptor Binding of Stability Engineered IgG Fc Antibodies

The effector function of the aglycosylated variant antibodies of the invention were characterized by their ability to bind Fc receptors or a complement molecule such as C1q.

### A. Solution Phase Competition Biacore Experiments

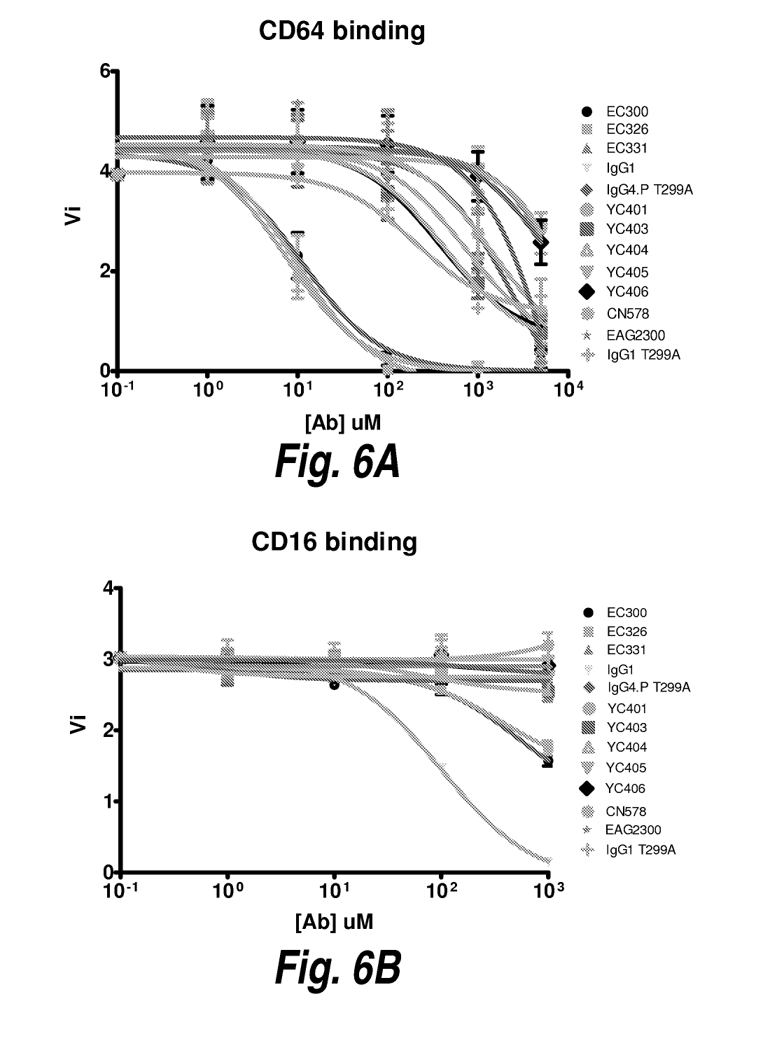

Binding to Fcγ receptors was analyzed using solution affinity surface plasmon resonance (ref Day E S, Cachero T G, Qian F, Sun Y, Wen D, Pelletier M, Hsu Y M, Whitty A. Selectivity of BAFF/BLyS and APRIL for binding to the TNF family receptors BAFFR/BR3 and BCMA. Biochemistry. 2005 Feb. 15; 44(6):1919-31.) The method utilizes conditions of so-called “mass-transport-limited” binding, in which the initial rate of ligand binding (protein binding to the sensor chip) is proportional to the concentration of ligand in solution (ref BIApplications Handbook (1994) Chapter 6: Concentration measurement, pp 6-1-6-10, Pharmacia Biosensor AB). Under these conditions, binding of the soluble analyte (protein flowing over chip surface) to the immobilized protein on the chip is fast compared to the diffusion of the analyte into the dextran matrix on the chip surface. Therefore, the diffusion properties of the analyte and the concentration of analyte in solution flowing over the chip surface determine the rate at which analyte binds to the chip. In this experiment, the concentration of free Fc receptor in solution is determined by the initial rate of binding to a CM5 Biacore chip containing an immobilized IgG1 MAb. Into these Fc receptor solutions were titrated the stability engineered constructs (see Table 6.1 below). The half maximal (50%) inhibitory concentration (IC₅₀) of these constructs was demonstrated by their ability to inhibit Fc receptor from binding to the immobilized IgG1 antibody immobilized on the surface of the sensorchip. Initial binding rates were obtained from raw sensorgram data (FIG. 5). The titration curves that were used to calculate IC50's are shown in FIG. 6A for CD64 (FcγRI) and FIG. 6B for CD16 (FcγRIIIa V158). The results are shown in Table 6.1 and reported as the average of two titrations.

TABLE 6.1
FcγR affinity characterization of Fc variants

IC₅₀ (uM)

| | CD64 | CD16 |
|---|---|---|
| EC300 | 11.19 | 563.5 |
| EC326 | 7.558 | 380.5 |
| EC331 | 2595 | >1000 |
| YC401 | 377.4 | >1000 |
| YC403 | 433.7 | >1000 |
| YC404 | >5000 | >1000 |
| YC405 | >5000 | >1000 |
| YC406 | >5000 | >1000 |
| CN578 | 1425 | >1000 |
| EAG2300 | 3021 | >1000 |
| IgG1 | 9.636 | 100.2 |
| IgG1 | 205.3 | >1000 |
| T299A | | |
| IgG4.P | 739 | >1000 |
| T299A | | |

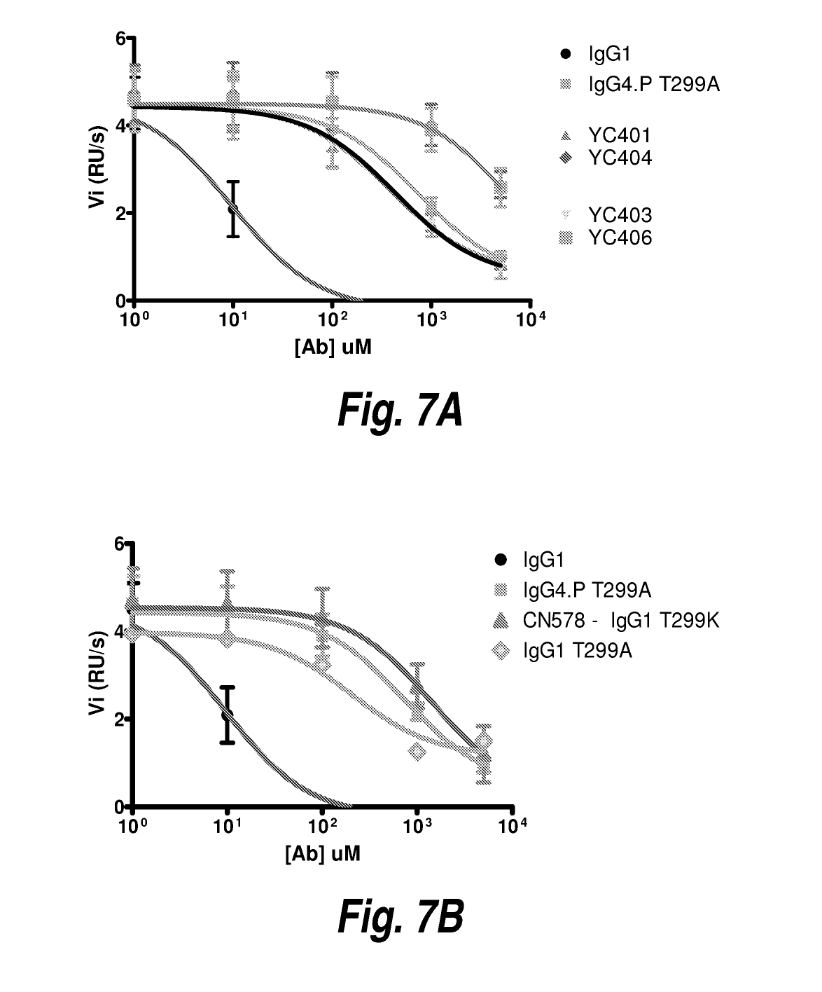

In the CD64 binding assay, the IgG1 control antibody had an IC₅₀ of 9.6 μM, while the IgG1 T299A (agly) and IgG4.P T299A (agly) had IC₅₀s of 205 and 739 μM respectively. As expected, the IgG1 molecules have greater affinity for CD64 than the IgG4 molecule, and the aglycosylated IgG1 showed a reduced affinity compared to the glycosylated IgG1. The stability engineered glycosylated IgG4.P molecules (EC300 and EC326) had IC₅₀ values at about 8 μM, compared to the stability engineered aglycosylated IgG4.P molecules (EC331 and YC400 series) which ranged from 440 to >5000 μM. The IC₅₀'s for the stability engineered IgG4.P glycosylated molecules (EC300, EC326) were equivalent to the glycosylated IgG1 control, and the stability engineered aglycosylated IgG4.P with T299A (YC401, YC403) had the log equivalent IC₅₀'s as the aglycosylated IgG4.P T299A control. The stability engineered aglycosylated IgG4.P with T299K, however, showed a 1 to 2 logs greater reduction in affinity compared to the equivalent molecules with a T299A substitution FIG. 7A. This result was also observed for the stability engineered aglycosylated IgG1 T299K (CN578) that showed a log reduction in affinity compared to the aglycosylated IgG1 T299A control (FIG. 7B). In fact, the T299K substitution shifts the aglycosylated IgG1 (T299A) molecule from having greater affinity for CD64 than the aglycosylated IgG4.P T299A control, to having reduced affinity for the aglycosylated IgG1 (T299K) compared to the aglycosylated IgG4.P control (FIG. 7B). In summary, the T299K mutation reduces the affinity for CD64 in both IgG1 and IgG4 molecules.

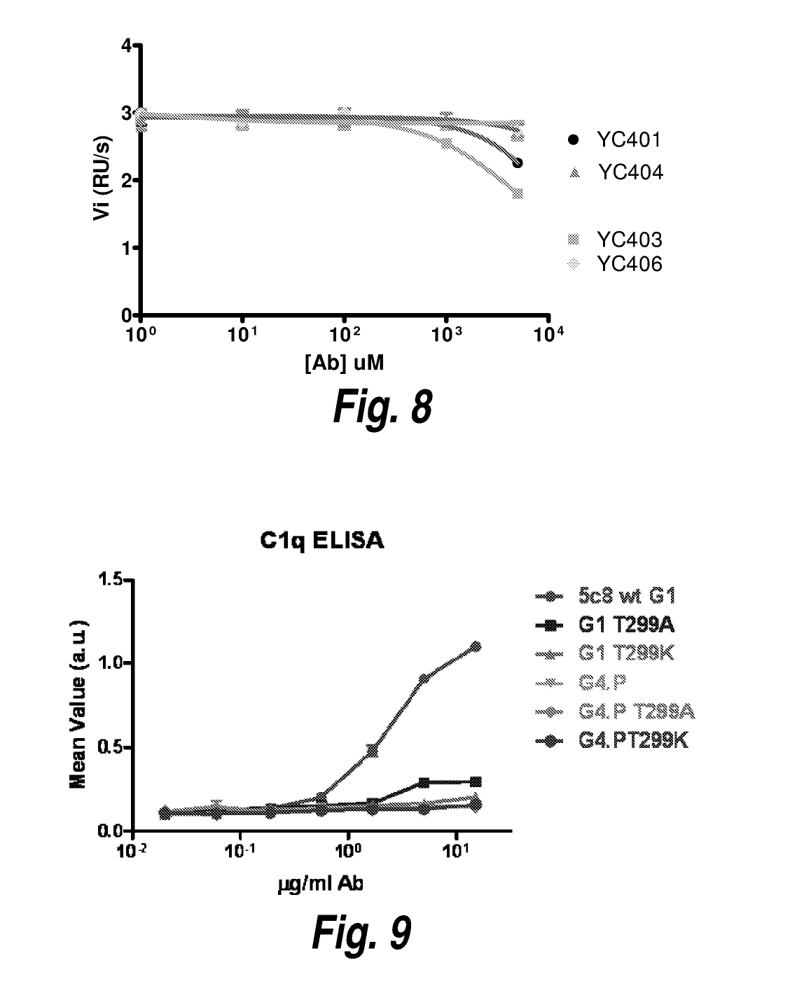

For the CD16 assay, the IgG1 control had an IC₅₀ of 105 μM, while the aglycosylated IgG4.P T299A and IgG1 T299A both had IC₅₀'s>1000 μM. The glycosylated stability engineered IgG4.P molecules had IC₅₀ values at the log equivalent to the IgG1 control, and all of the stability engineered aglycosylated molecules (both IgG4.P and IgG1) had IC₅₀'s>1000 μM. To investigate whether T299K further reduced affinity to CD16, two sets of constructs with the T299K substitution as the only difference (YC401, YC404 and YC403, YC406) were tested at high concentrations of antibody (5 μM). The binding curves show a reduction in the affinity to CD16 caused by the T299K mutation at the high concentration (FIG. 8). In summary, the T299K mutation reduces the affinity for CD16 in IgG molecules.

### B. C1q Binding ELISA

The C1q binding assay was be performed by coating 96 well Maxisorb ELISA plates (Nalge-Nunc Rochester, N.Y., USA) with 50 μl recombinant soluble human CD40 ligand at 10 ug/ml overnight at 4° C. in PBS. The wells were aspirated and washed three times with wash buffer (PBS, 0.05% Tween 20) and blocked for 1 hour with 200 μl/well of block/diluent buffer (0.1 M Na2HPO4, pH 7.0, 1 M NaCl, 0.05% Tween 20, 1% gelatin). The antibodies were diluted in block/diluent buffer with 3-fold dilutions and incubated for 2 hours at room temperature. After aspirating and washing as above, 50 μl/well of 2 J. gel of Sigma human C1q (C0660) diluted in block/diluent buffer were added and incubated for 1.5 h at room temperature.

After aspirating and washing as above, 50 J. well of sheep anti C1q (Serotec AHP033), diluted 3, 560-fold in block/diluent buffer, were added. After incubation for 1 h at room temperature, the wells were aspirated and washed as above. 50 pll/well of donkey anti-sheep IgG HRP conjugate (Jackson ImmunoResearch 713-035-147) diluted to 1:10,000 in block/diluent were then added, and the wells incubated for 1 h at room temperature.

After aspirating and washing as above, 100 all TMB substrate (420 μM TMB, 0.004% H₂O₂ in 0.1 M sodium acetate/citric acid buffer, pH 4.9) were added and incubated for 2 min before the reaction was stopped with 100 ul 2 N sulfuric acid. The absorbance was read at 450 nm with a Softmax PRO instrument, and Softmax software was used to determine the relative binding affinity (C value) with a 4-parameter fit.

The results of the experiment show both CN578 (IgG1 T299K) and YC406 (aglycosylated IgG4.P T299K, T307P, L309K, and D399S) have no measurable binding of C1q (FIG. 9) while IgG1 T299A has some residual binding.

### Example 8

### IgG1 CH3 Stabilizes Aglycolsylated IgG4 CH2 with No Effector Function

The proteins described in section derive from the 5c8 antibody and, unless indicated otherwise, comprise a CH1 region from IgG4, a CH2 domain from IgG4 and a CH3 domain from an IgG1 or IgG4 antibody (as indicated). Protein was produced and purified as described in Example 3. The thermostability of the CH2 and CH3 domains of the modified antibodies were measured by DSC at pH 6.0 and pH 4.5 (detailed in Example 4). The effect of agitation stress was measured by analytical SEC and by turbidity measurements at A320 nm (Example 5). The effector function of the aglycosylated variant antibodies of the invention were characterized by their ability to bind Fc receptors or a complement molecule such as C1q. Binding to Fcγ receptors was analyzed using solution affinity surface plasmon resonance and binding to complement factor C1q was analyzed by ELISA (Example 6). Finally, the serum half-life was determined by pharmacokinetic studies conducted in Sprague-Dawley rats (Example 7).

The IgG4-CH2/IgG1-CH3 aglycosylated constructs were expressed in CHO as detailed in Example 3, with yields ranging from 7 to 14 mg per 1 liter culture. The introduction of the IgG1-CH3 seems to impart a greater yield (−1.5×) compared to the same construct with the CH3 from IgG4 (Table 8.1). In addition, the IgG1aglycosyl IgG4-CH2/IgG1-CH3 had increased thermal stability in the CH3 domain to (Tₘ=85° C. from) compared to the stability of the CH3 domain of the wild-type aglycosyl IgG4 (Tₘ=74° C., Table 8.2 and 4.2). An interesting observation is that the IgG1 CH3 is the determining feature in agitation stability (Table 8.3) because it had been previously thought that the lost of the glycans in the CH2 domain would be the dominating factors in stability.

It is observed that the EAG2412 construct (N297Q IgG4-CH2/IgG1-, i.e., 5c8 variable region (IgG1 framework), IgG4 CH1, IgG4 CH2, IgG1 CH3 with N297Q and Ser228Pro substitutions) shows a better effector function profile, with the lowest binding for CD64 and CD32, compared to the T299A and T299K IgG4-CH2/IgG1-CH3. The the IgG1-CH3 was found to have no effect on the binding to the Fc y receptors. All of the aglycosyl IgG4-CH2 domain-containing constructs do not bind to C1q.

Pharmacokinetic studies were conducted in Sprague-Dawley rats to address the stability and serum half-life of the stability engineered IgG4/IgG1 molecules. Rats were maintained in accordance with the Biogen Idec Institutional Animal Care and Use Committee, and city, state, and federal guidelines for the humane treatment and care of laboratory animals. A single bolus injection of 1 mg/kg (1 mg/ml) of the antibody diluted in phosphate-buffered saline (PBS) was administered by IV into male Sprague-Dawley rats. Rats were sacrificed at 0, 0.25, 0.5, 1, 2, 6, 24, 48, 96, 168, 216, 264, and 336 hours post-injection. Serum samples were prepared for analysis to quantify levels of the antibody. The samples were diluted in DAB supplemented with 5% normal mouse serum (Jackson ImmunoResearch 015-000-120), and the detection reagent was an Eu-labeled mouse anti-Human Fc antibody (Perkin Elmer 1244-330) used at a final concentration of 250 ng/ml. Quantitation was performed by using Excel's TREND function in comparison to a standard curve of purified antibody.

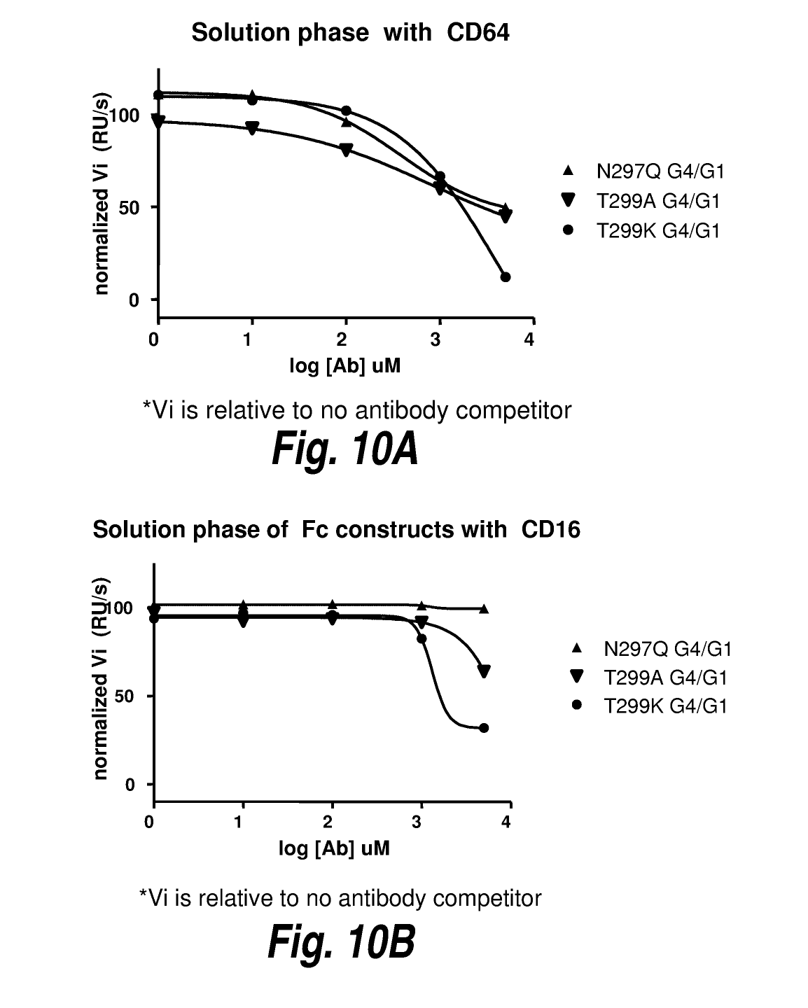

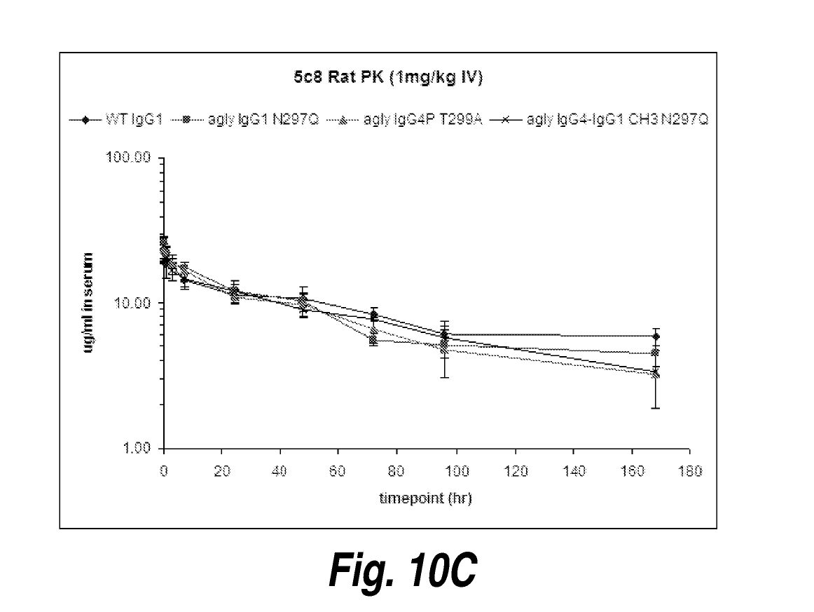

N297Q IgG4-CH2/IgG1-CH3 had the same half-life as the T299A IgG4 antibody which, as expected, was slightly shorter than the aglycolsylated IgG1 (Table 8.5). The data is plotted in FIG. 10.C.

TABLE 8.1
Protein yield from 1 L culture and % Monomer as measured by
Analytical Size-Exclusion Chromatography

| | Final AA Substitution | yield (mg) | % monomer |
|---|---|---|---|
| pEAG2296 | S228P/T299A/IgG1-CH3 | 7.24 | 98% |
| pEAG2287 | S228P/T299K/IgG1-CH3 | 14.2 | 100% |
| EAG2412 | S228P/N297Q/IgG1-CH3 | 13.75 | 99.30% |
| YC407 | S228P, T299A | 4.07 | 96.90% |
| CN579 | S228P, T299K | 11.55 | 90% |
| EAG2391 | N297Q | 7.8 | 100% |

TABLE 8.2
Melting Temperatures of IgG4-CH2/IgG1-CH3 constructs as measured by DSC

| | | pH 6.0 | | | pH 4.5 | | | |
|---|---|---|---|---|---|---|---|---|
| | Final AA Substitution | CH2 | CH3 | Fab | CH2 | CH3 | Fab | Source |
| EAG2296 | S228P/T299A/IgG1-CH3 | 54.6 | 85.2 | 76.4 | 35.1 | 77.5 | 68.1 | CHO |
| EAG2287 | S228P/T299K/IgG1-CH3 | 60 | 85.2 | 76.4 | 41.4 | 77.4 | 68.1 | CHO |
| EAG2412 | S228P/N297Q/IgG1-CH3 | 53 | 85 | 76 | 35 | 78 | 68 | CHO |

TABLE 8.3
Turbidity and % Monomer of Constructs at Time Points During Agitation

| | | Turbidity | | | | % Monomer | | | |
|---|---|---|---|---|---|---|---|---|---|
| | Time | 0 hr | 6 hr | 24 hr | 48 hr | 0 hr | 6 hr | 24 hr | 48 hr |
| EAG2296 | S228P/T299A/IgG1-CH3 | 0 | 0.007 | 0.16 | 0.12 | 100 | 100 | 96.2 | 95.3 |
| EAG2287 | S228P/T299K/IgG1-CH3 | 0 | 0.005 | 0.077 | 0.045 | 100 | 100 | 100 | 95.7 |
| EAG2412 | S228P/N291Q/IgG1-CH3 | 0 | 0.006 | 0.18 | 0.14 | 100 | 100 | 97.3 | 95.2 |

TABLE 8.4
FcyR affinity characterization of IgG4/IgG1 variants (NB indicates no
binding)

IC₅₀ (uM)

| | CD64 | CD32 | CD16 |
|---|---|---|---|
| EAG2296 | >5000 | >7000 | 1324 |
| EAG2287 | 4040 | >7000 | NB^{a} |
| EAG2412 | >5000 | NB^{a} | >5000 |

^{a}No binding observed

TABLE 8.5
Pharmacokinetics of Stability Engineered Constructs in Rats
Pharmacokinetics of Stability Engineered Constructs in Male
Sprague-Dawley Rats after a Single IV Bolus Injection of 1 mg/kg

| | | C_{0, extraplated} | t_{1/2} | AUC | CL | Vss |
|---|---|---|---|---|---|---|
| Compound_ID | Animal_Info | μg/mL | Hr | Hr*mg/L | mL/hr/kg | mL/kg |
| IgG1 | Rat #1 | 26 | 149 | 2,900 | 0.34 | 73 |
| | Rat #2 | 18 | 143 | 2,425 | 0.41 | 84 |
| | Rat #3 | 25 | 83 | 1,918 | 0.52 | 63 |
| | N | 3 | 3 | 3 | 3 | 3 |
| | Mean | 23 | 125 | 2,414 | 0.43 | 73 |
| | SE | 2 | 21 | 284 | 0.05 | 6 |
| | CV % | 18 | 29 | 20 | 21 | 15 |
| N297Q IgG1 | Rat #4 | 24 | 134 | 1,919 | 0.52 | 86 |
| | Rat #5 | 22 | 128 | 2,360 | 0.42 | 76 |
| | Rat #6 | 30 | 66 | 1,557 | 0.64 | 60 |
| | N | 3 | 3 | 3 | 3 | 3 |
| | Mean | 25 | 109 | 1,945 | 0.53 | 74 |
| | SE | 2 | 22 | 232 | 0.06 | 7 |
| | CV % | 15 | 35 | 21 | 21 | 18 |
| T299A | Rat #7 | 26 | 78 | 1,709 | 0.59 | 64 |
| IgG4.P | Rat #8 | 20 | 49 | 1,046 | 0.96 | 66 |
| | Rat #9 | 26 | 98 | 1,964 | 0.51 | 69 |
| | N | 3 | 3 | 3 | 3 | 3 |
| | Mean | 24 | 75 | 1,573 | 0.68 | 66 |
| | SE | 2 | 14 | 273 | 0.14 | 1 |
| | CV % | 15 | 33 | 30 | 35 | 3 |
| N297Q | Rat #10 | 21 | 87 | 1,802 | 0.55 | 70 |
| IgG4.P | Rat #11 | 25 | 75 | 1,574 | 0.64 | 67 |
| CH2/IgG1- | Rat #12 | 29 | 70 | 1,552 | 0.64 | 65 |
| CH3 | N | 3 | 3 | 3 | 3 | 3 |
| | Mean | 25 | 78 | 1,643 | 0.61 | 67 |
| | SE | 2 | 5 | 80 | 0.03 | 1 |
| | CV % | 16 | 11 | 8 | 8 | 4 |

### Example 9

### T299 is a Determinant of Stability and Effector Function

The proteins described in this section are all derived from the 5c8 antibody and, unless indicated otherwise, comprise a CH1, CH2 and CH3 domain of an IgG1 antibody. Protein was produced and purified as described in Example 3. The effects of the mutations on the melting temperatures of the CH2 and CH3 domains were measured by DSC at pH 6.0 and pH 4.5 (detailed in Example 4). The effector function of the aglycosylated variant antibodies of the invention was characterized by their ability to bind Fc receptors or a complement molecule such as C1q. Binding to Fcγ receptors was analyzed using solution affinity surface plasmon resonance and binding to complement factor C1q was analyzed by ELISA (Example 6).

The IgG1 T299X and N297X/T299K aglycosylated constructs were expressed in CHO as detailed in Example 3, with yields ranging from 7 to 30 mg per 1 liter culture (Table 9.1). The addition of secondary mutations at position N297 in combination with T299K did decrease the thermal stability of the CH2 domain by 1.5 to 4.4° C. (Table 9.2). In addition, the T299X mutations showed the greatest gain in stability from the positively charged side chains of Arg (T299R) and Lys (T299K) (Table 9.2). The two polar side chains, Asn (T299N) and Gln (T299Q), both showed a greater stability compared to T299A but not as great as the positively charged side chains. Proline (T299P) showed a small decrease in stability compared to T299A and the larger hydrophobic side chain Phe (T299F) decreased the thermal stability of the CH2 domain by 2.4° C. Finally, the negatively charged side chain Glu (T299E) had very little effect on the CH2 thermal stability. These results demonstrate the novel properties of substituting a positively charged side chain at position T299 to increase thermal stability in the CH2 domain.

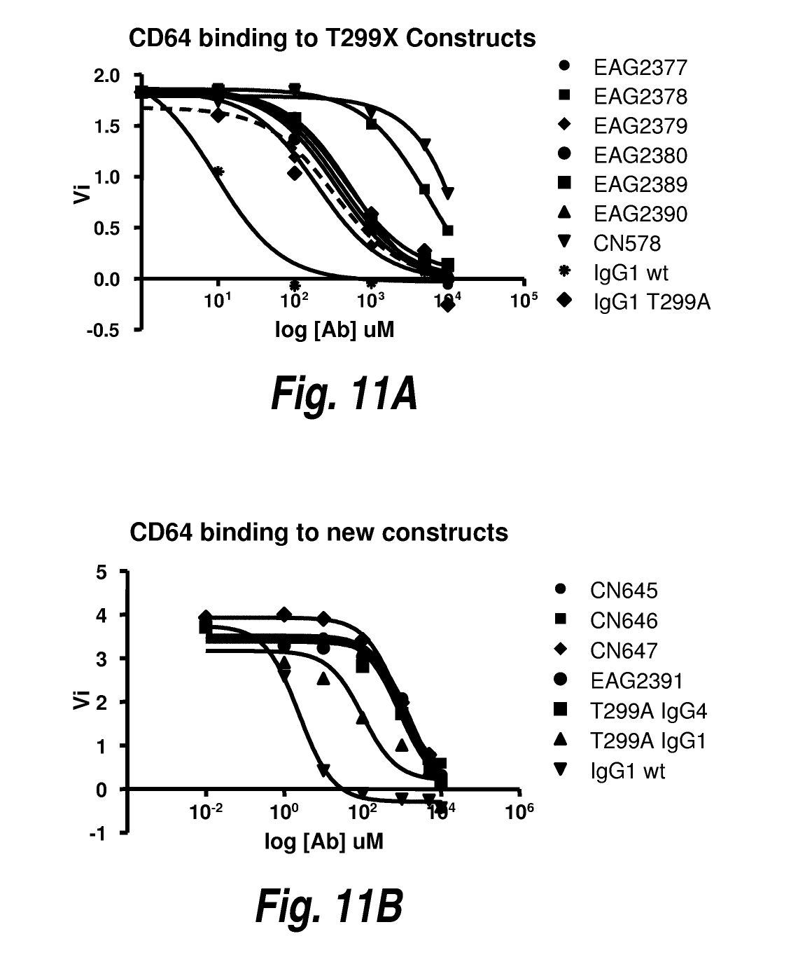

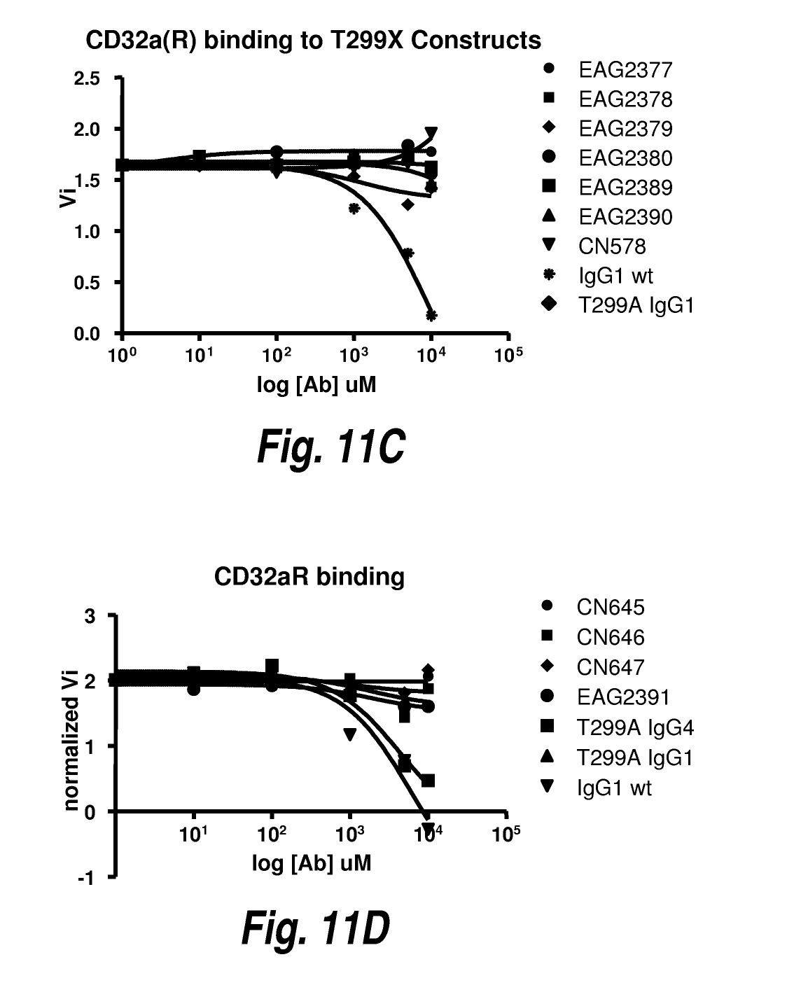

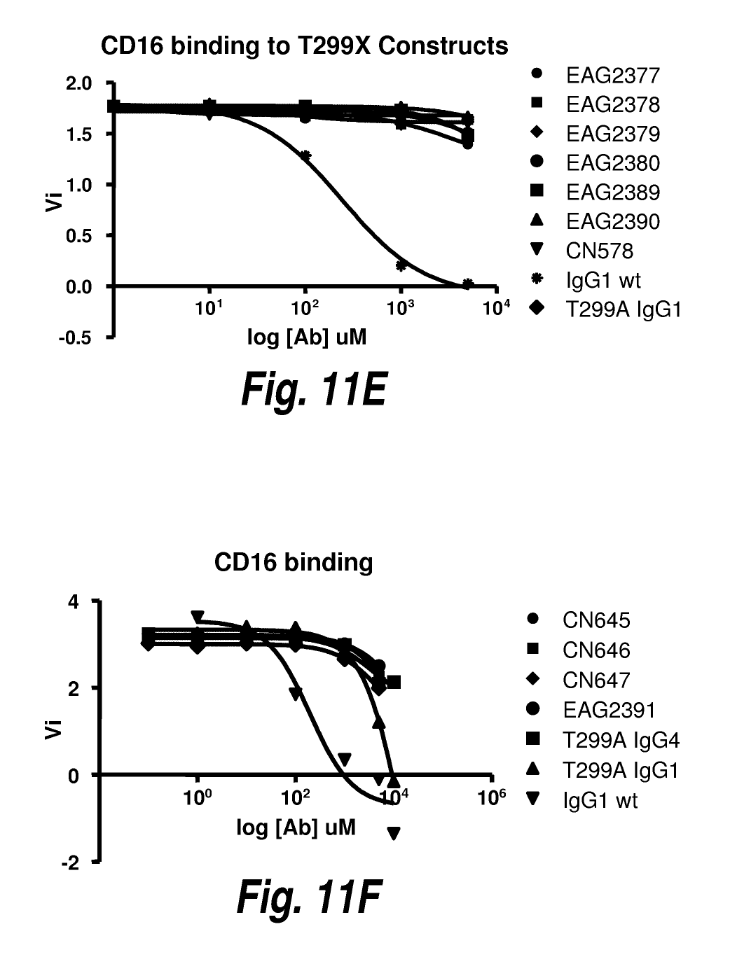

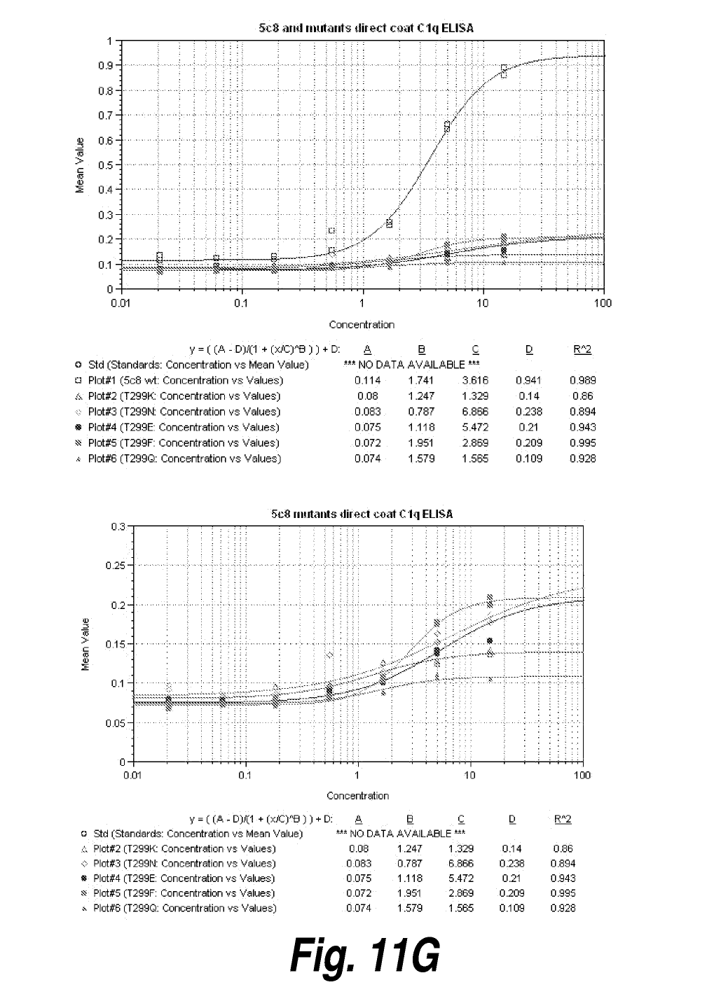

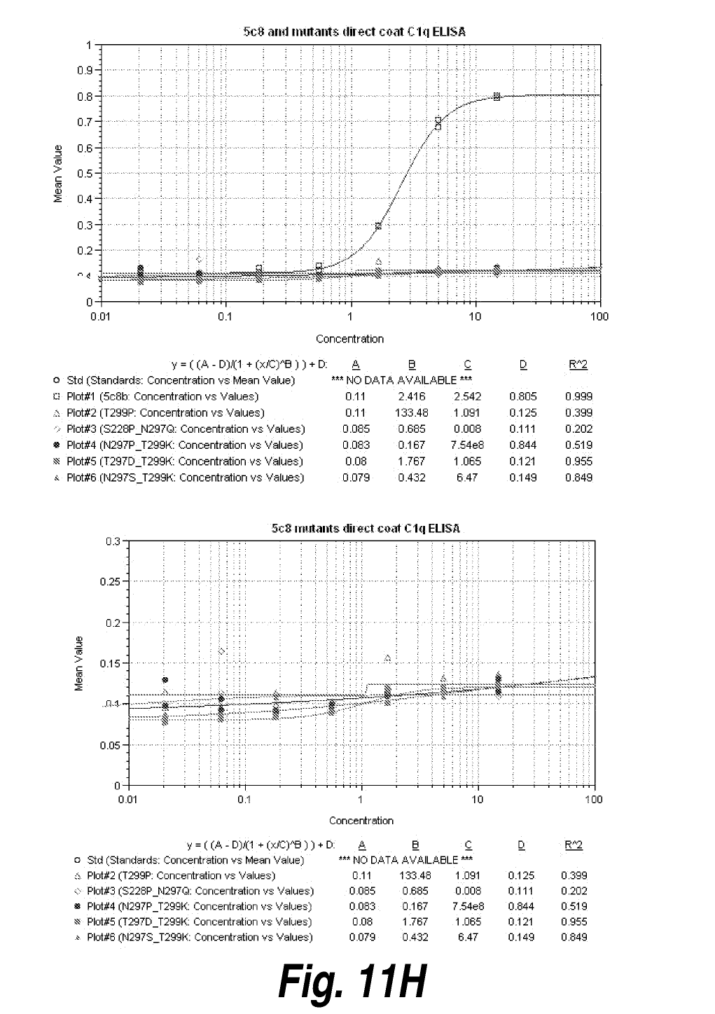

It is observed that the N297X, T299K mutations (CN645, CN646, and CN647) all slightly increased the affinity for CD64 while maintaining the very low affinity for CD32a and CD16 (FIGS. 11.B, 11.D and 11.F). The T299X mutations showed a consistently low affinity to CD16, however, the low affinity for CD32a was increased in the case of T299E (Table 9.3 and FIGS. 11.C, 9.E). It is also interesting to note, that only the positively charged side chains T299R and T299K impart low affinities for CD64 (Table 9.3 and FIG. 11.A). Finally, T299K, T299P and T299Q are dead to trace C1q binding; T299N, T299E, T299F show slightly elevated but still very low binding to C1q (FIGS. 11.G and 11.H). N297P/T299K, N297D/T299K, and N297S/T299K show no binding to C1q (FIG. 11.H).

TABLE 9.1
Protein yield from 1 L culture and % Monomer as measured by
Analytical Size-Exclusion Chromatography

| | Final | | |
|---|---|---|---|
| | AA Substitution | yield (mg) | % monomer |
| CN647 | N297D, T299K | 7.4 | 100% |
| CN646 | N297S, T299K | 30.47 | 98.50% |
| CN645 | N297P, T299K | 9.3 | 100% |
| EAG2389 | T299Q | 12.6 | 100% |
| EAG2390 | T299P | 22.3 | 100% |
| EAG2377 | T299N | 8.7 | 100% |
| EAG2378 | T299R | 14.1 | 100.00% |
| EAG2379 | T299E | 10.1 | 100% |
| EAG2380 | T299F | 12.6 | 100% |

TABLE 9.2
Melting Temperatures of T299X constructs as measured by DSC

| | | pH 6.0 | | | pH 4.5 | | | |
|---|---|---|---|---|---|---|---|---|
| | Final AA Substitution | CH2 | CH3 | Fab | CH2 | CH3 | Fab | Source |
| | IgG1 agly (T299A) | 58.8 | 85.3 | 77.2 | 45.1 | 77 | 68.4 | CHO |
| | IgG1 wt | 71.5 | 84.9 | 77.48 | 60 | 75.5 | 69 | CHO |
| CN578 | T299K | 65.4 | 85.22 | 77.7 | 47.6 | 72.22 | 67.8 | CHO |
| CN647 | N297D, T299K | 63.9 | 85.2 | 77.5 | 49.3 | 74 | 69.5 | CHO |
| CN646 | N297S, T299K | 61 | 84.3 | 77.5 | 44.5 | 74.2 | 70.1 | CHO |
| CN645 | N297P, T299K | 62.1 | 85.3 | 77.6 | 45.6 | 73.5 | 70 | CHO |
| EAG2389 | T299Q | 61.4 | 85.1 | 76.8 | | | | CHO |
| EAG2390 | T299P | 58.2 | 85 | 76.9 | | | | CHO |
| EAG2377 | T299N | 61.9 | 85 | 76.7 | | | | CHO |
| EAG2378 | T299R | 64.9 | 85.3 | 77.7 | | | | CHO |
| EAG2379 | T299E | 59.4 | 85.1 | 76.8 | | | | CHO |
| EAG2380 | T299F | 56.4 | 85.1 | 77.5 | | | | CHO |

TABLE 9.3
FcyR affinity characterization of T299X variants (NB indicates no
binding)

| | IC₅₀ (uM) | | |
|---|---|---|---|
| | CD64 | CD32a | CD16 |
| CN578 | >6000 | >6000 | 1324 |
| CN645 | 4389 | >6000 | >1000 |
| CN646 | 3165 | >6000 | >1000 |
| CN647 | 4476 | >6000 | >1000 |
| EAG2389 | 455 | >6000 | >1000 |
| EAG2390 | 392 | >6000 | >1000 |
| EAG2377 | 586 | >6000 | >1000 |
| EAG2378 | 5966 | >6000 | >1000 |
| EAG2379 | 196 | 1279 | >1000 |
| EAG2380 | 345 | >6000 | >1000 |

### Example 10

### Stabilized Fc Constructs Show the Application of Stability Mutants are Independent of Fab

The proteins described in this section comprise binding sites derived from the 5c8 antibody. The EAG2476 construct comprises Fc moieties from an IgG4 immunoglobulin molecule and EAG2478 comprises Fc moieties from an IgG1 molecule (EAG2476 and EAG2478 are the Fc versions (no Fab) of YC406 and CN578 constructs, respectively).

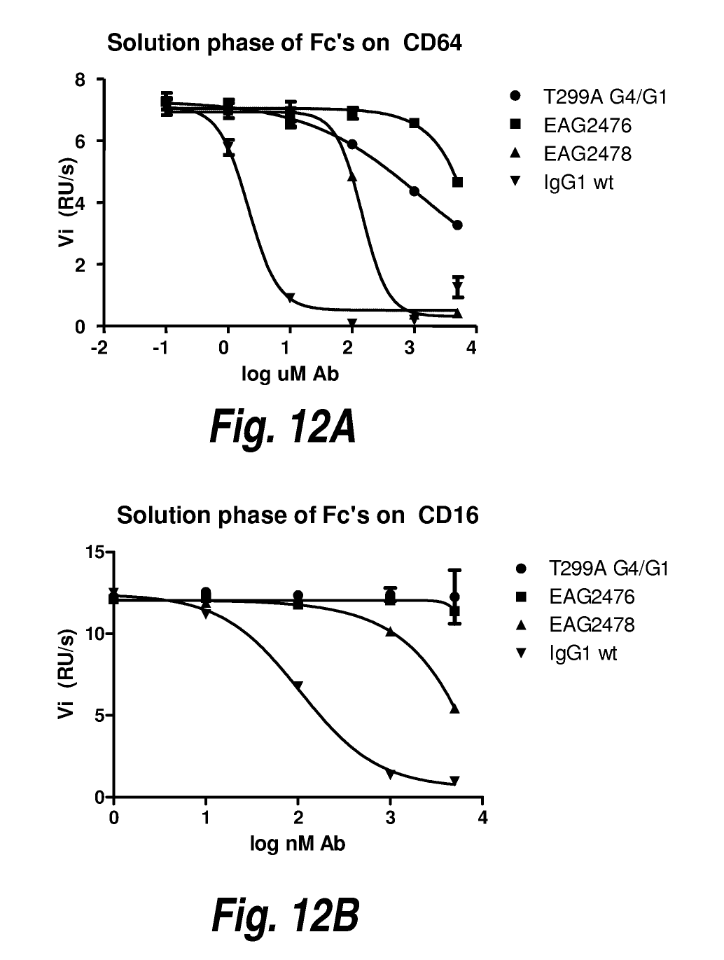

Protein was produced and purified as described in Example 3. The effects of the mutations on the melting temperatures of the CH2 and CH3 domains were measured by DSC at pH 6.0 (detailed in Example 4). The effector function of the aglycosylated variant antibodies of the invention are shown in FIG. 12. The antibodies were characterized by their ability to bind Fc receptors. Binding to Fcγ receptors was analyzed using solution affinity surface plasmon resonance (Example 6).

The stabilized Fc aglycosylated constructs were expressed in CHO as detailed in Example 3, with yields detailed in (Table 10.1). The mutations in the CH2 domain (T299K, T307P and L309K) showed the same thermal stability in the presence or absence of the Fab (Table 10.2) as well as having the same Fc y receptor binding affinities (Table 10.3). Taken together, the stabilizing mutations detailed in this invention are Fab independent as expected and are applicable to stabilizing the Fc domain regardless of the Fab contribution.

TABLE 10.1
Protein yield from 4 L culture and % Monomer as measured by
Analytical Size-Exclusion Chromatography

| | Final | | |
|---|---|---|---|
| | AA Substitution | yield (mg) | % monomer |
| EAG2476 | YC406-Fc | 207.4 | 96.50% |
| EAG2478 | CN578-Fc | 184.8 | 96.30% |

TABLE 10.2
Melting Temperatures of Fc constructs as measured by DSC

| | | pH 6.0 | | |
|---|---|---|---|---|
| | Final AA Substitution | CH2 | CH3 | Fab |
| EAG2476 | YC406-Fc | 65 | 67 | |
| YC406 | S228P, T299K, T307P, D399S, L309K | 66.2 | 74.1 | 77.23 |
| EAG2478 | CN578-Fc | 66 | 84 | |
| CN578 | T299K (IgG1) | 65.4 | 85.22 | 77.7 |

TABLE 10.3
FcyR affinity characterization of T299X variants (NB indicates no
binding)

IC₅₀ (uM)

| | CD64 | CD16 |
|---|---|---|
| EAG2476 | >5000 | 1324 |
| YC406 | >5000 | >1000 |
| EAG2478 | >5000 | ~6000 |
| CN578 | >5000 | >5000 |

### Example 11

### Protein Conformation, Dynamics and Structure of Stabilized Effectorless Antibodies as Determined by Hydrogen/Deuterium Exchange Mass Spectroscopy and X-Ray Crystallography

Structure and dynamics contribute significantly to the function of proteins. Understanding the underlying structural mechanisms is critical to explaining observed functional effects. For this reason, we have examined the effects of the previously detailed gain-in-stability mutations on protein structure and dynamics by both hydrogen/deuterium exchange mass spectroscopy ((H/DX MS) and x-ray crystallography.

### A. Hydrogen/Deuterium Exchange Mass Spectroscopy

Detecting hydrogen/deuterium exchange by mass spectroscopy is an approach for characterizing protein dynamics and conformation. Protein dynamics/conformation affects the rate of exchange of deuterium for hydrogen in proteins, therefore measuring the deuteration of proteins over time can illuminate changes to conformation when a protein structure is modified (such as with mutations). Therefore, we examined the effects of the stabilizing mutations on the hydrogen/deuterium exchange of our aglycosylated antibody Fc backbone.

Antibody (in 50 mM sodium phosphate, 100 mM sodium chloride H2O, pH 6.0) was diluted 20-fold with 50 mM sodium phosphate, 100 mM sodium chloride, D2O, pD 6.0 and incubated at room temperature for various amounts of time (10 s, 1, 10, 60, and 240 min). The exchange reaction was quenched by reducing the pH to 2.6 with a 1:1 dilution with 200 mM sodium phosphate, 0.5 M TCEP and 4 M guanidine HCl, H2O, pH 2.4. Quenched samples were digested, desalted and separated online using a Waters HPLC system based on a nanoACQUITY platform. Approximately 20 pmoles of exchanged and quenched antibody was injected into an immobilized pepsin column. The online digestion was performed over 2 min in water containing 0.05% formic acid at 15° C. at a flow rate of 0.1 mL/min. The resulting peptic peptides were trapped on an ACQUITY HPLC BEH C18 1.7 μm peptide trap (Waters, Milford, Mass.) maintained at 0° C. and desalted with water, 0.05% formic acid. Flow was diverted by a switching valve, and the trapped peptides eluted from the trap at 40 μL/min onto a Waters ACQUITY HPLC BEH C18 1.7 μm, 1 mm×100 mm column held at 0° C. (average back-pressure was approximately 9000 psi). A 6 min linear acetonitrile gradient (8-40%) with 0.05% formic acid was used to separate the peptides. The eluate was directed into a Waters Synapt mass spectrometer with electrospray ionization and lock-mass correction (using Glu-fibrinogen peptide). Mass spectra were acquired over the m/z range 260-1800. Pepsin fragments were identified using a combination of exact mass and MS/MS, aided by Waters IdentityE software. Peptide deuterium levels were determined as described by Weis et al. using the Excel based program HX-Express.

H/DX-MS data for intact IgG4.P versus N297Q IgG4.P, N297Q IgG4.P versus N297Q IgG4.P-CH2/IgG1-CH3 and T299A IgG4.P versus YC406 (T299K, T207P, L309K, D399S) were collected as described above. Comparison of the intact (glycosylated) IgG4 and the aglycosylated N297Q IgG4 shows regions of sequence in which the aglycosylated form shows greater exchange. More H/D exchange is observed in peptides L235-F241, F241-D249, I253-V262, V263-F275, and H310-E318. Higher exchange in IgG4 peptides M358-L365, T411-V422 and A431-S442 compared to the same peptides in the N297Q IgG4.P-CH2/IgG1-CH3 construct shows the gain in stability generated from the IgG1-CH3 in combination with the N297Q IgG4-CH2. In this case, the CH3 domain from IgG4 shows greater exchange in 3 distinct region of the CH3 compared to the IgG1-CH3. Finally, peptides L235-F241, F241-M252, V263-F275, V266-F275, and V282-F296 show the gain in stability by the mutant construct YC406 compared to aglycosylated IgG4 (T299A) in the sequence regions specifically more prone to exchange because of deglycosylation. Interestingly, the D399S mutation in the CH3 domain, while generating a slight increase in thermal stability, imparts greater exchange than the wild type sequence. Overall, H/D exchange MS showed that changes in conformation as a result of deglycosylation were either partially or fully recovered by the stability mutations.

### B. X-Ray Crystallography of Stability Enhanced Fc Constructs

The EAG2476 construct (agly IgG4-Fc T299K, T307P, L309K, D399S) was crystallized and data collected to 2.8 Å resolution (data completeness overall 92%; high resolution shell 66%). The structure was built into the electron density and refined to an R/Rfree of 27.7/33.9% respectively. The structure reveals the two Fc chains in the asymmetric unit (ASU) superimposable with very little deviation between the two chains. Loops V266-E272 and in particular P291-V302 are quite different than that observed in wild type IgG4 crystal structure (pdb 1ADQ). This may be a direct result of the mutation T299K.

The crystal structure of the EAG2478 construct (agly IgG1 Fc T299K) was solved to 2.5 Å resolution (data completeness overall 92%; high resolution shell 66%). The structure was built and refined to an R/Rfree of 27.4/35.8% respectively. Unlike the structure of EAG2476, the two Fc chains in ASU are not identical in the EAG2478 structure. Chain A is observed to be more similar to the structure of an enzymatically deglycosylated IgG1 Fc (pdb 3DNK). The CH2 domains in the EAG2478 structure are closer together than observed in the enzymatically deglycosylated IgG1 Fc (pdb 3DNK) and a murine aglycosylated IgG1 Fc (pdb 3HKF). The CH2 domains are more open in the EAG2476 structure than observed in the EAG2478 structure. The structures reveal that in both cases the T299K mutation is directed towards the Y129 side chain of a docked Fc gamma III receptor, which would explain the decreased affinity for the receptor observed for this mutation.

### EQUIVALENTS

For one skilled in the art, using no more than routine experimentation, there are many equivalents to the specific embodiments of the invention described herein. Such equivalents are intended to be encompassed by the following claims.

1-66. (canceled)

67. A stabilized polypeptide comprising a chimeric Fc region, wherein said stabilized polypeptide comprises
i) at least one CH2 moiety from an IgG antibody of the IgG4 isotype and at least one CH3 moiety from an IgG antibody of the IgG1 isotype, wherein the stabilized polypeptide comprises one or more stabilizing Fc amino acids at one or more amino acid positions selected from the group consisting of 297, 299, 307, 309, 399, 409 and 427 (EU Numbering Convention);
ii) a CH2 moiety from an Fc region of an IgG4 antibody, wherein said stabilized polypeptide comprises one or more stabilizing amino acids at one or more amino acid positions selected from the group consisting of 40F, 262L, 264T, 266F, 297Q, 299A, 299K, 307P, 309K, 309M, 309P, 323F, 399S, and 427F (EU Numbering Convention); or
iii) a CH2 moiety from an Fc region of an IgG1 antibody, wherein said stabilized polypeptide comprises one or more stabilizing amino acids at one or more amino acid positions selected from the group consisting of 299K and 297D (EU Numbering Convention)

68. The stabilized polypeptide of claim 67, which comprises
i) a CH2 moiety from an IgG antibody of the IgG4 isotype and further comprises a hinge, and CH1 moiety from an IgG antibody of the IgG4 isotype and a CH3 domain from an IgG antibody of the IgG1 isotype, and wherein the antibody further comprises a proline at amino acid position 228, EU numbering;
ii) a CH2 moiety from an Fc region of an IgG4 antibody which comprises a Gln at amino acid position 297;
iii) a CH2 moiety from an Fc region of an IgG1 antibody which comprises a Lys at amino acid position 299; or
iv) a CH2 moiety from an Fc region of an IgG1 antibody which comprises a Lys at amino acid position 299 and an Asp at amino acid position 297.

69. The stabilized polypeptide of claim 67, wherein the Fc region is an aglycosylated Fc region.

70. The stabilized polypeptide of claim 67, wherein the melting temperature (Tm) of the stabilized Fc polypeptide is
i) enhanced by about 1° C. or more, about 2° C. or more, about 3° C. or more, about 4° C. or more, about 5° C. or more, about 6° C. or more, about 7° C. or more, about 8° C. or more, about 9° C. or more, about 10° C. or more, about 15° C. or more, and about 20° C. or more;
ii) enhanced at a neutral pH (about 6.5 to about 7.5); or
iii) enhanced at a acidic pH of about 6.5 or less, about 6.0 or less, about 5.5 or less, about 5.0 or less, about 4.5 or less, and about 4.0 or less
relative to a parental polypeptide lacking the stabilizing amino acid

71. The stabilized polypeptide of claim 67, wherein the stabilized polypeptide is expressed at higher yield relative to a parental polypeptide lacking the stabilizing mutation.

72. The stabilized polypeptide of claim 67, wherein the turbidity of the stabilized polypeptide
i) is reduced relative to a parental polypeptide lacking the stabilizing amino acid or
ii) is reduced by a factor selected from the group consisting of about 1-fold or more, about 2-fold or more, about 3-fold or more, about 4-fold or more, about 5-fold or more, about 6-fold or more, about 7-fold or more, about 8-fold or more, about 9-fold or more, about 10-fold or more, about 15-fold or more, about 50-fold or more, and about 100-fold or more.

73. The stabilized polypeptide of claim 67, wherein said stabilized polypeptide has reduced effector function as compared to a parental Fc polypeptide lacking the stabilizing mutation and wherein the reduced effector function is:
i) reduced ADCC activity;
ii) reduced binding to an Fc receptor (FcR) selected from the group consisting of FcγRI, FcγRII, and FcγRIII; or
iii) reduced by a factor selected from the group consisting of about 1-fold or more, about 2-fold or more, about 3-fold or more, about 4-fold or more, about 5-fold or more, about 6-fold or more, about 7-fold or more, about 8-fold or more, about 9-fold or more, about 10-fold or more, about 15-fold or more, about 50-fold or more, and about 100-fold or more.

74. The stabilized polypeptide of claim 67, wherein said stabilized polypeptide has enhanced half-life as compared to a parental Fc polypeptide.

75. The stabilized polypeptide of claim 67, wherein the Fc region is a dimeric Fc region comprising two polypeptide chains.

76. The stabilized polypeptide of claim 67, wherein the Fc region is a single chain Fc region.

77. The stabilized polypeptide of claim 67, wherein all of the Fc moieties of the Fc region are aglycosylated.

78. The stabilized polypeptide of claim 67, wherein the Fc region is aglycosylated owing to
i) a substitution at position 299 of the Fc region (EU numbering convention);
ii) as a result of its production in a bacterial host cell; or
iii) as a result of deglycosylation by chemical or enzymatic means.

79. The stabilized polypeptide of claim 69, wherein the Fc region is aglycosylated and the polypeptide comprises a chimeric hinge domain comprising a substitution with proline residue at amino acid position 228 (EU numbering convention).

80. The stabilized polypeptide of claim 67, wherein the stabilizing amino acid(s) are independently selected from the group consisting of (i) an uncharged amino acid at position 297, ii) a positively charged amino acid at position 299, (iii) a polar amino acid at position 307, (iv) a positively charged or polar amino acid at position 309, (v) a polar amino acid at position 399, (vi) a positively charged or polar amino acid at position 409, and (vii) a polar amino acid at position 427.

81. The stabilized polypeptide of claim 67, wherein at least one stabilizing amino acid is a Gln at amino acid position 297 (EU numbering).

82. The stabilized polypeptide of claim 67, wherein at least one of the stabilizing amino acids is:
i) a lysine (K) or tyrosine (Y) at position 299;
ii) a proline (P) or methionine (M) at position 307;
iii) a proline (P), methionine (M) or lysine (K) at position 309; or
iv) serine (S) at position 399.

83. The stabilized polypeptide of claim 67, wherein the Fc region is operably linked to a binding site.

84. The stabilized polypeptide of claim 83, wherein the binding site is selected from an antigen binding site, a ligand binding portion of a receptor, a receptor binding portion of a ligand, a modified antibody, an scFv, a Fab, a minibody, a diabody, a triabody, a nanobody, a camelid antibody, and a Dab

85. The stabilized polypeptide of claim 83, w comprises a stabilized full length antibody.

86. The stabilized polypeptide of claim 85, wherein the stabilized full length antibody is fused to a conventional or stabilized scFv molecule.

87. The stabilized polypeptide of claim 83, which is a stabilized immunoadhesin.

88. The stabilized polypeptide of claim 83, wherein a binding site is veneered onto the surface of the Fc region of the stabilized polypeptide.

89. The stabilized polypeptide of claim 83, wherein said binding site is derived from a non-immunoglobulin binding molecule.

90. The stabilized polypeptide of claim 89, wherein said non-immunogloublin binding molecule is selected, from the group consisting of an adnectin, an affibody, a DARPin and an anticalin.

91. A composition comprising a stabilized polypeptide of claim 67, and a pharmaceutically acceptable carrier.

92. A nucleic acid molecule comprising a nucleotide sequence encoding a stabilized binding polypeptide of claim 67.

93. A nucleic acid molecule comprising a nucleotide sequence encoding a polypeptide chain of a stabilized binding polypeptide of claim 67.

94. A vector comprising the nucleic acid molecule of claim 92.

95. A vector comprising the nucleic acid molecule of claim 93.

96. A host cell expressing the vector of claim 94.

97. A host cell expressing the vector of claim 95.

98. A method of producing a stabilized Fc polypeptide of the invention comprising culturing the host cell of claim 96 in culture medium such that the stabilized Fc polypeptide is produced.

99. A method of producing a stabilized Fc polypeptide of the invention comprising culturing the host cell of claim 97 in culture medium such that the stabilized Fc polypeptide is produced.

100. A method for stabilizing a parental Fc polypeptide comprising an aglycosylated, chimeric Fc region or portion thereof, the method comprising substituting an elected amino acid in at least one Fc moiety of the Fc region with a stabilizing amino acid to produce a stabilized Fc polypeptide with enhanced stability relative to said starting polypeptide, wherein the substitution is made an amino acid position of the Fc moiety selected from the group consisting of:
i) 297, 299, 307, 309, 399, 409 and 427 (EU Numbering Convention).

101. The method of claim 100, wherein the chimeric Fc region comprises a CH2 domains from an IgG antibody of the IgG4 isotype and a CH3 domain from an IgG antibody of the IgG1 isotype.

102. The method of claim 100, wherein the amino acid position and the amino acid present in the stabilized Fc polypeptide is selected from the group consisting of 297Q, 299A, 299K, 307P, 309K, 309M, 309P, 323F, 399E, 399S, 409K, 409M and 427F.

103. The method of claim 100, wherein the stabilized Fc polypeptide comprises a Gln at position 297 (EU numbering).

104. A method for large scale manufacture of a polypeptide comprising a stabilized Fc region, the method comprising:
(a) genetically fusing at least one stabilized Fc moiety to a polypeptide to form a stabilized fusion protein;
(b) transfecting a mammalian host cell with a nucleic acid molecule encoding the stabilized fusion protein,
(c) culturing the host cell of step (b) in 10 L or more of culture medium under conditions such that the stabilized fusion protein is expressed;
to thereby produce a stabilized fusion protein.

105. The method of claim 104, wherein the stabilized Fc region:
i) is a chimeric Fc region comprising a CH2 domains from an IgG antibody of the IgG4 isotype and a CH3 domain from an IgG antibody of the IgG1 isotype or
ii) comprises a Gln at amino acid position 297 (EU numbering).

106. A method thr treating or preventing a disease or disorder in a subject, comprising administering the composition of claim 91 to a subject suffering from said disease or disorder to thereby treat or prevent a disease or disorder.
